(19) United States
(12) Patent Application Publication
(10) Pub. No.: US 2010/0130731 A1
(43) Pub. Date: May 27, 2010

(54) CRYSTAL STRUCTURE OF P53 MUTANTS AND THEIR USE

(75) Inventors: Alan Fersht, Cambridgeshire (GB); Andreas Joerger, Cambridgeshire (GB)

Correspondence Address: NIXON & VANDERHYE, PC, 901 NORTH GLEBE ROAD, 11TH FLOOR, ARLINGTON, VA, 22203, US

(73) Assignee: MEDICAL RESEARCH COUNCIL, London (GB)

(21) Appl. No.: 12/309,954

(22) PCT Filed: Aug. 9, 2007
(86) PCT No.: PCT/GB2007/003056

§ 371 Date: Feb. 4, 2009

(30) Foreign Application Priority Data
Aug. 10, 2006 (GB) 0615934.7

(51) Int. Cl. C07K 1/04; G01N 33/566; G06F 19/00

(52) U.S. Cl. 530/406; 436/501; 702/19

(57) ABSTRACT

The invention relates to crystals of p53 which have mutations in the β-sandwich region at positions 220, 143 or 270. The structures may be used for computer-based drug design to identify ligands which can bind within the β-sandwich region in order to stabilize the proteins.

### FIELD OF THE INVENTION

The present invention relates to the crystals of variants of the tumour suppressor protein p53, their structures and their use.

### BACKGROUND TO THE INVENTION

The tumour suppressor protein p53 is a 393 amino acid transcription factor that regulates the cell cycle and plays a key role in the prevention of cancer development. In response to cellular stress, such as UV irradiation, hypoxia and DNA damage, p53 induces the transcription of a number of genes that are connected with G1 and G2 cell cycle arrest and apoptosis (1-3). In about 50% of human cancers, p53 is inactivated as result of a mis-sense mutation in the p53 gene (4,5).

The multi-functionality of p53 is reflected in the complexity of its structure. Each chain in the p53 tetramer is composed of several domains. There are well-defined DNA-binding and tetramerization domains and highly mobile, largely unstructured regions (6-11). Most p53 cancer mutations are located in the DNA-binding core domain of the protein (4). This domain has been structurally characterized in complex with its cognate DNA by X-ray crystallography (6) and in its free form in solution by NMR (12). It consists of a central β-sandwich of two anti-parallel β-sheets that serves as basic scaffold for the DNA-binding surface. The DNA-binding surface is composed of two β-turn loops (L2 and L3) that are stabilized by a zinc ion and a loop-sheet-helix motif. Together, these structural elements form an extended DNA-binding surface that is rich in positively charged amino acids and makes specific contacts with the various p53 response elements. The six amino acid residues that are most frequently mutated in human cancer are located in or close to the DNA-binding surface (cf. release R10 of the p53 mutation database at www-p53.1arc.fr)(4). These residues have been classified as ‘contact’ (Arg248, Arg273) or ‘structural’ (Arg175, Gly245, Arg249, Arg282) residues, depending on whether they directly contact DNA or play a role in maintaining the structural integrity of the DNA-binding surface (6).

There is growing evidence that p53, which is only marginally stable at body temperature, has evolved to be highly dynamic and intrinsically unstable (12,22,35), a trait also shared for example also observed for the tumour suppressor protein p16 (36).

Urea denaturation studies have shown that the contact mutation R273H has no effect on the thermodynamic stability of the core domain, whereas structural mutations substantially destabilize the protein, ranging from 1 kcal/mol for G245S and 2 kcal/mol for R249S to up to more than 3 kcal/mol for R282W (13). The destabilization has severe implications for the folding state of these mutants in the cell. Since the wild-type core domain is only marginally stable and has a melting temperature of only slightly above body temperature, the highly destabilized mutants such as R282W are largely unfolded under physiological conditions and, hence, are no longer functional (14).

Because many p53 mutants are unfolded it is not possible to produce protein crystals of these mutants. To overcome this problem, a functional thermostable synthetic variant of p53, referred to as “T-p53C” has been used. This variant has the substitutions M133L, V203A, N239Y and N268D. The variant was used in introduce the cancer hot-spot mutants R273H and R249S and the structures of these two mutants were determined by X-ray crystallography (18). These structural studies established R273H as a pure DNA-contact mutant where a crucial DNA-contact is lost but the overall architecture of the DNA-binding surface is conserved. In contrast, the R249S mutation induces substantial conformational changes in the L3 loop, which is directly involved in DNA binding via Arg248 and forms part of the interface between different core domains in the DNA-bound form. Further, it could be shown that the second-site suppressor mutation H168R rescues the function of R249S in a specific manner by mimicking the structural role of Arg249 in wild type (18).

Cancer-associated mutations are not, however, restricted to the DNA-binding surface but are also found in the β-sandwich region of the protein. The most common mutation outside the DNA-binding surface is Y220C. It is located at the far end of the (3-sandwich at the start of the turn connecting β-strands S7 and S8. The benzene moiety of Tyr220 forms part of the hydrophobic core of the β-sandwich, whereas the hydroxyl group is pointing toward the solvent.

Other mutations away from the DNA-binding surface include the V143A cancer mutation, which is located on β-strand S3 and F270L. The former is the classic example of a temperature-sensitive p53 mutant. At body temperature, the mutant is inactive and unfolded, whereas it retains transactivation activity at lower temperature (15).

Recently, a large number of temperature-sensitive mutants have been identified, by screening a comprehensive missense mutation library (16). Most of the mutations are clustered in the β-sandwich. Qualitative NMR studies have shown that hotspot mutants evince characteristic local structural changes (17).

### DISCLOSURE OF THE INVENTION

The present invention relates to the structure of p53 mutants which have changes to the β-sandwich region outside the DNA-binding surface. Using T-p53C we have found structural changes to particular mutants which result in changes to p53 such that potential binding cavities in the protein are created. These cavities provide targets for stabilization and rescue of p53 mutants.

In one aspect, we have found that the Y220C mutant causes structural changes to p53 which results in the creation of a solvent-accessible crevice at the far end of the β-sandwich domain. The structural changes upon mutation link two rather shallow surface clefts that pre-exist in wild type to form a long extended crevice in T-p53C-Y220C (residues 109, 145-157, 202-204, 219-223, 228-230 and 257). This mutation-induced crevice has its deepest point at the mutation site, Cys220, thus providing a binding pocket for a small molecule drug, particularly one with a moiety that selectively targets mutant Y220C and/or residues of the cavity.

In a further aspect, we have found that two separate mutations—V143A and F270L—to residues which line either side of the hydrophopic core of the β-sandwich region result in the creation of a large hydrophobic cavity. While the cavity in each case does not appear to cause a collapse of the surrounding structure, the creation of the increased void volume causes a loss of stability in the protein reflected by the lower melting point of these mutants. The structures of these mutants thus permits targeted drug discovery to identify molecules which can be used to stabilize the cavities caused by these mutations.

Thus in general aspects, the present invention is concerned with the provision of structures of p53 mutants and their use in modelling the interaction of molecular structures, e.g. potential and existing pharmaceutical compounds, or fragments of such compounds, with this structure.

These and other aspects and embodiments of the present invention are discussed below.

### BRIEF DESCRIPTION OF THE TABLES

Table 1 (FIG. 1) sets out the coordinate data of the structure of T-p53C-Y220C.

Table 2 (FIG. 2) sets out the coordinate data of the structure of T-p53C-V143A.

Table 3 (FIG. 3) sets out the coordinate data of the structure of T-p53C-F270L.

Table 4 sets out the sequences crystallized in the present invention. Residue numbers are indicated with reference to the wild-type human p53 (SWISS PROT P04637). Residues in bold are those which are altered compared to wild-type. As used herein (unless explicitly specified to the contrary) the numbering of p53 residues is by reference to wild-type numbering shown in Table 4, as opposed to the numbering of the sequence listing.

Table 5 sets out data collection and refinement statistics.

Table 6 sets out changes in free energy of urea-induced unfolding of p53 core domain mutants.

Table 7 sets out volumes of mutation-induced internal cavities.

### BRIEF DESCRIPTION OF THE DRAWINGS

FIG. 1 sets out Table 1.

FIG. 2 sets out Table 2.

FIG. 3 sets out Table 3.

FIG. 4 shows a wire frame model of p53 core domain bound to gadd45 consensus DNA (PDB ID code 1TSR, molecule B). Secondary structure elements are highlighted by semi-transparent ribbons and cylinders. The two strands of bound consensus DNA are shown at the top of the model. Side chains of cancer mutation sites that were structurally studied in this work and Joerger et al. 2005 are shown in orange. The dark spheres indicate the location of the mutation sites in the superstable quadruple mutant M133L/V203A/N239Y/N268D (T-p53C). Residues of “hotspot” mutation regions are shown, together with those of the (3-sandwich region at 220, 143 and 270.

FIG. 5 shows a stereo view of the mutation site at the periphery of the β-sandwich in T-p53C-Y220C (molecule A) superimposed onto the structure of T-p53C (PDB ID code 1UOL, molecule A). Several water molecules close to Cys220 in T-p53C-Y220C that fill the cleft created by the mutation are shown as spheres.

FIG. 6A shows a stereo view of the structure of T-p53C-V143A superimposed onto T-p53C (PDB ID code 1UOL, molecule A). All residues in the hydrophobic core of the β-sandwich within a 4.5-Å radius of the Val143 side chain in T-p53C are shown. FIG. 6B is a stereo view of the structure of Tp53C-F270L superimposed on T-p53C (PDB ID code 1UOL, molecule A). All residues within a 6-Å radius of the Phe270 side-chain in T-p53C are shown.

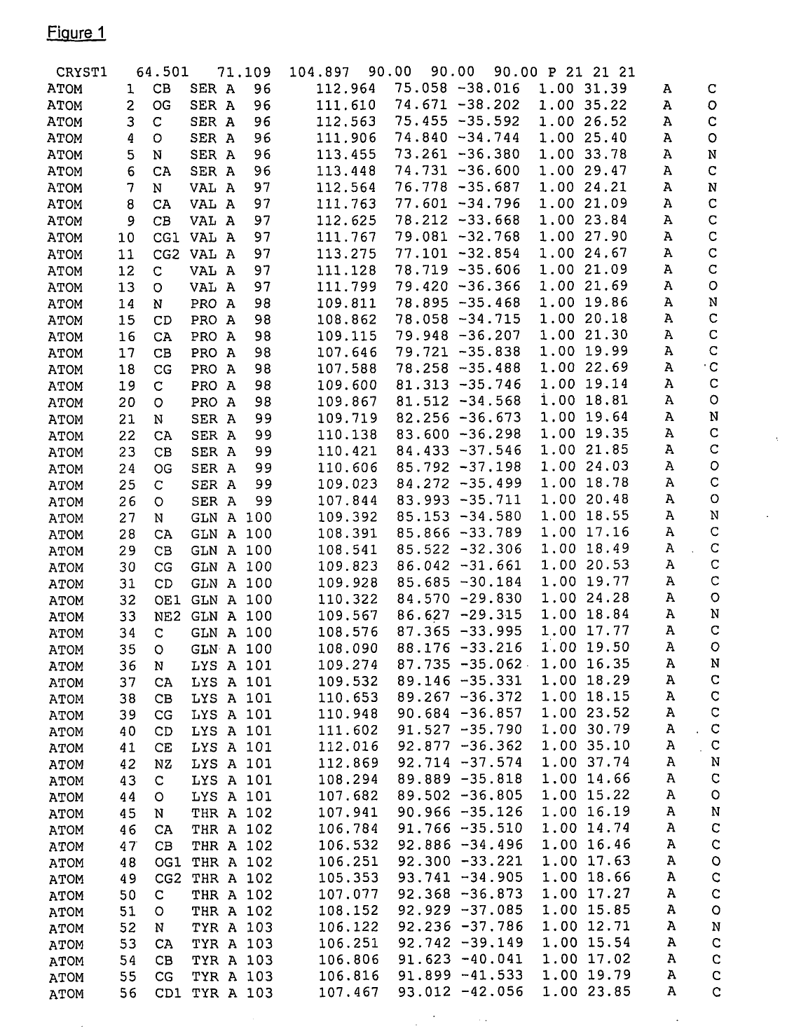

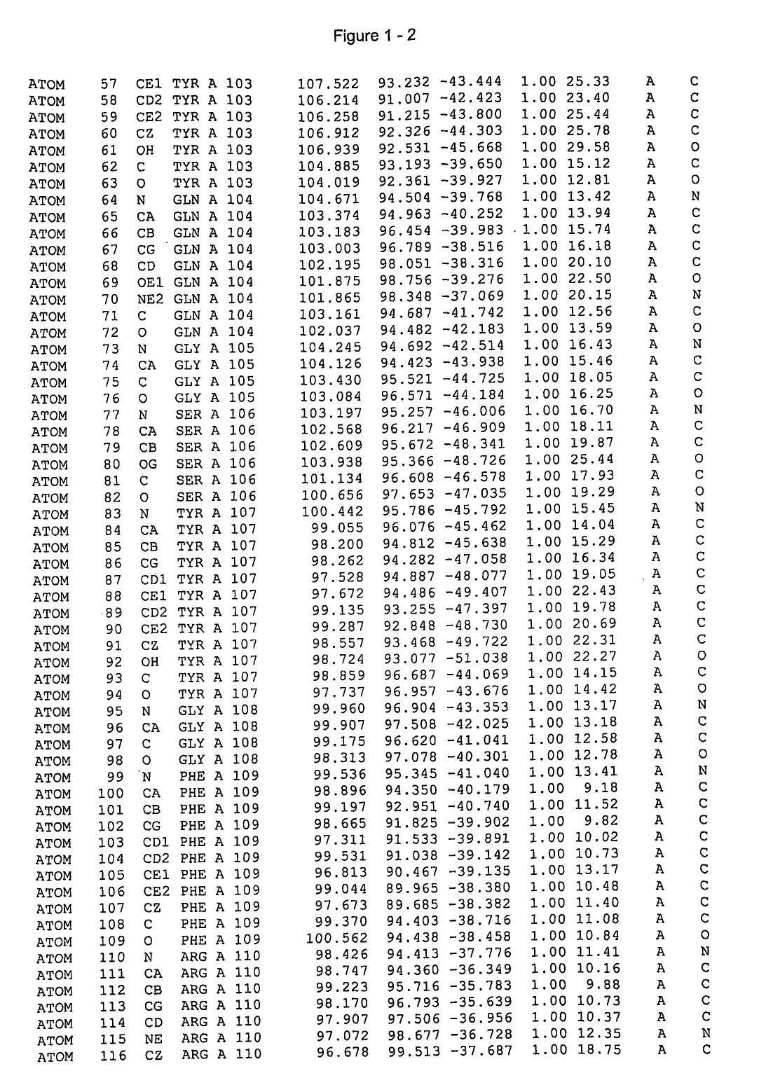

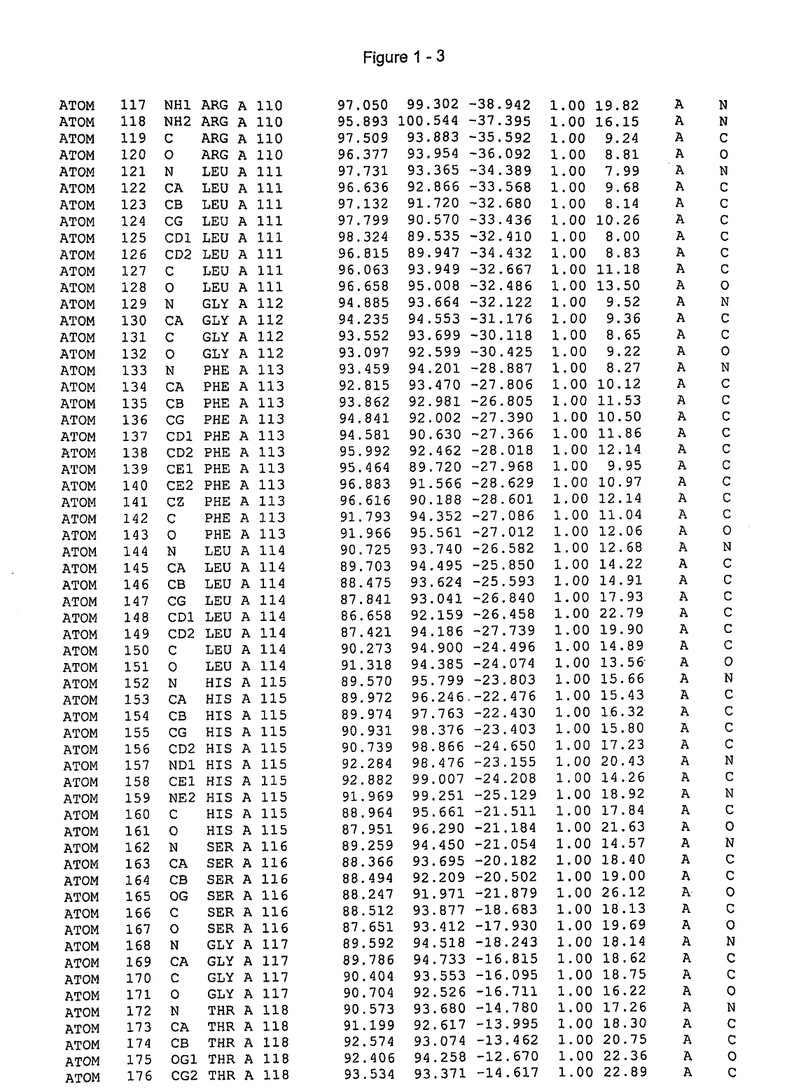

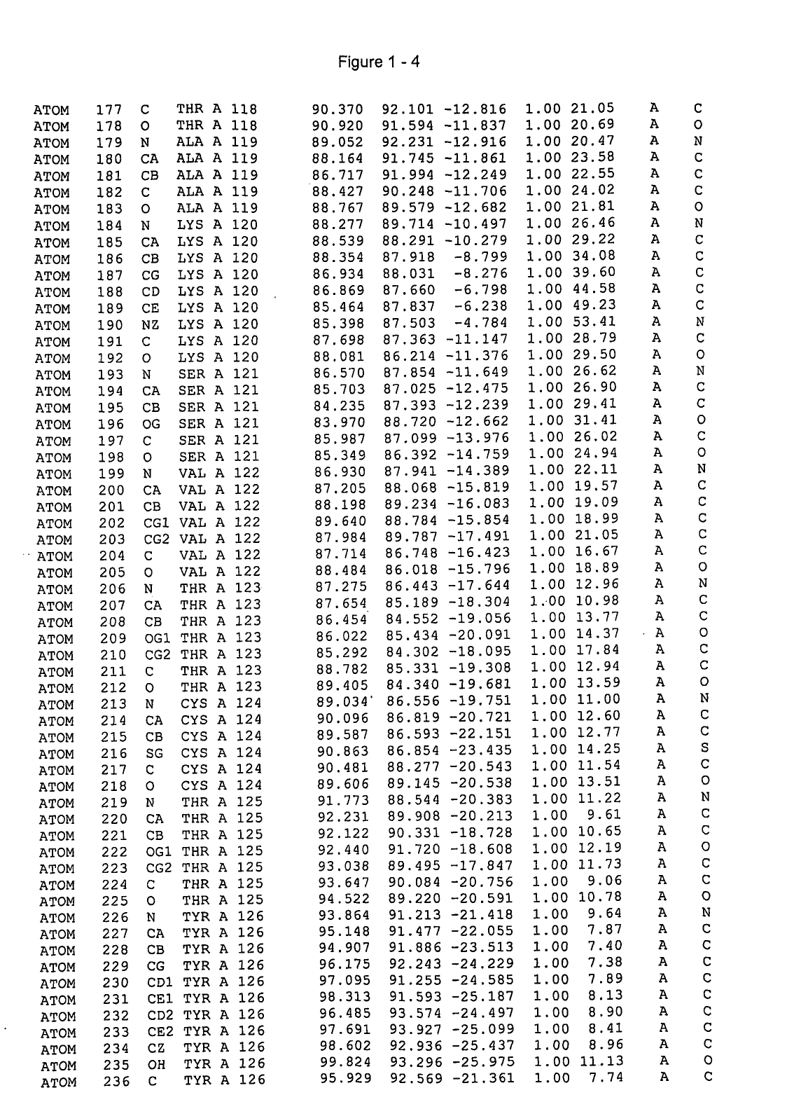

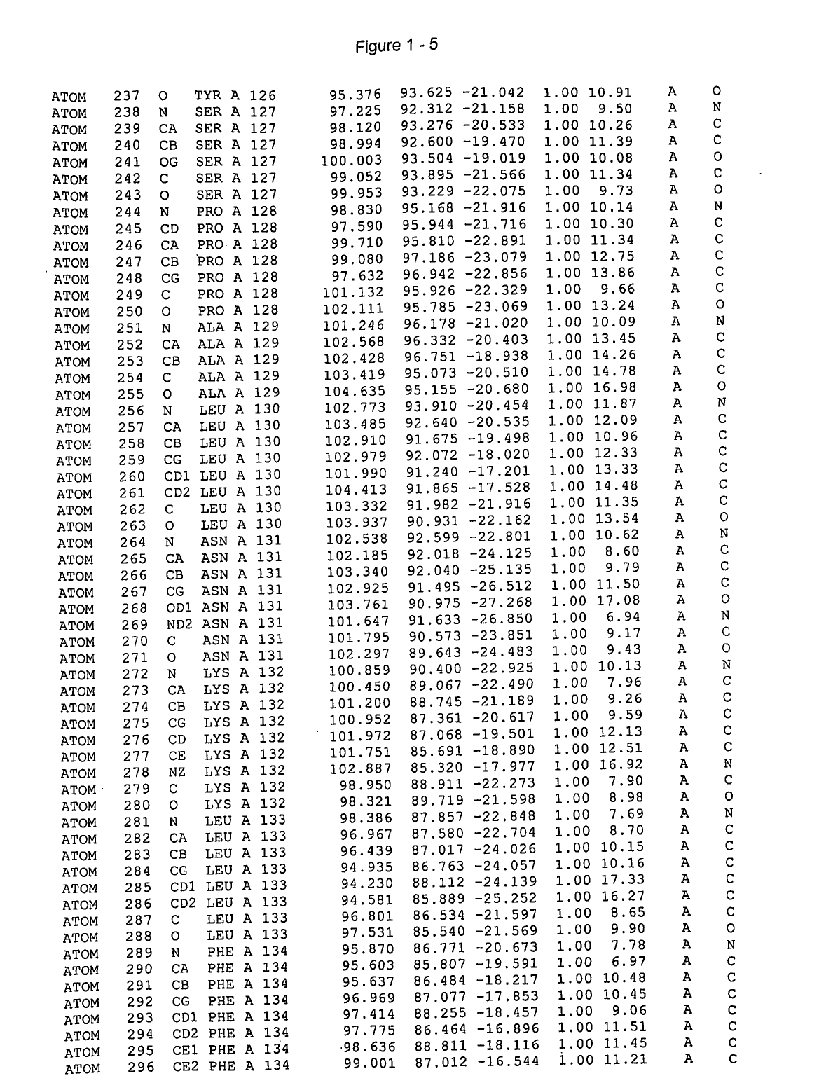

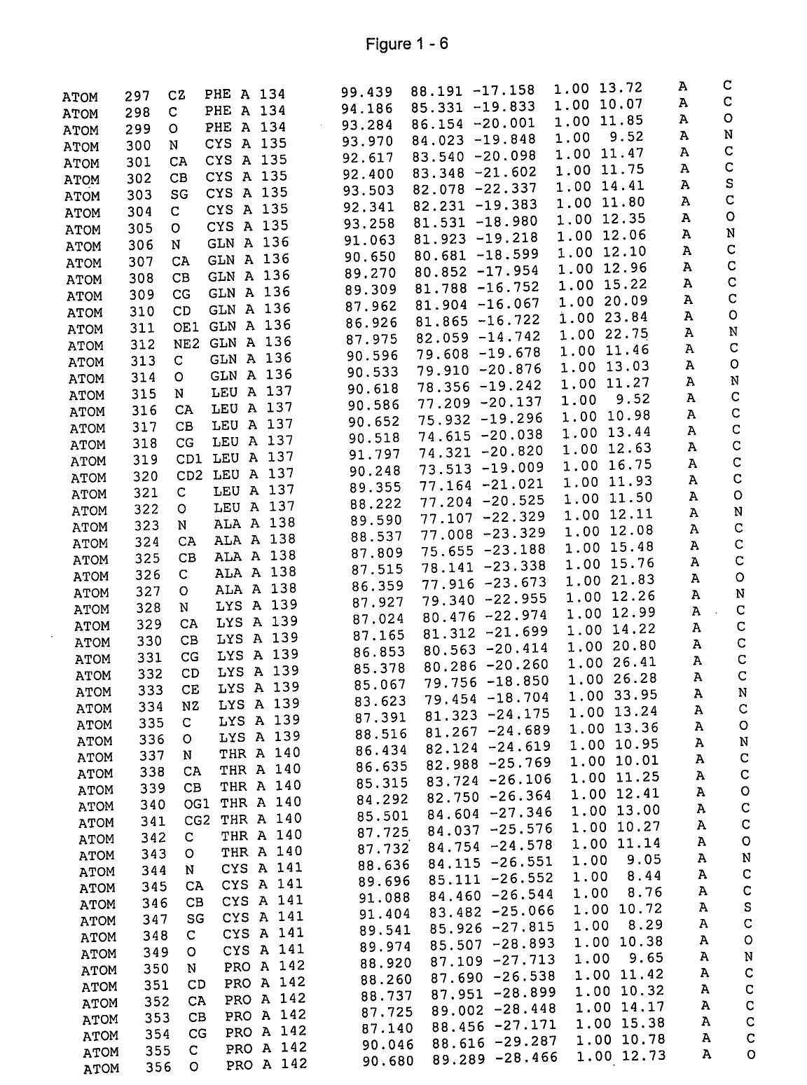

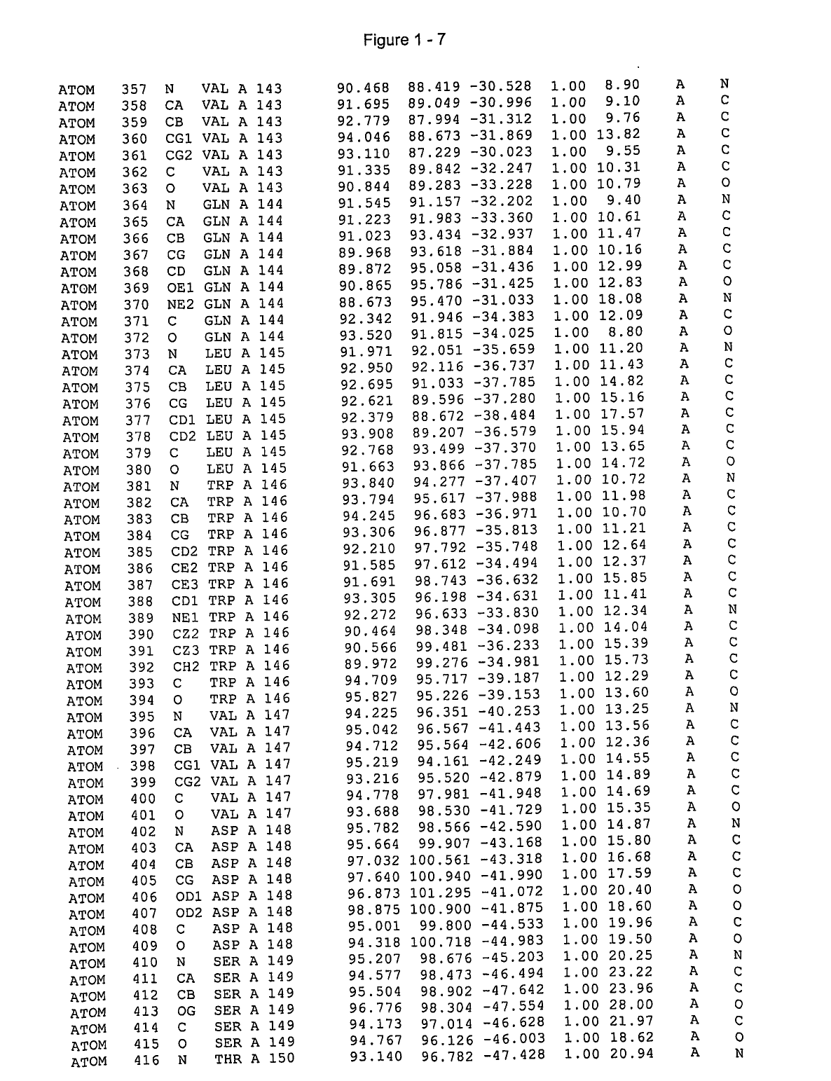

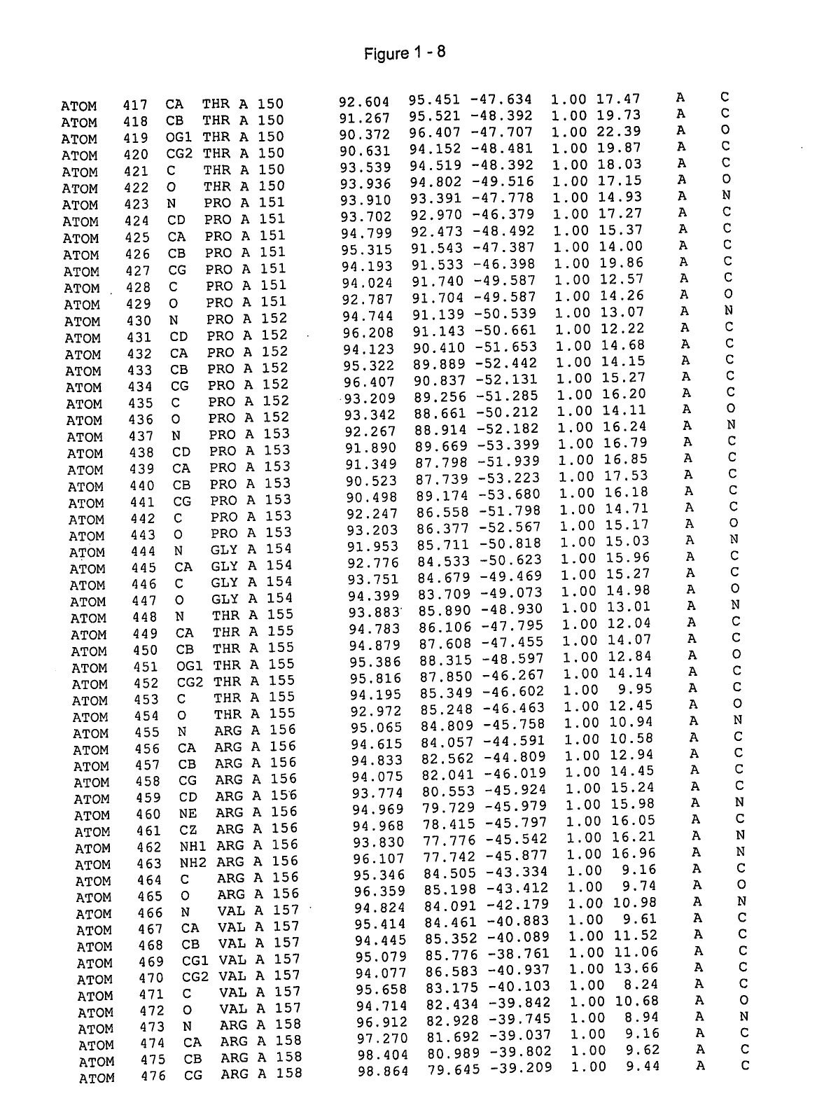

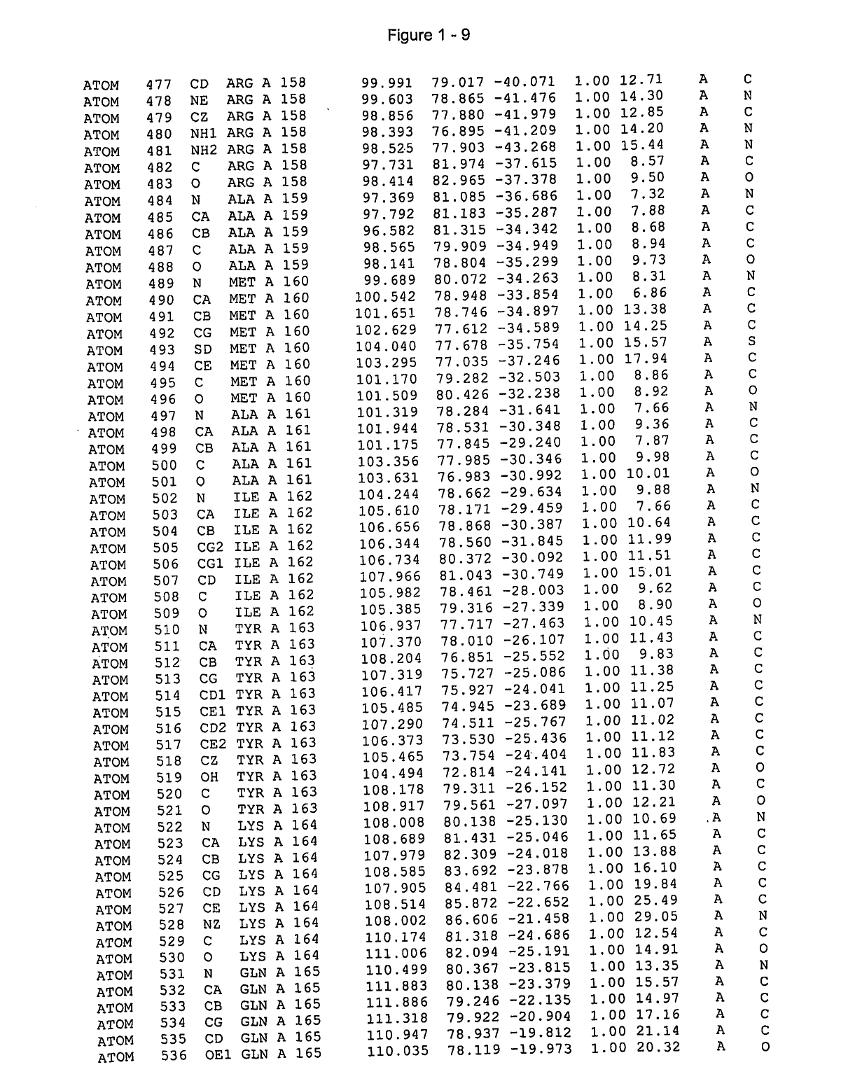

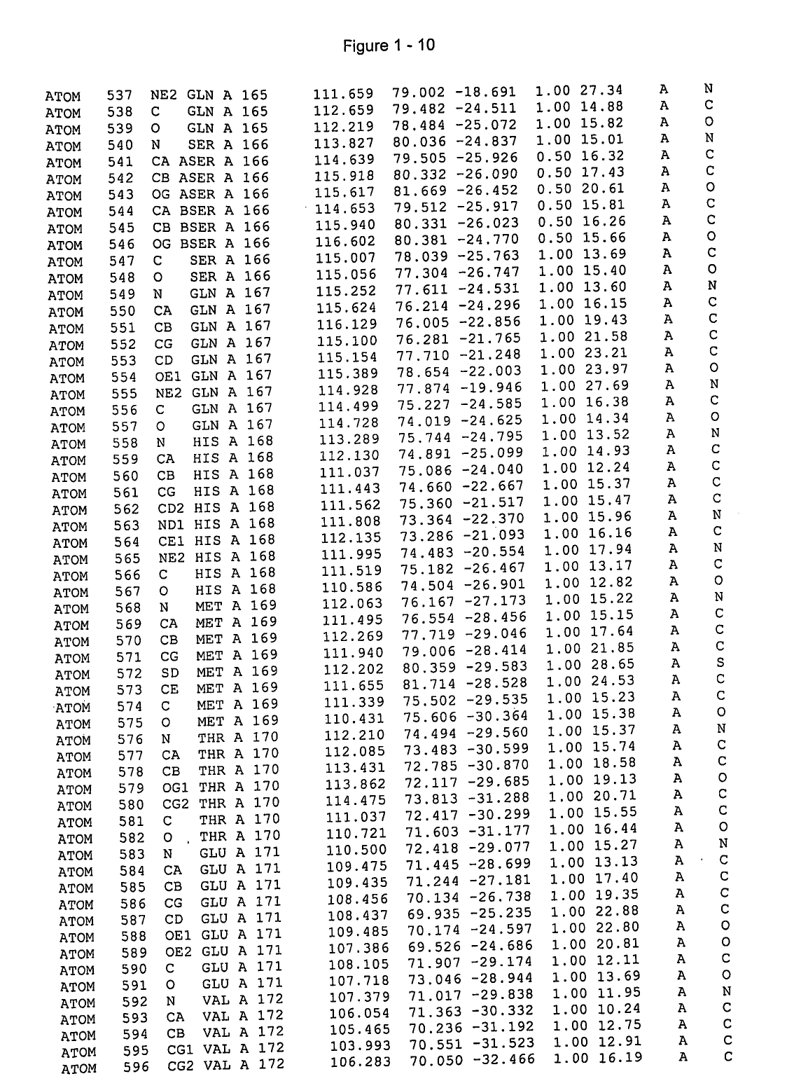

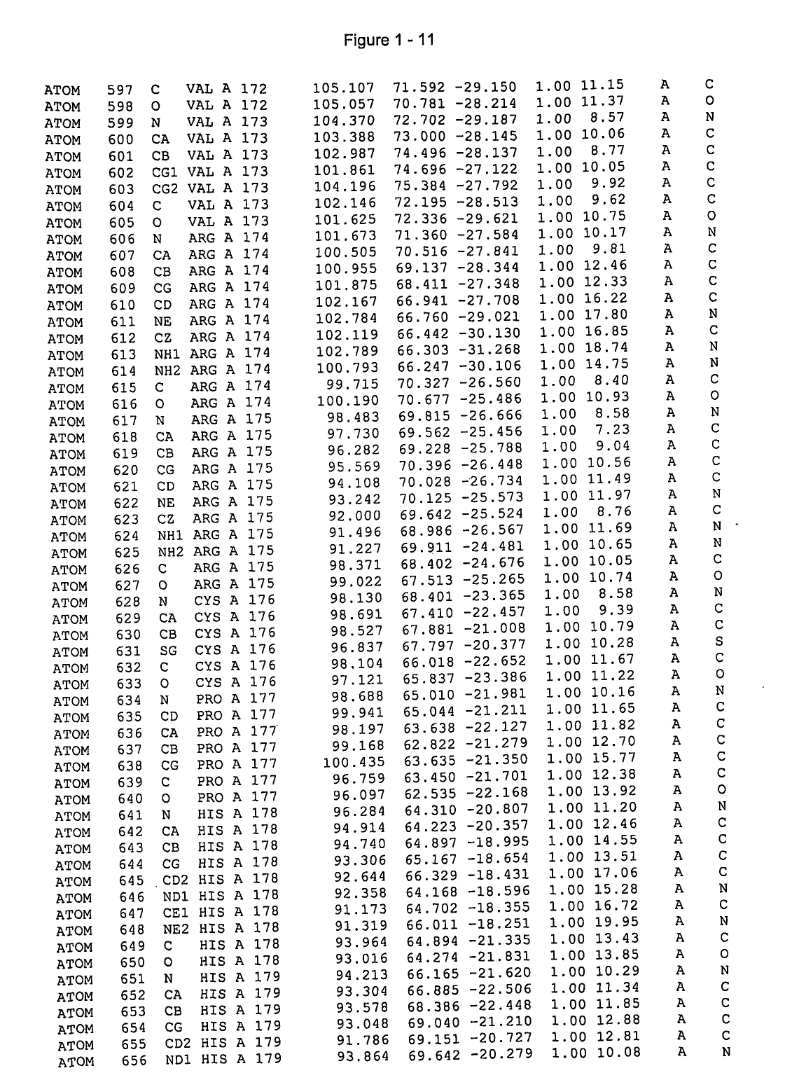

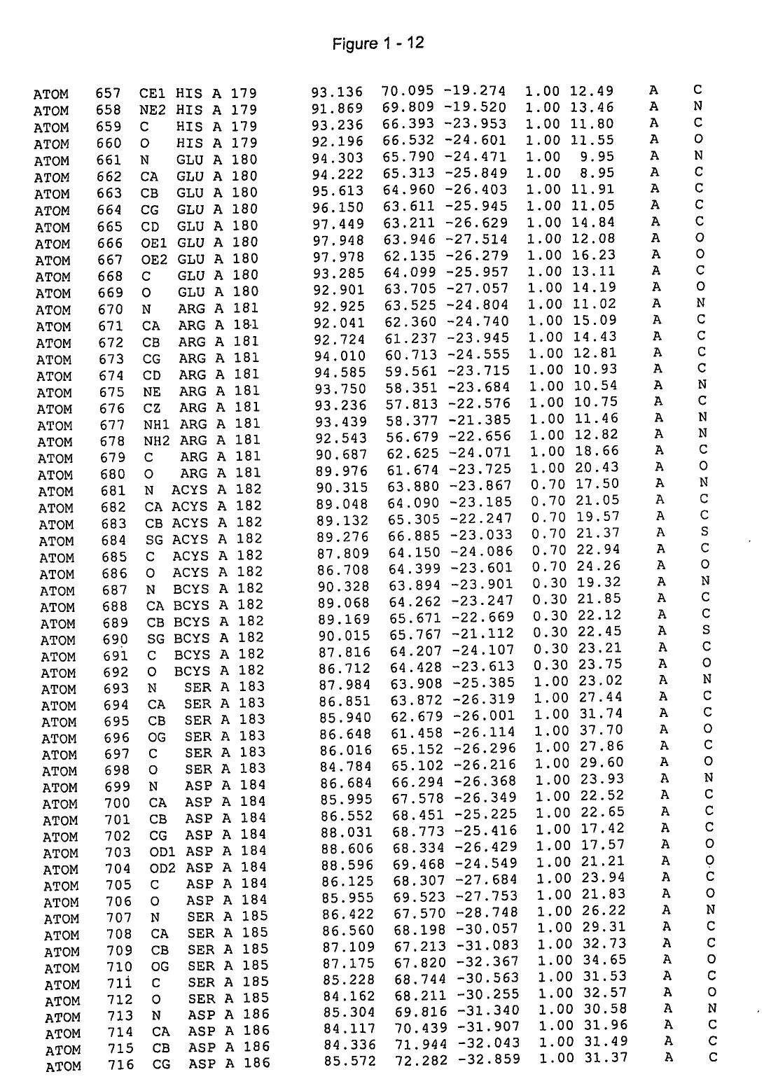

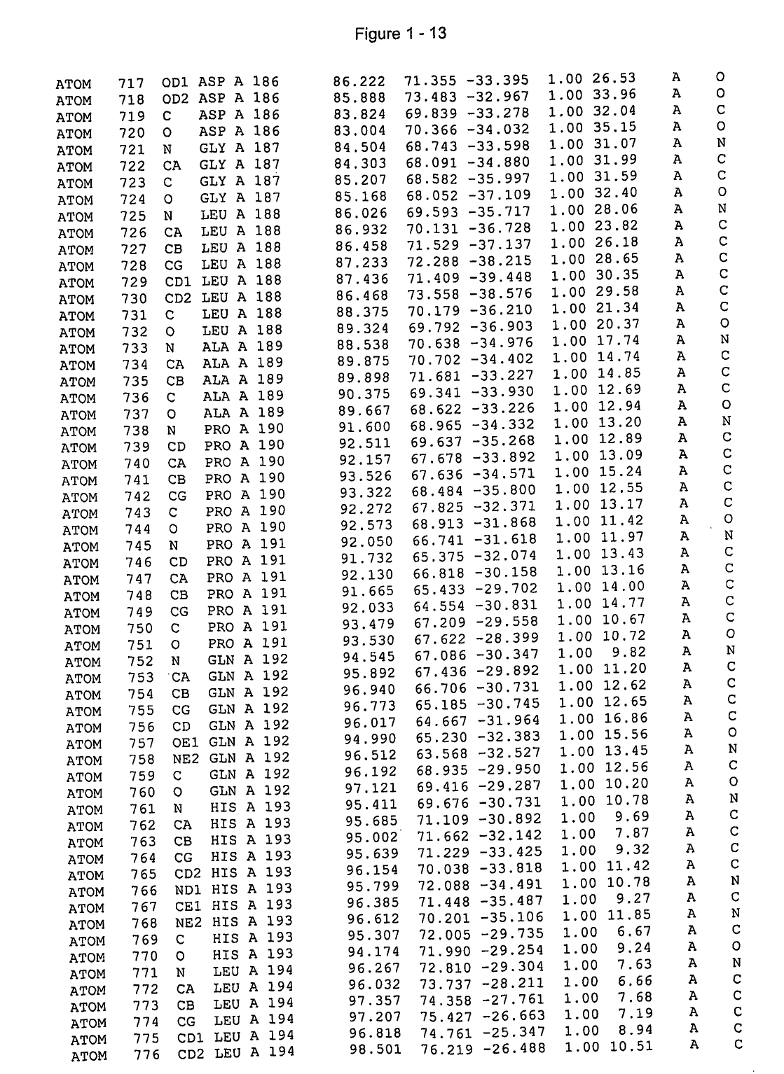

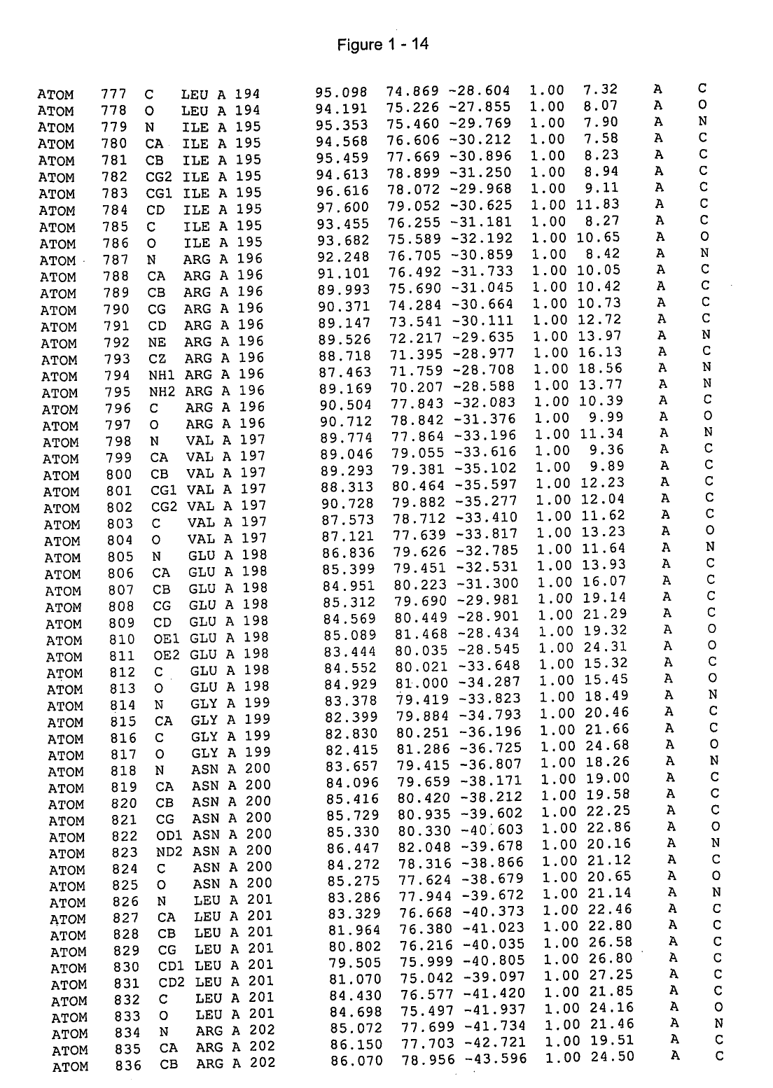

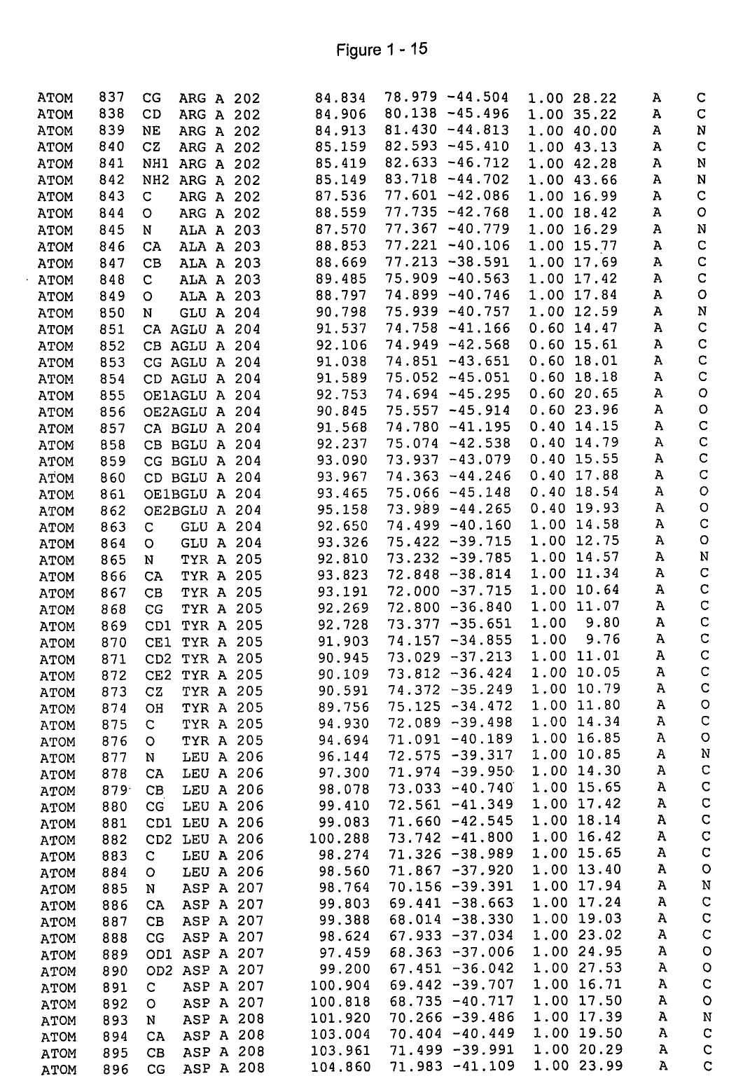

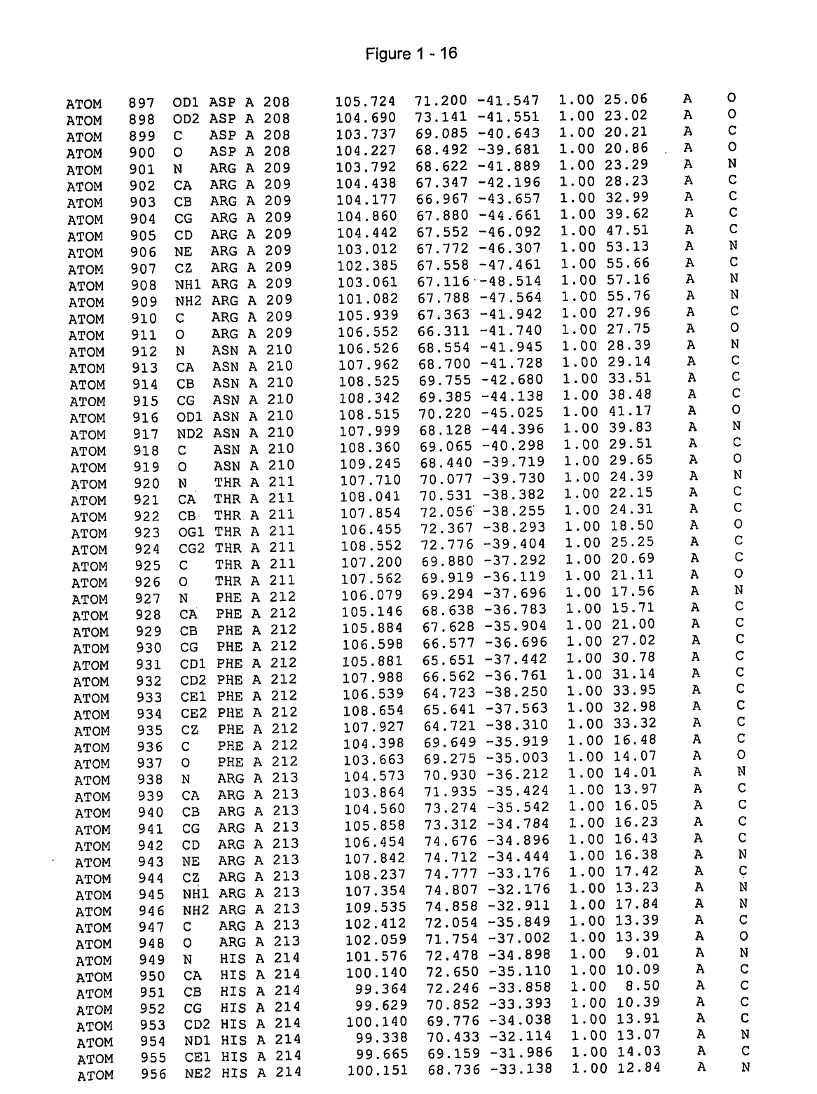

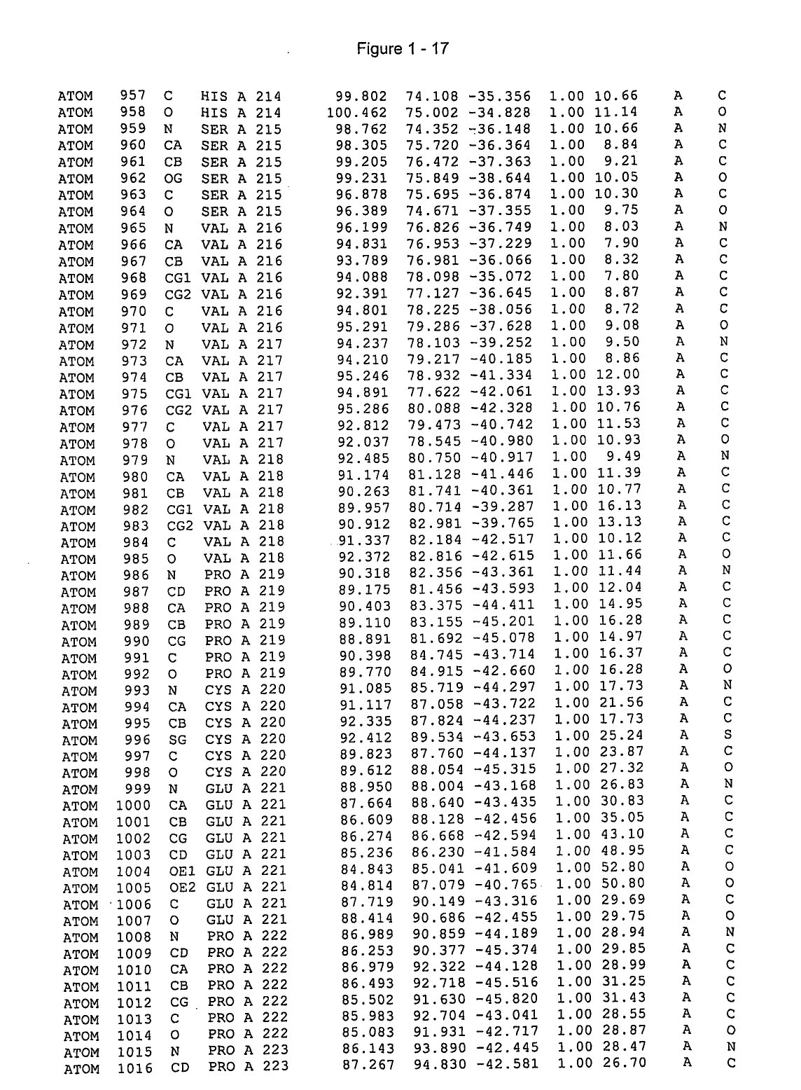

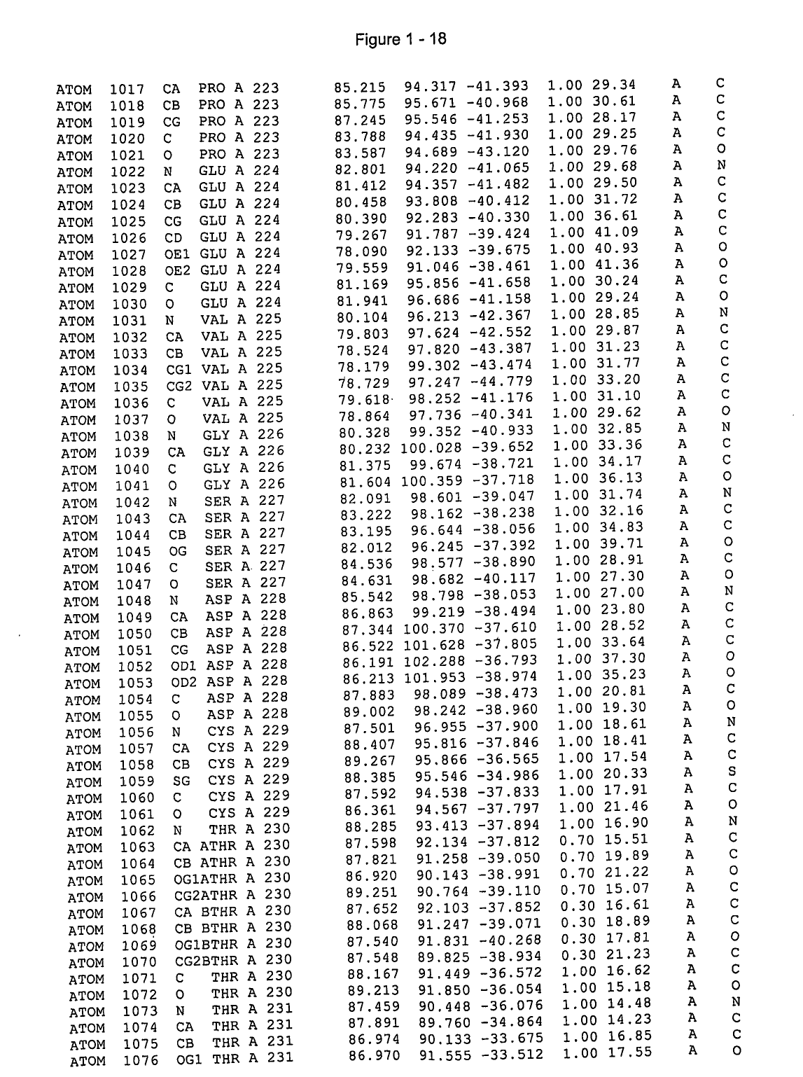

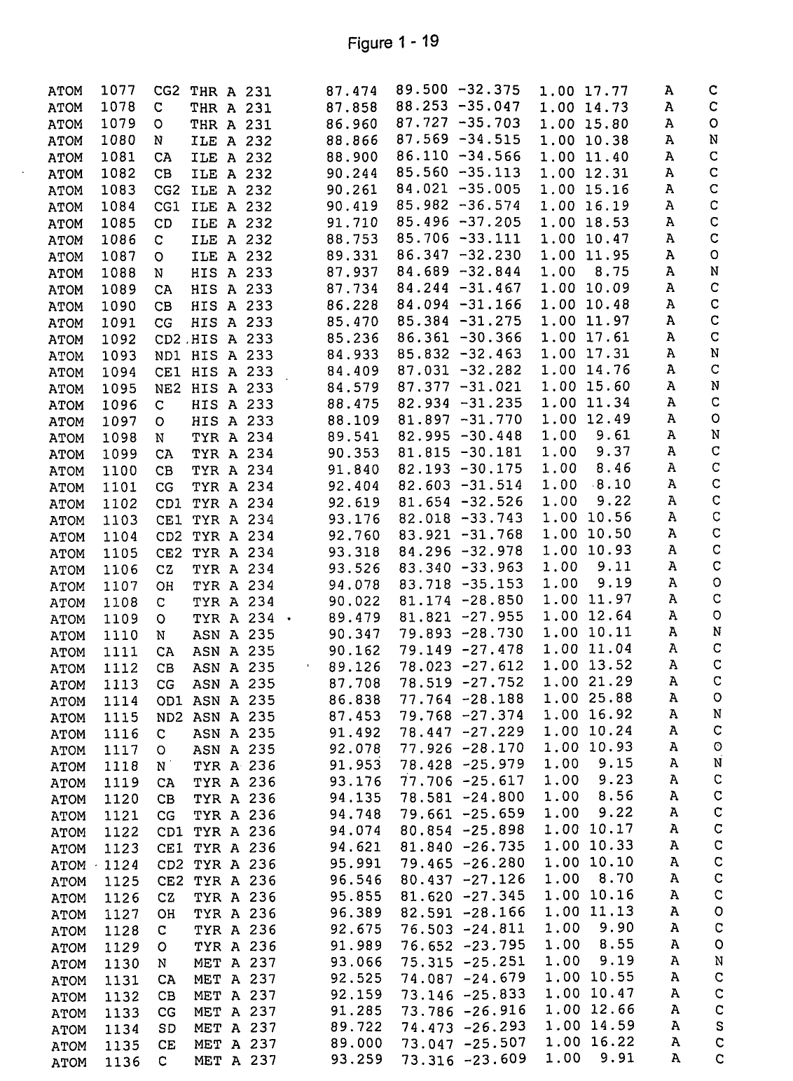

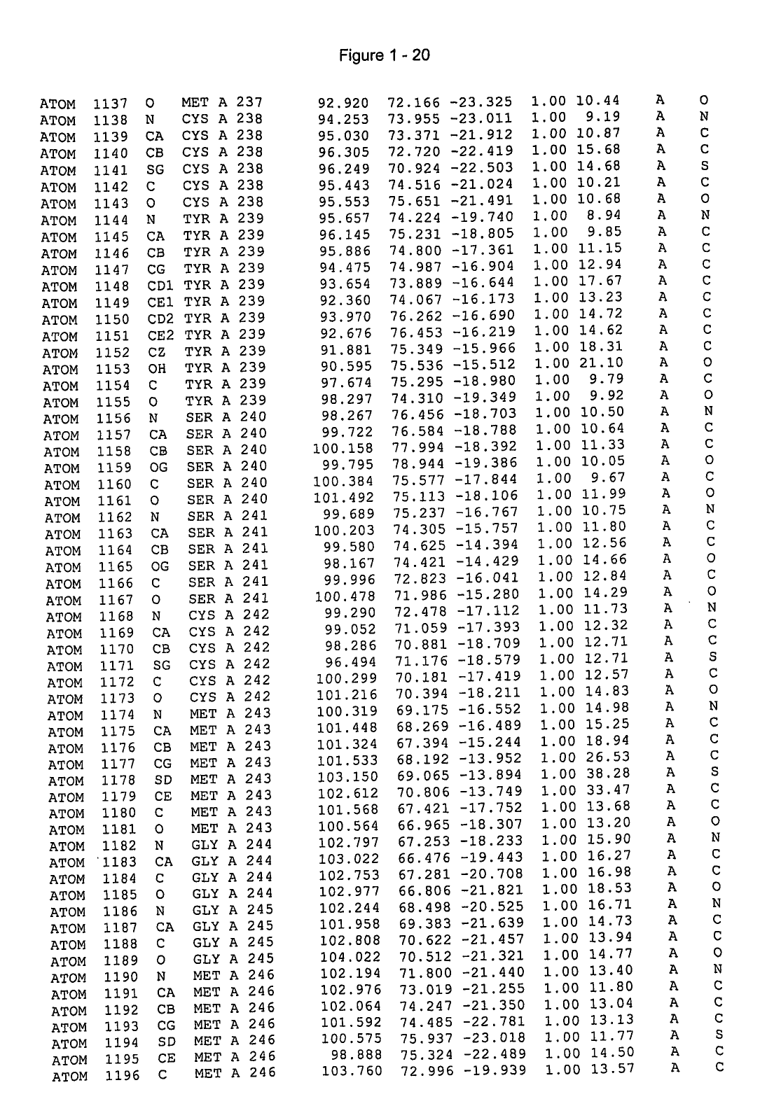

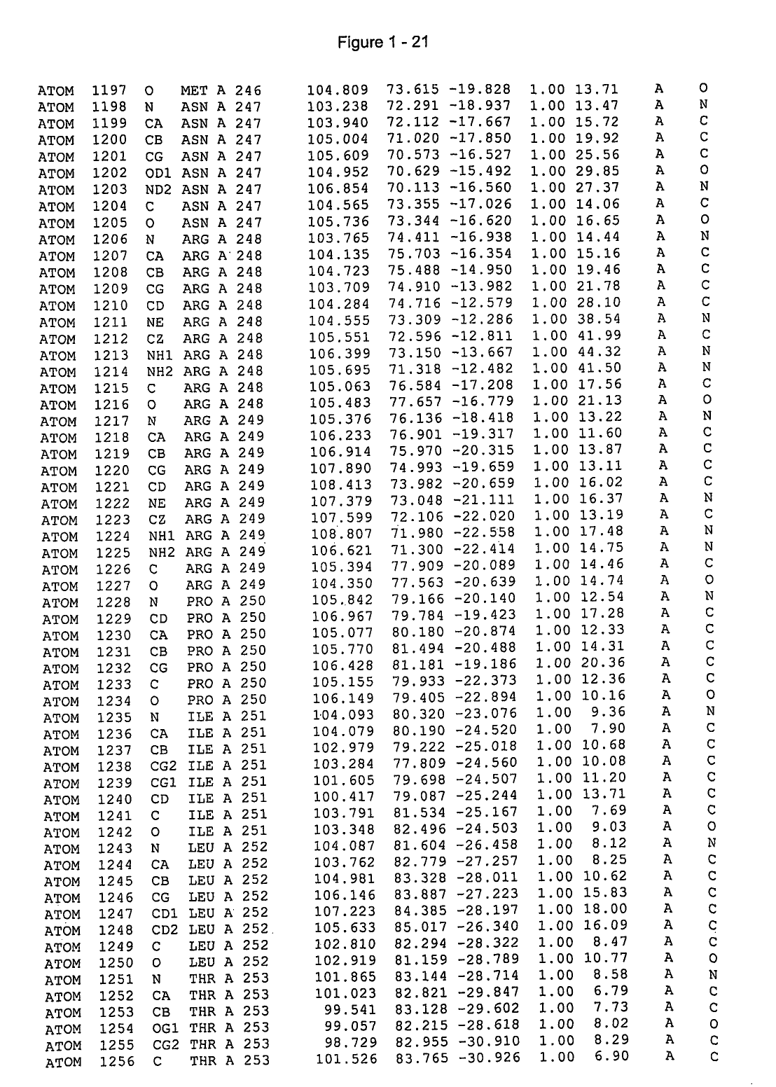

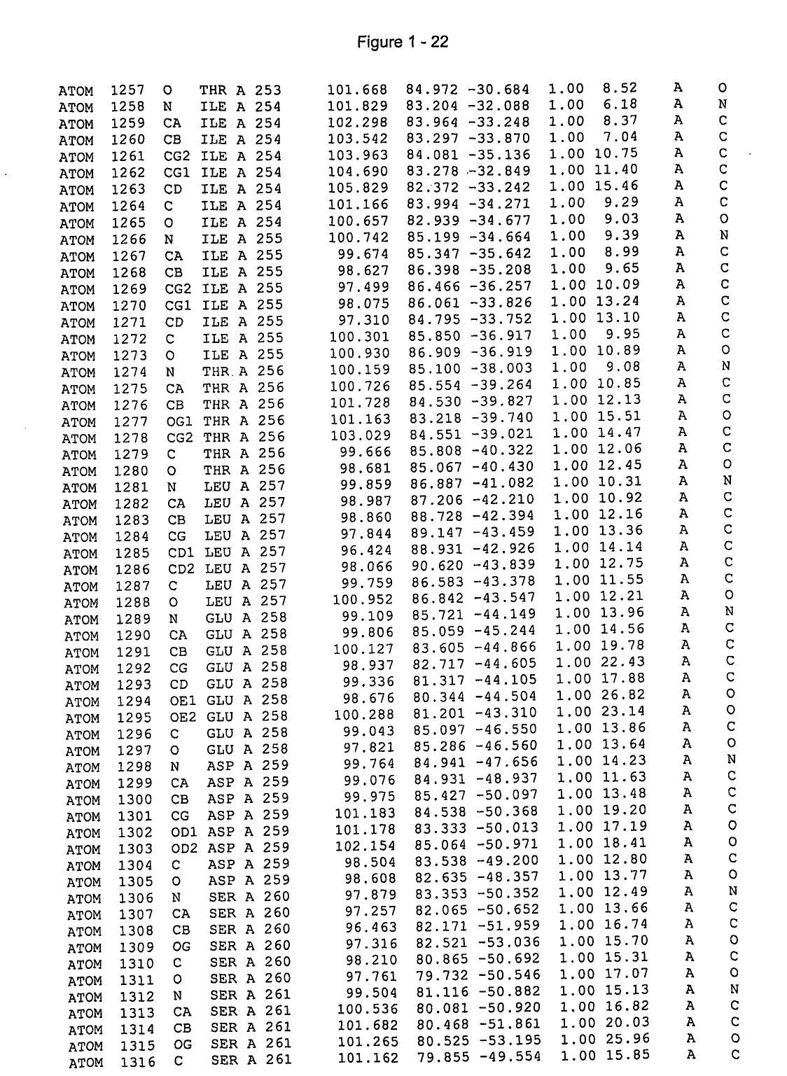

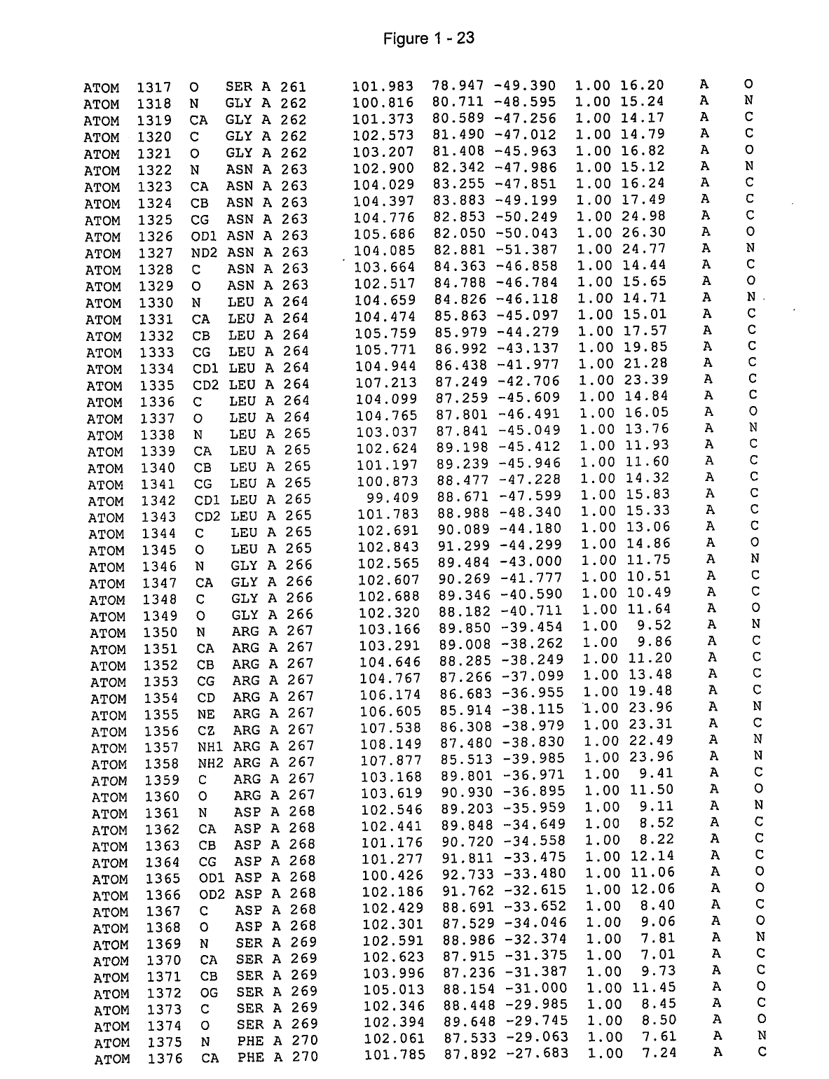

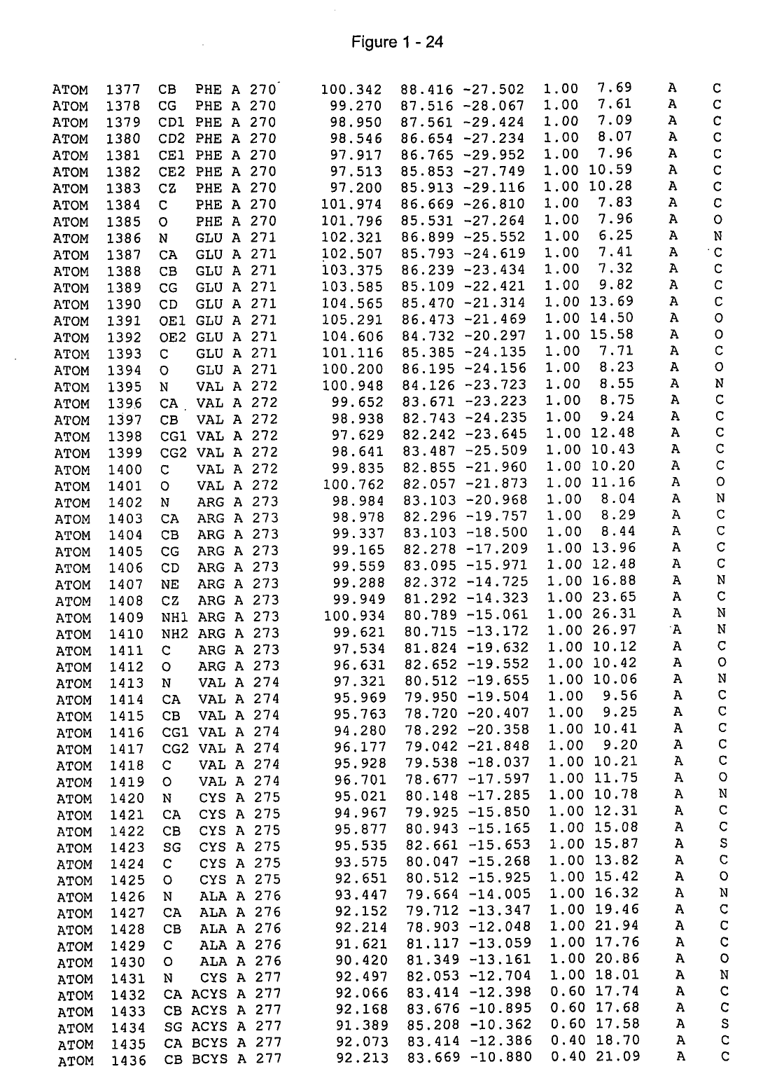

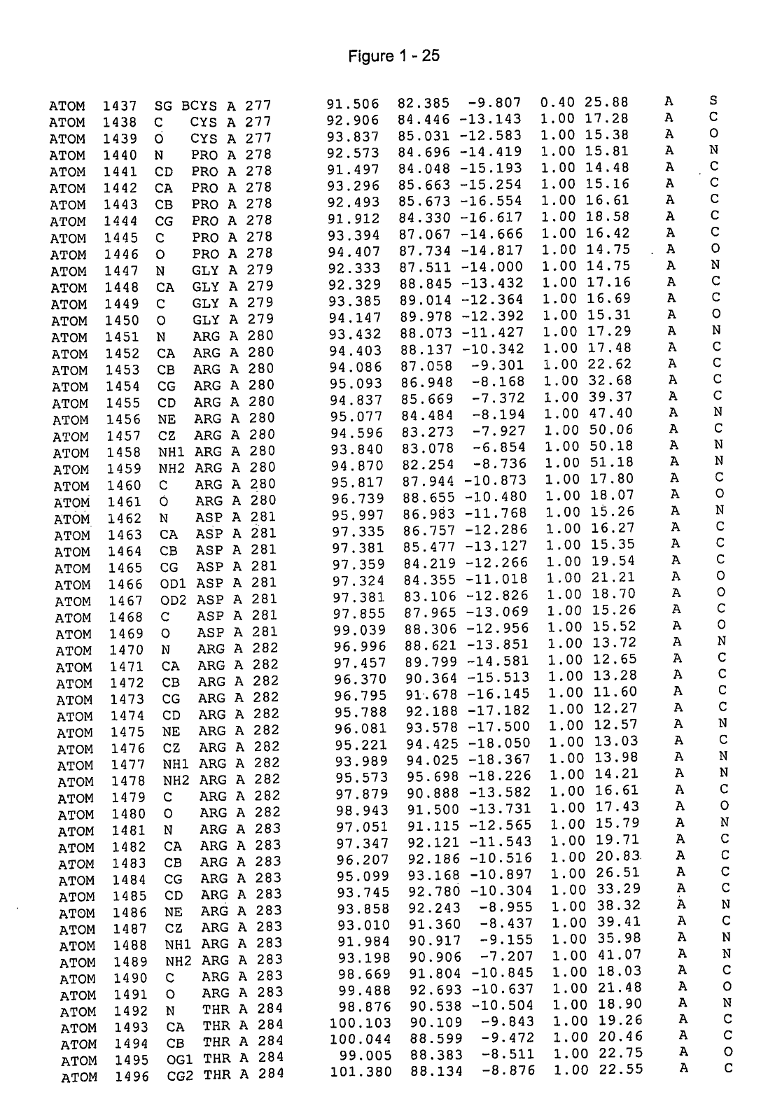

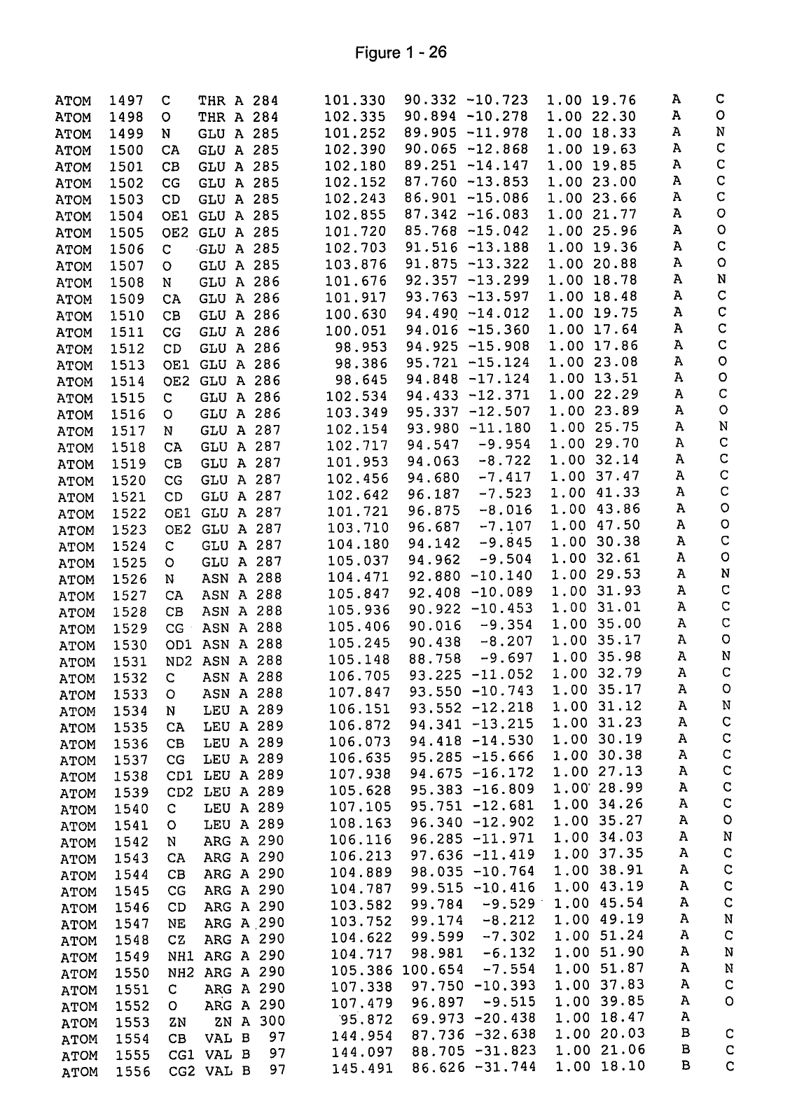

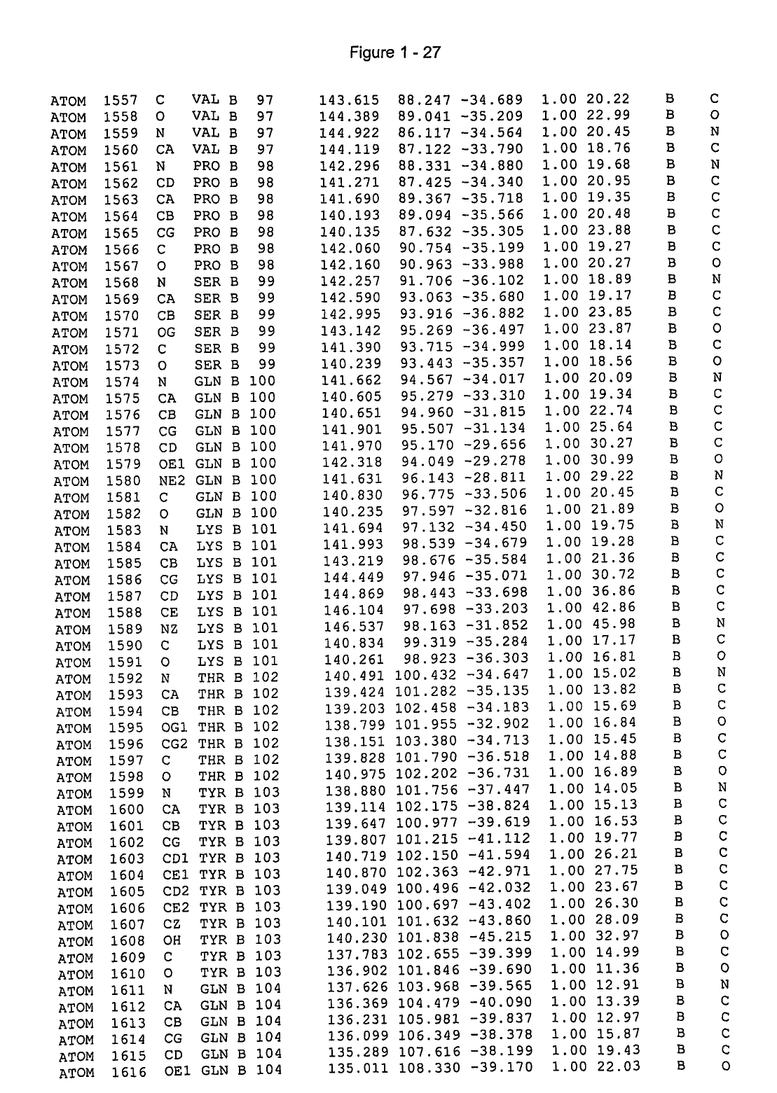

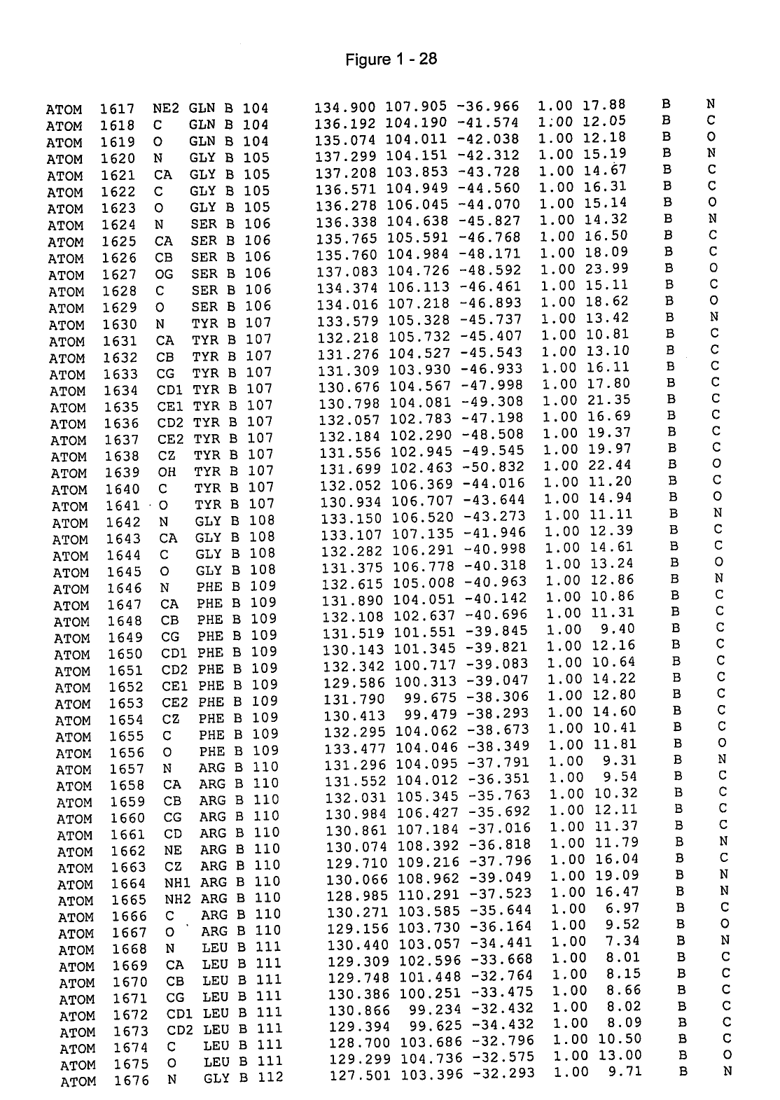

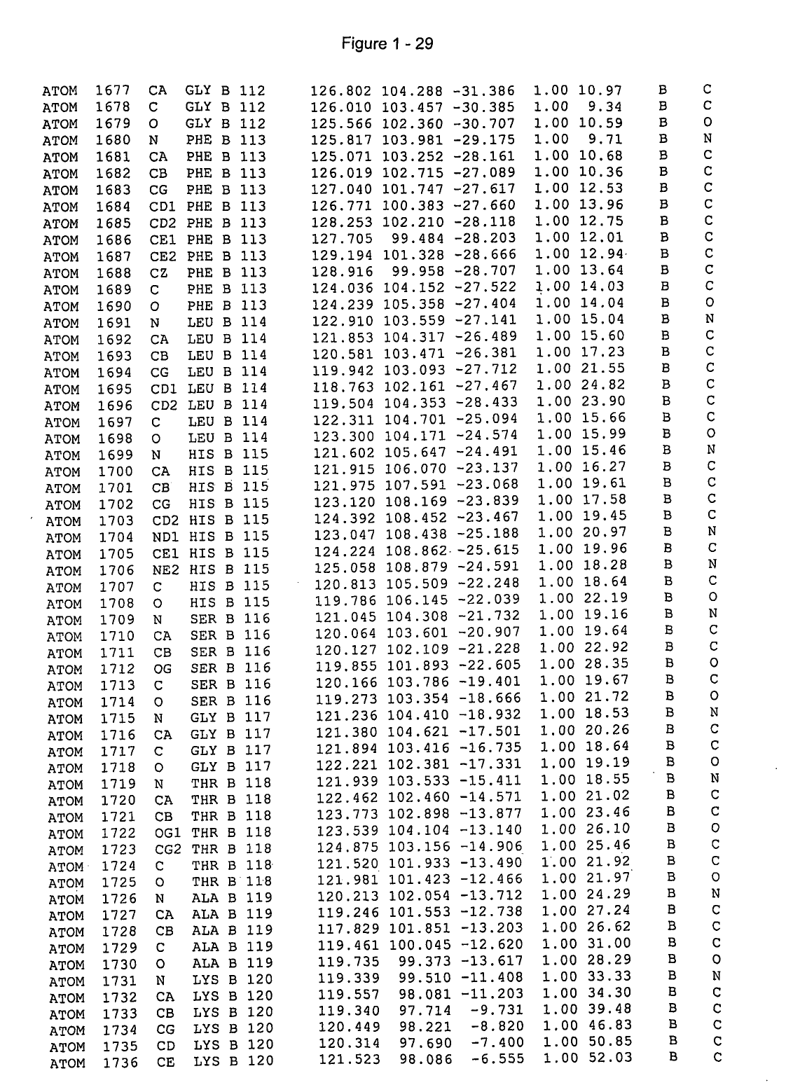

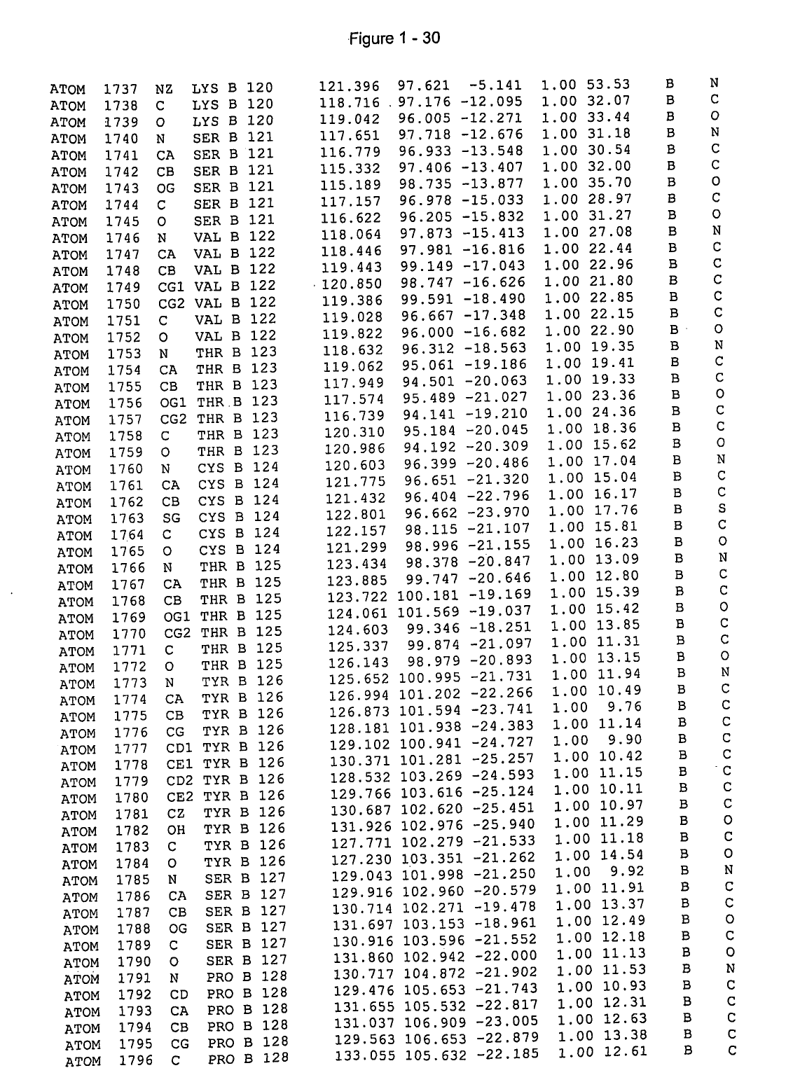

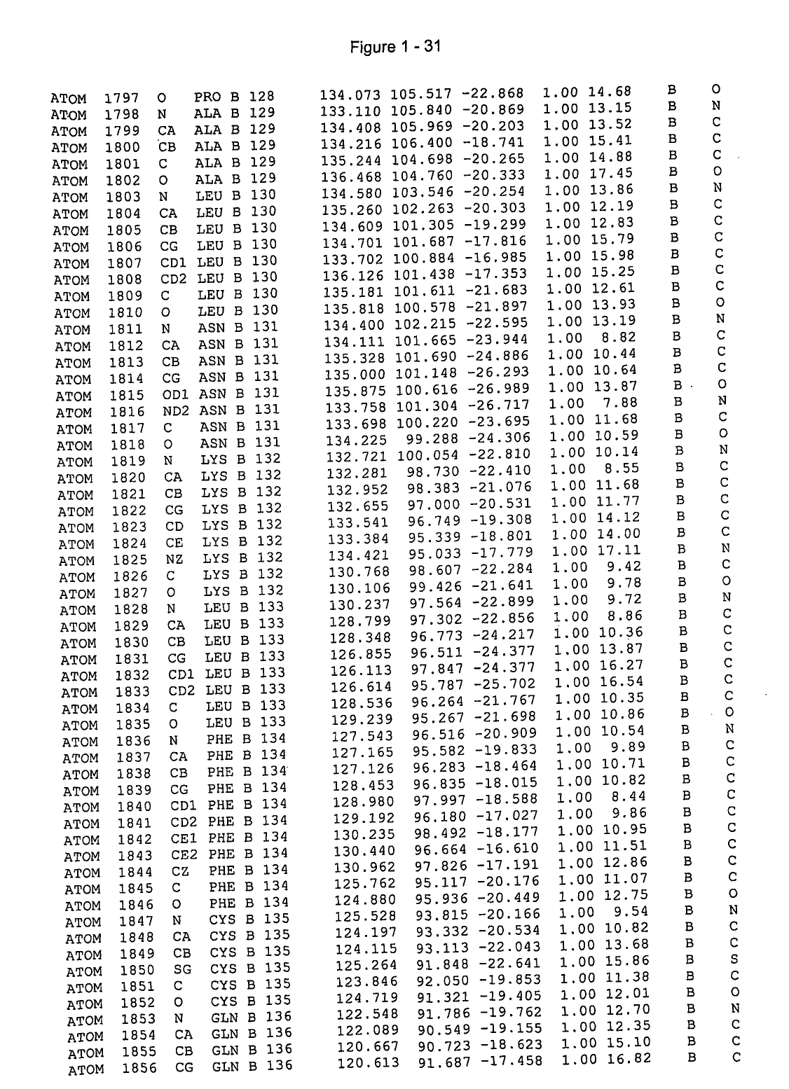

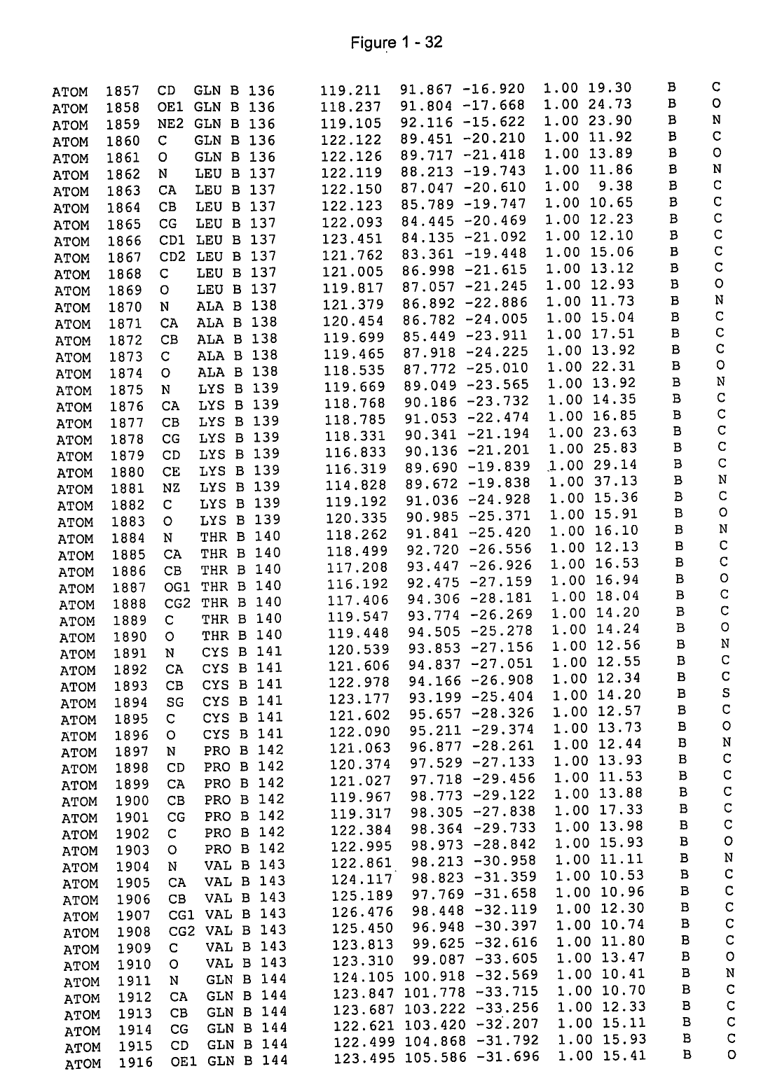

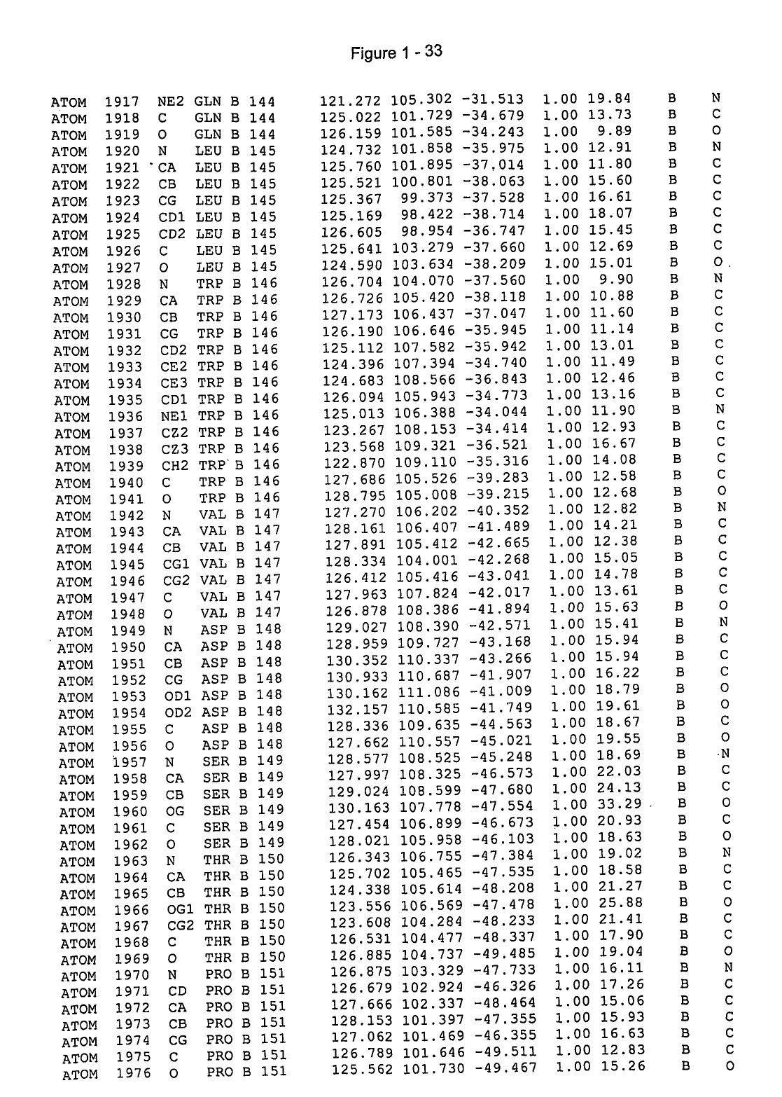

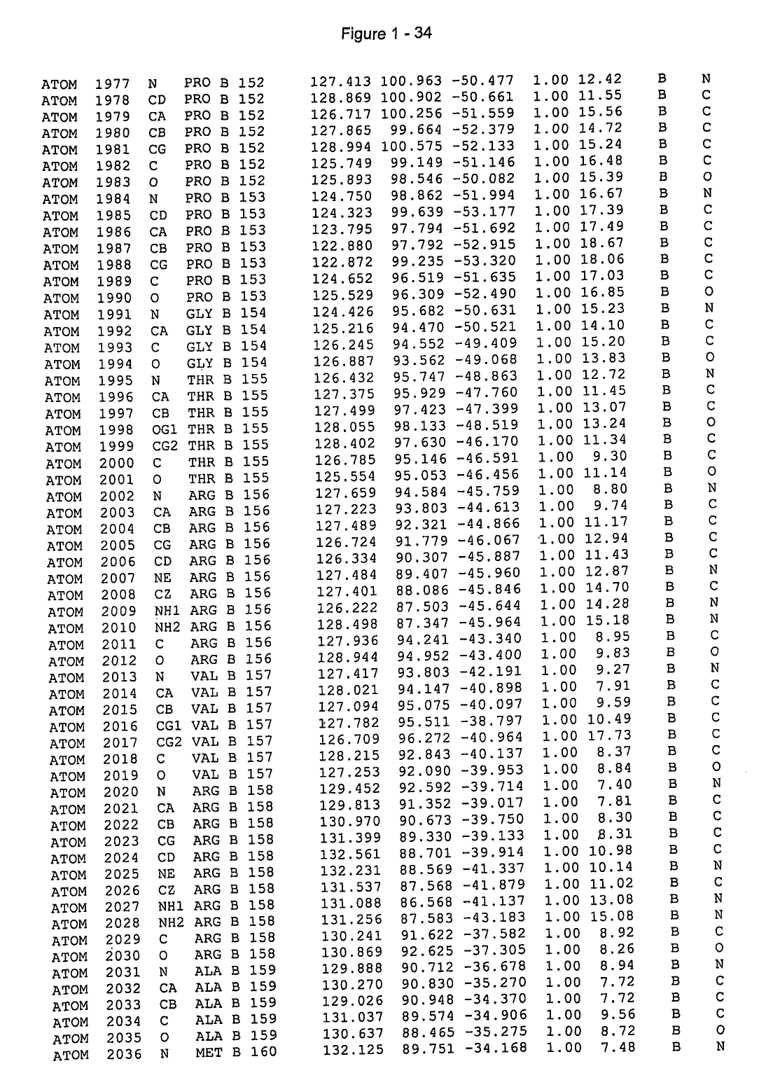

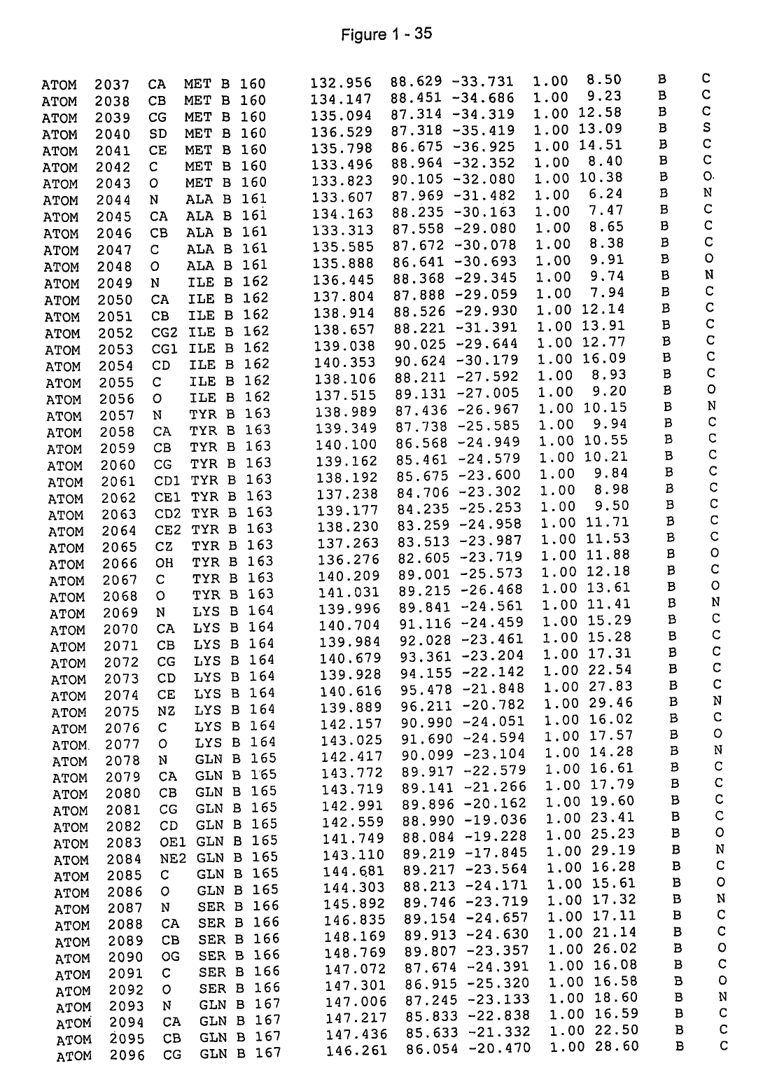

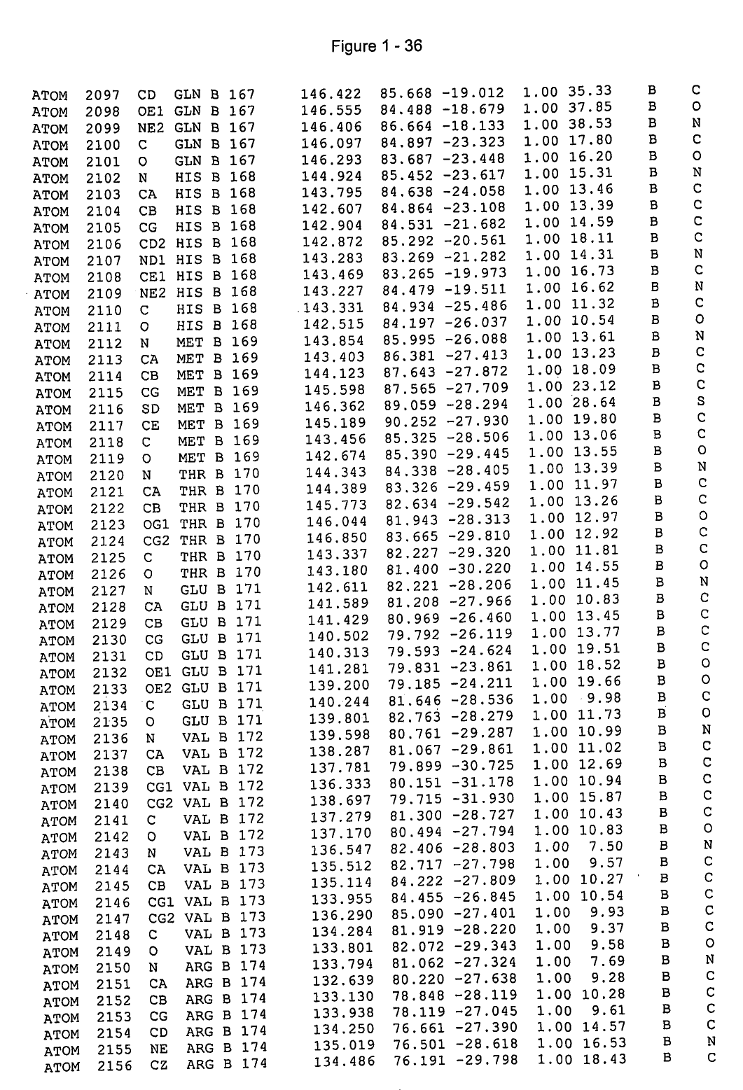

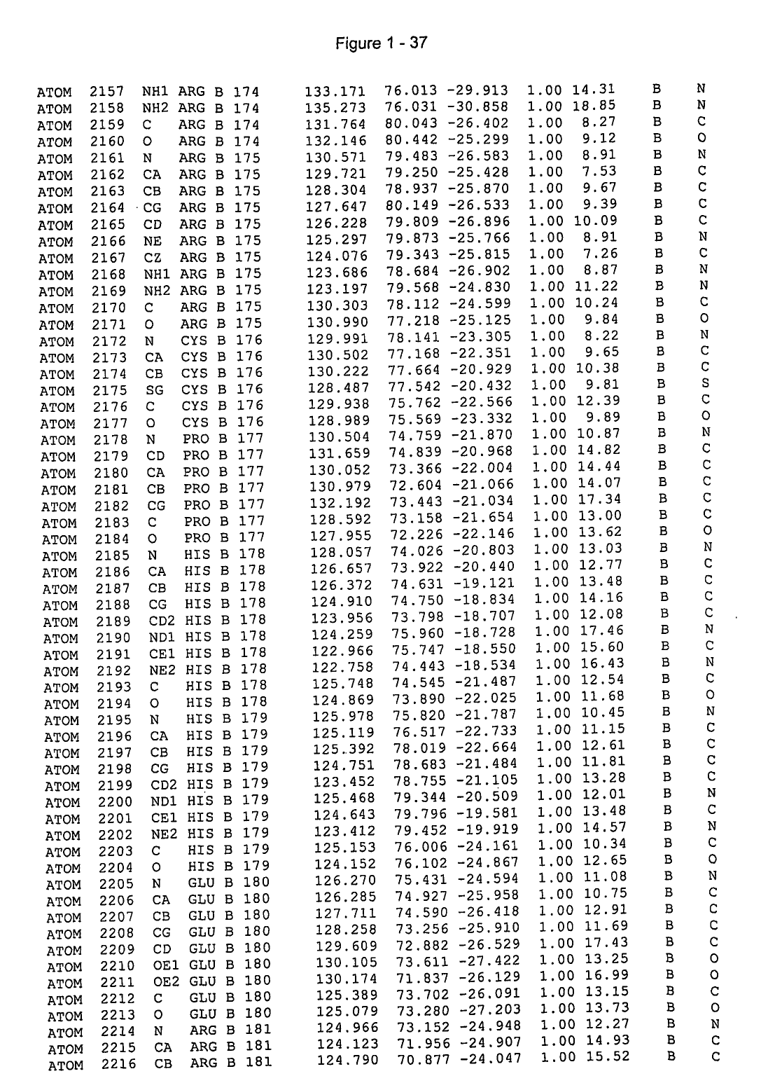

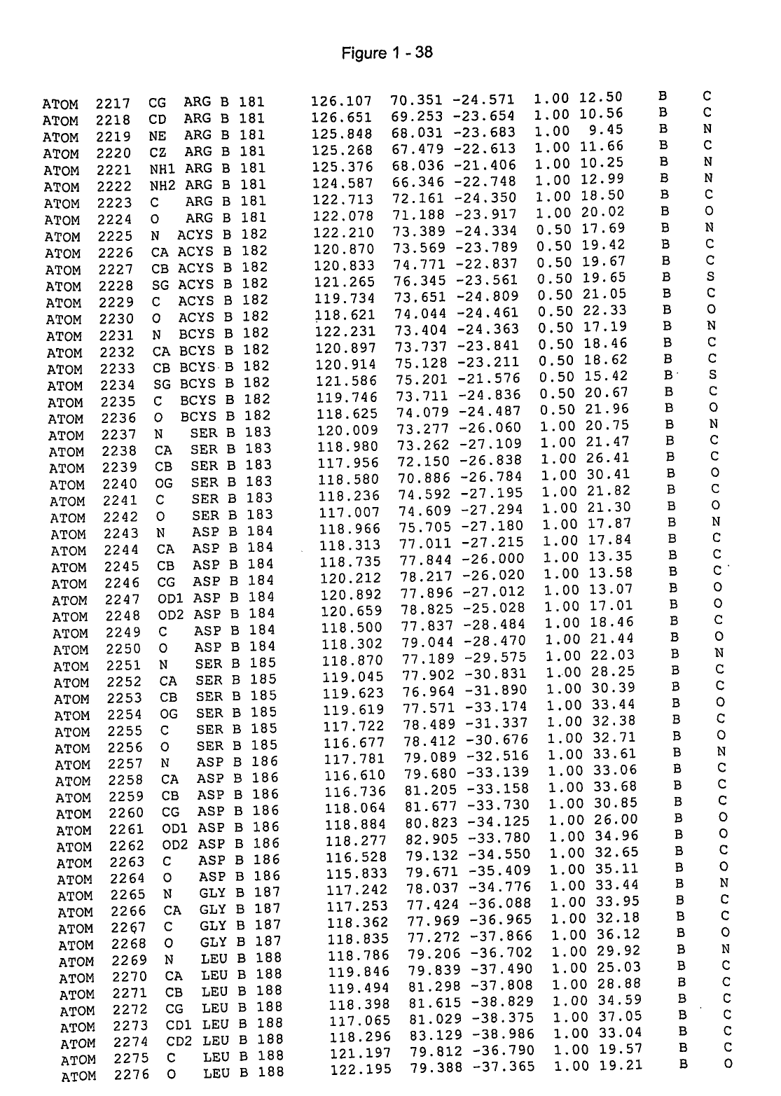

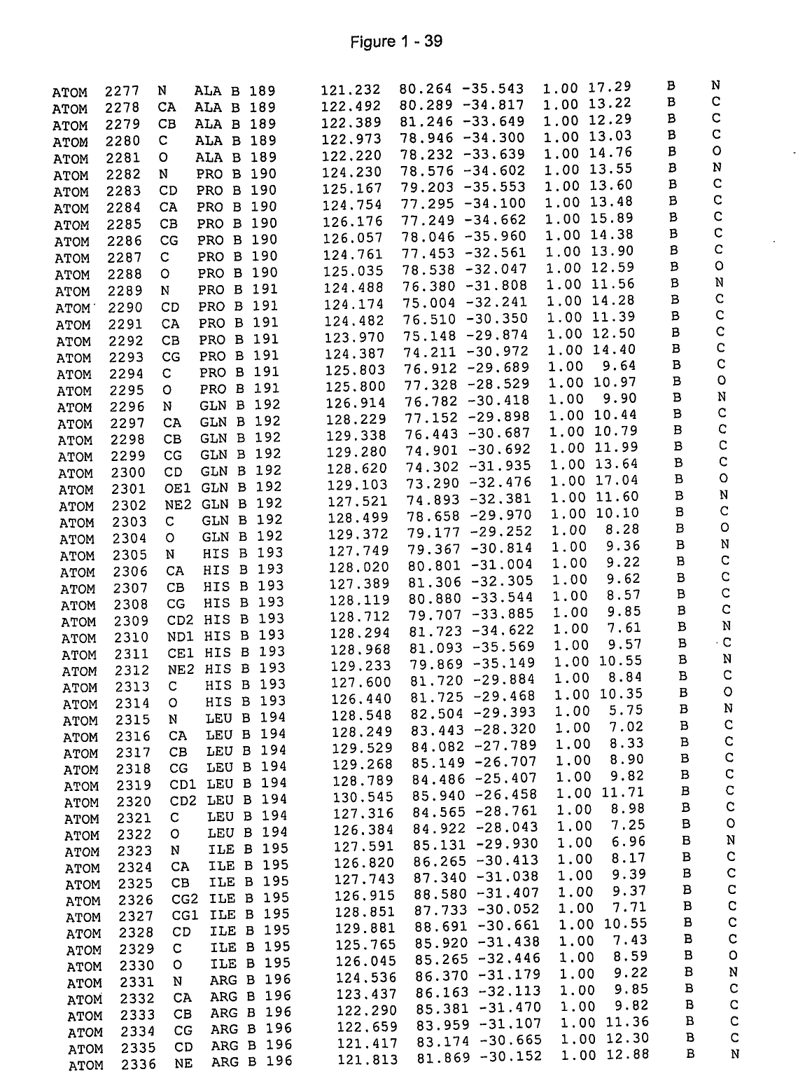

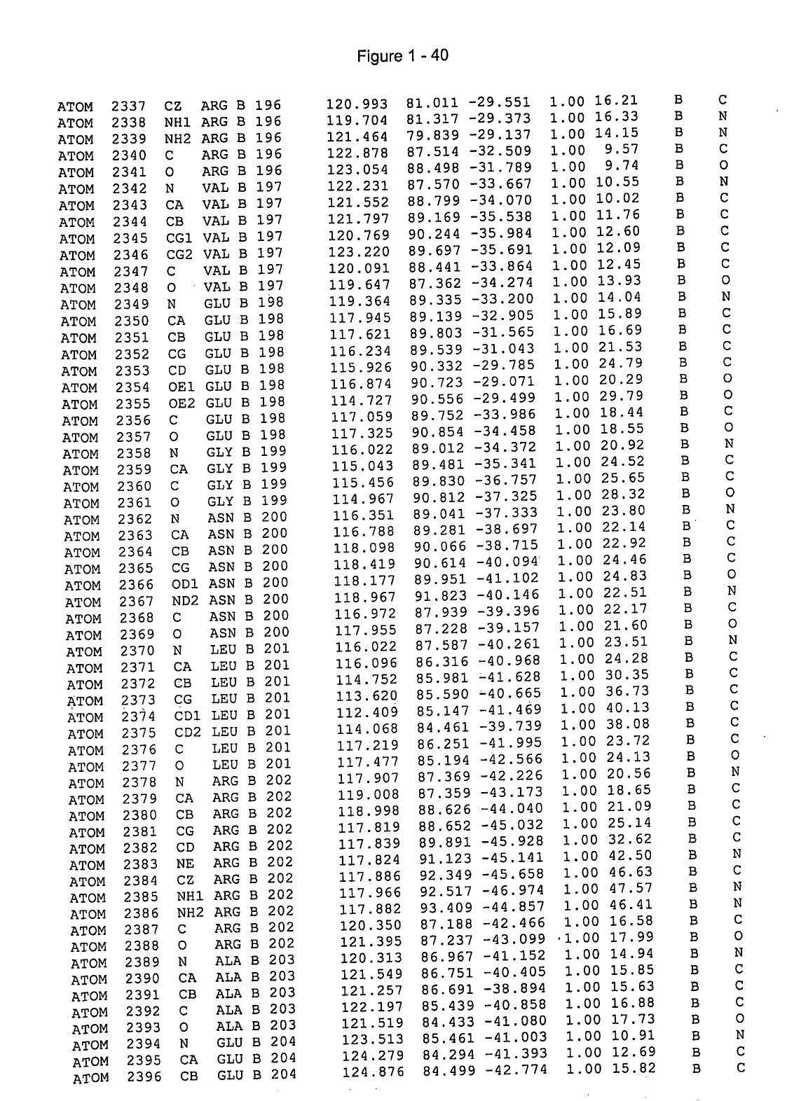

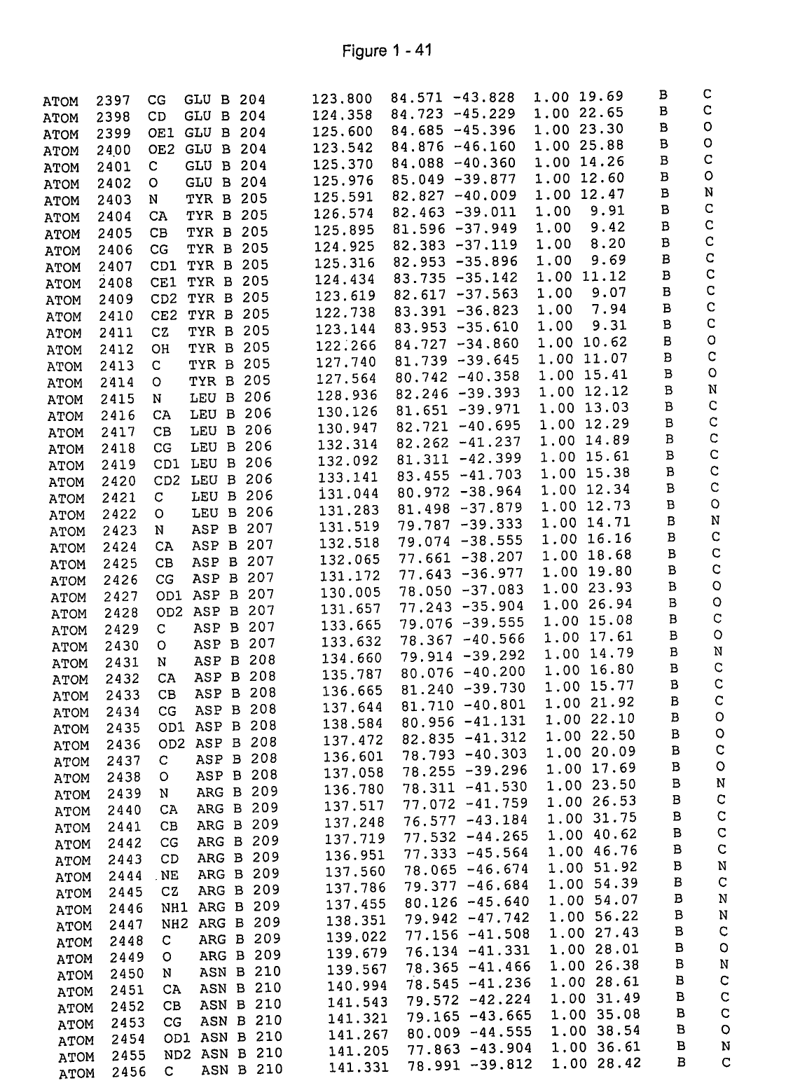

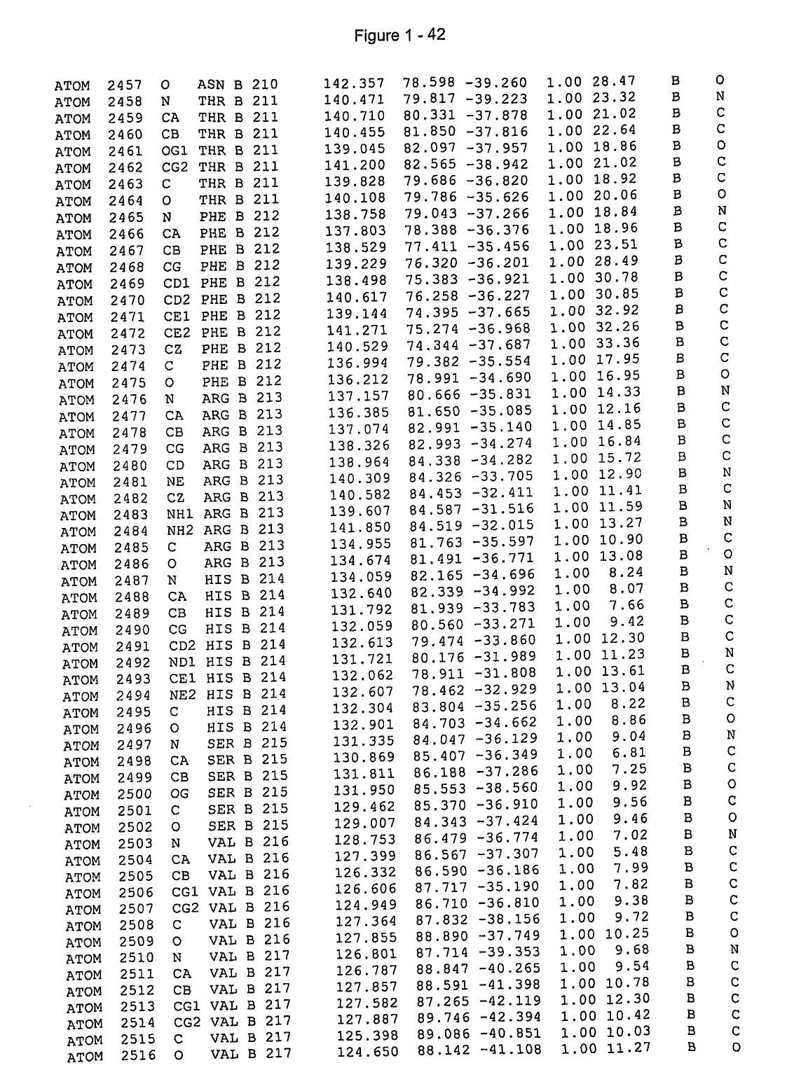

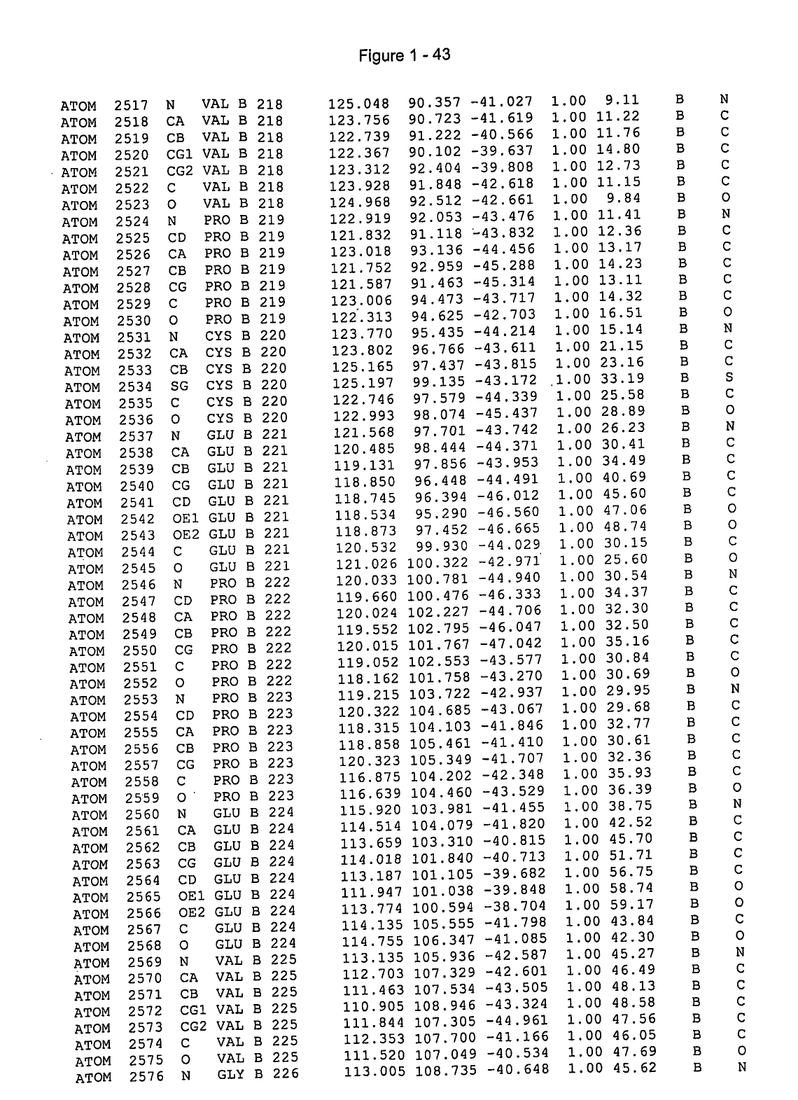

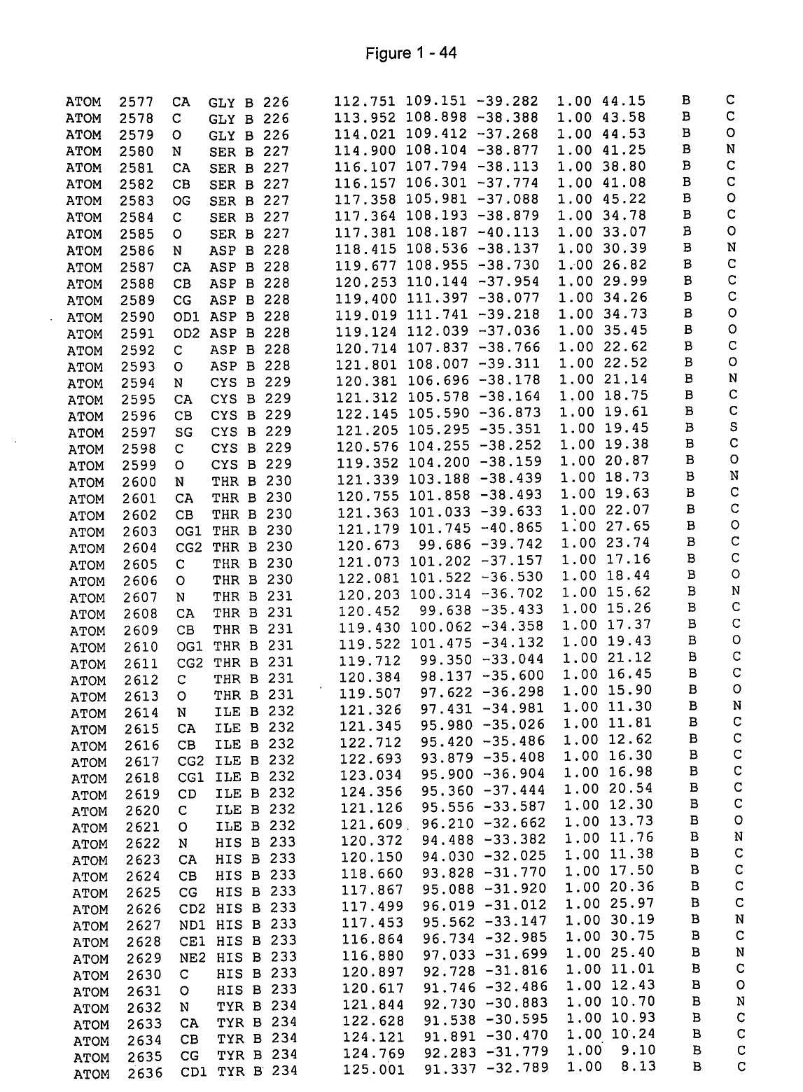

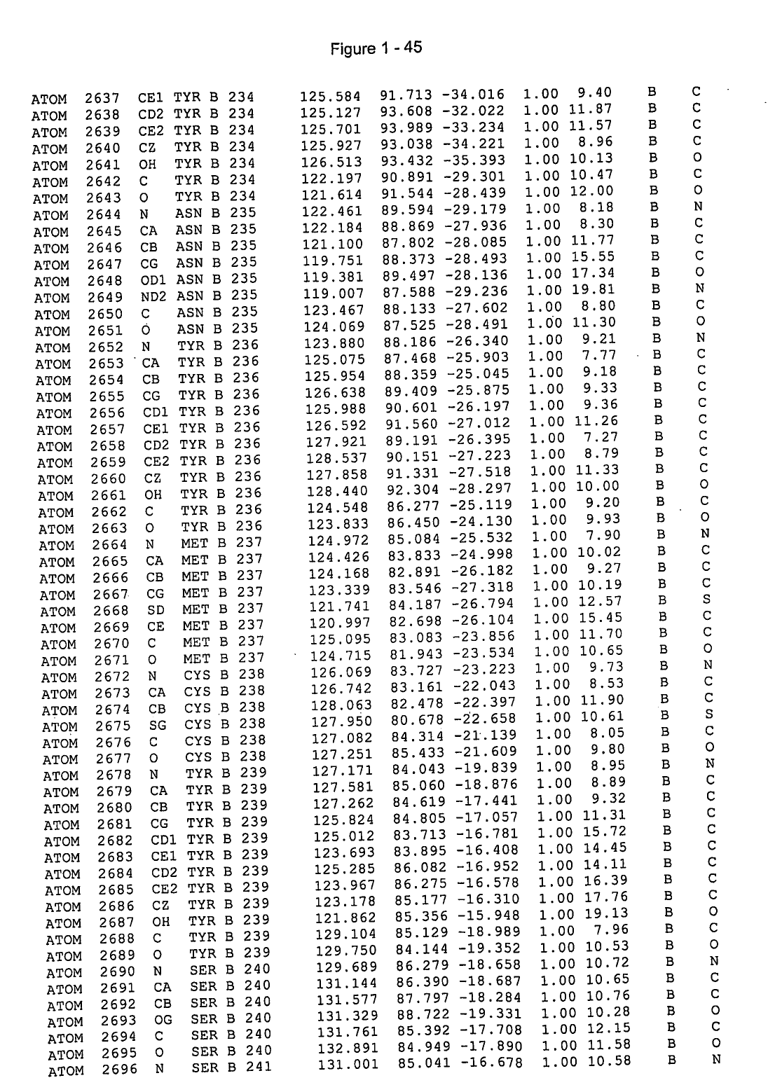

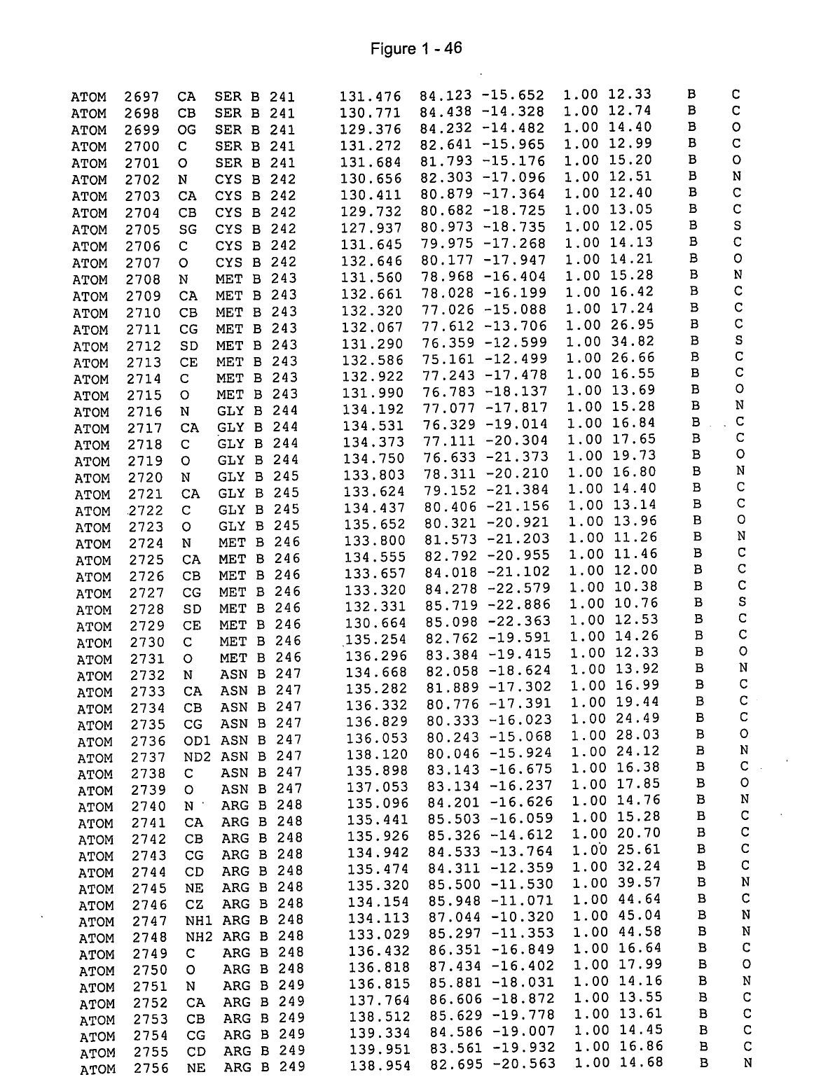

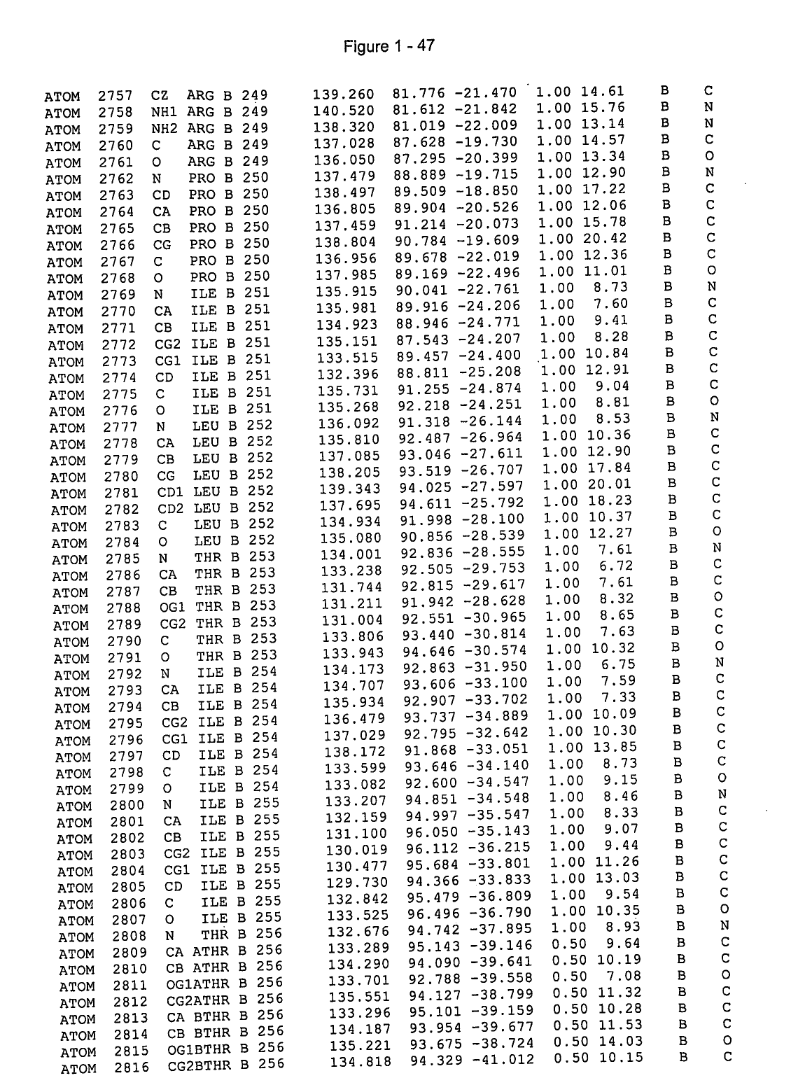

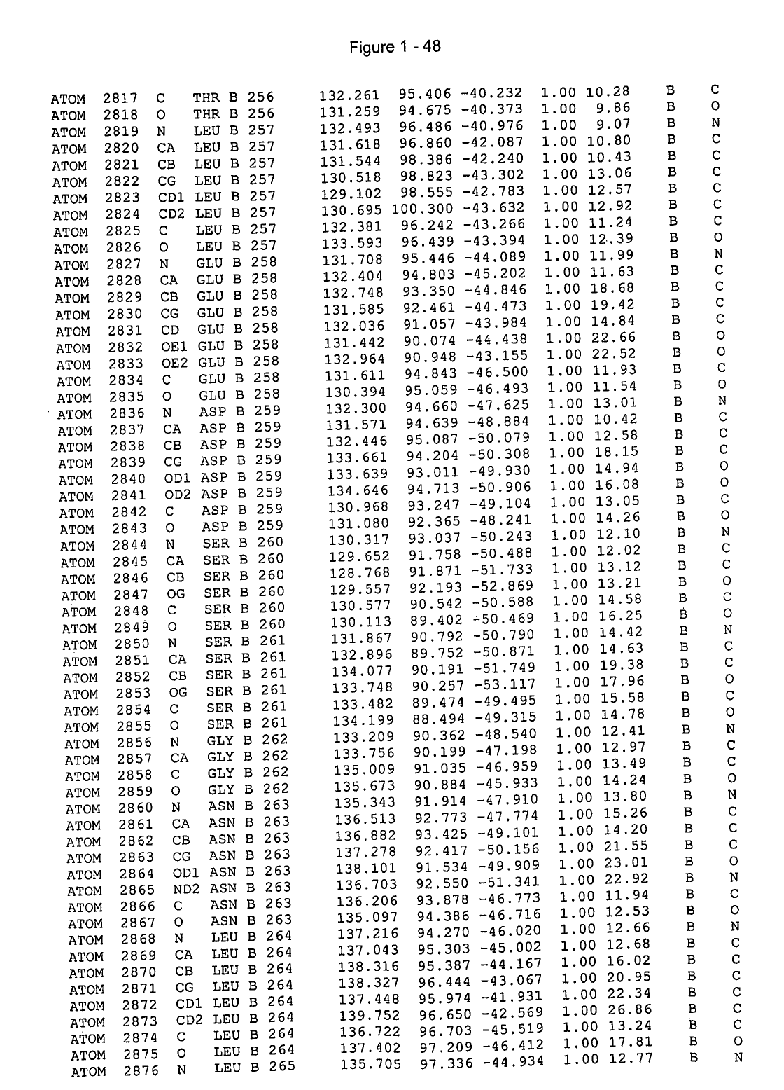

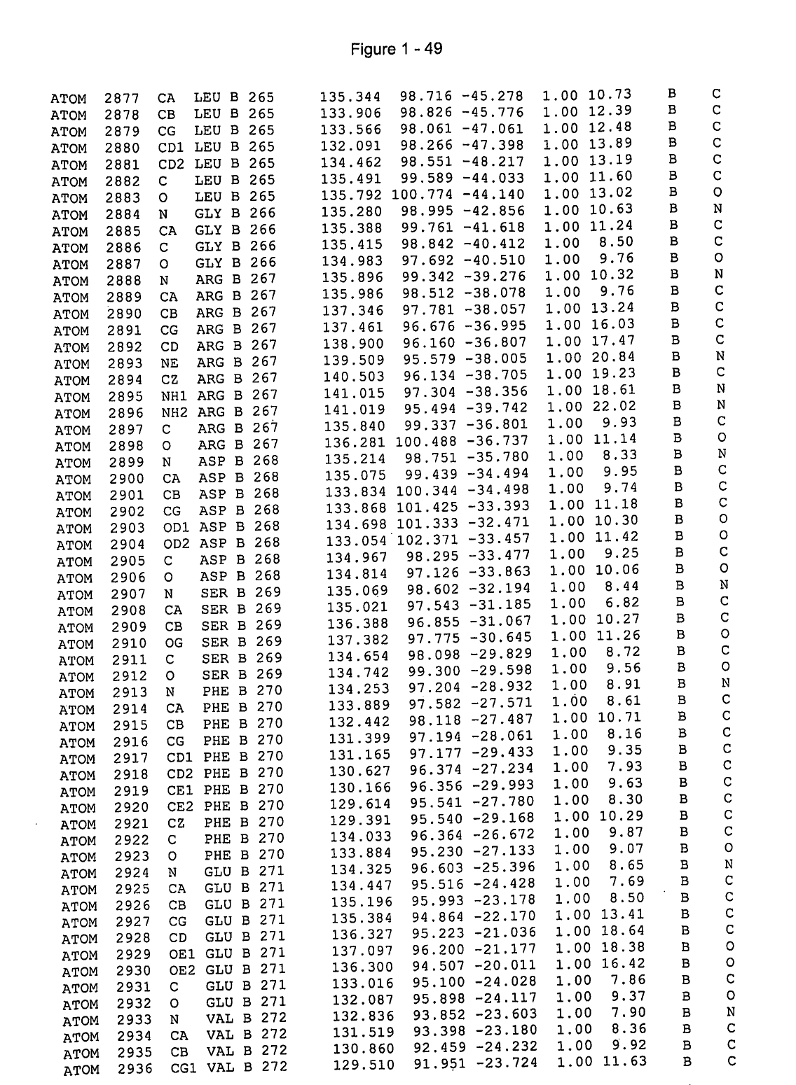

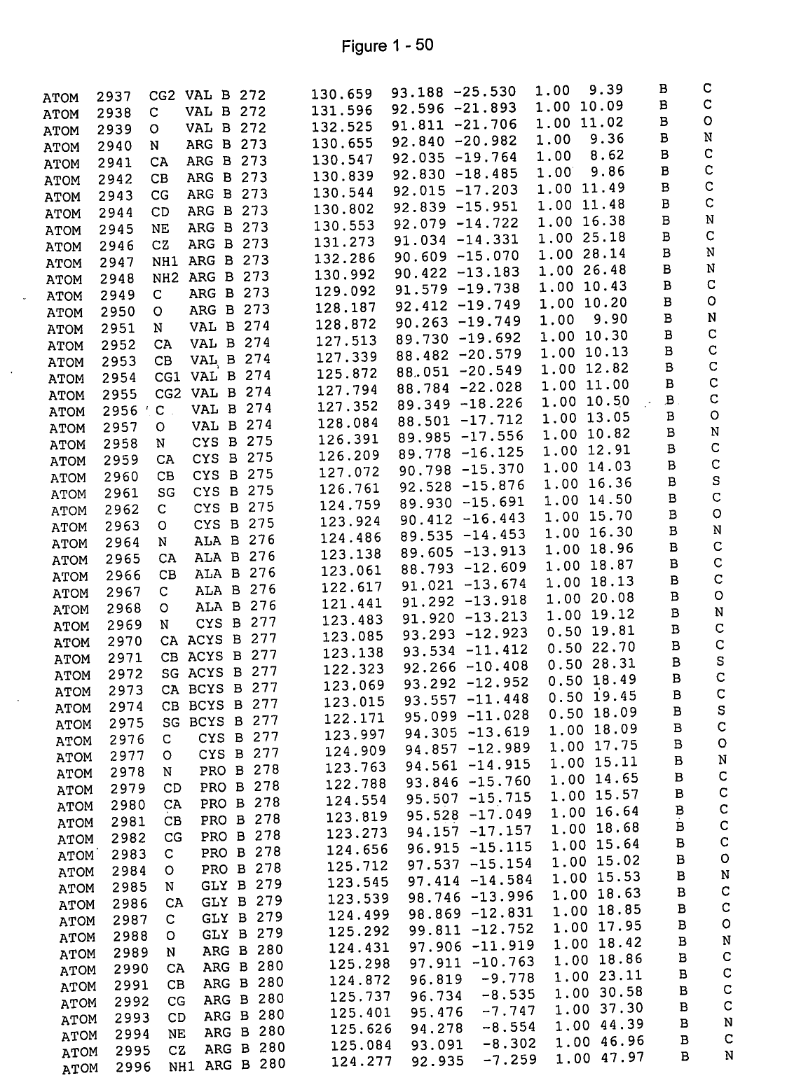

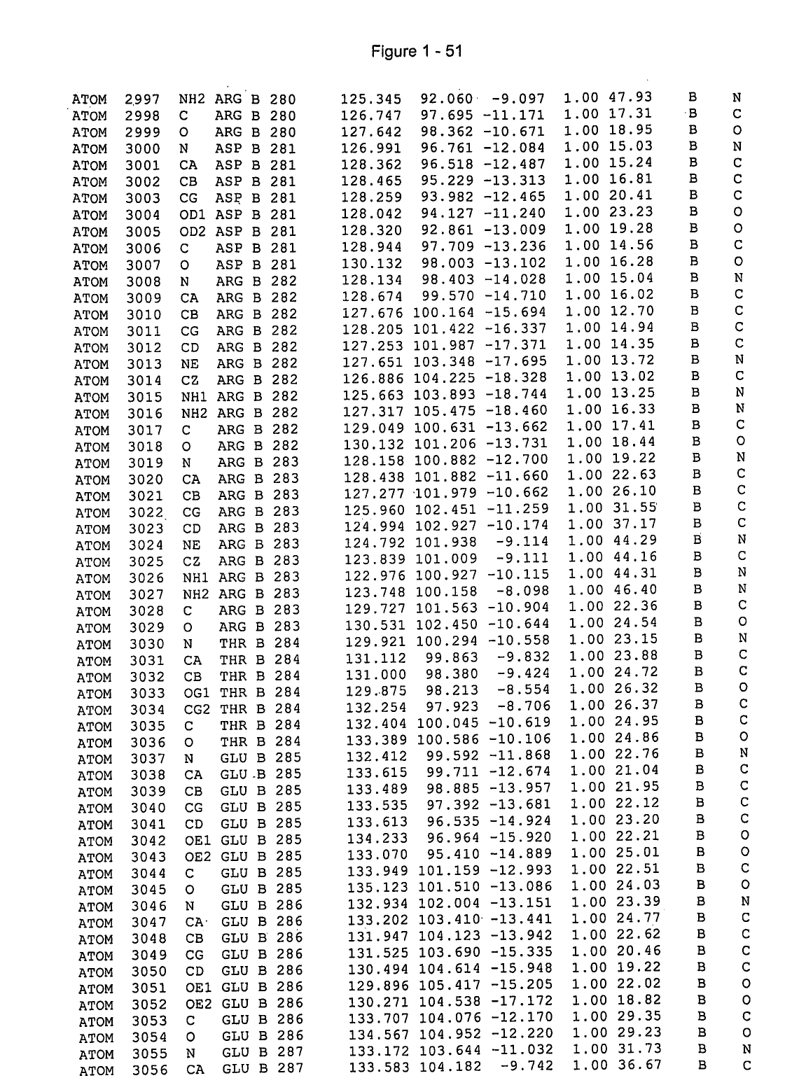

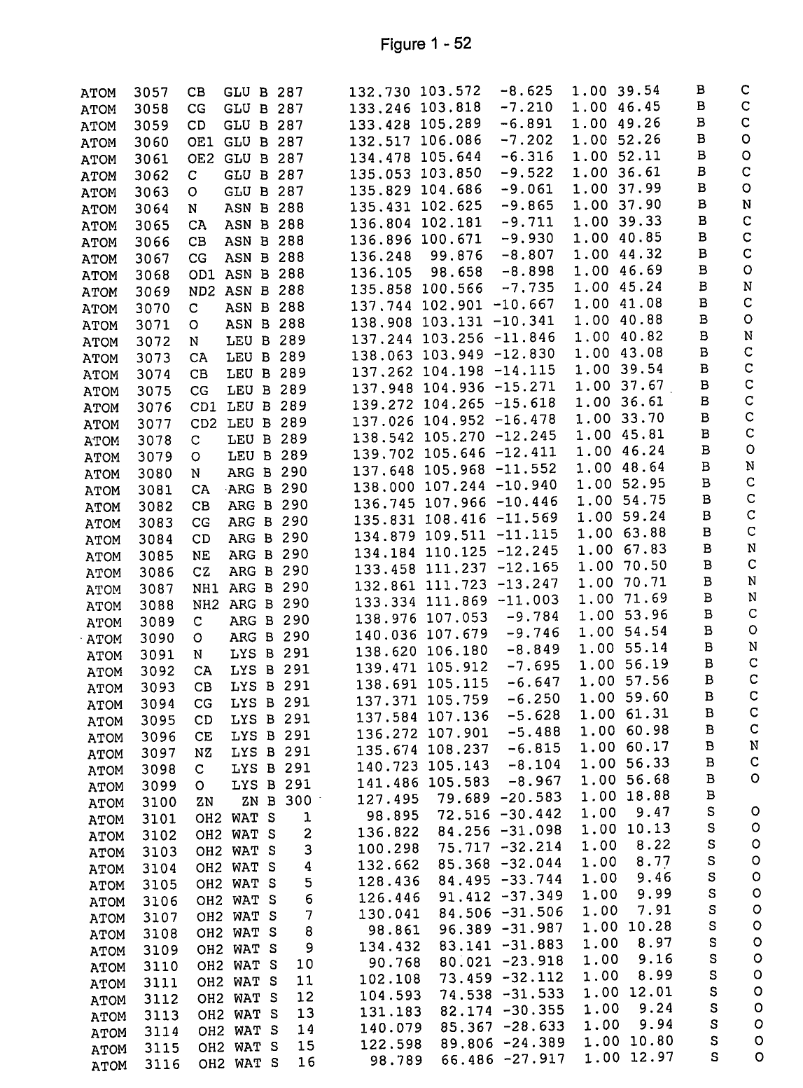

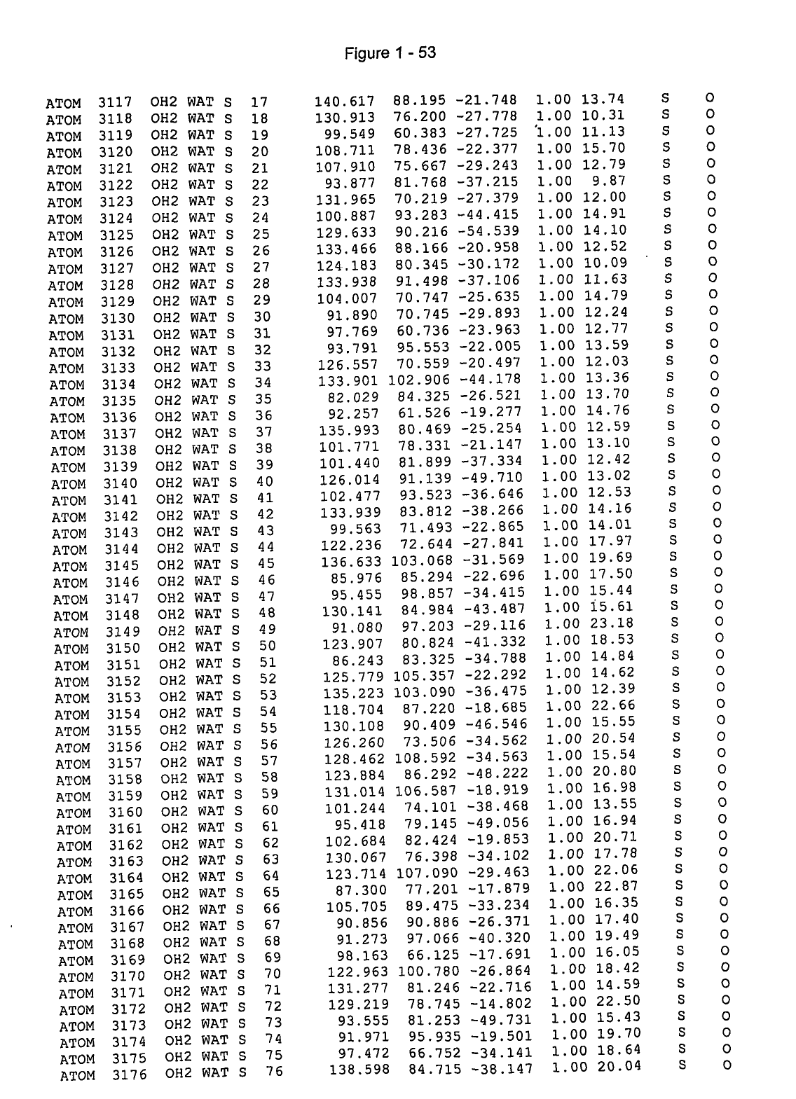

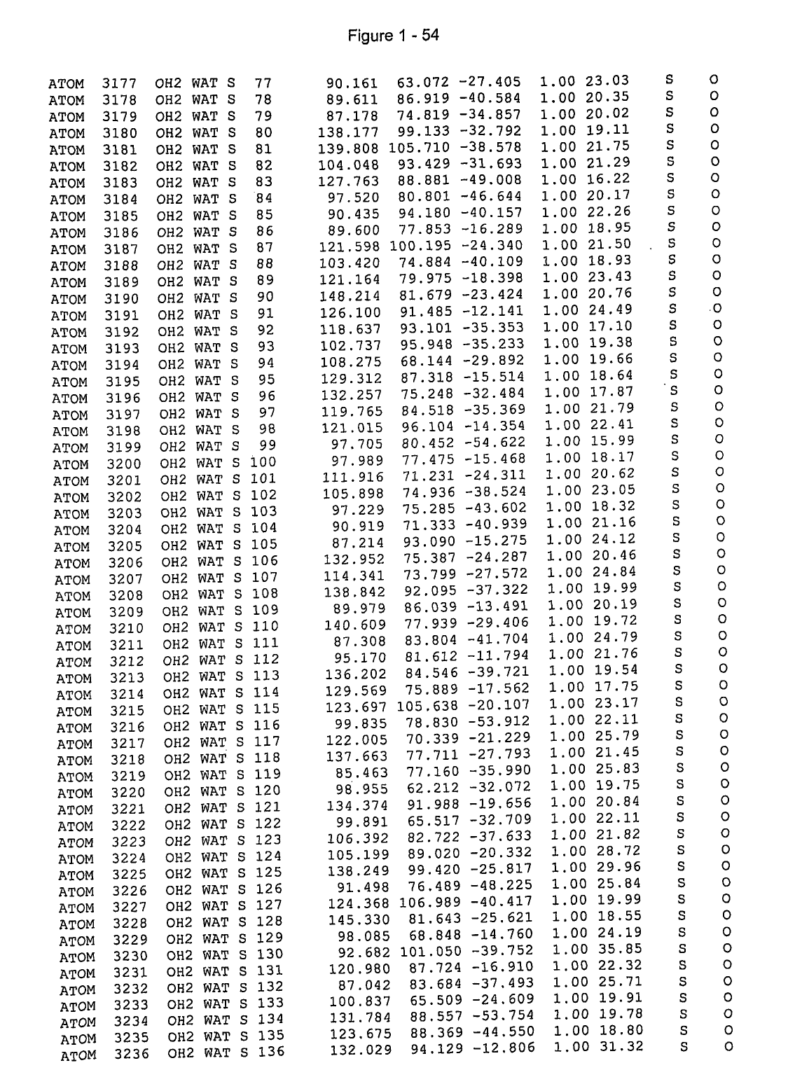

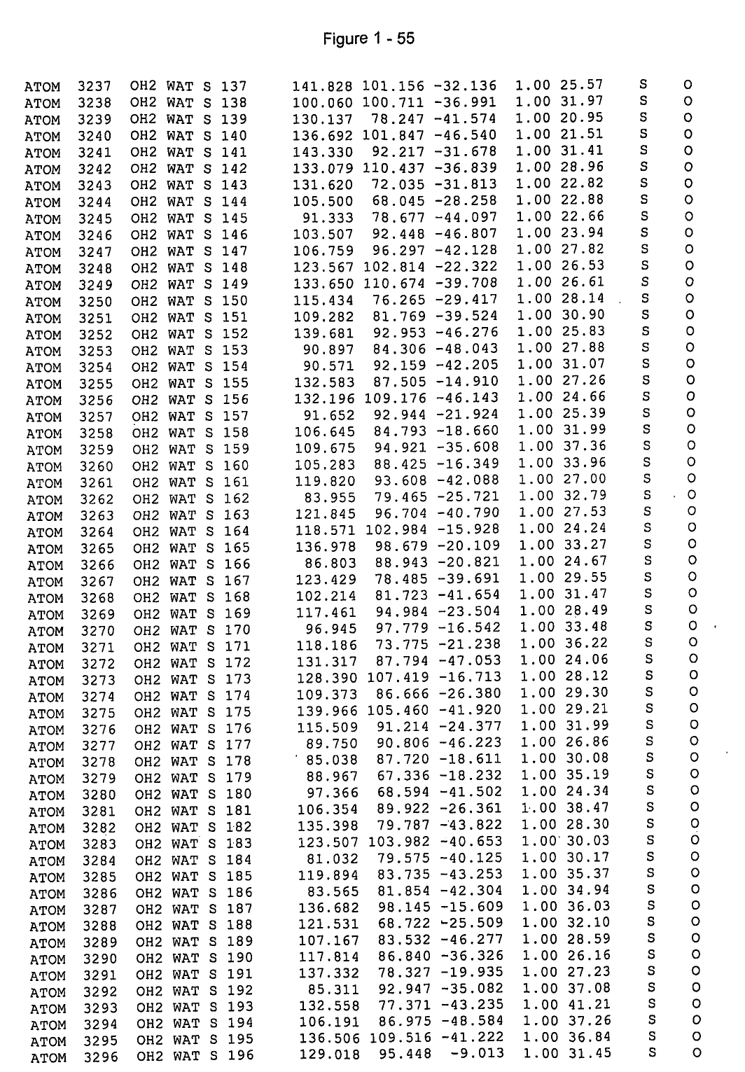

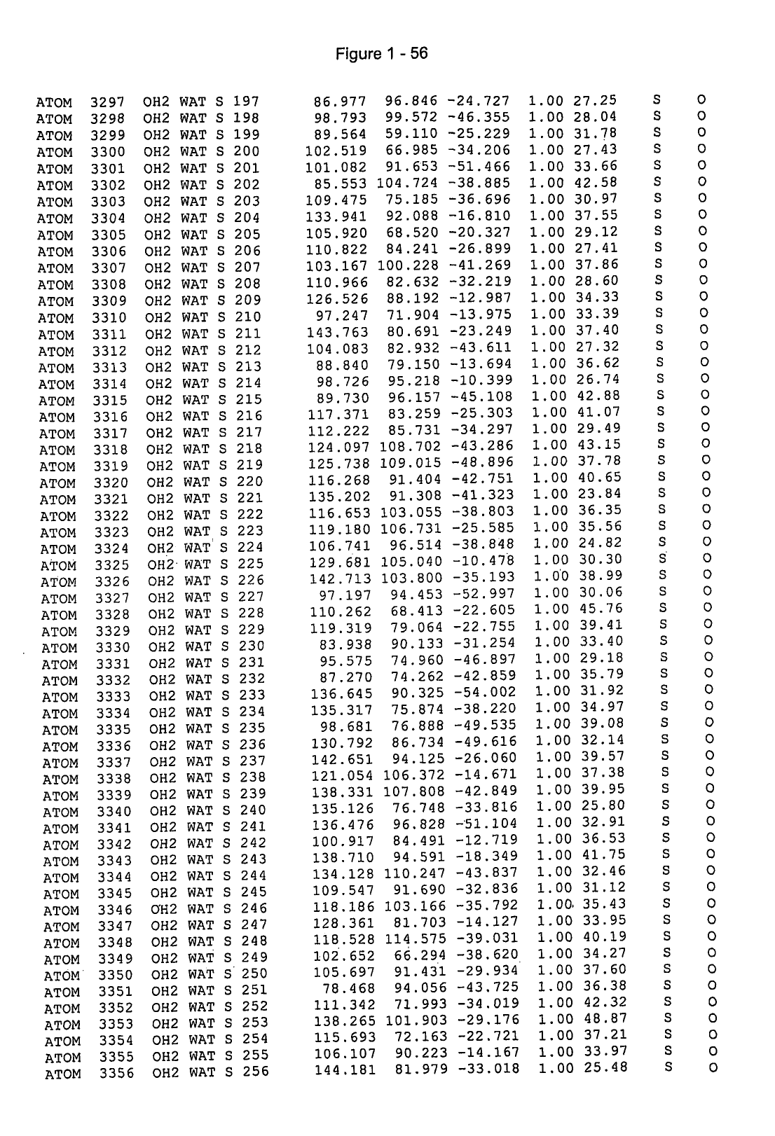

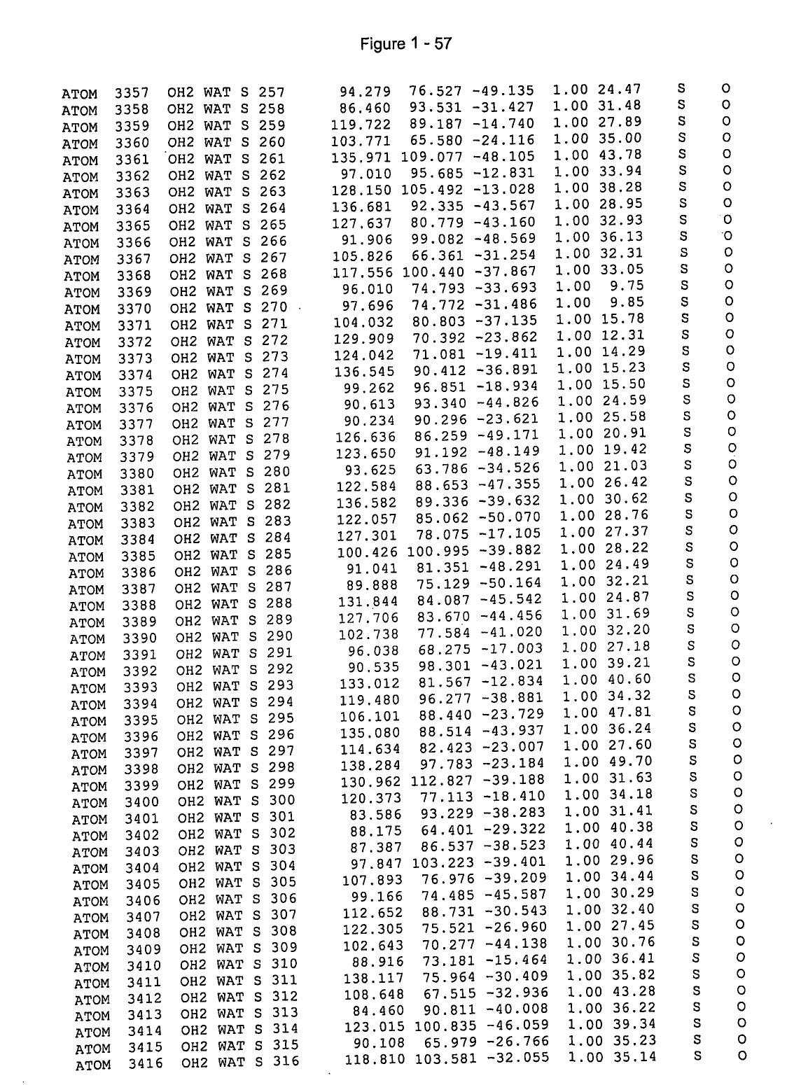

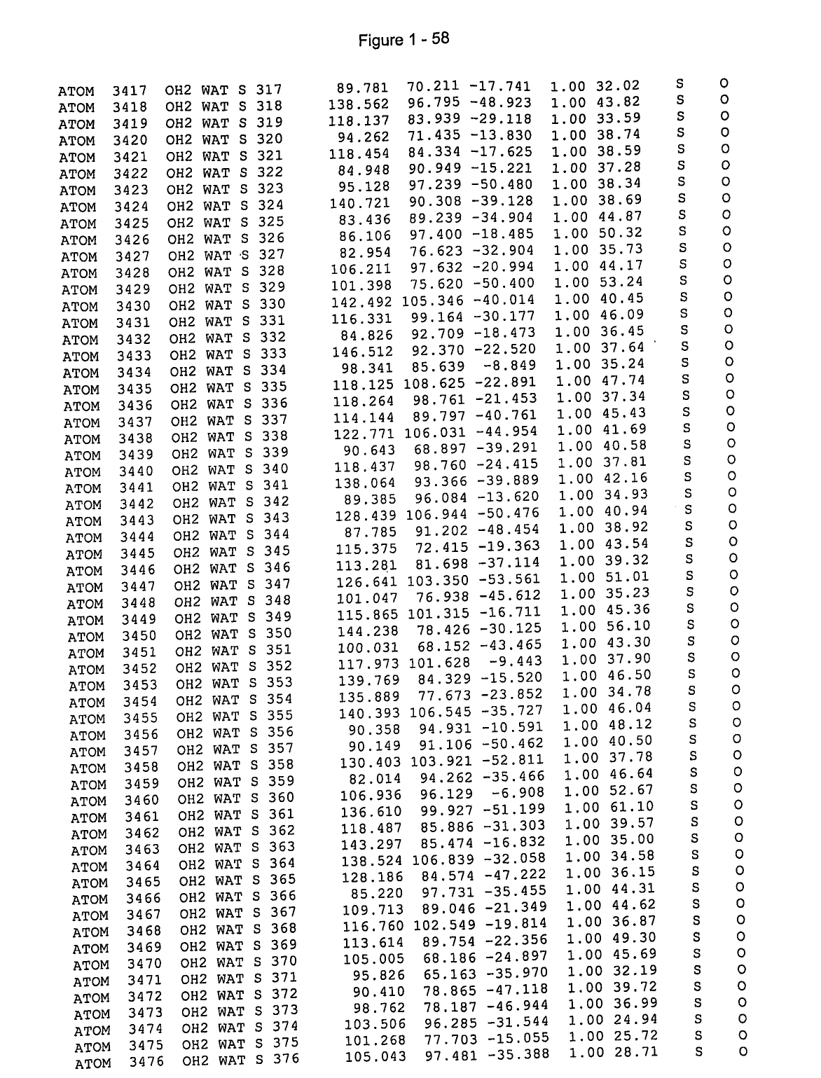

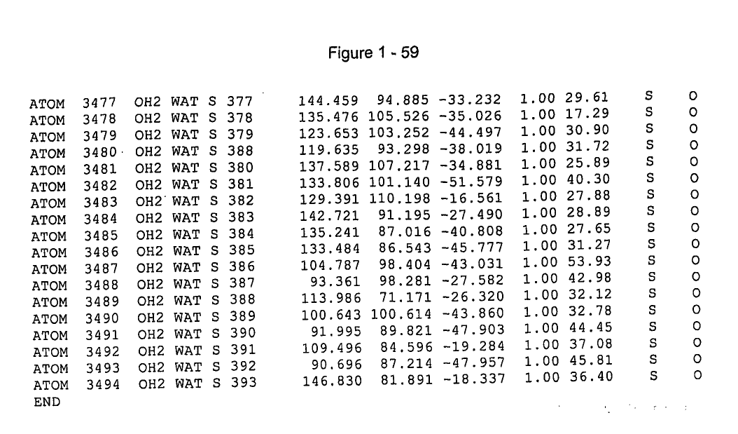

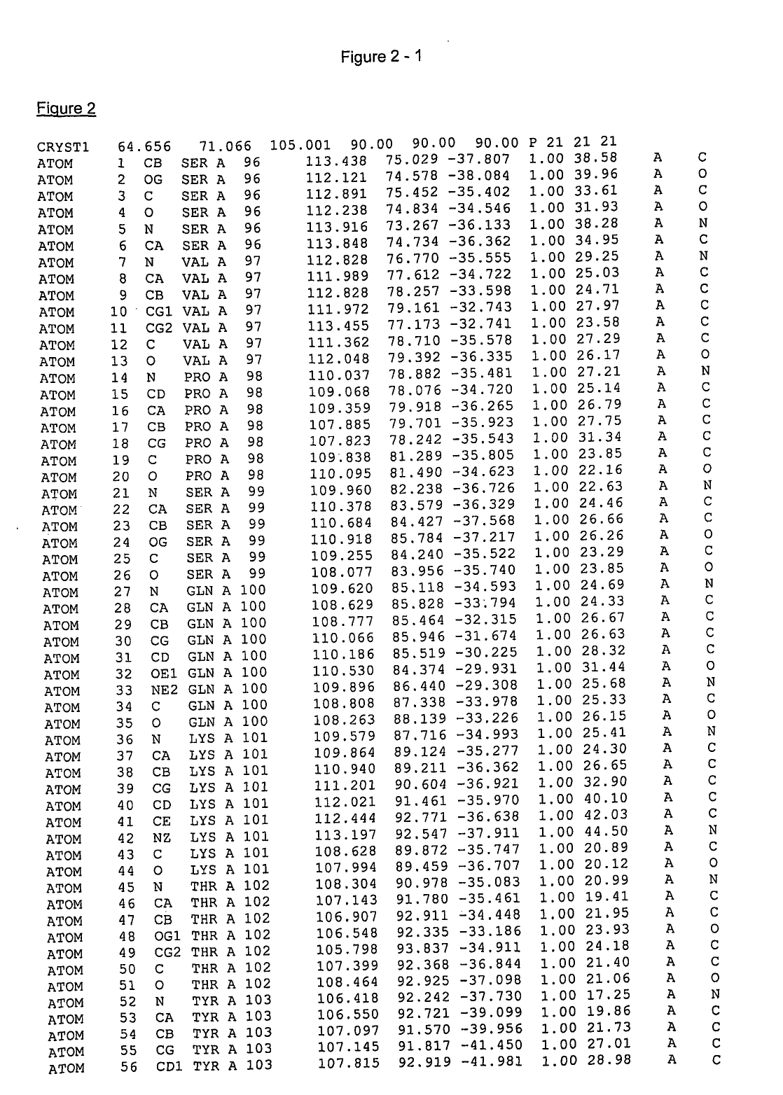

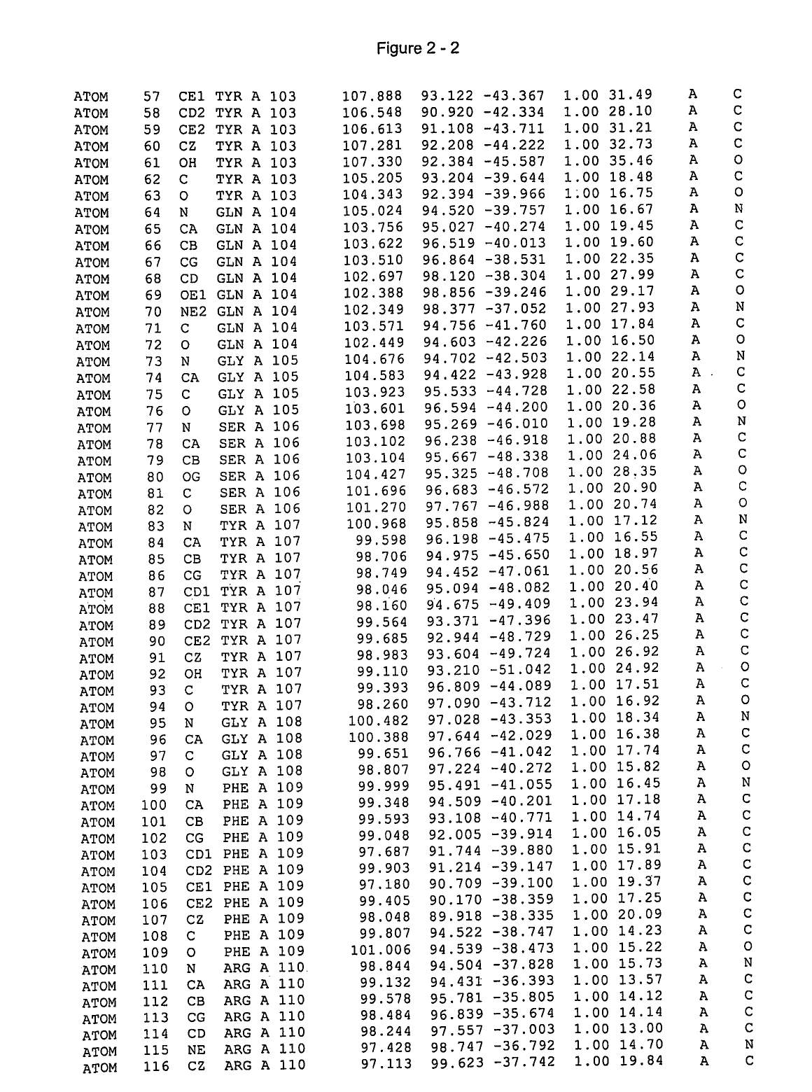

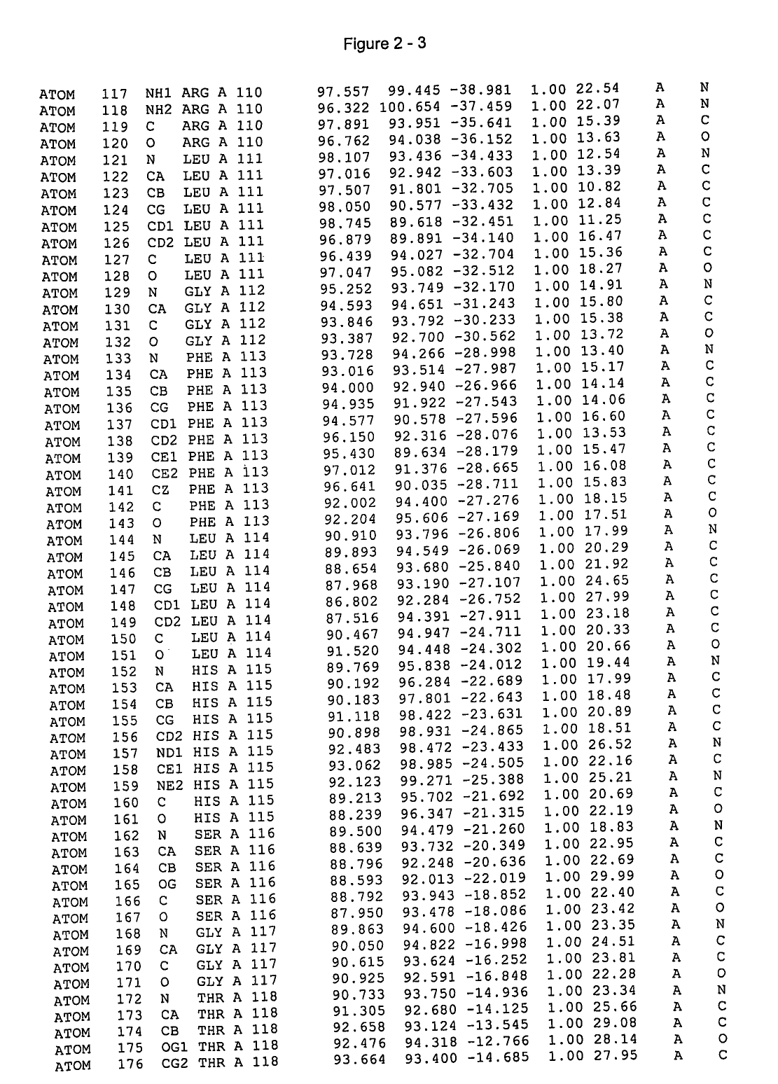

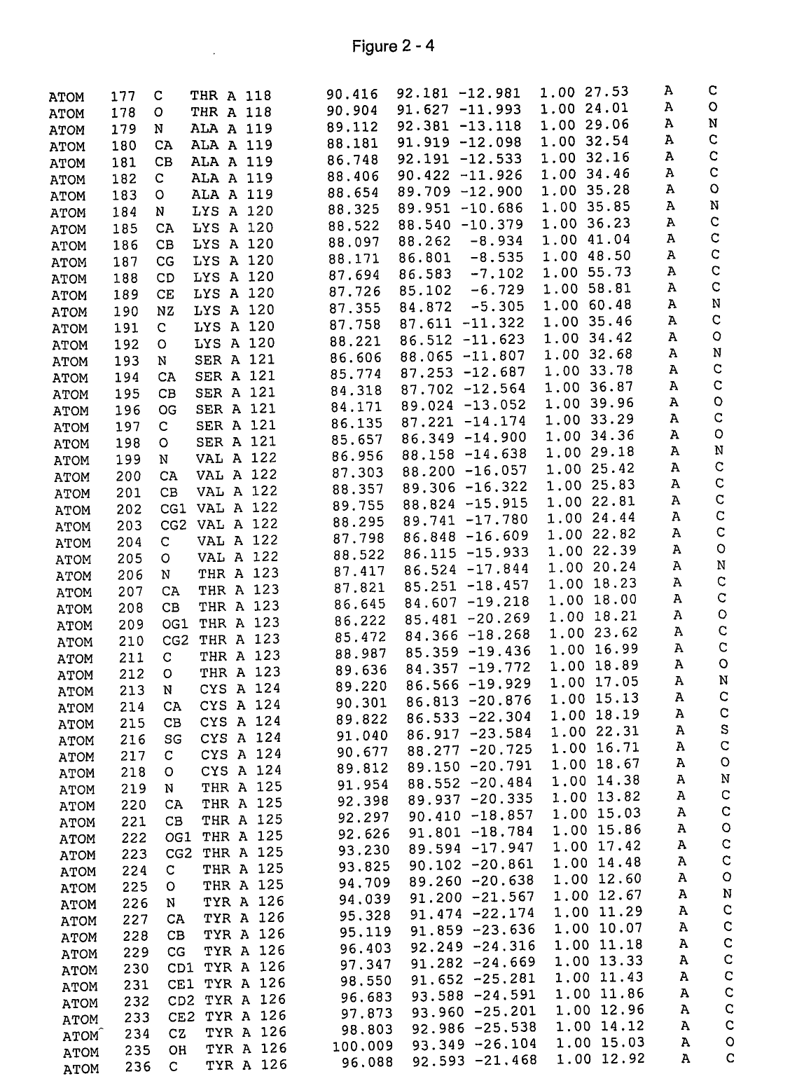

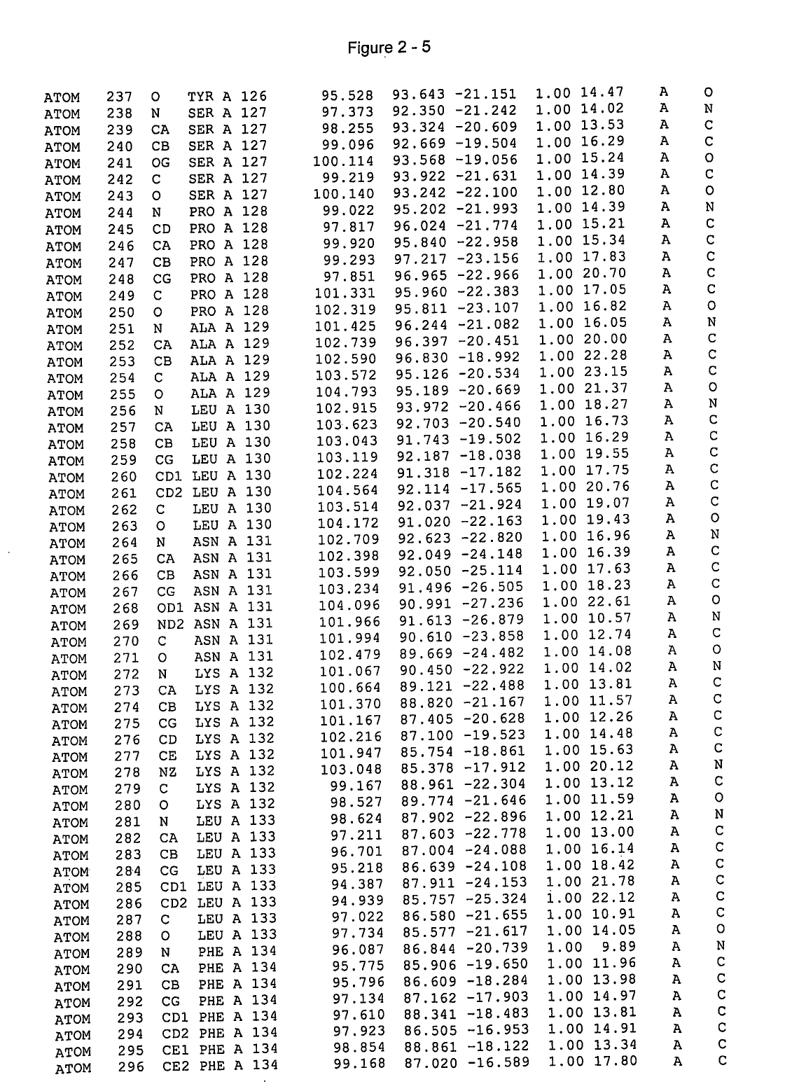

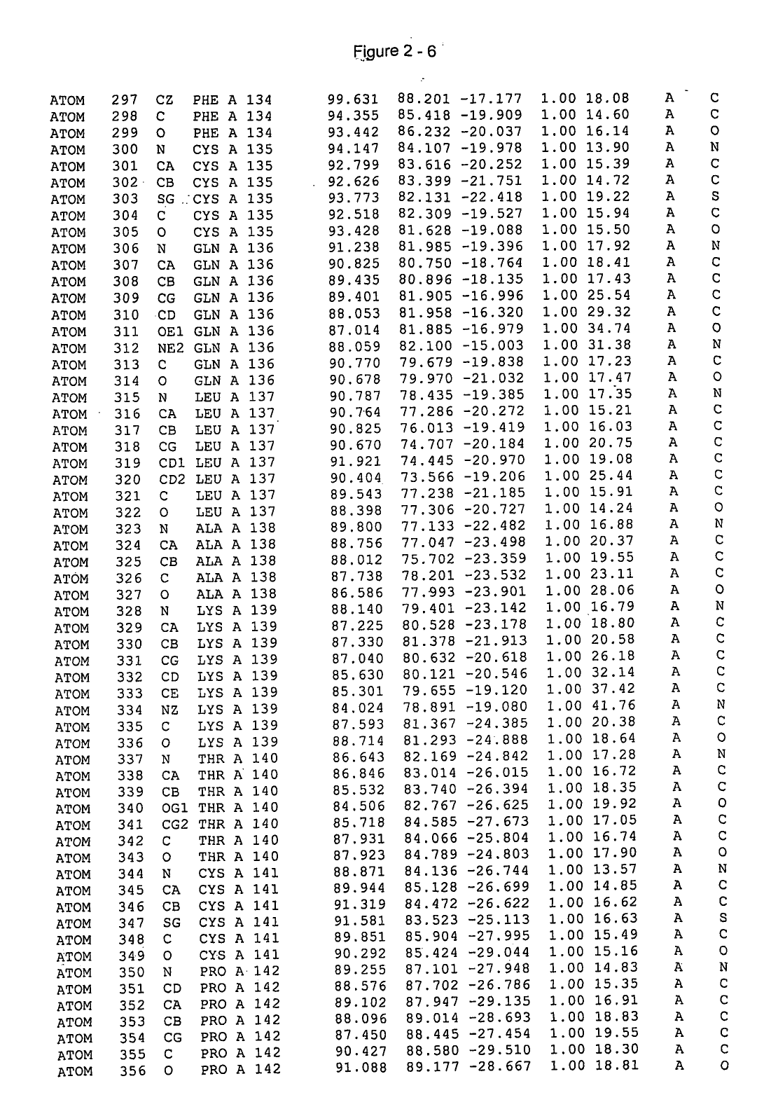

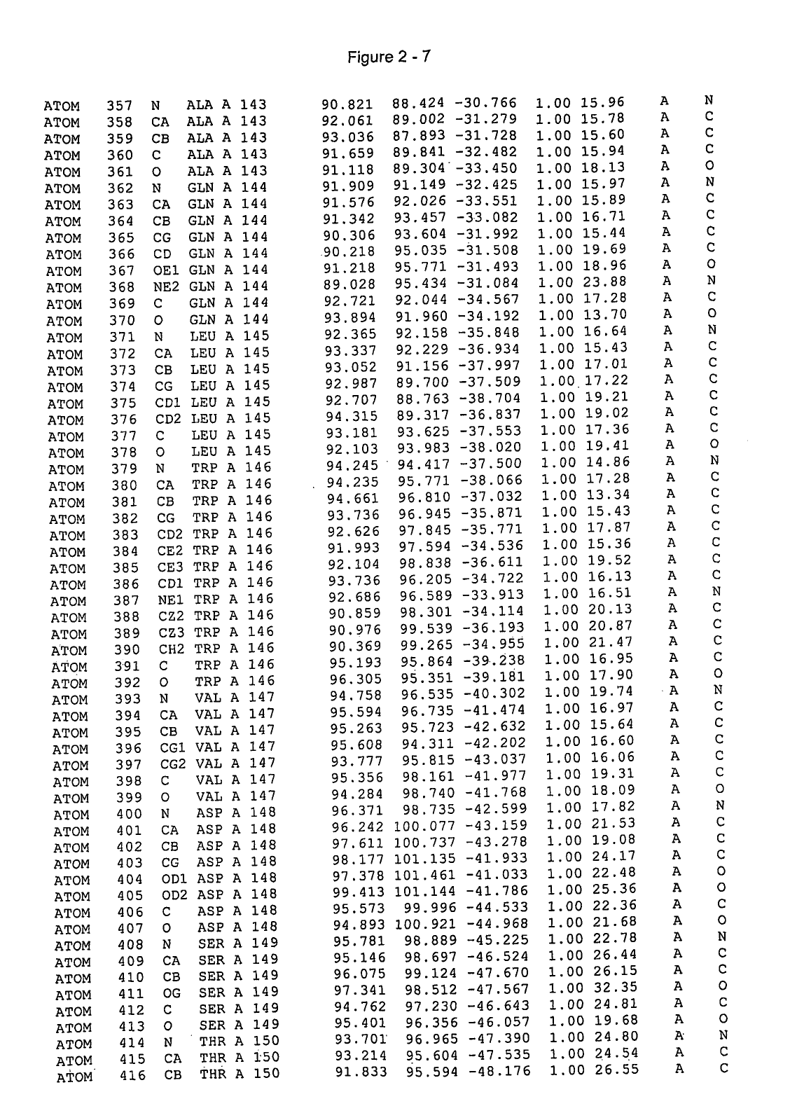

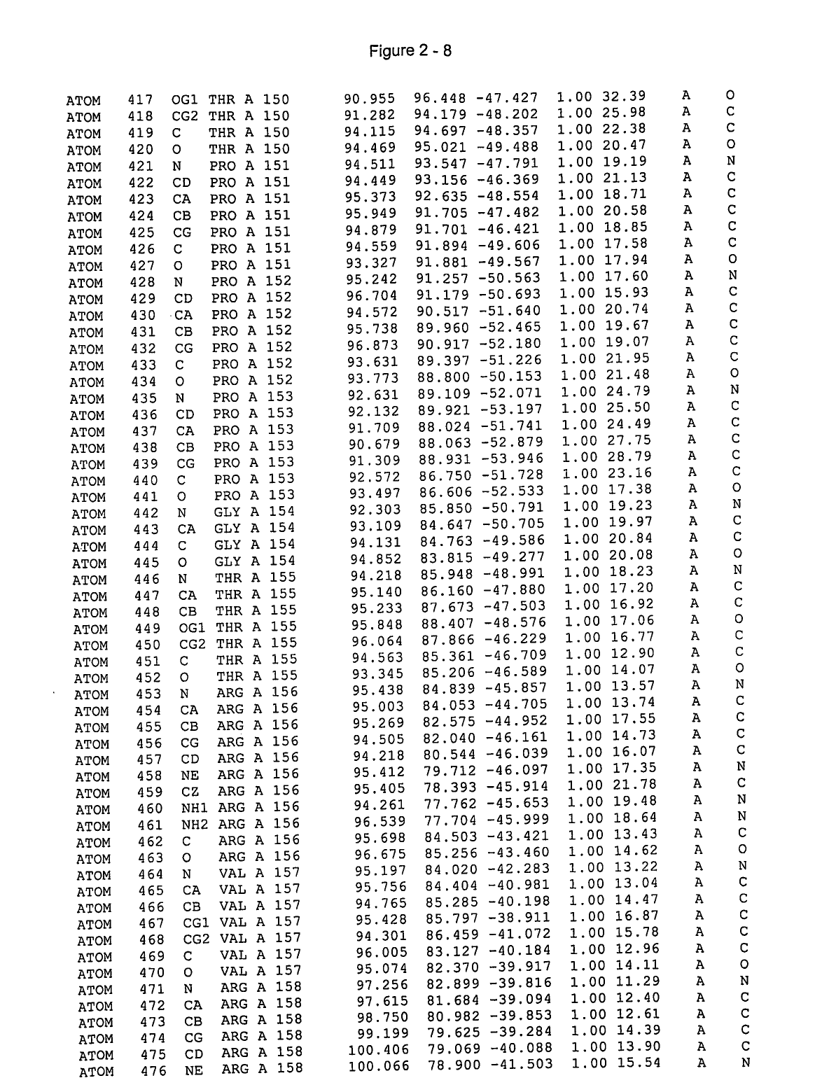

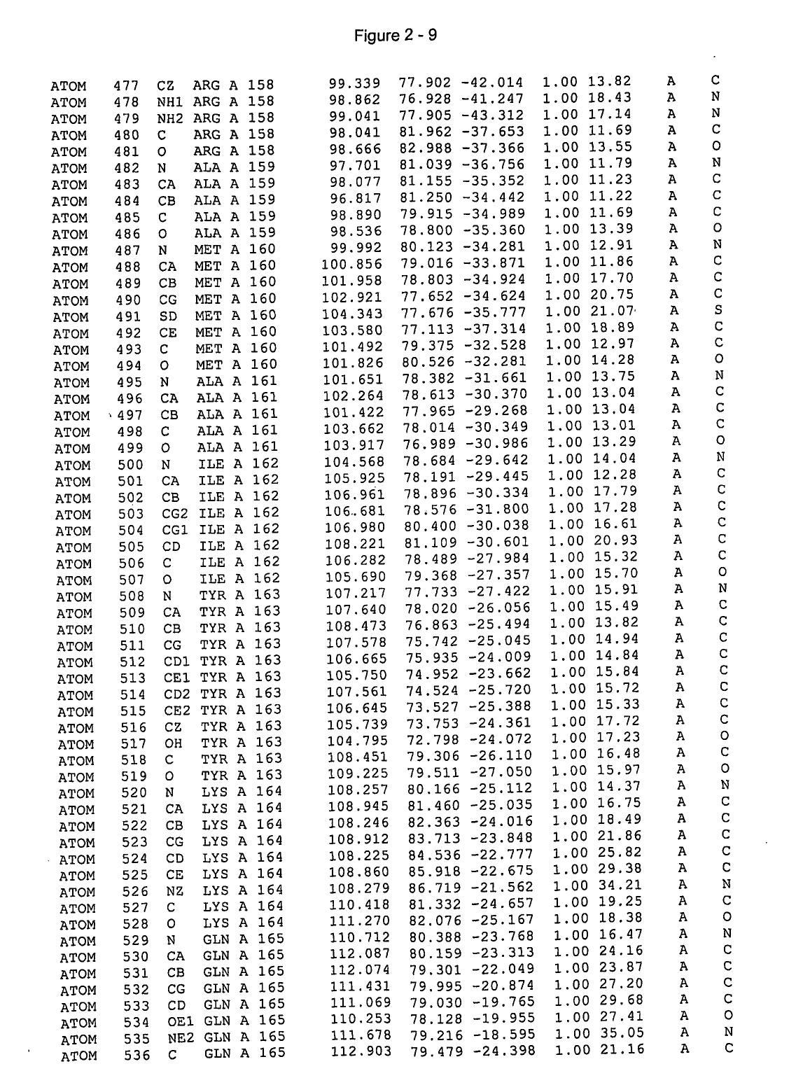

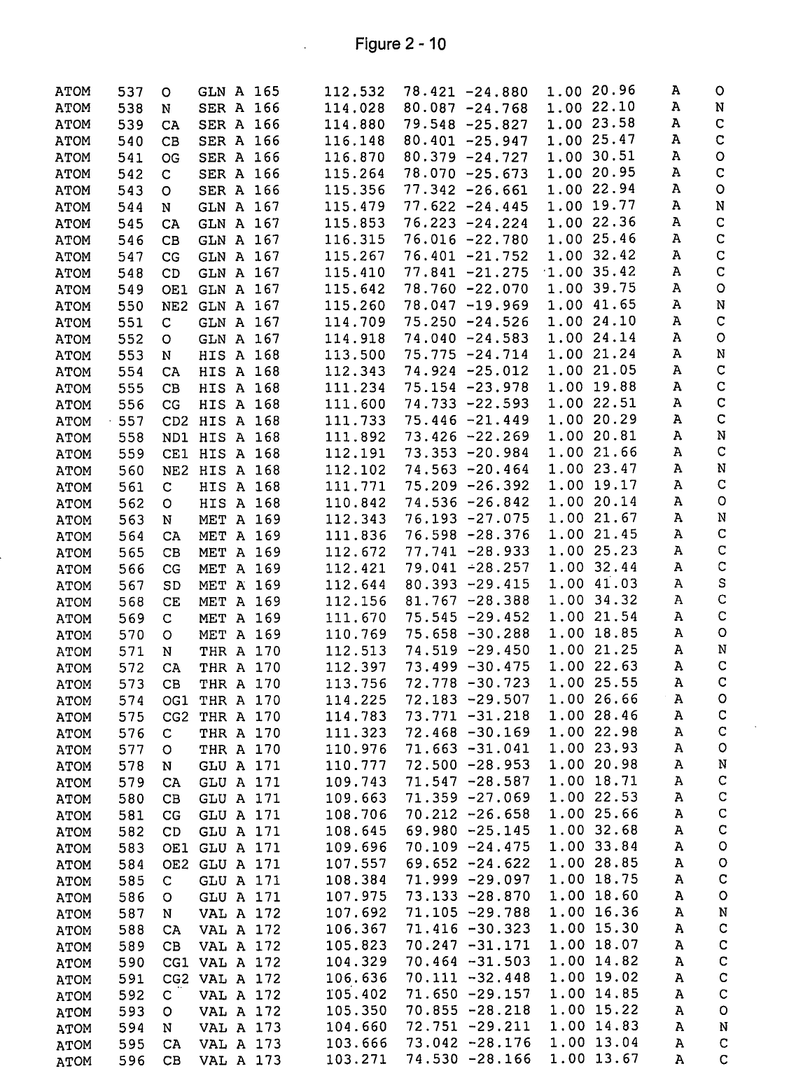

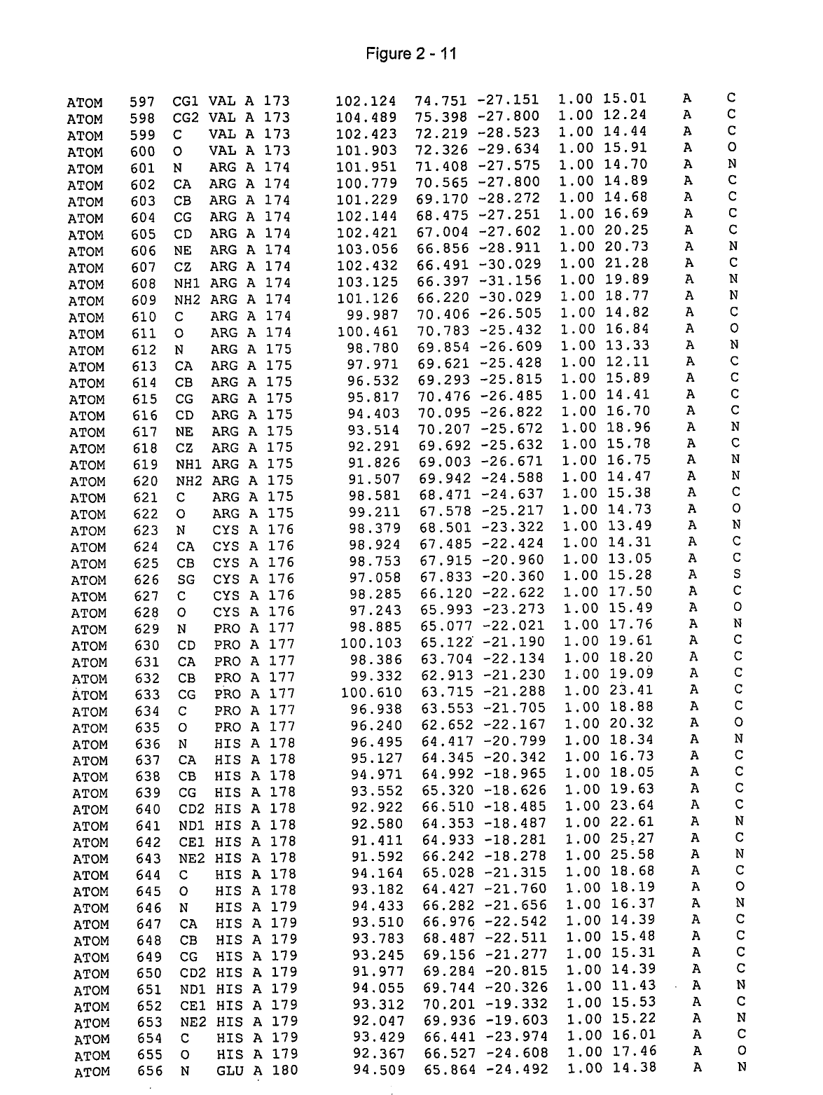

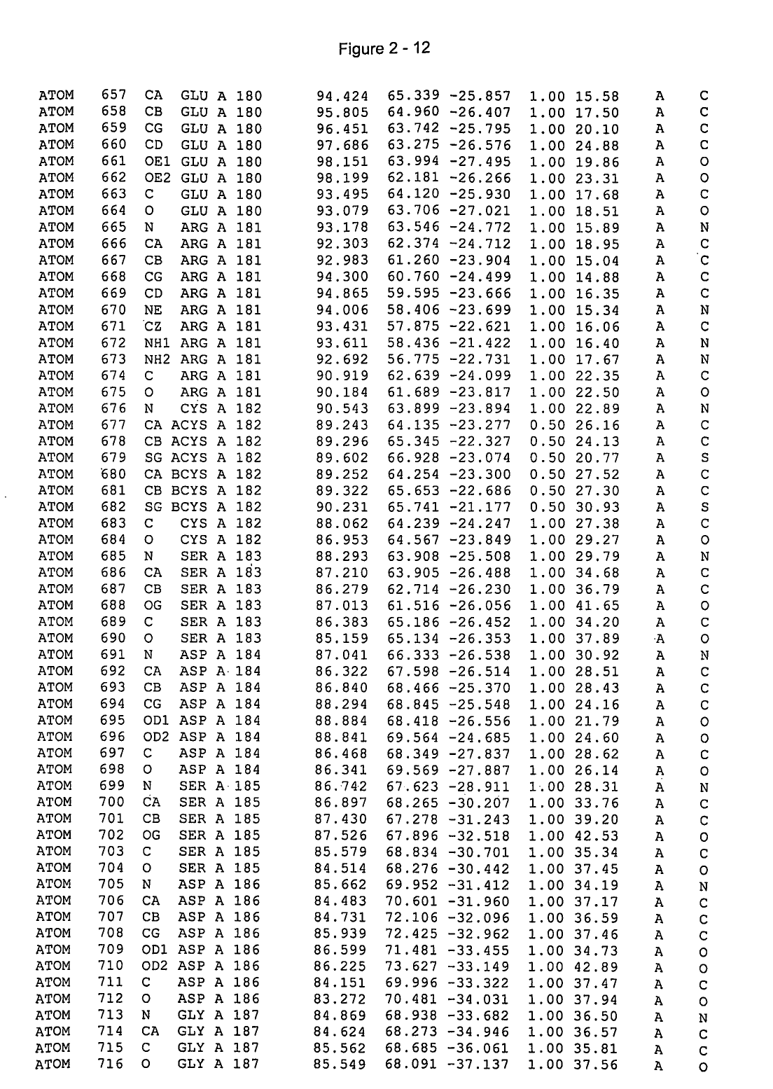

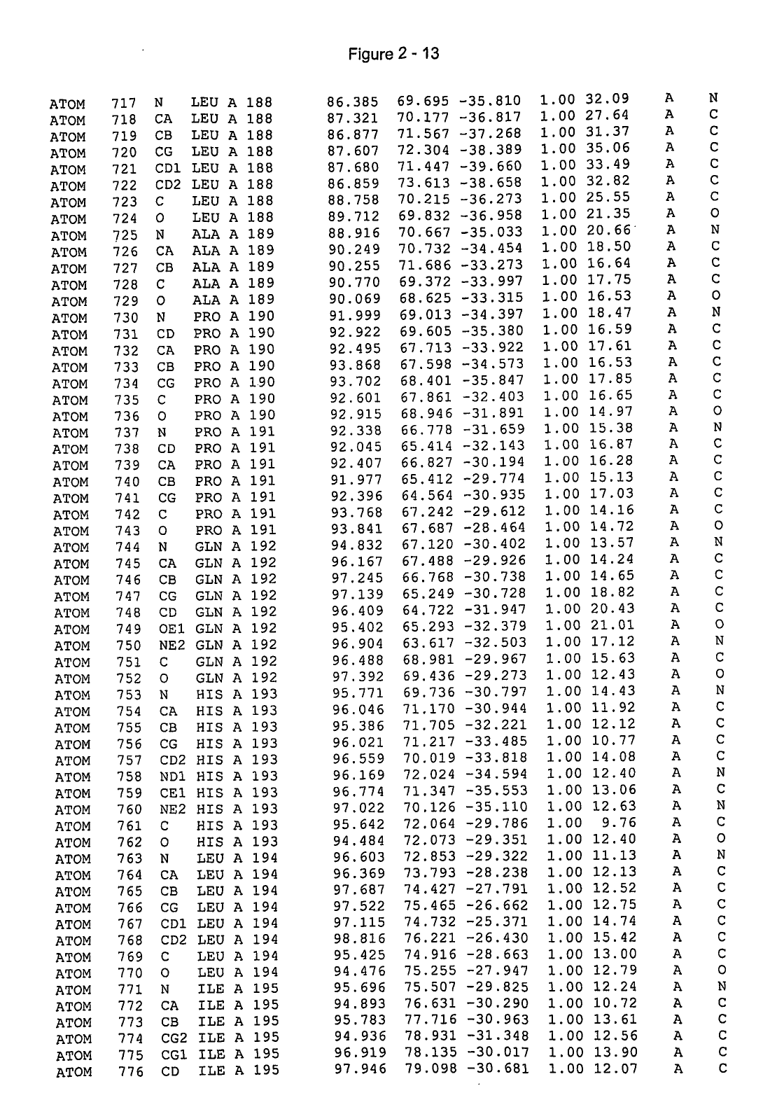

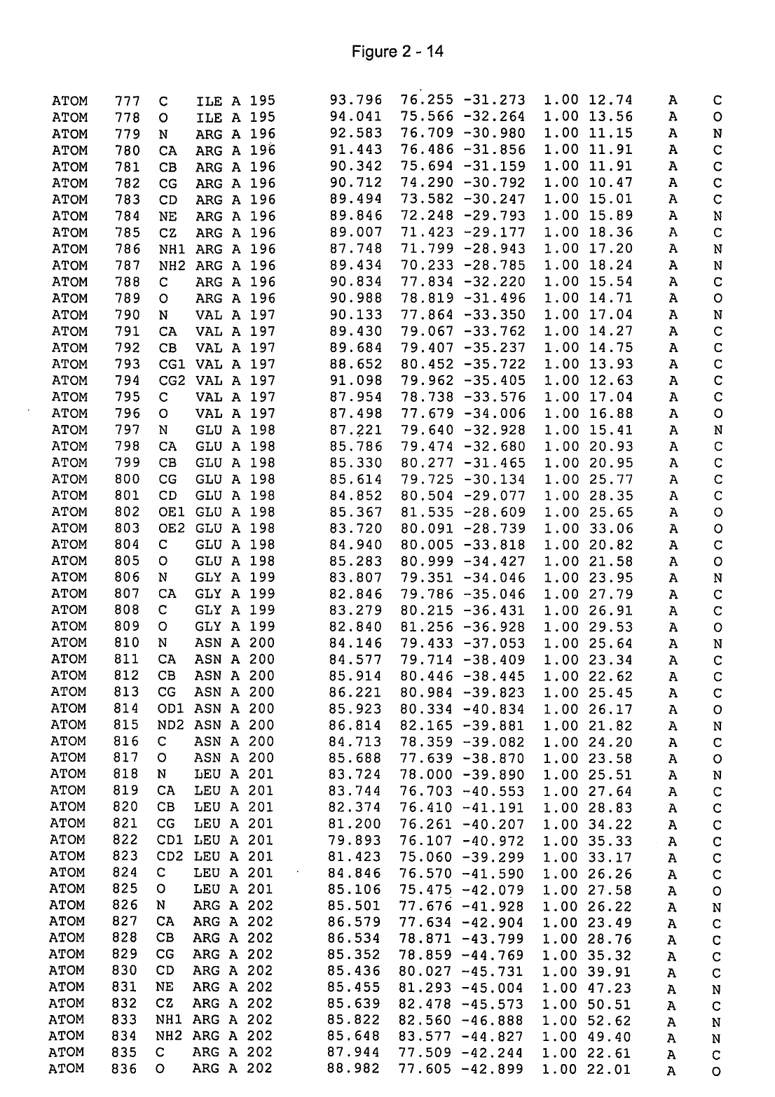

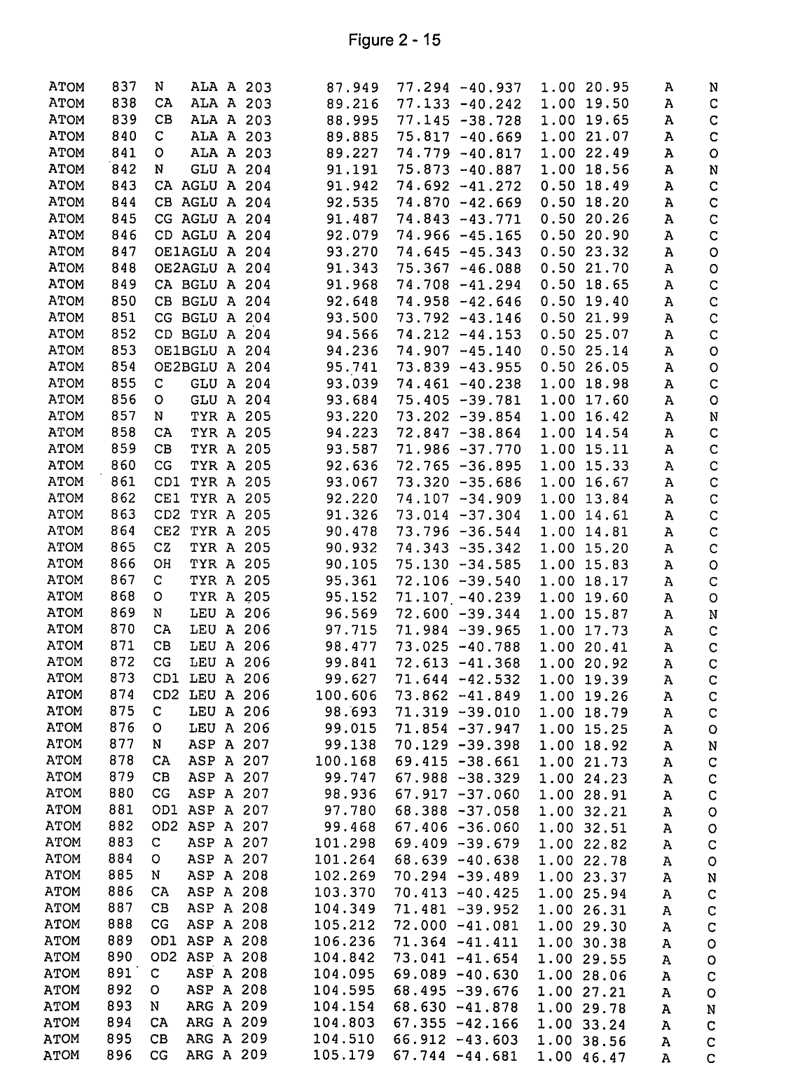

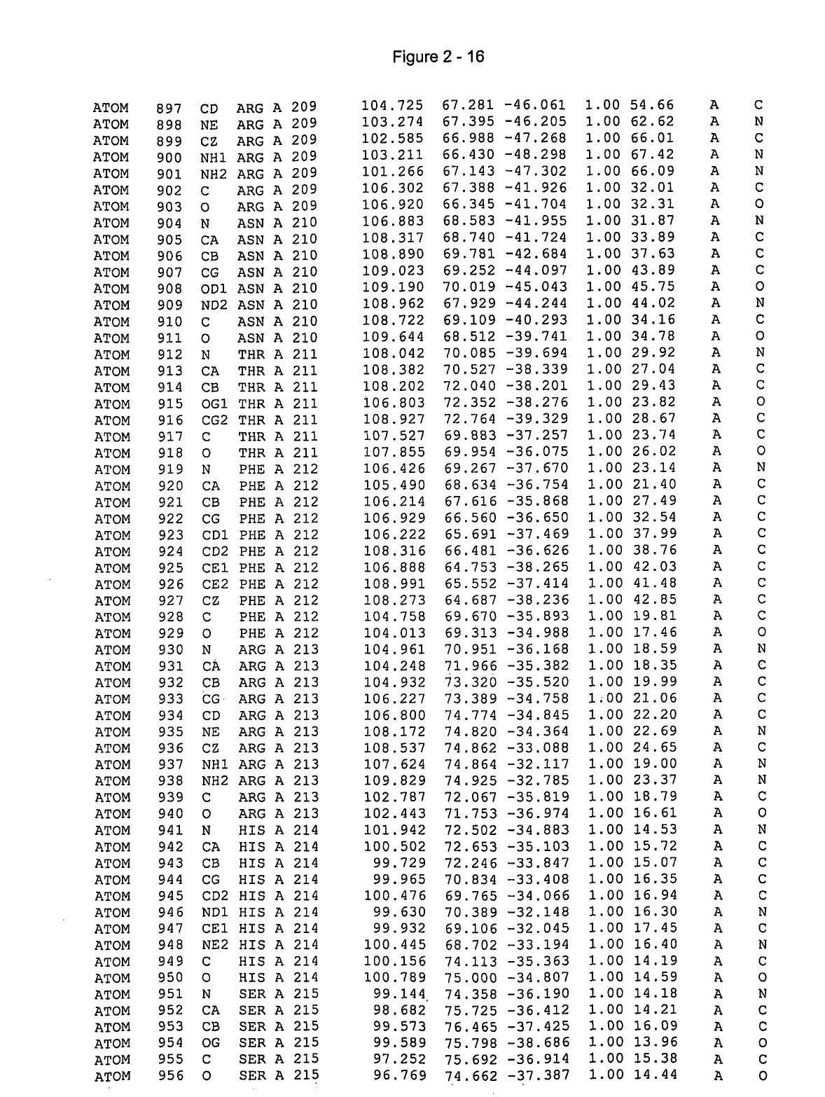

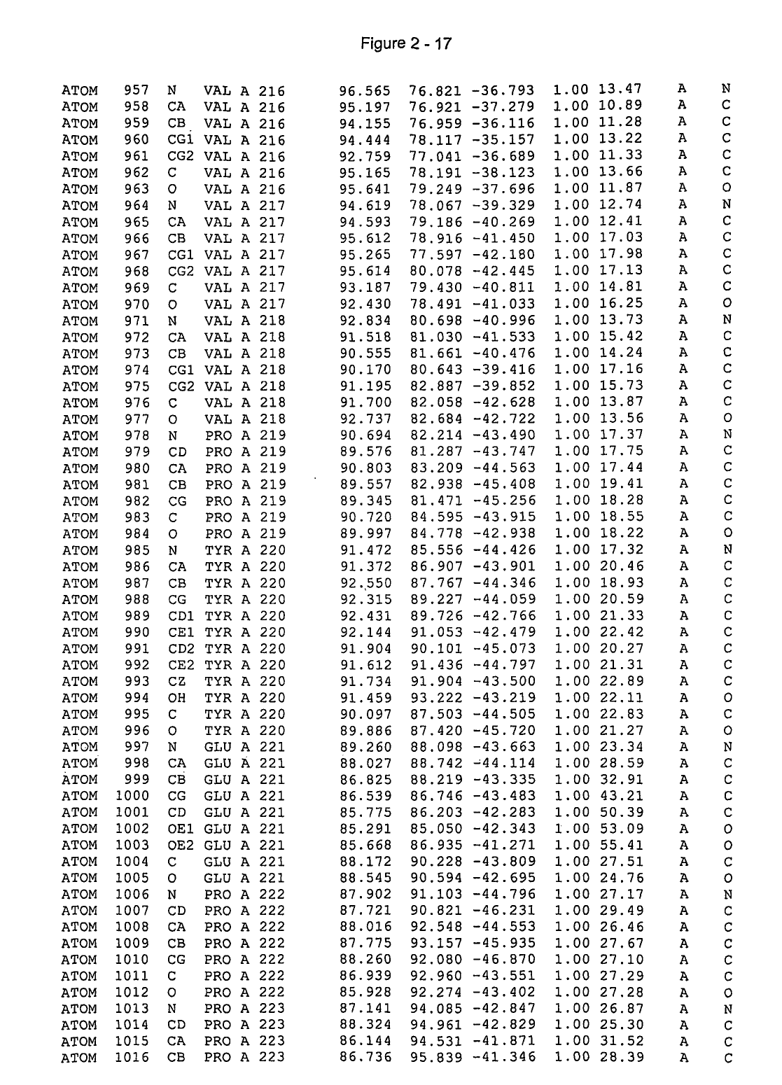

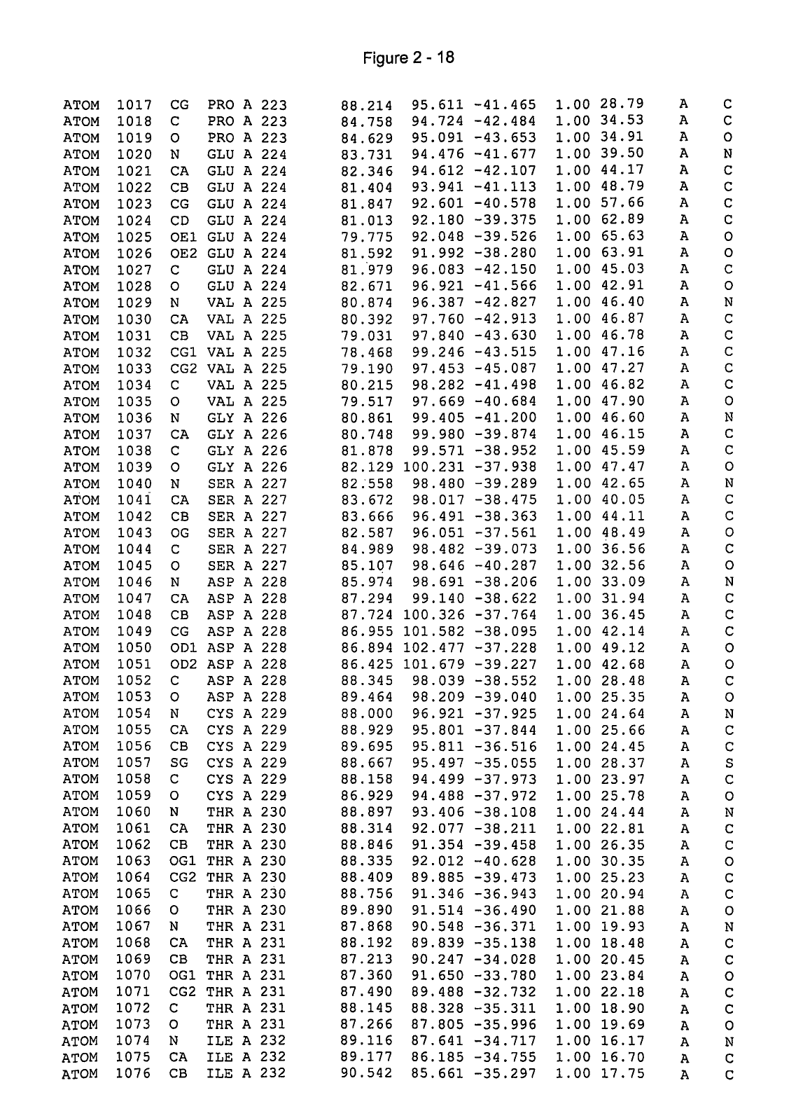

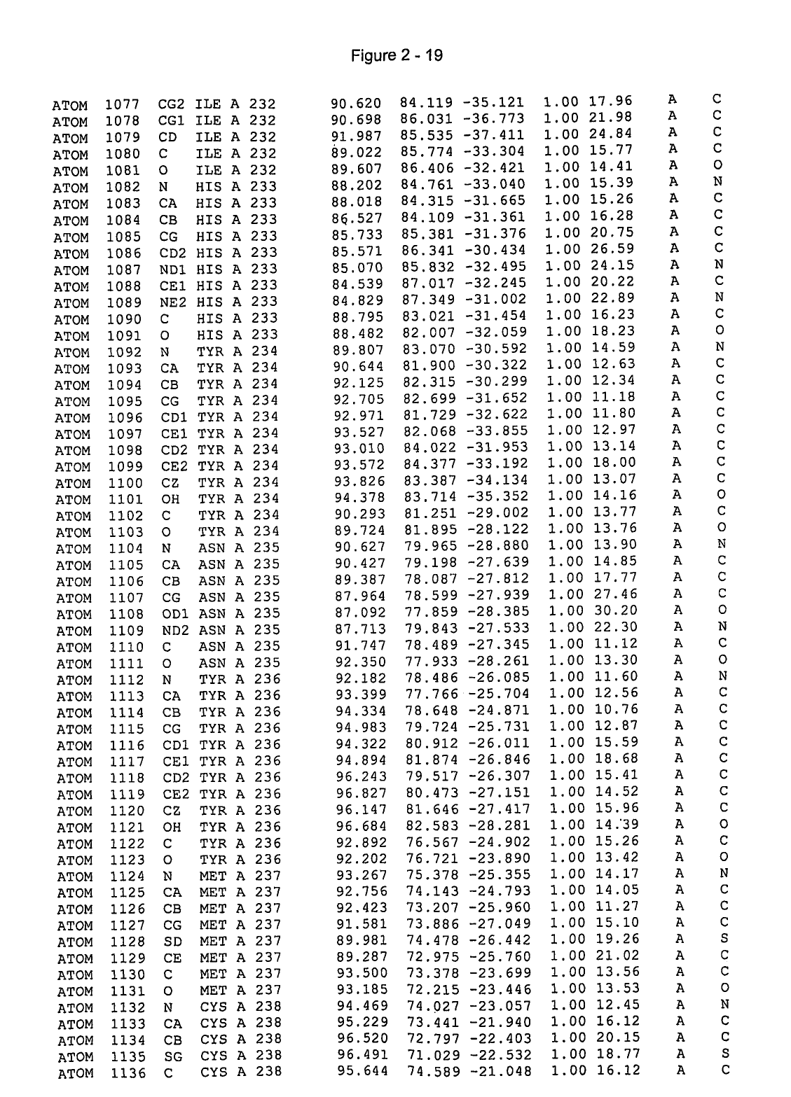

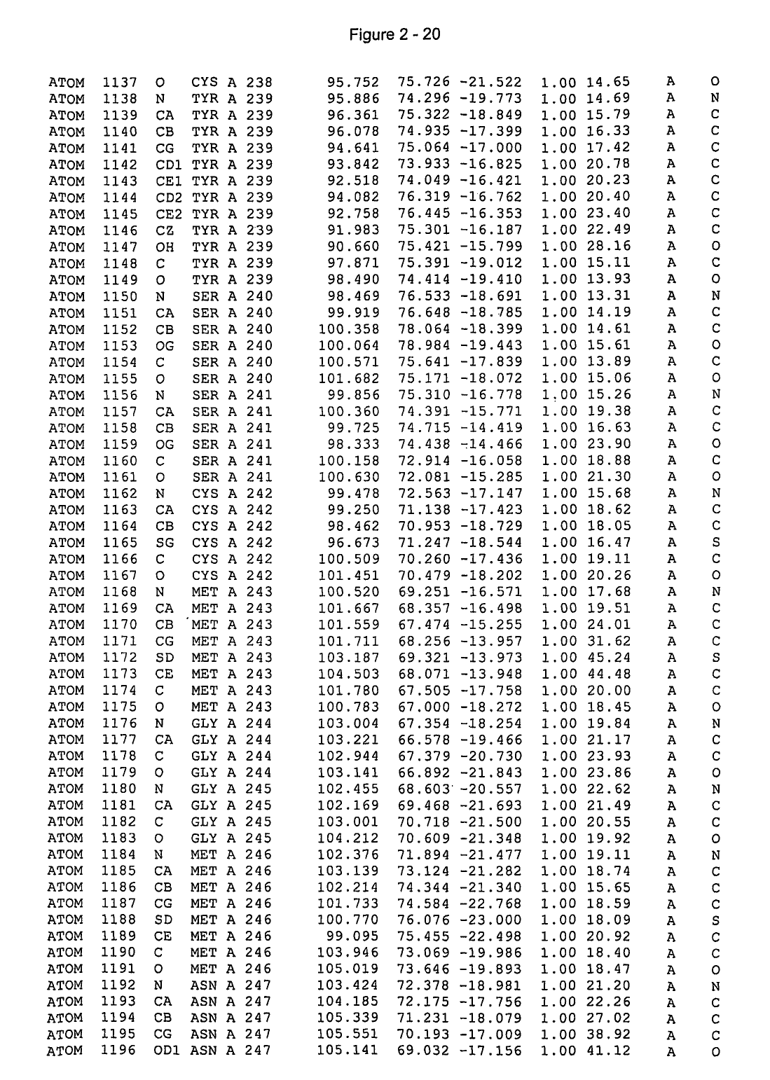

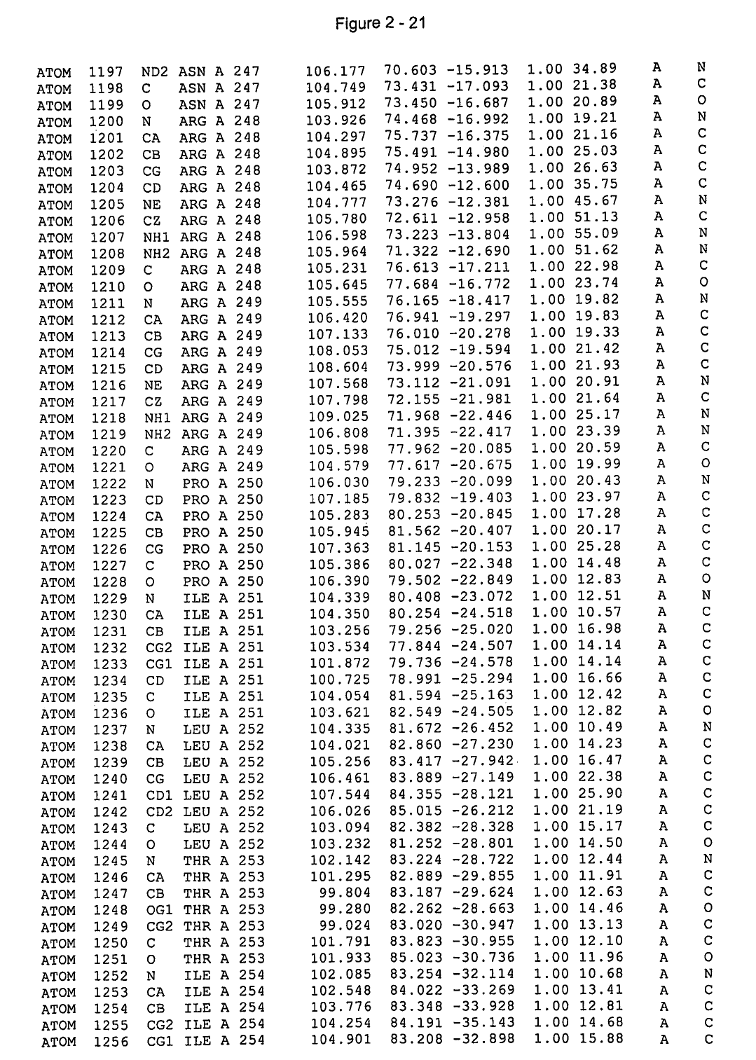

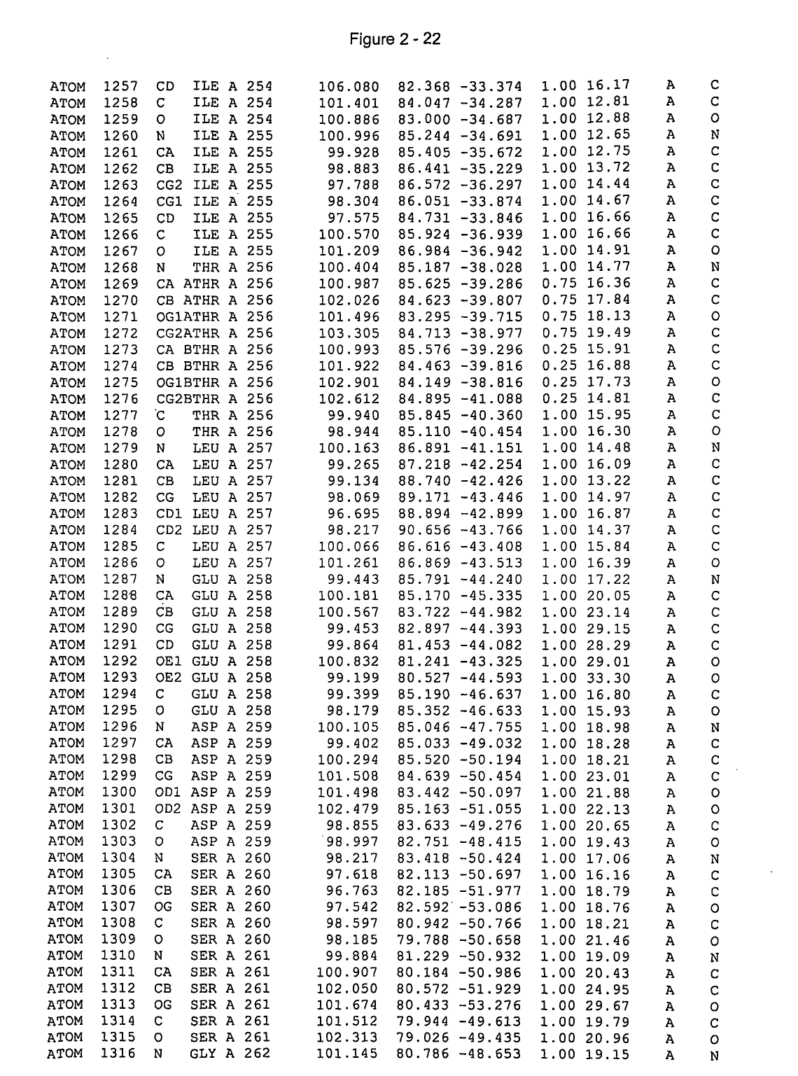

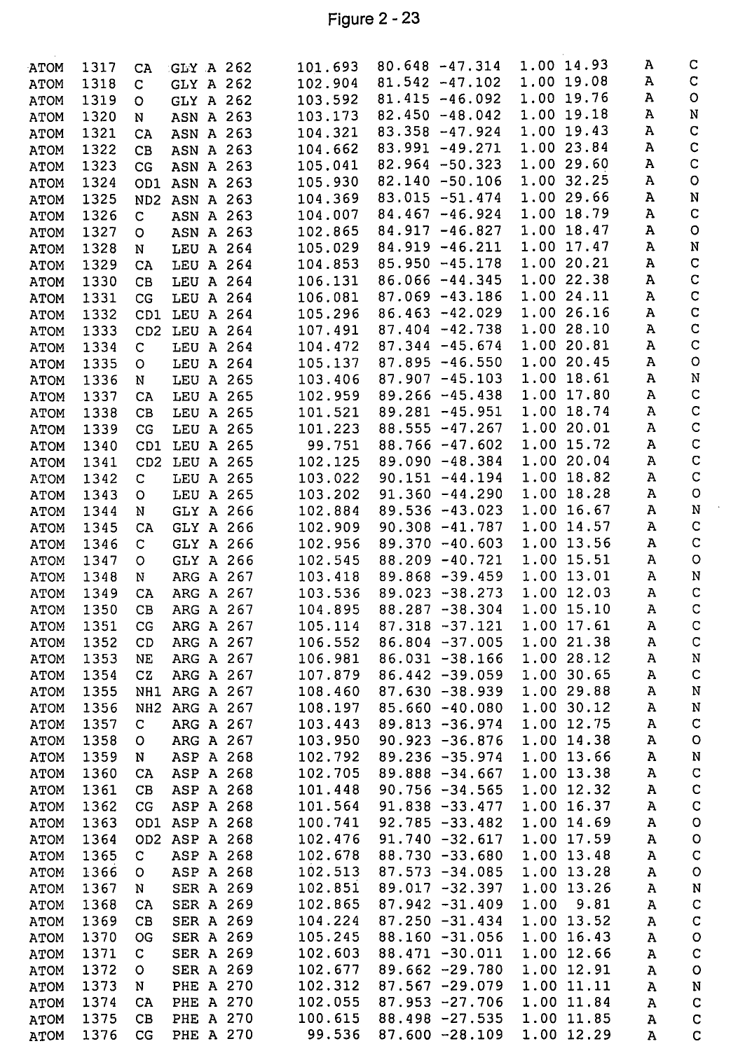

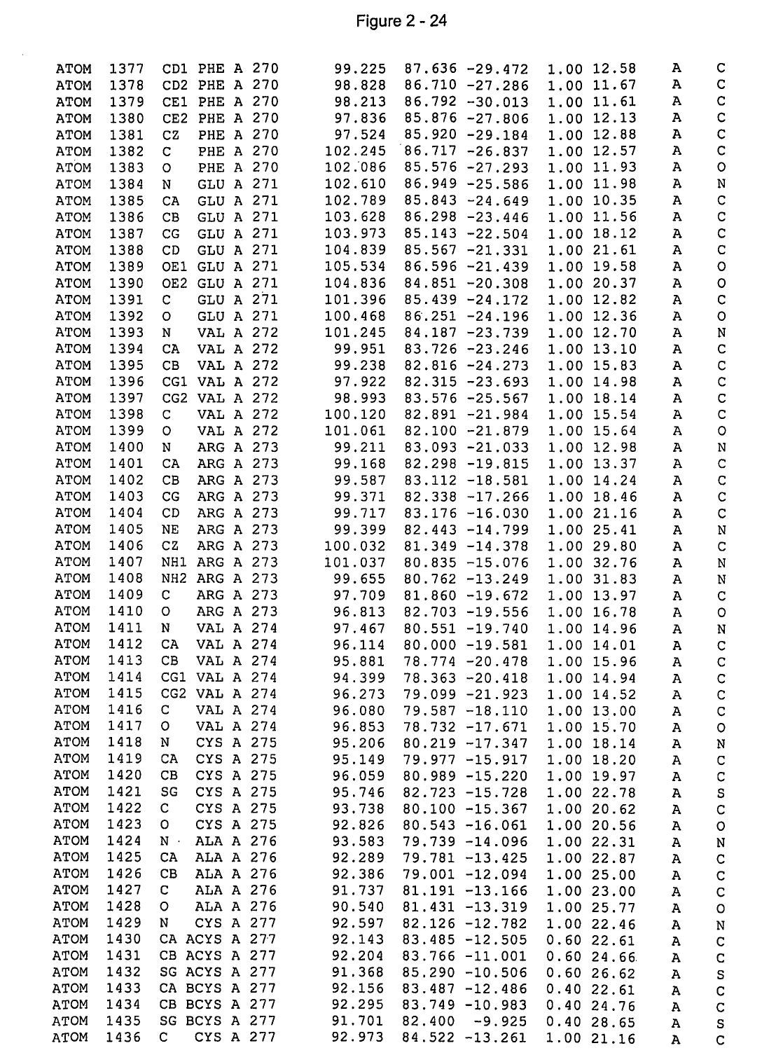

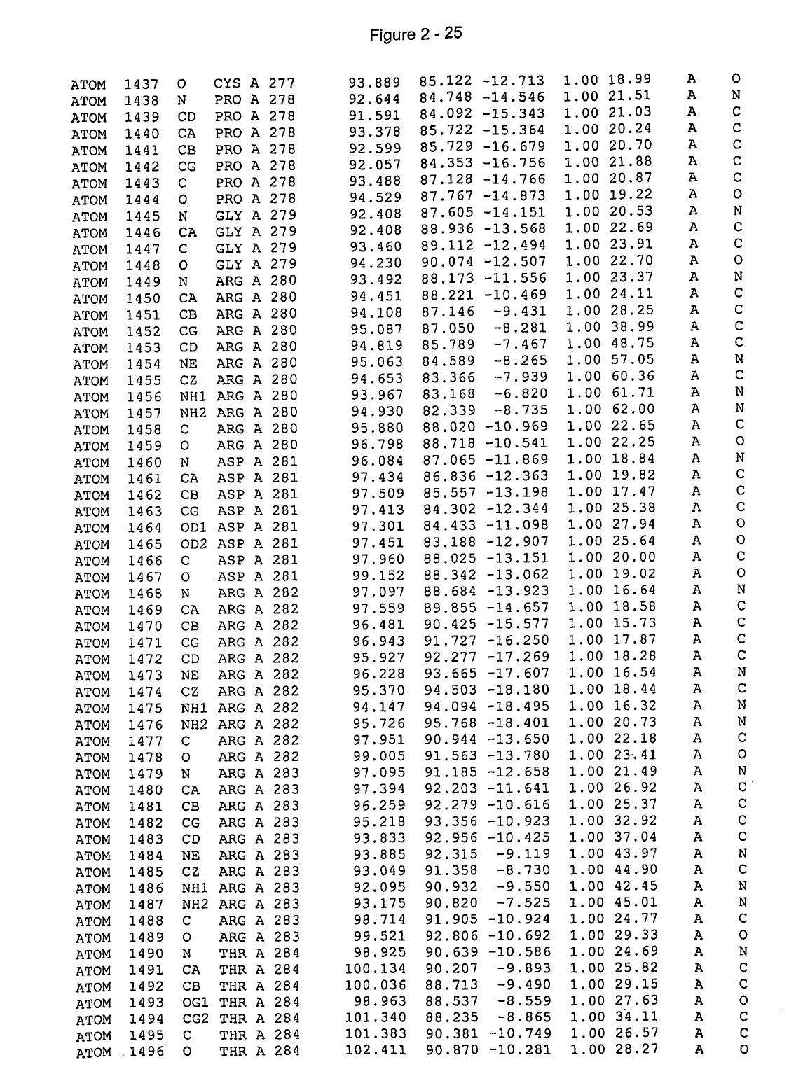

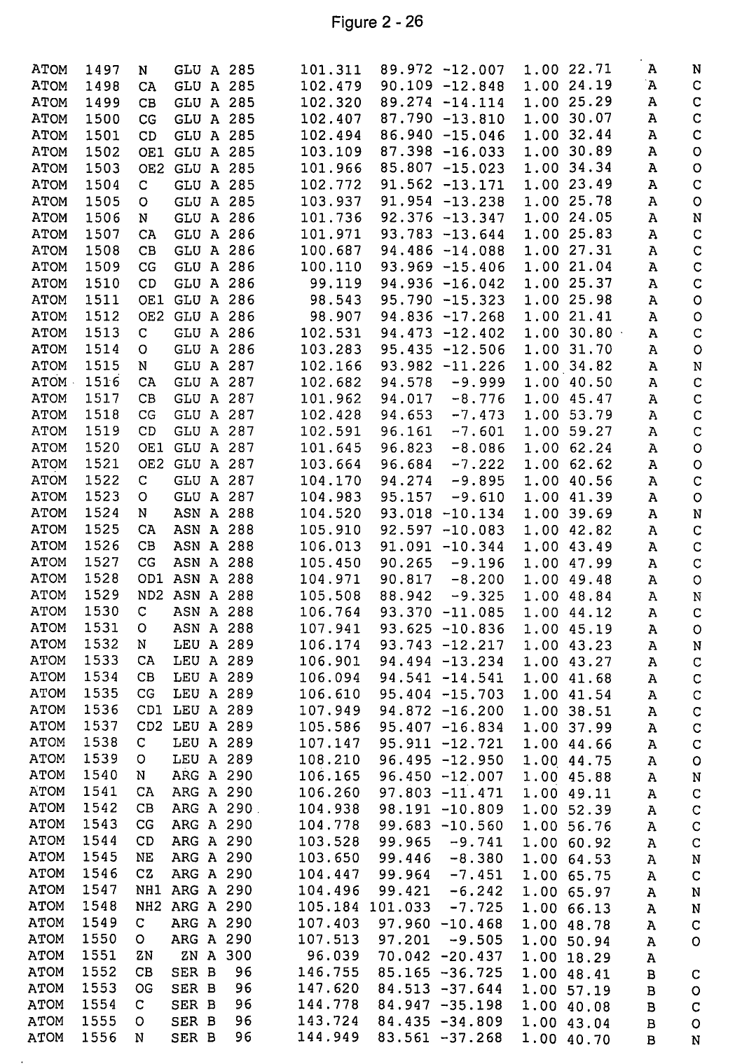

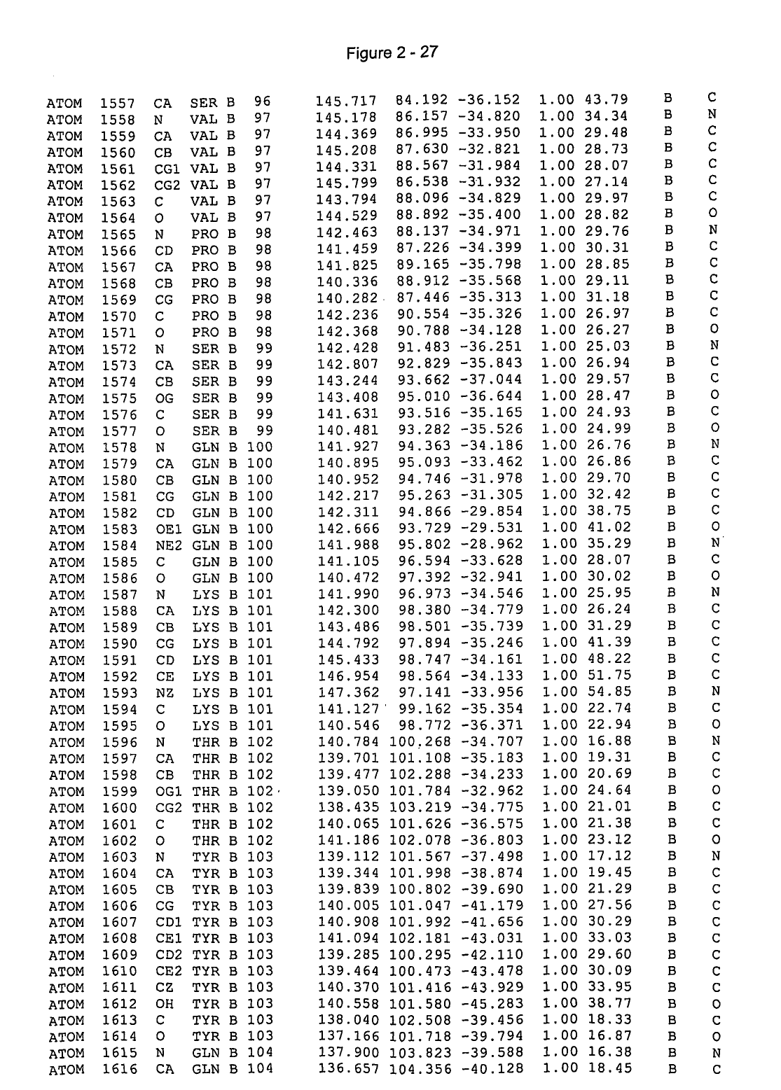

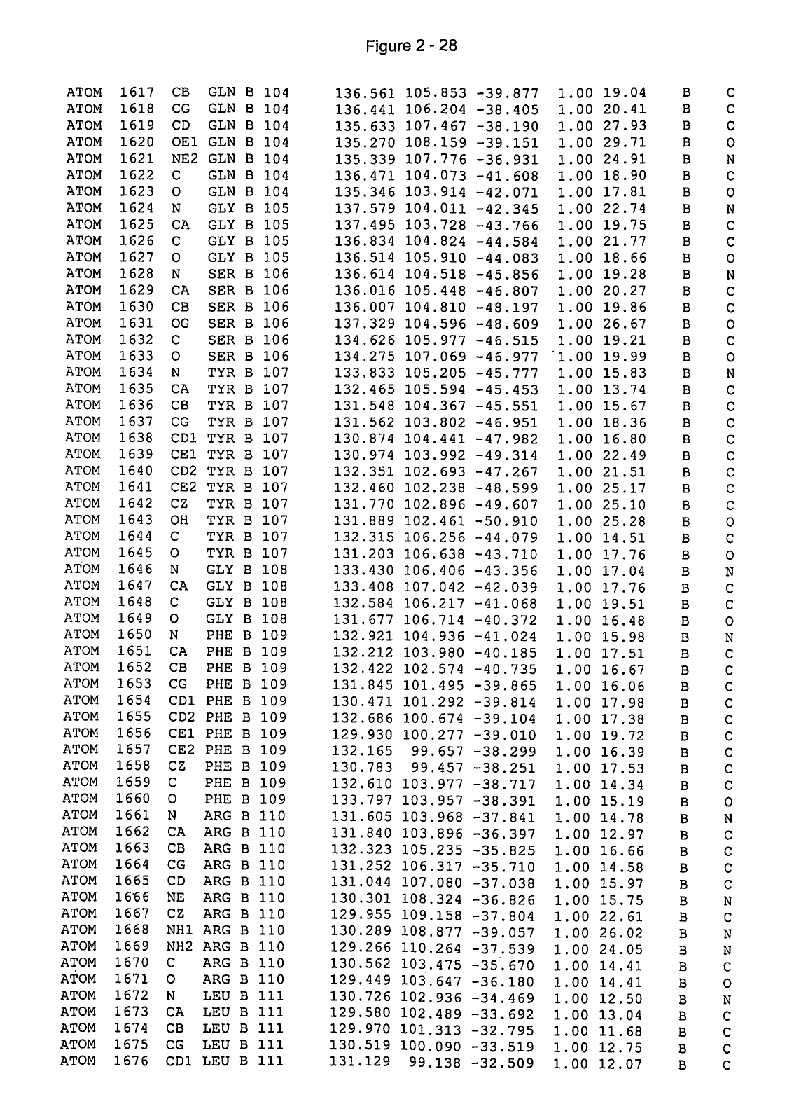

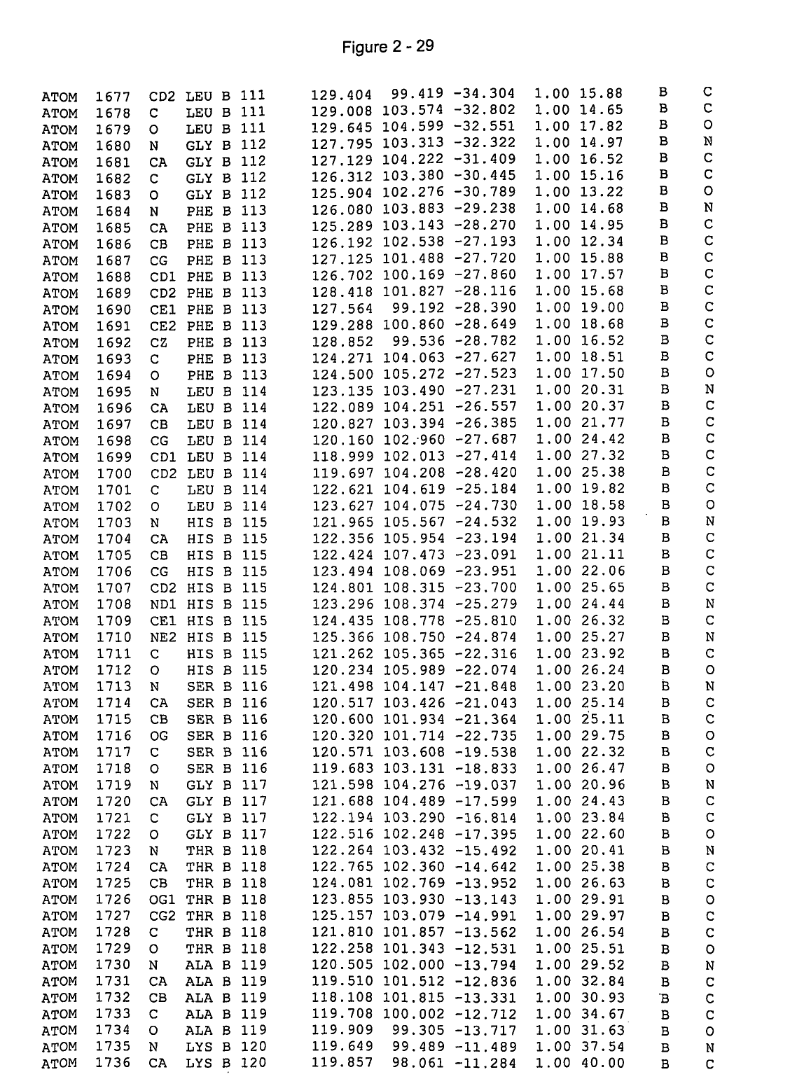

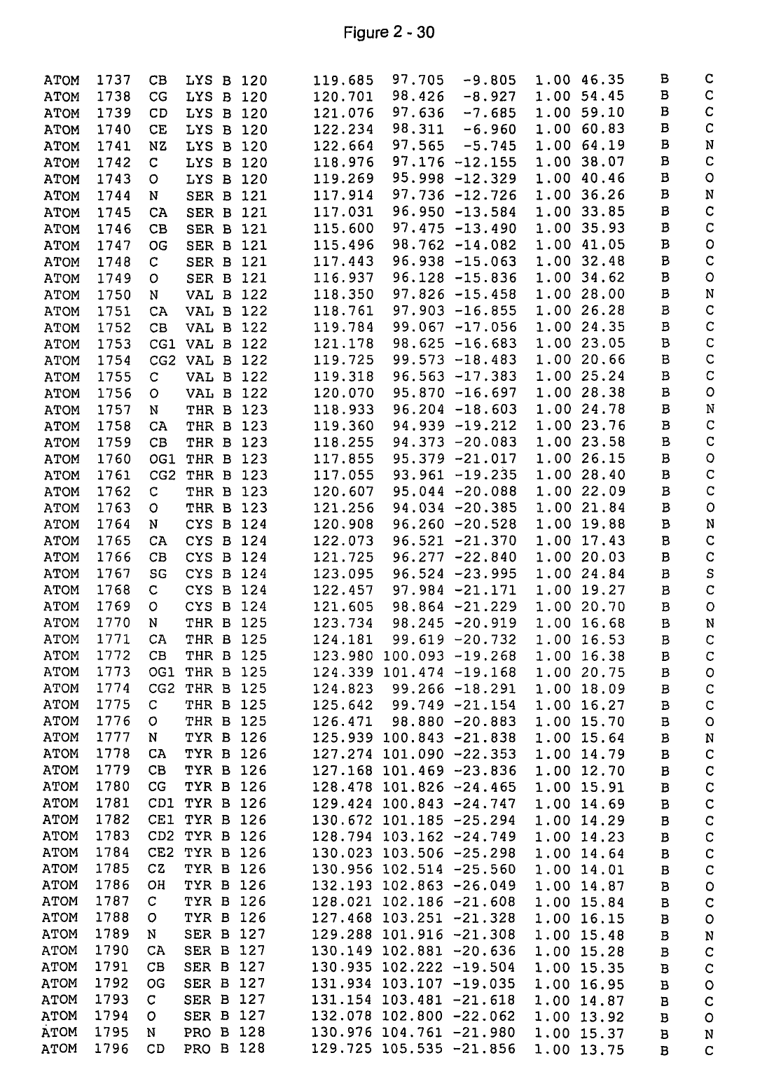

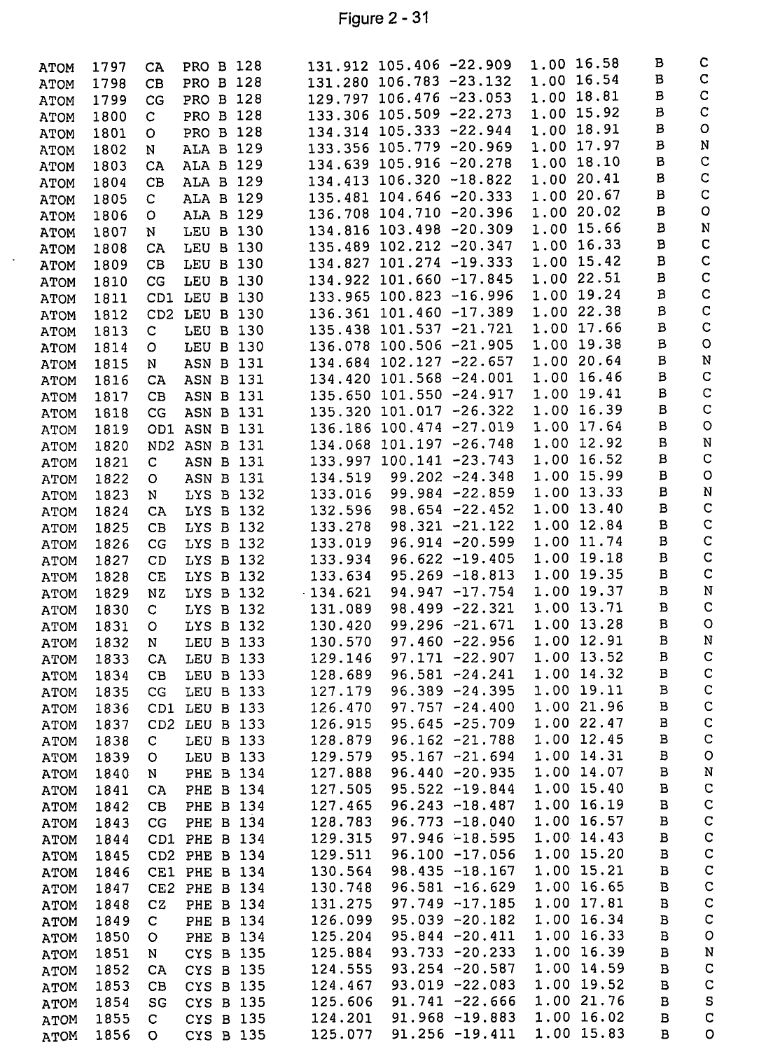

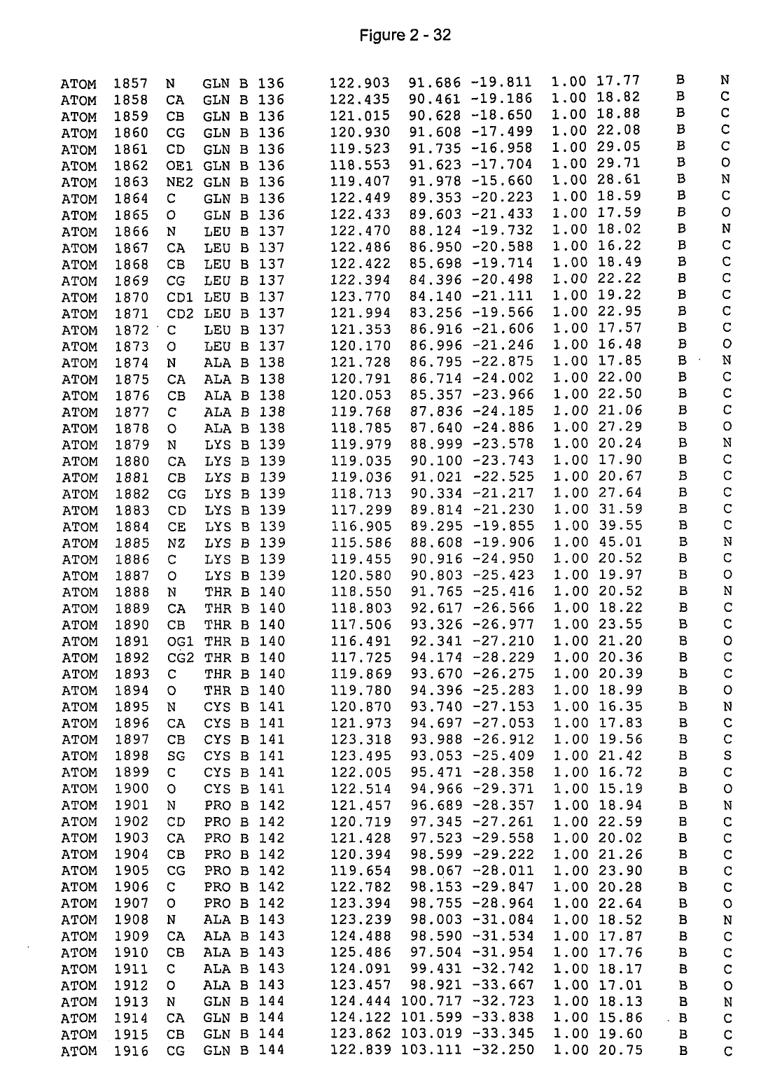

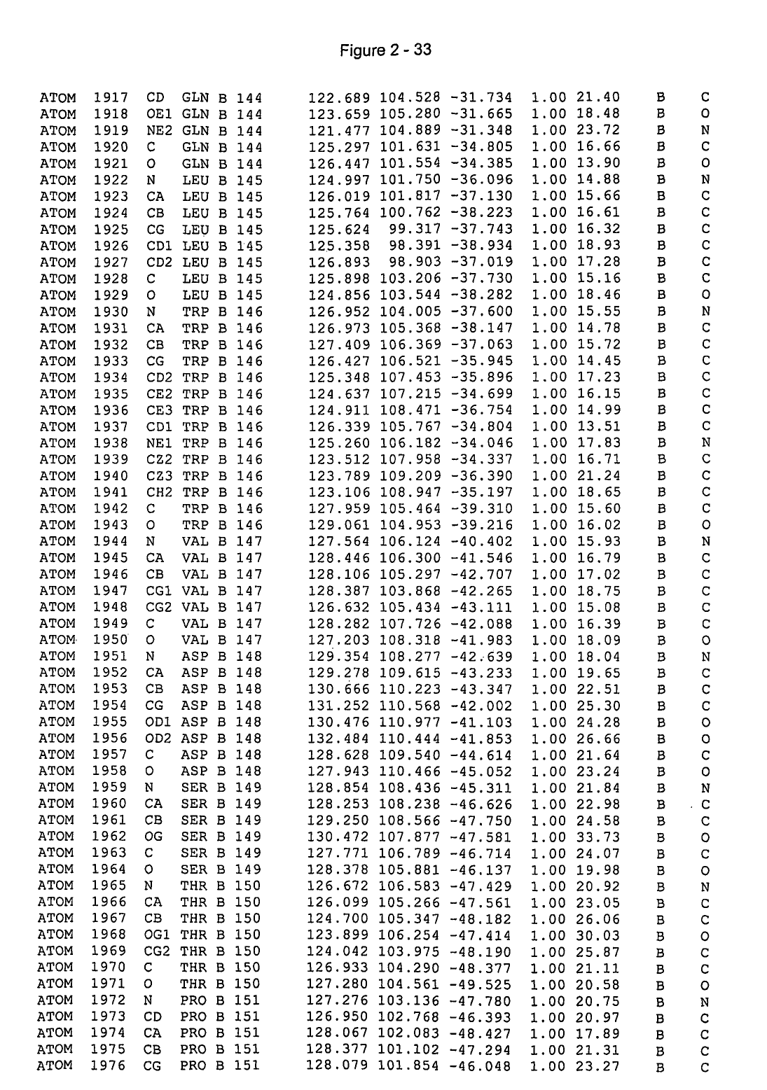

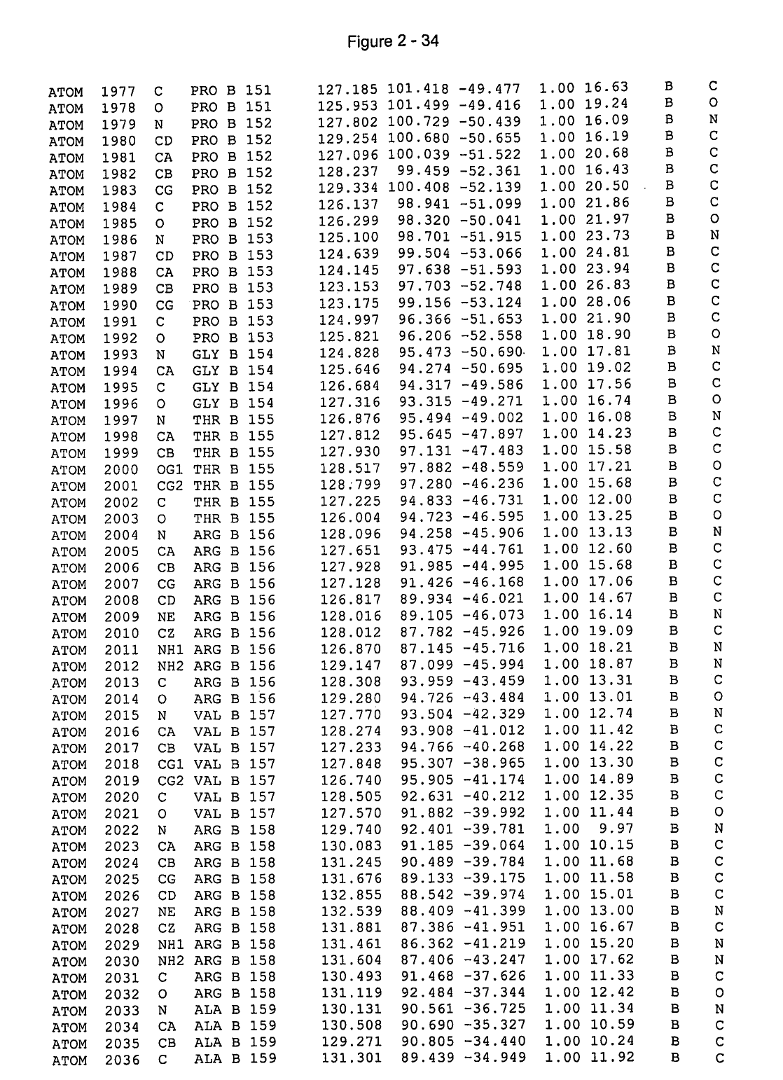

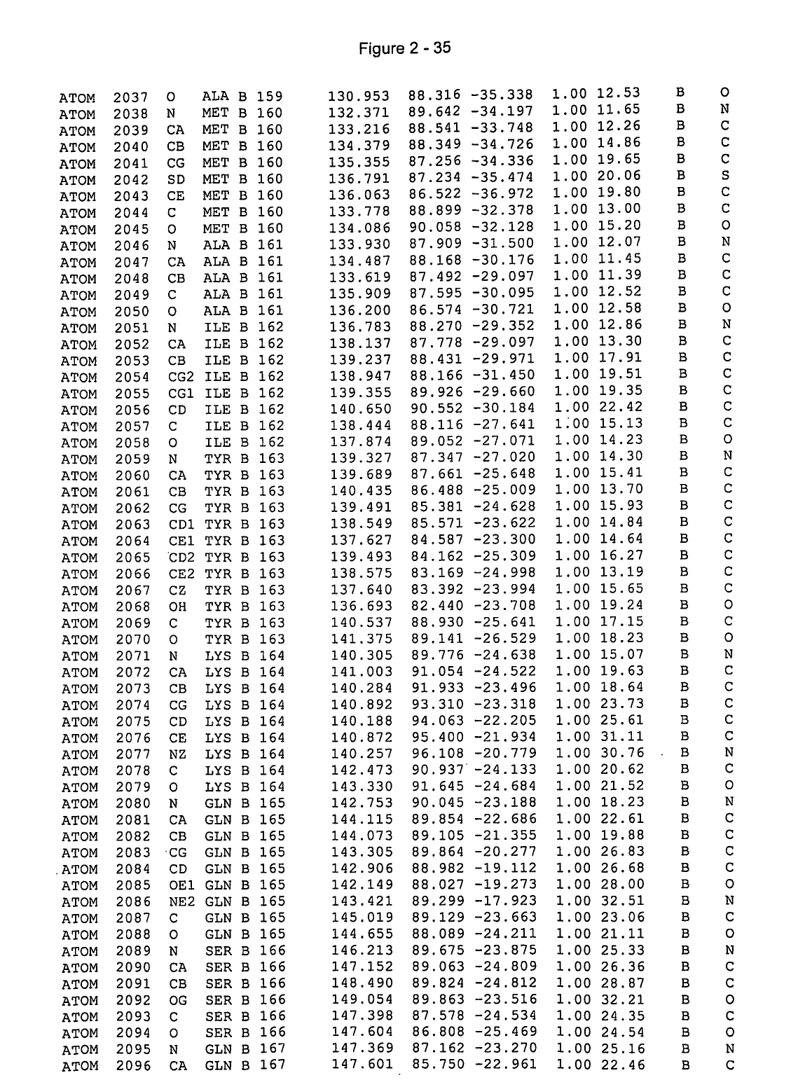

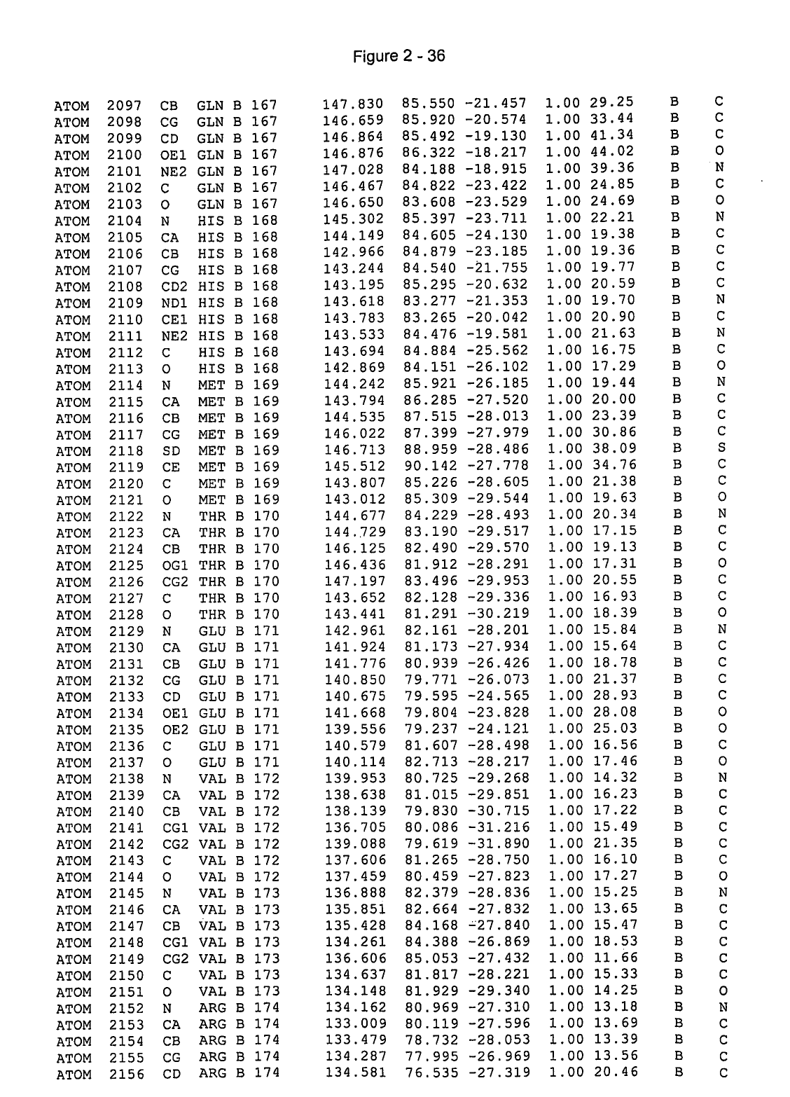

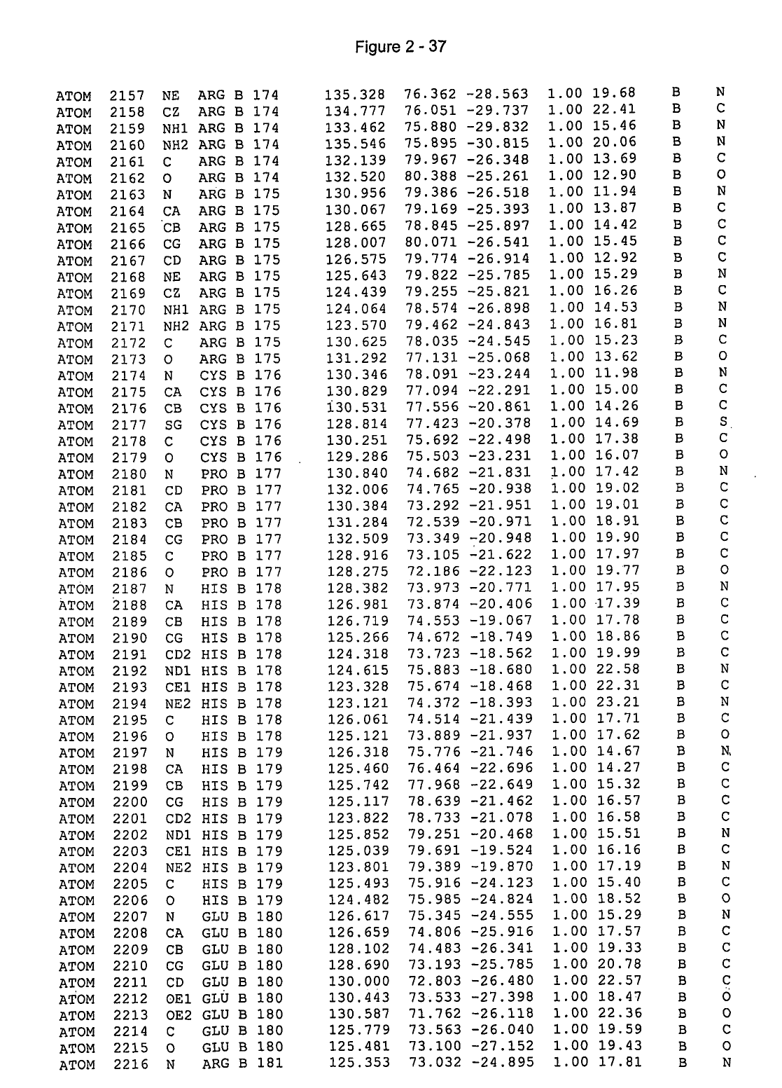

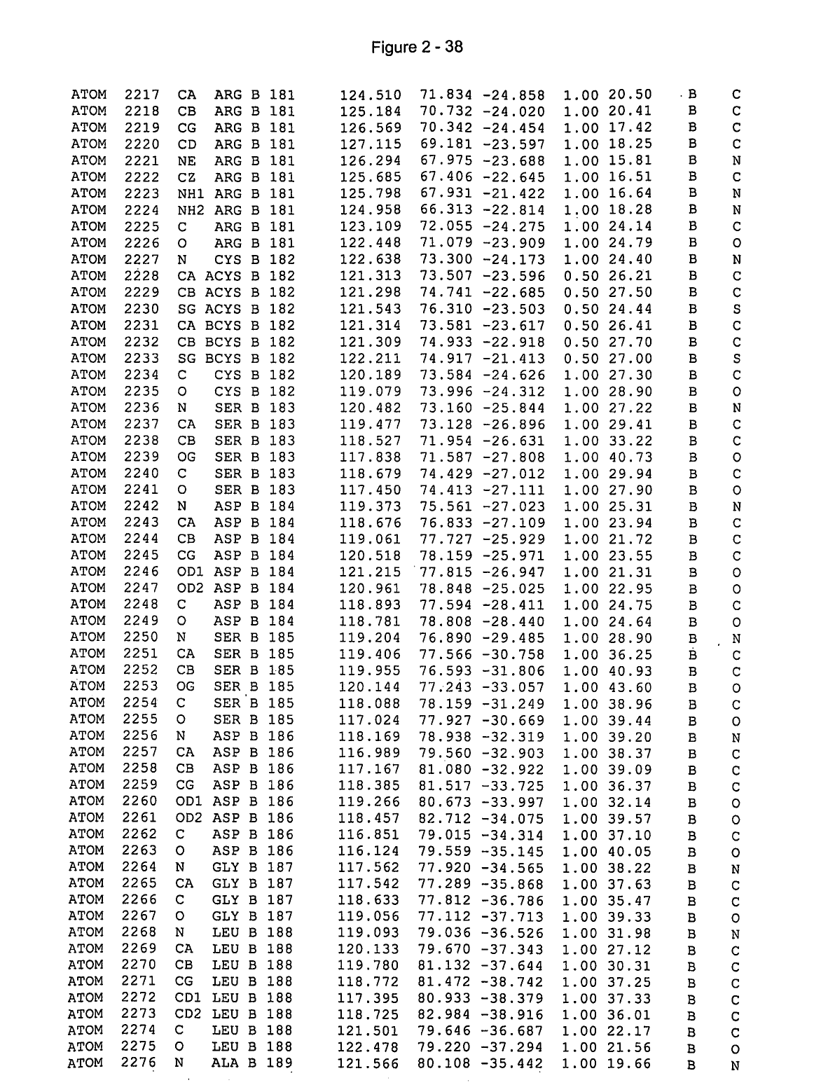

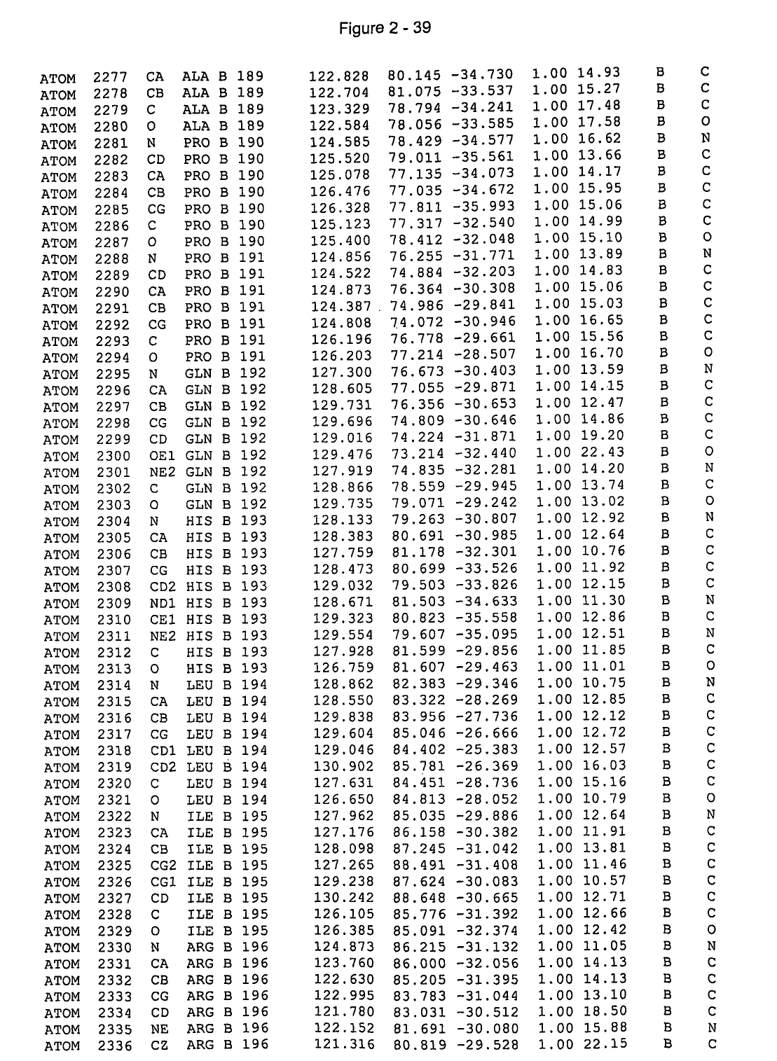

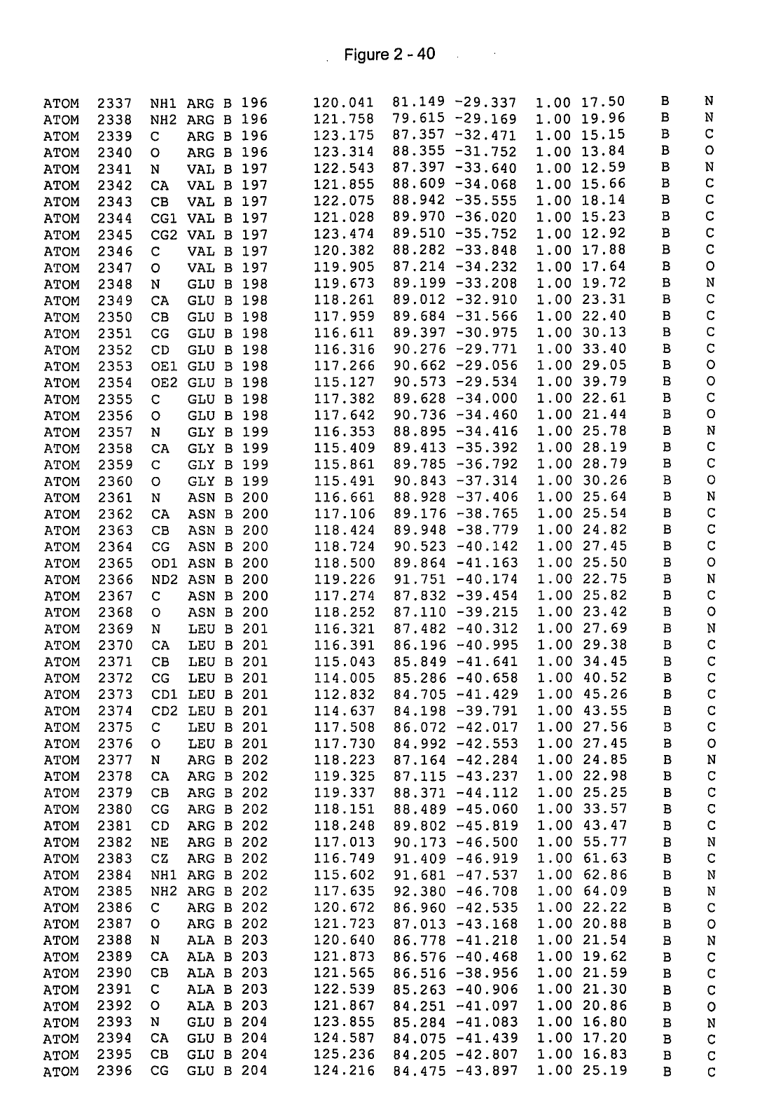

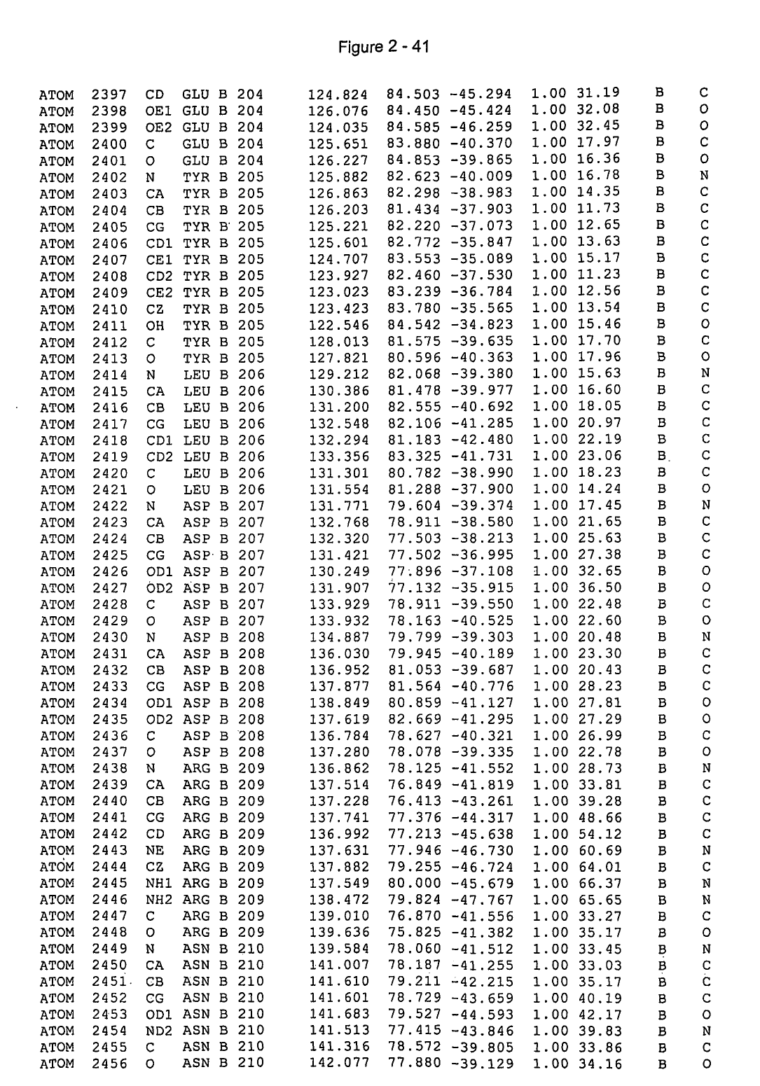

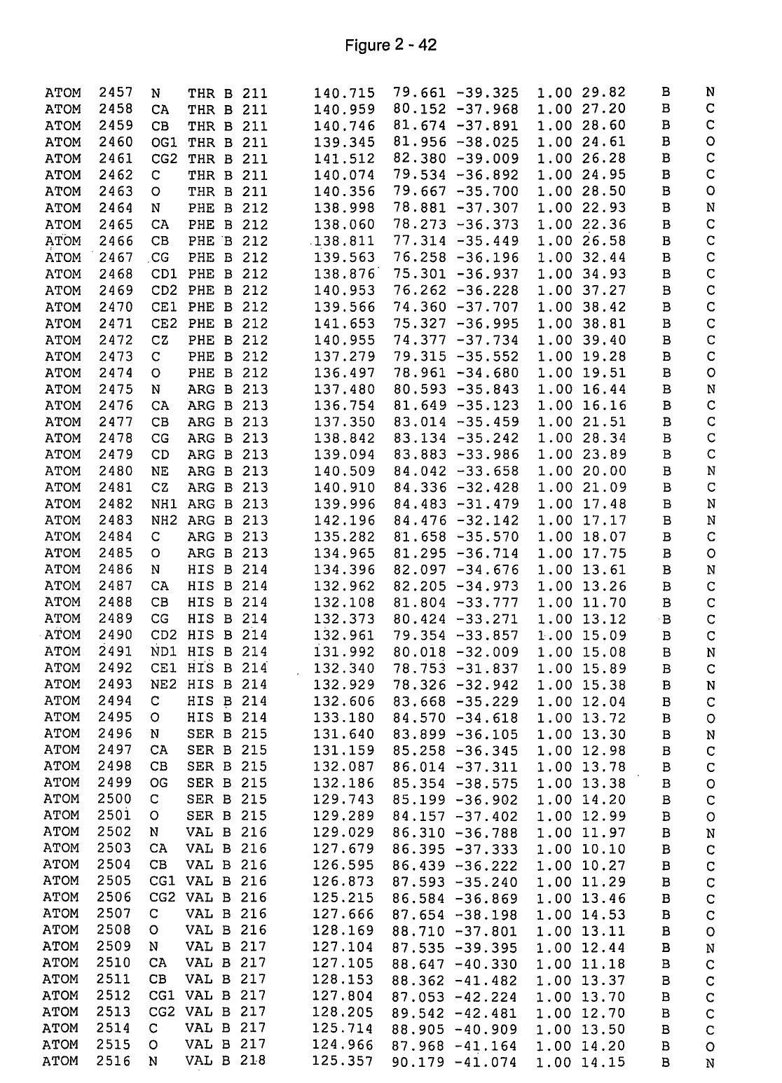

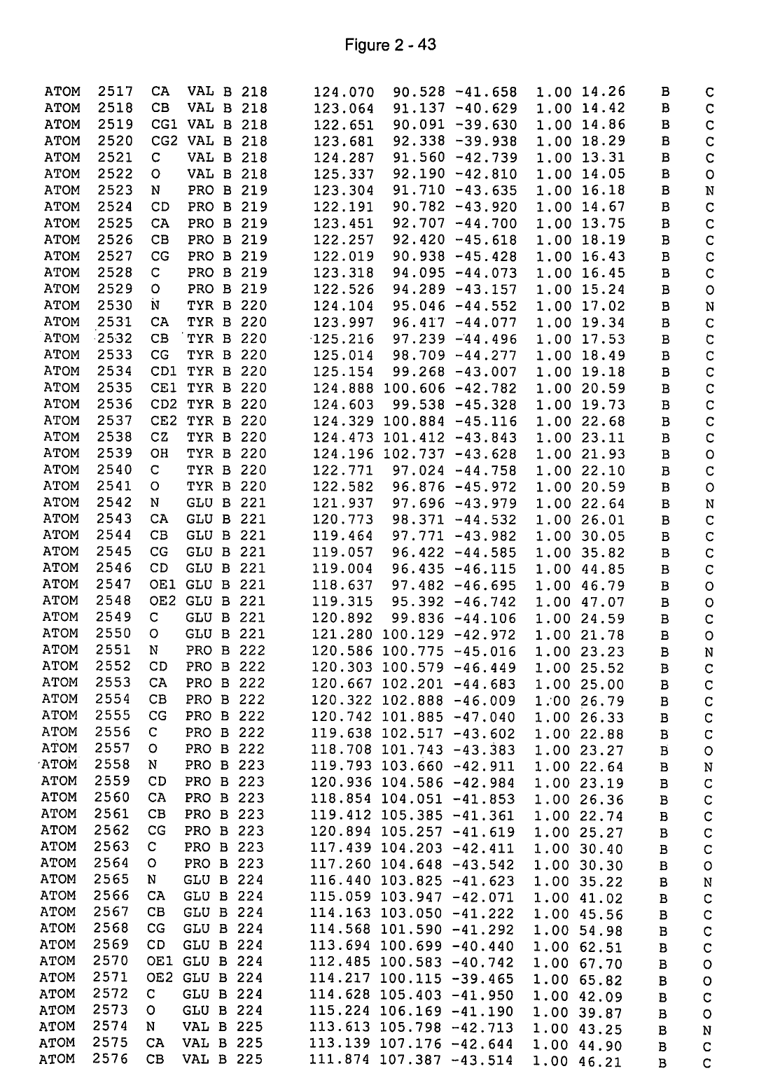

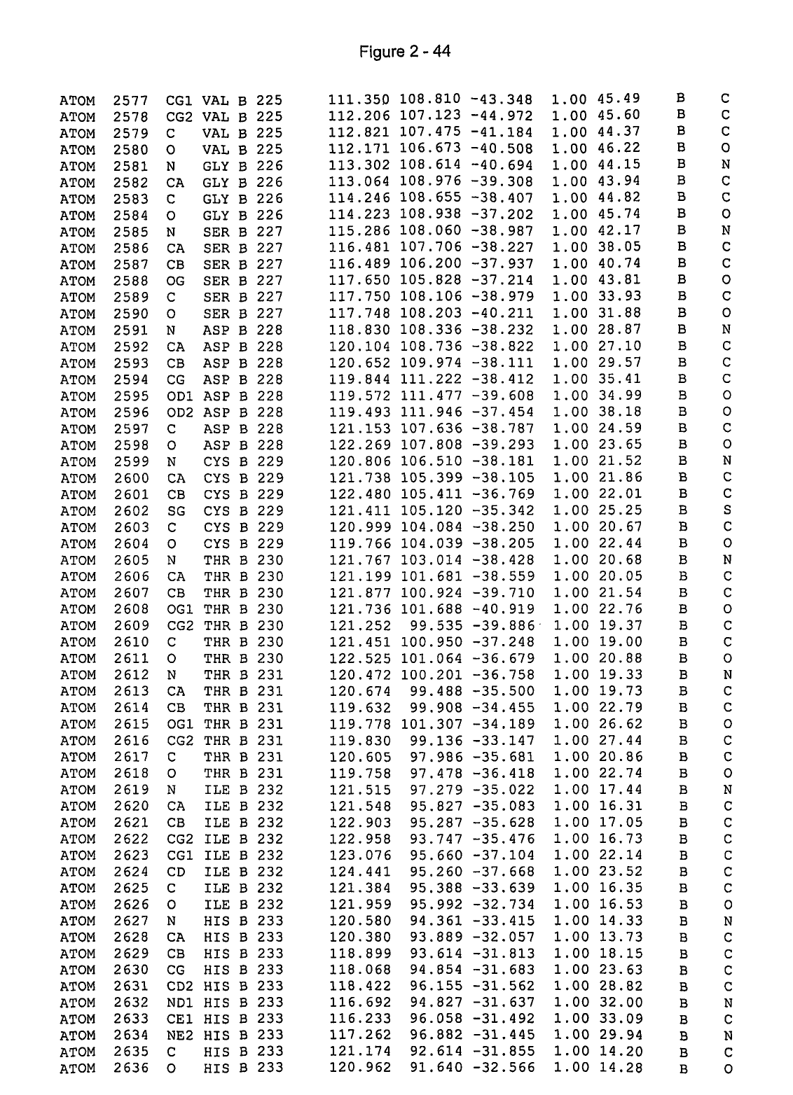

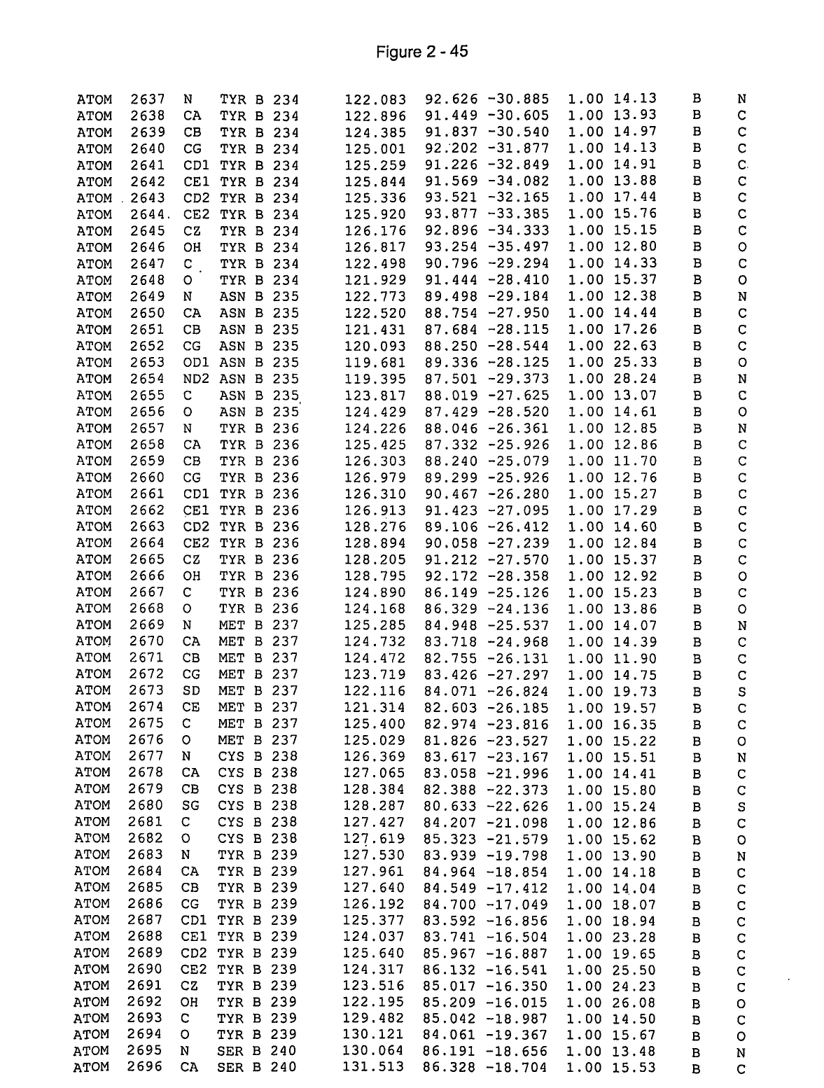

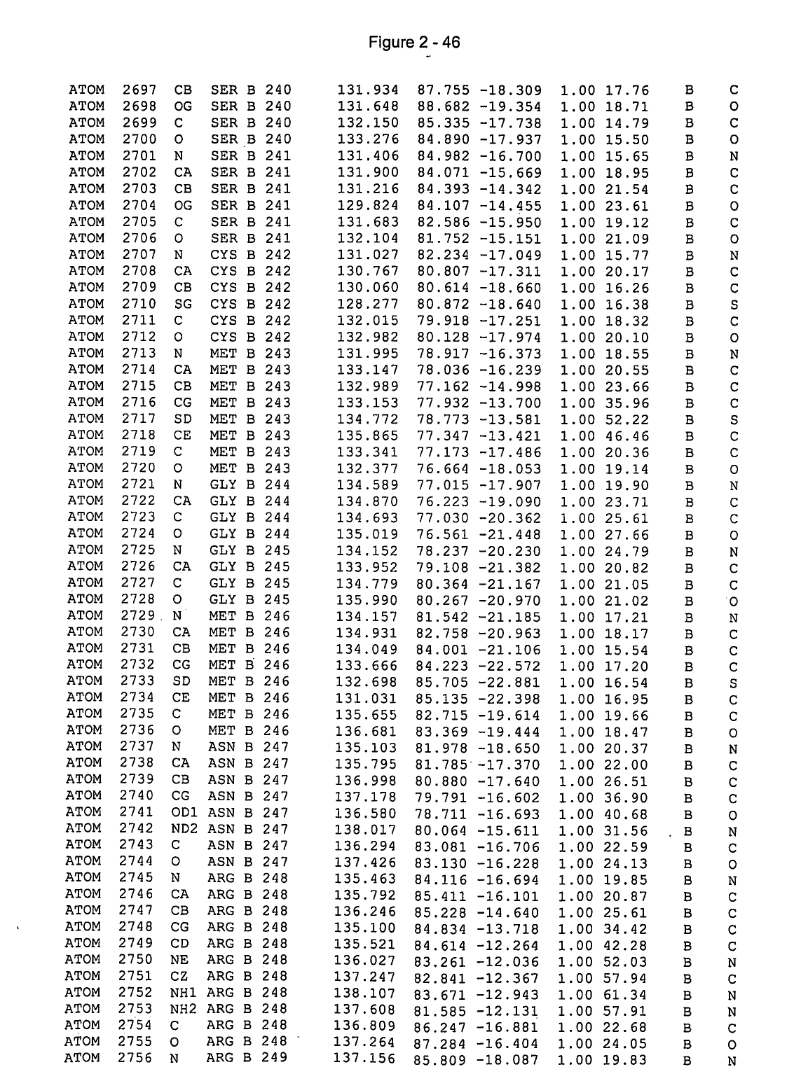

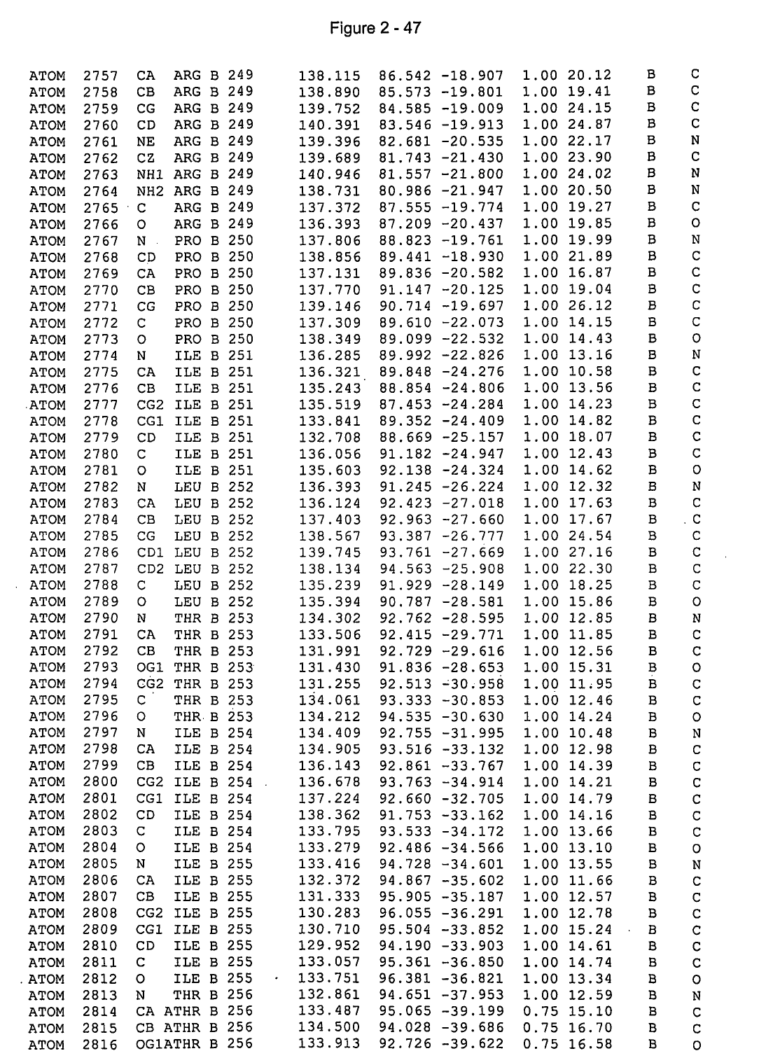

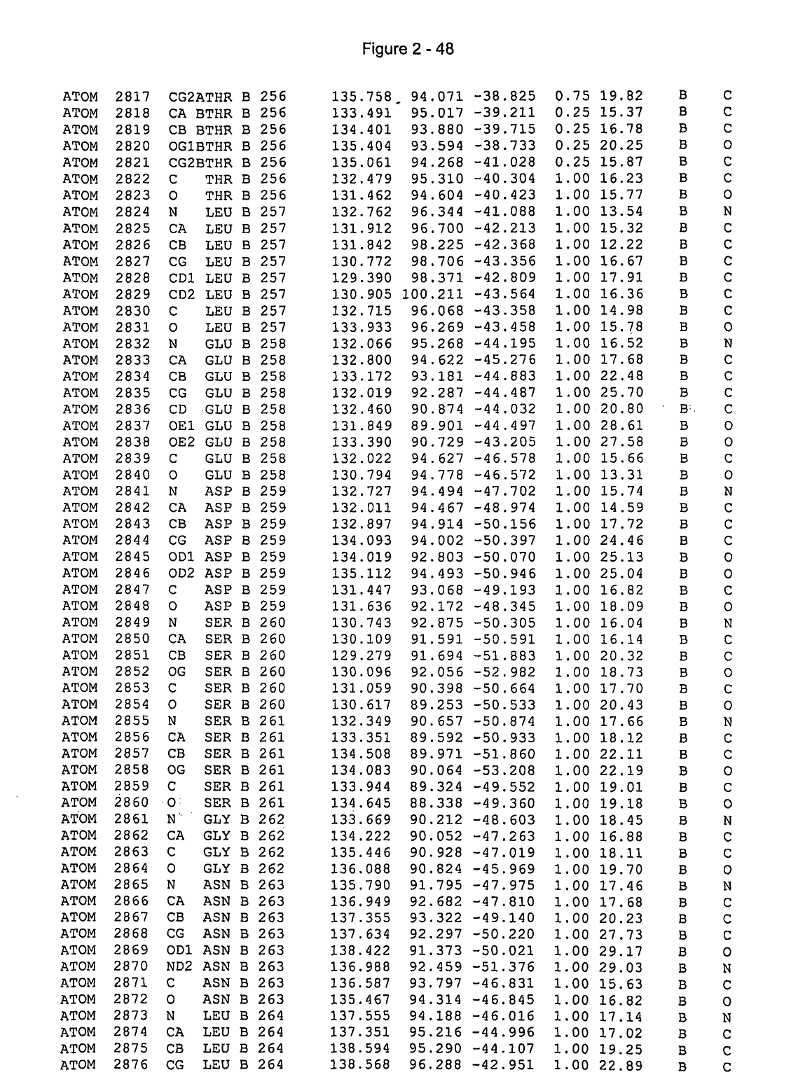

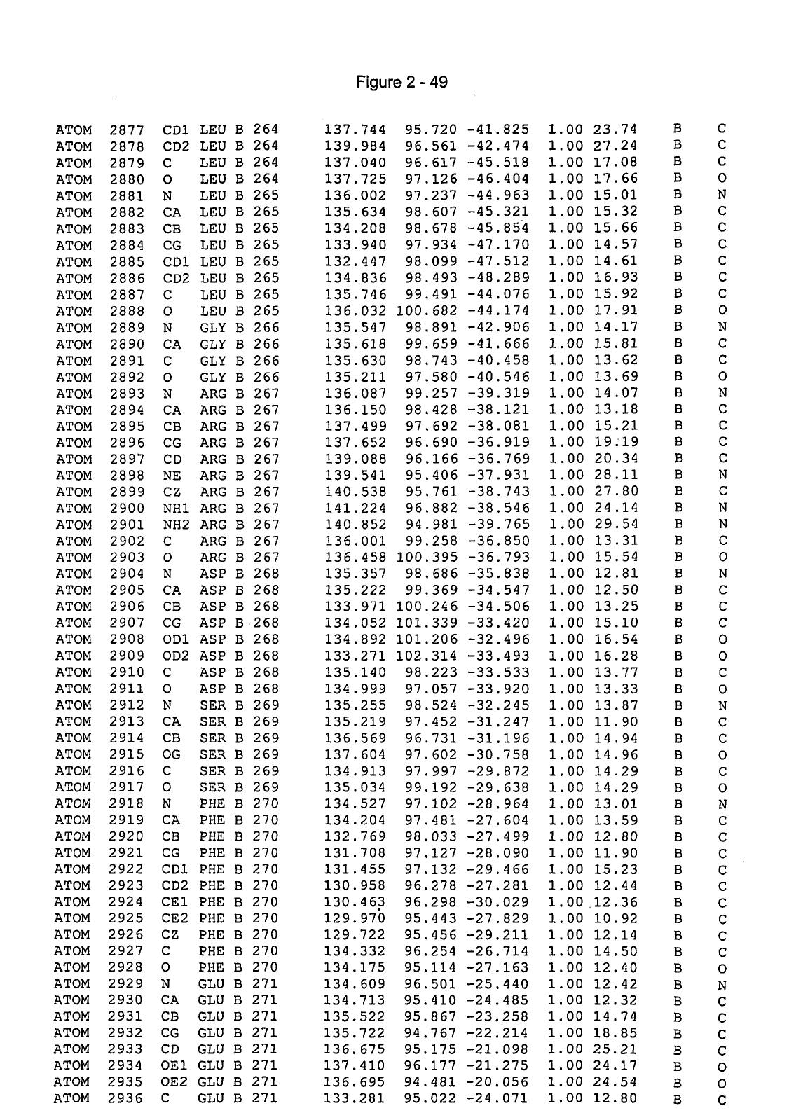

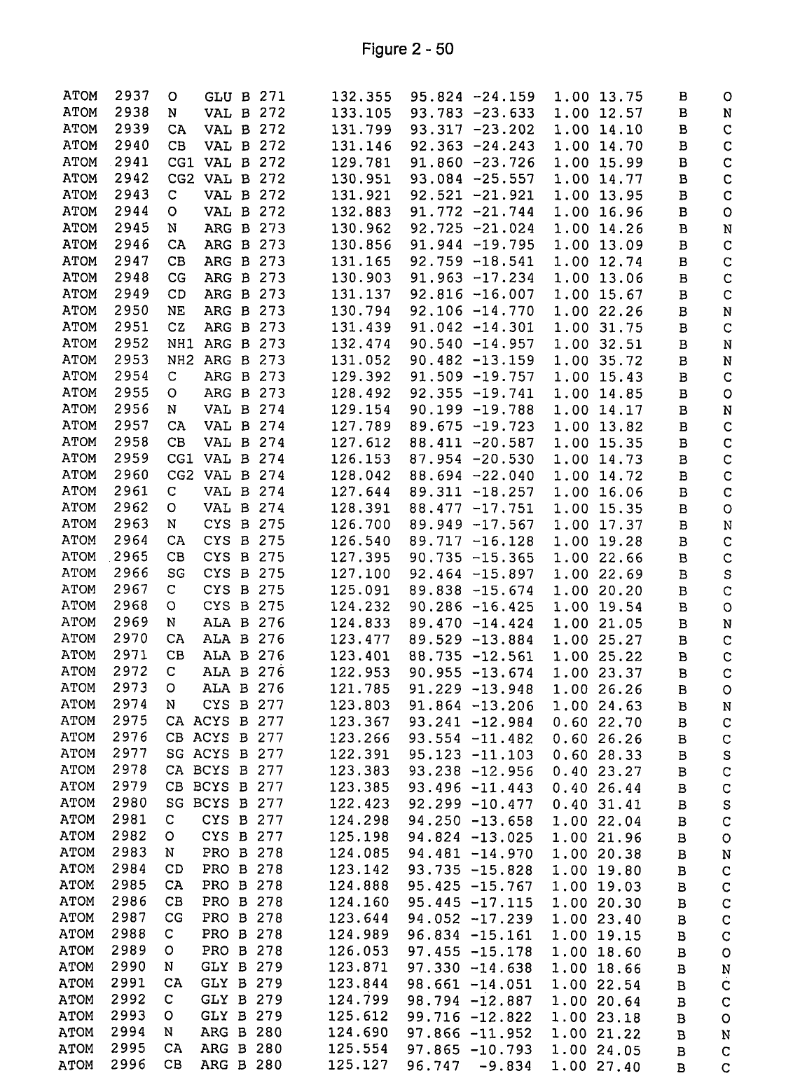

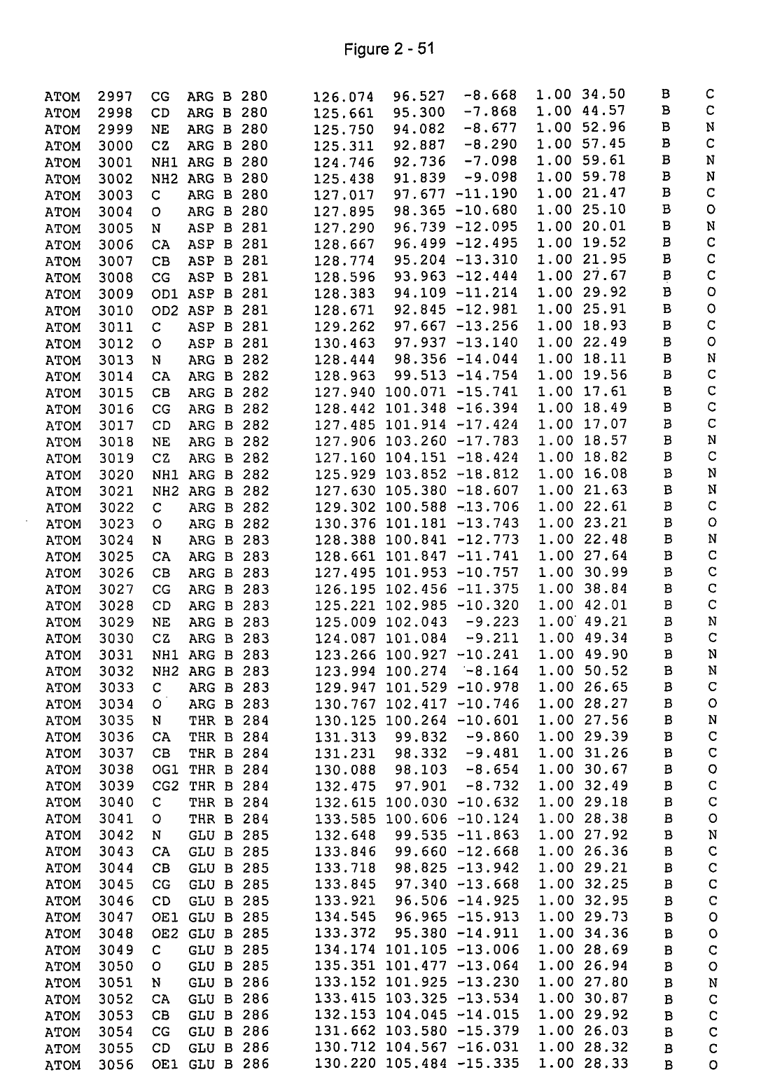

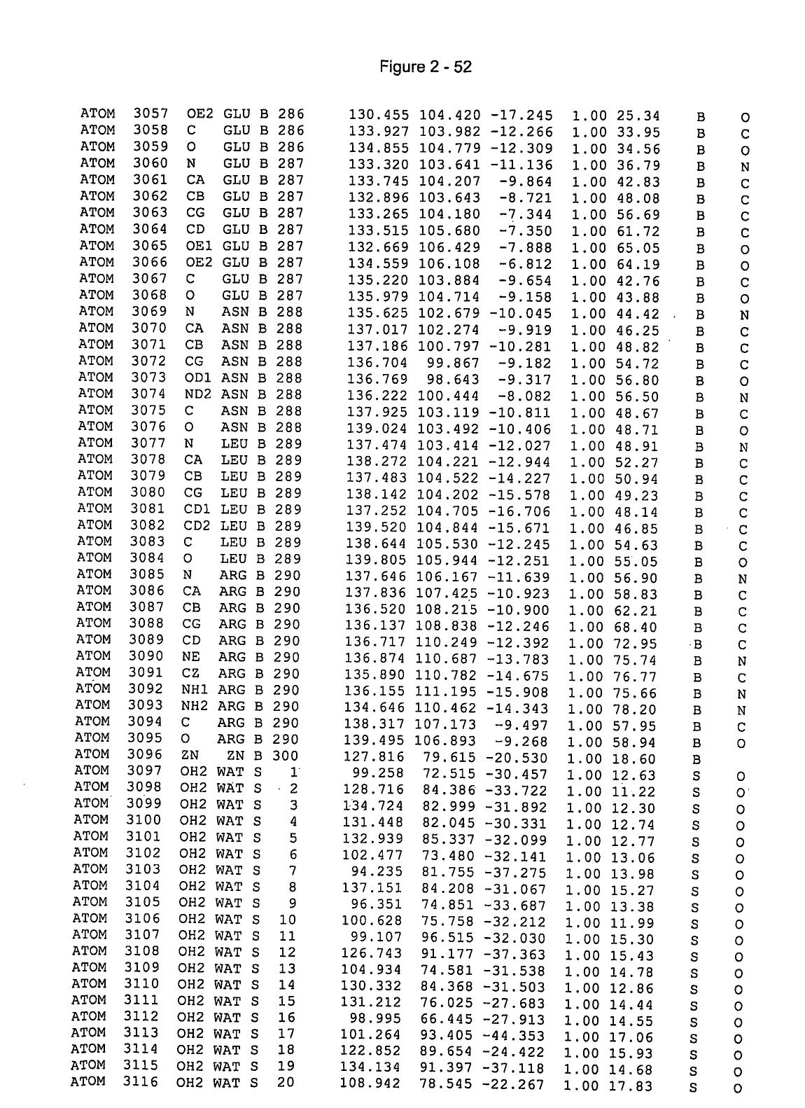

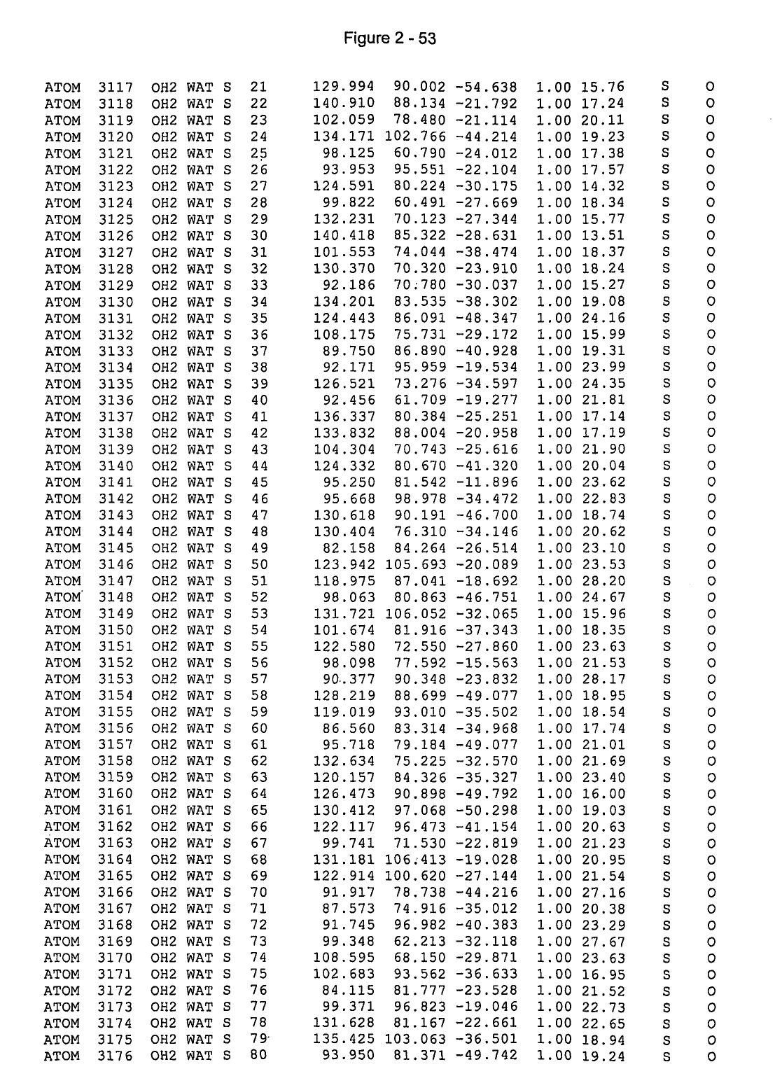

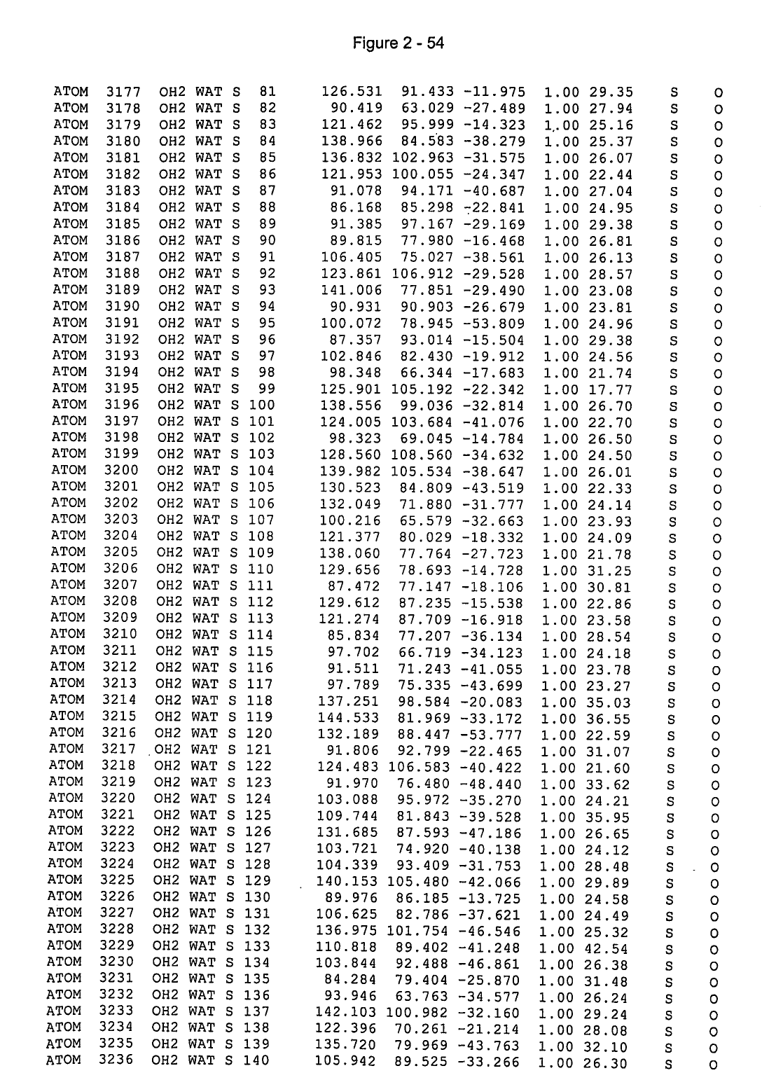

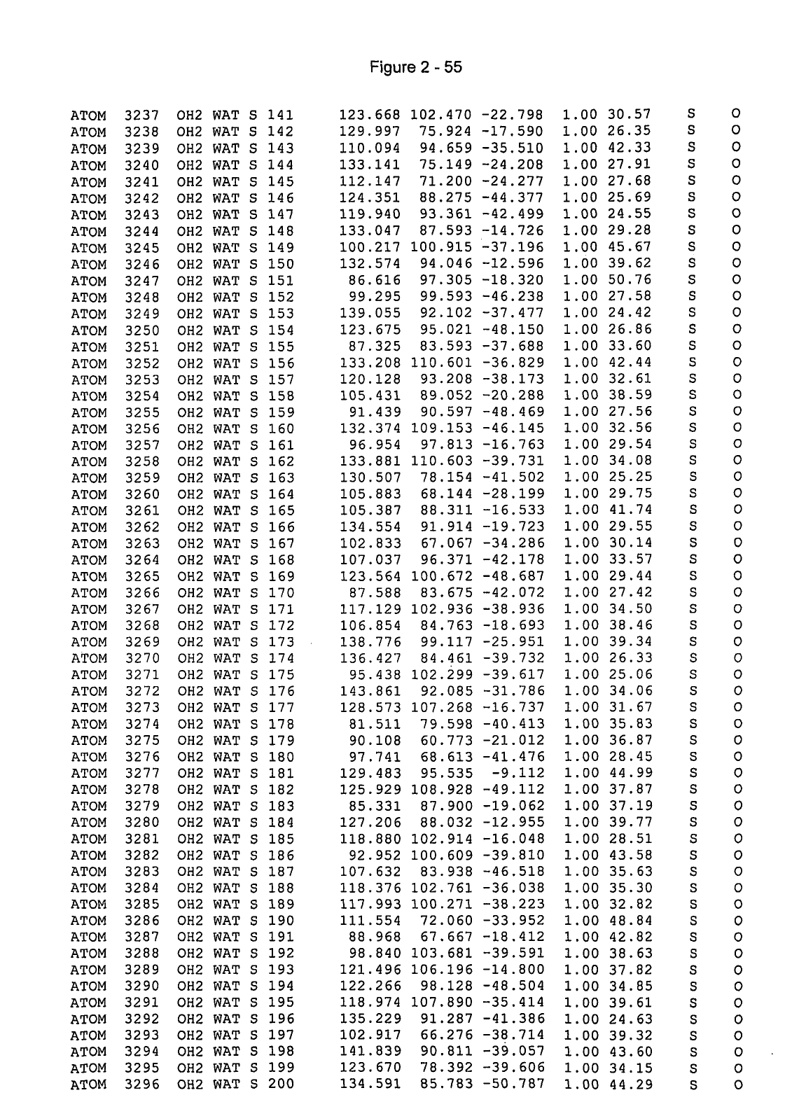

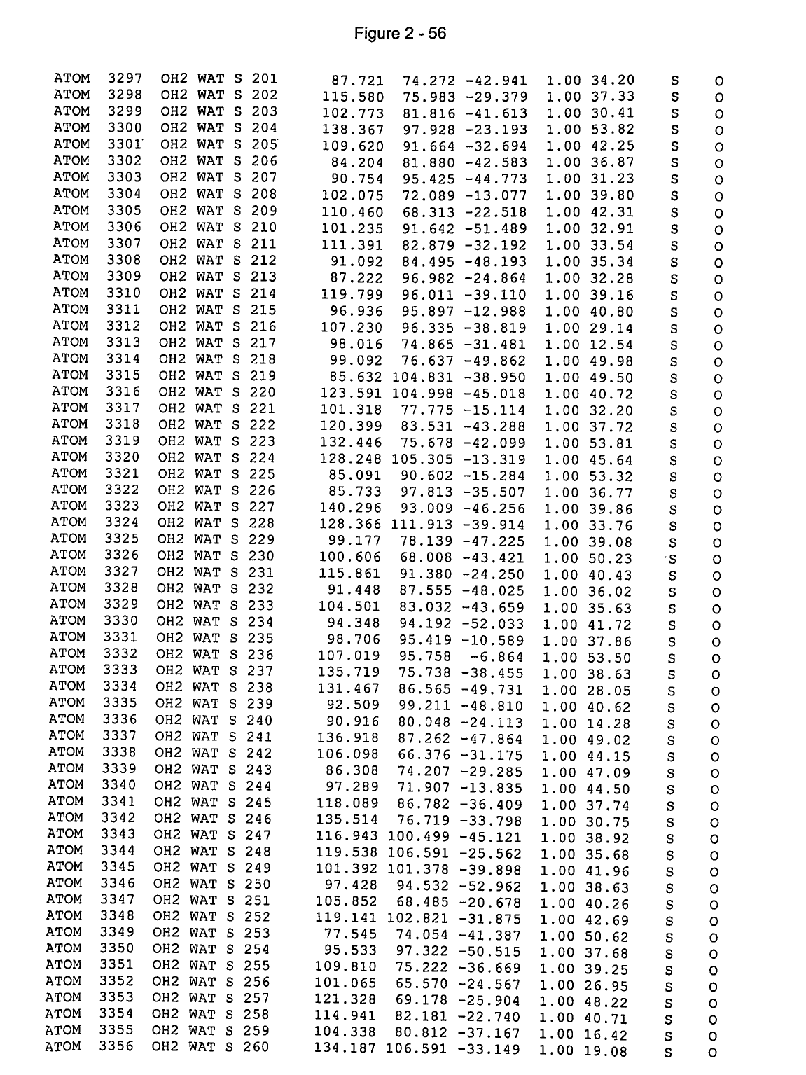

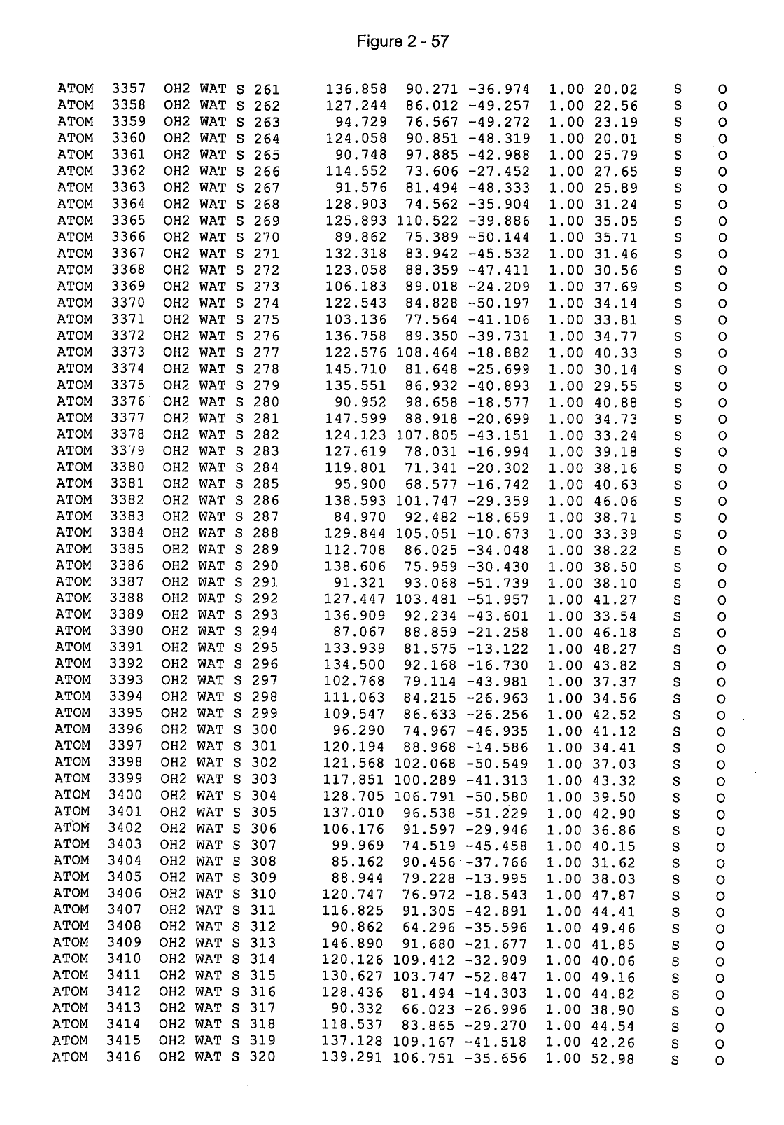

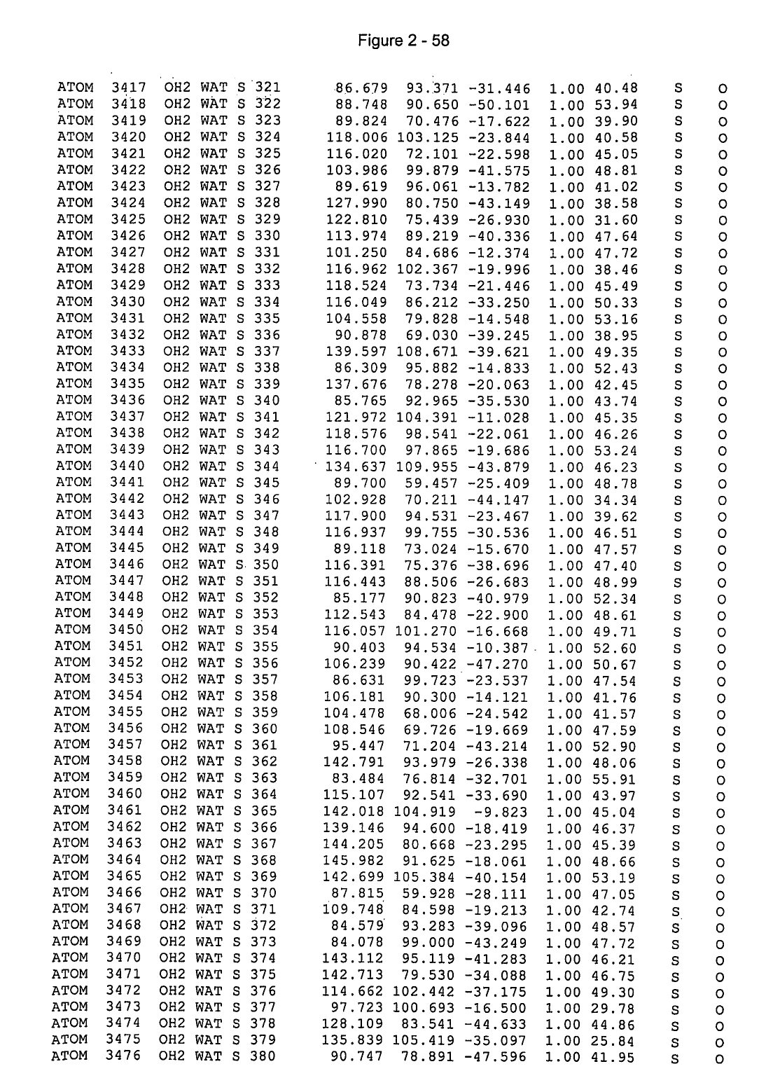

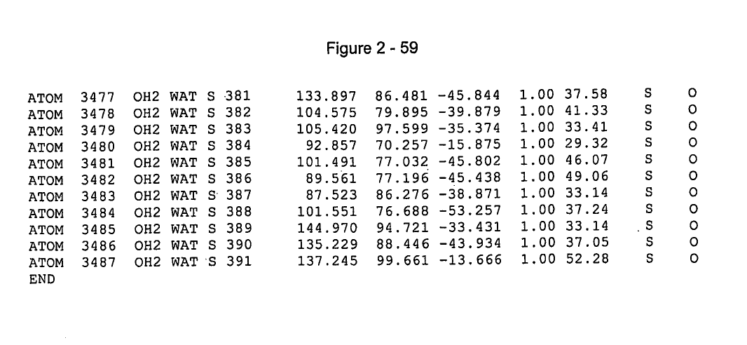

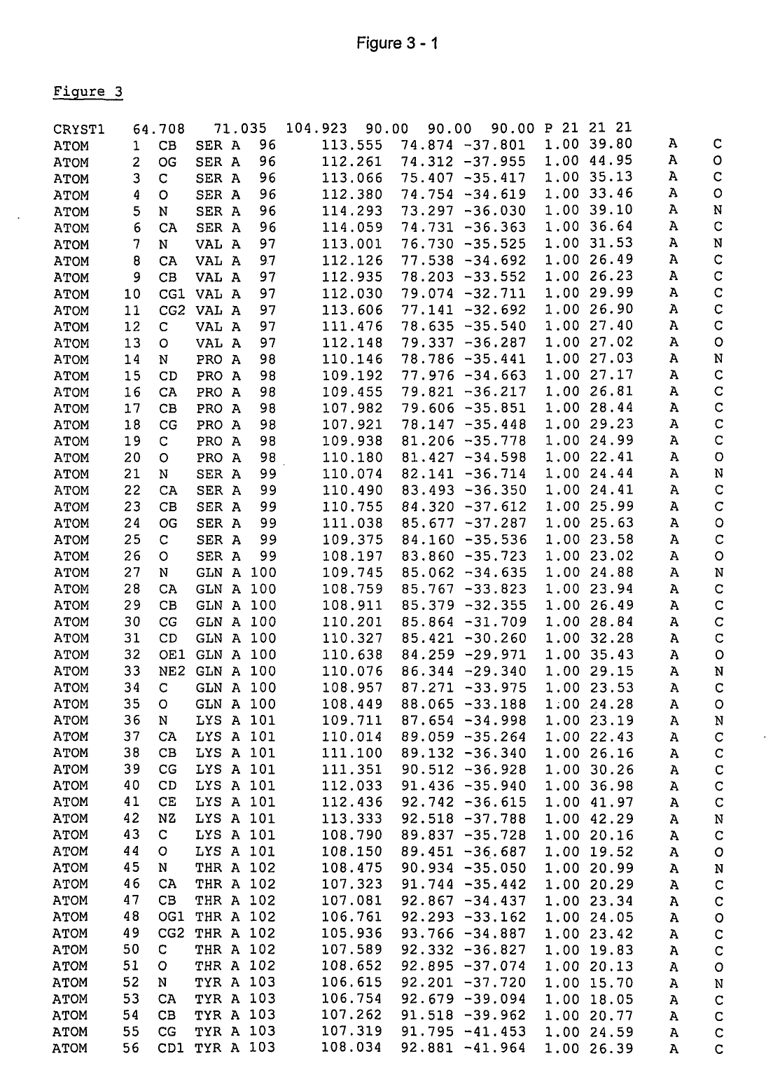

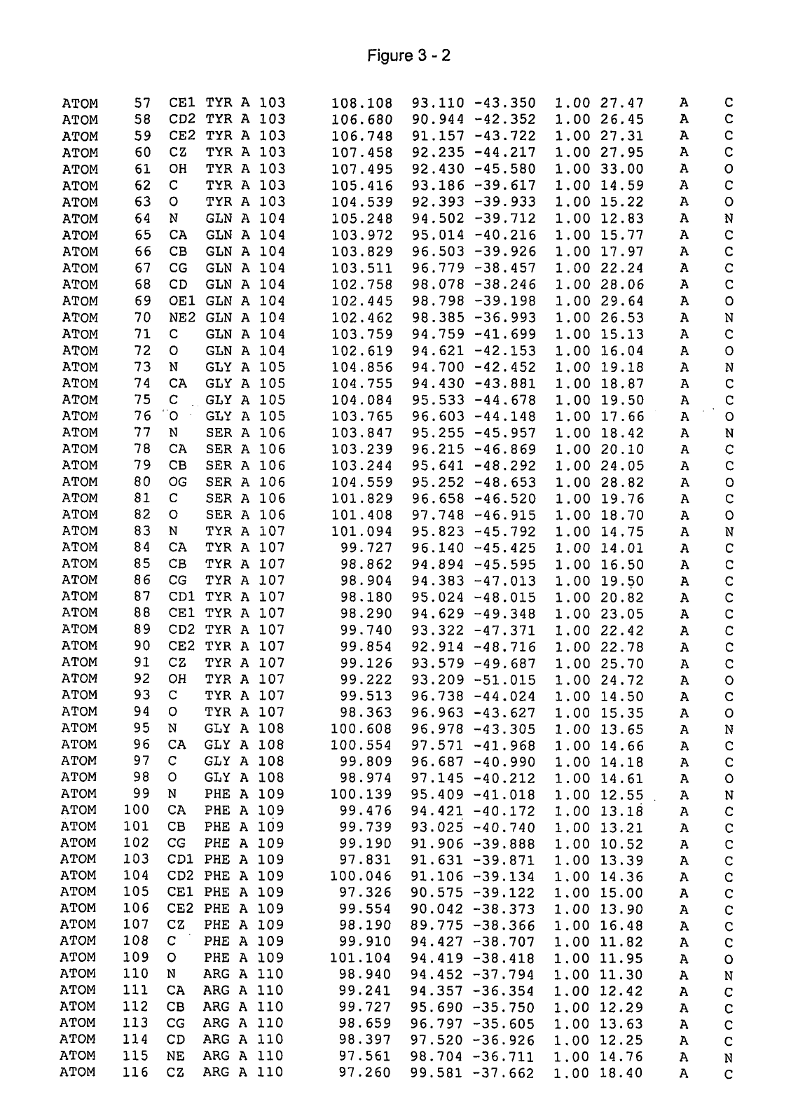

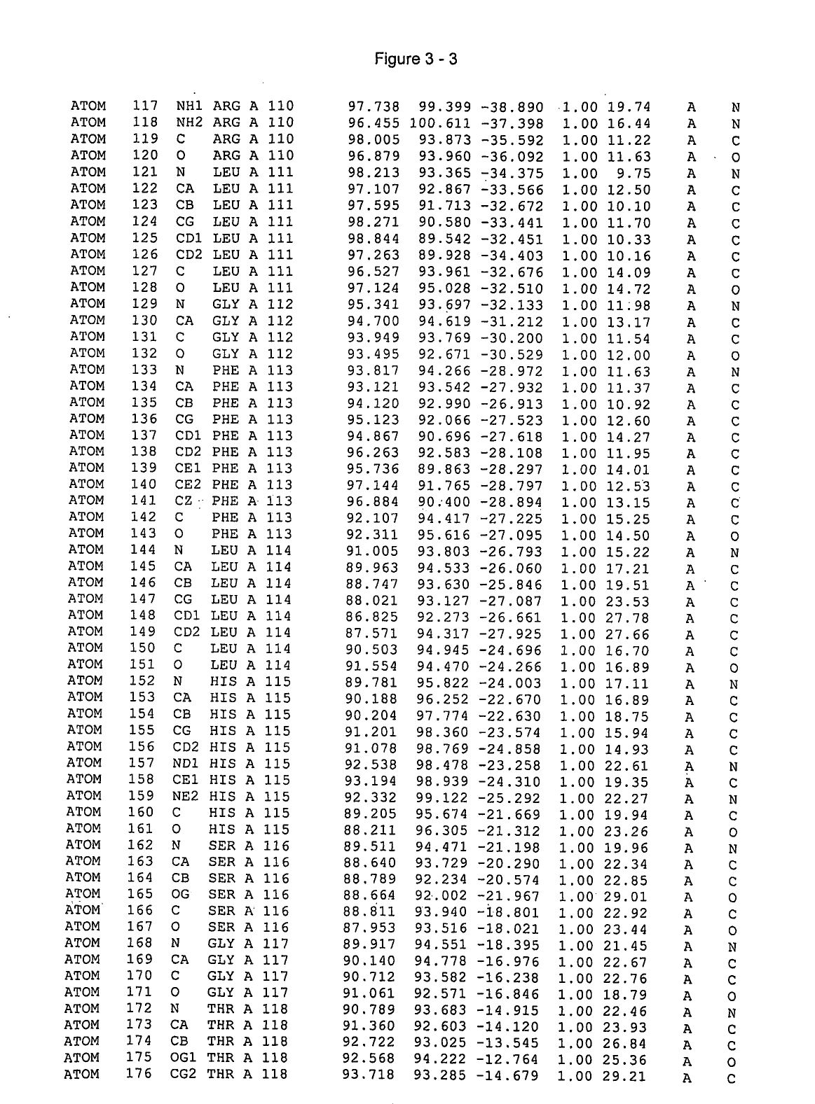

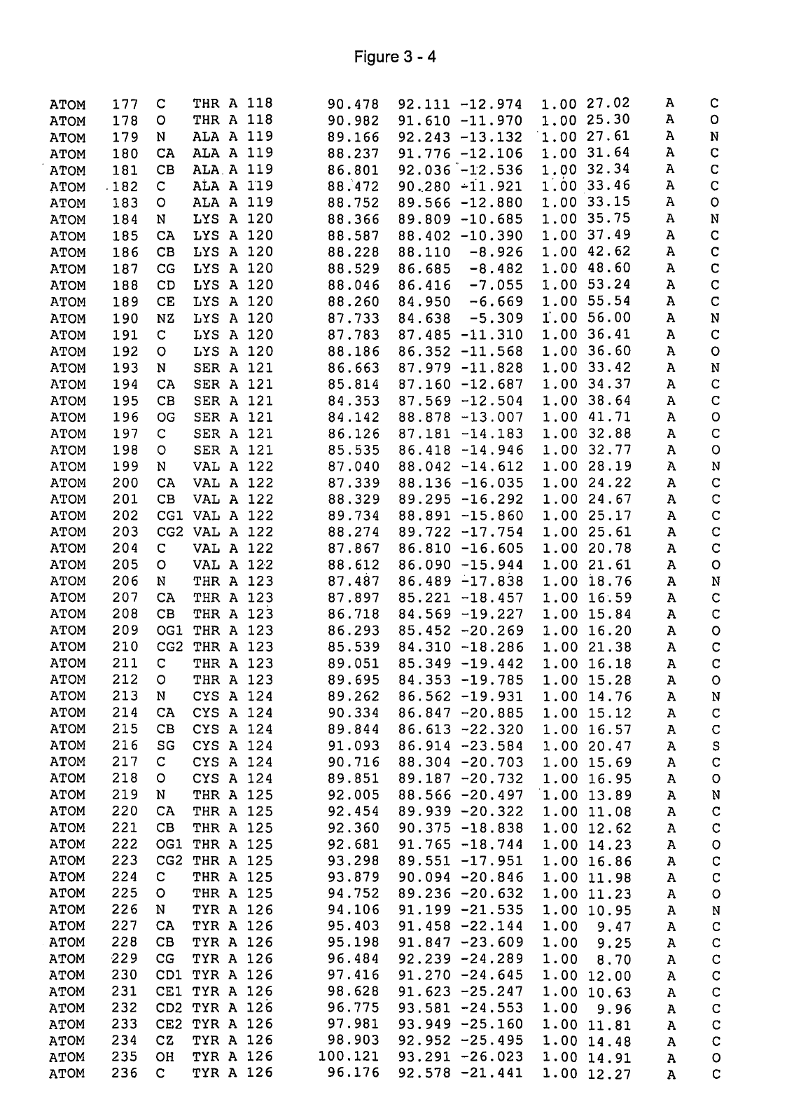

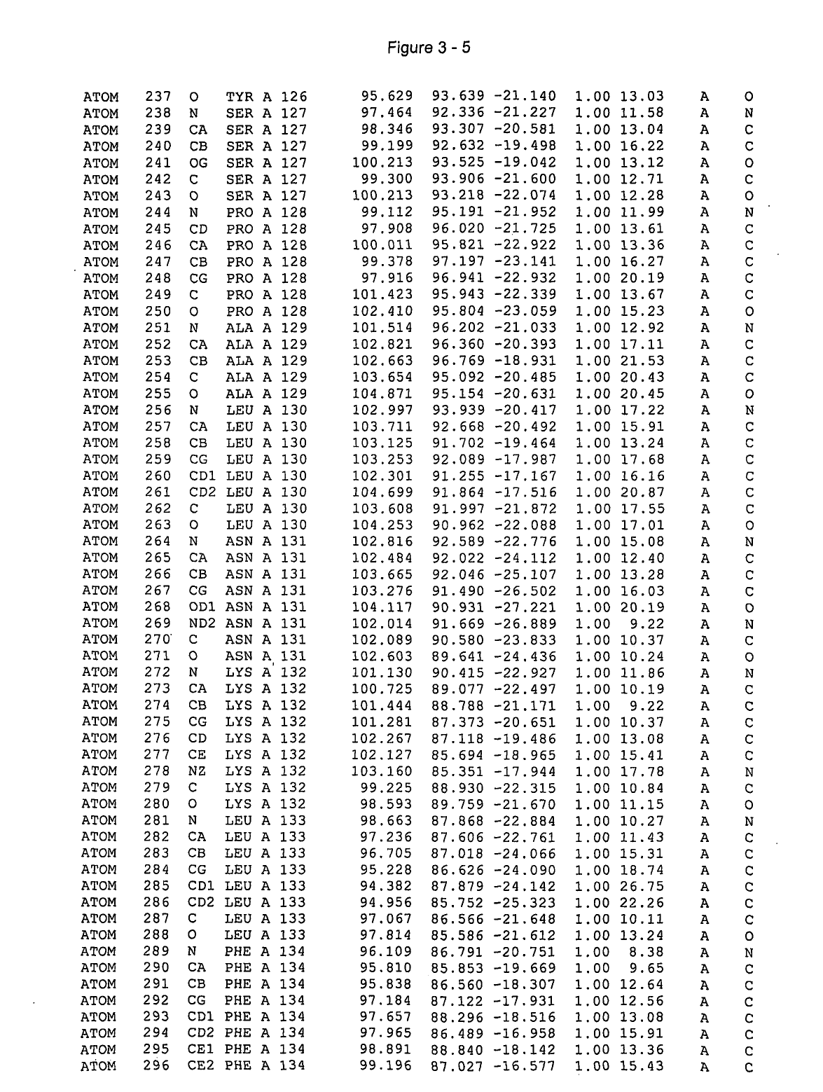

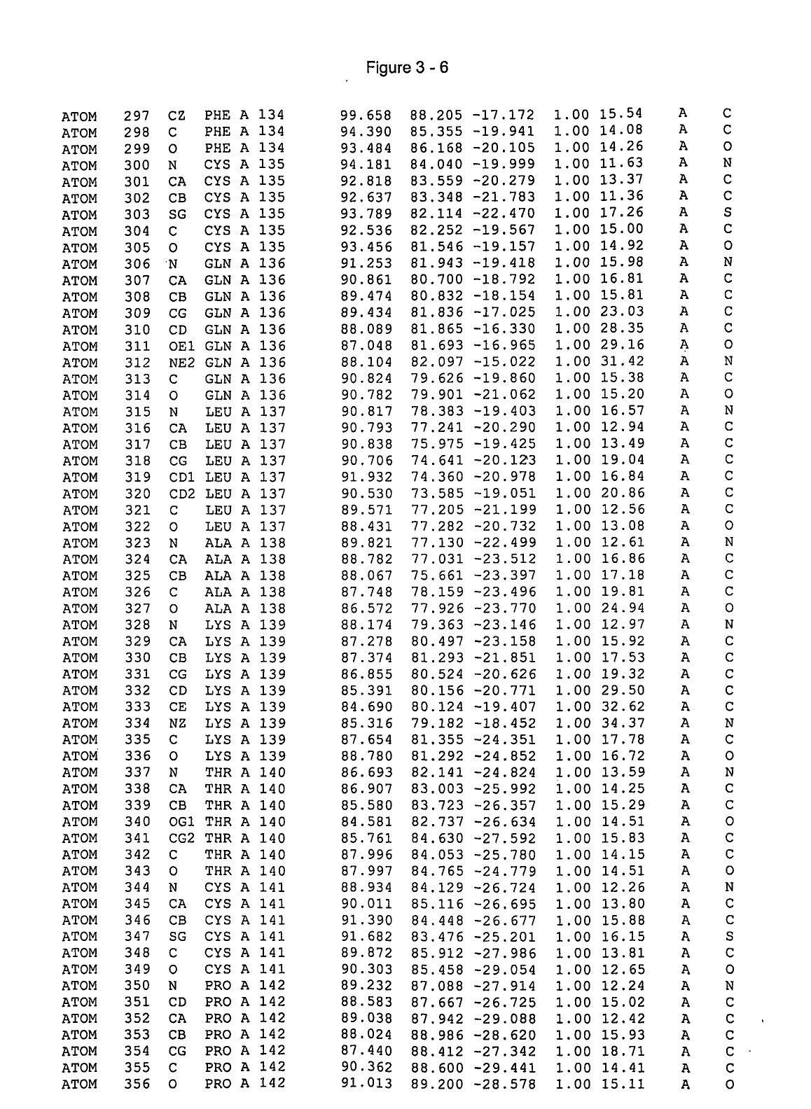

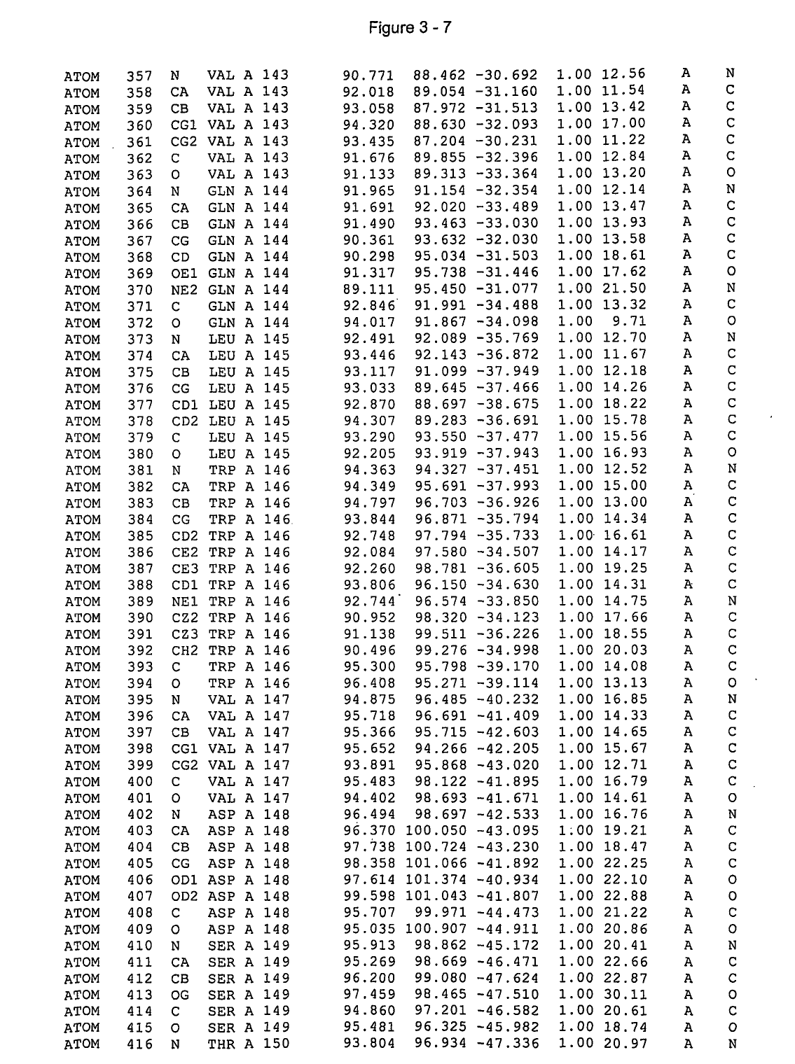

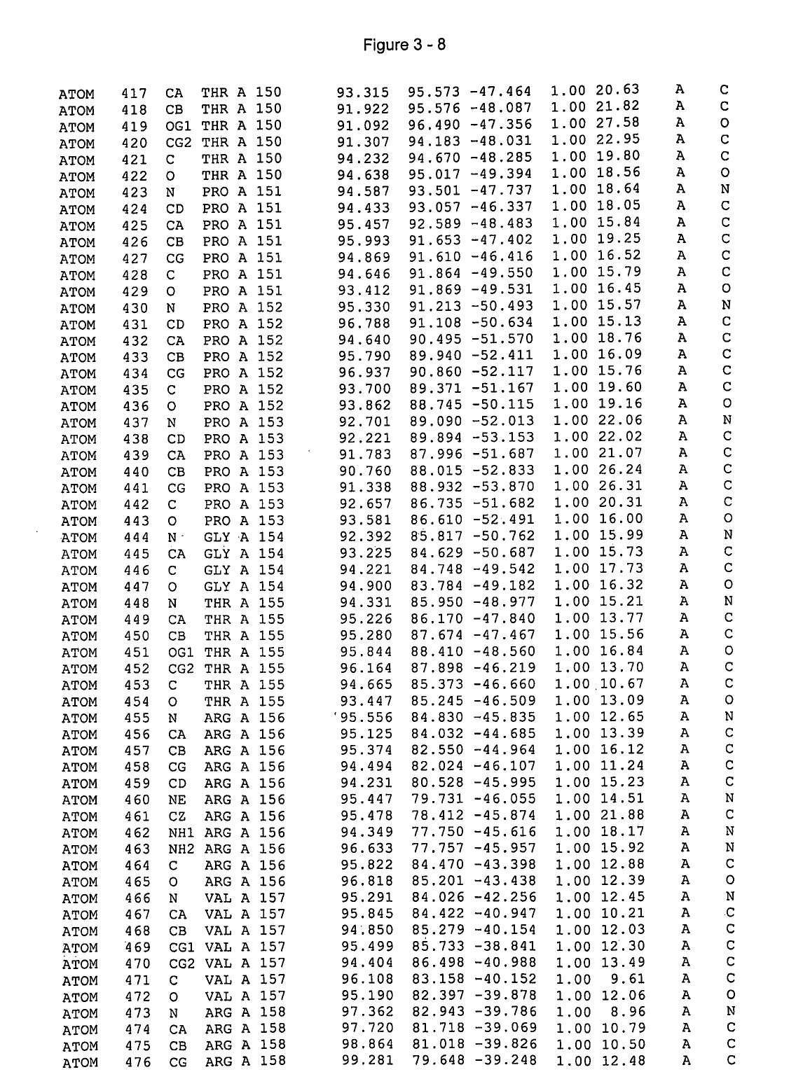

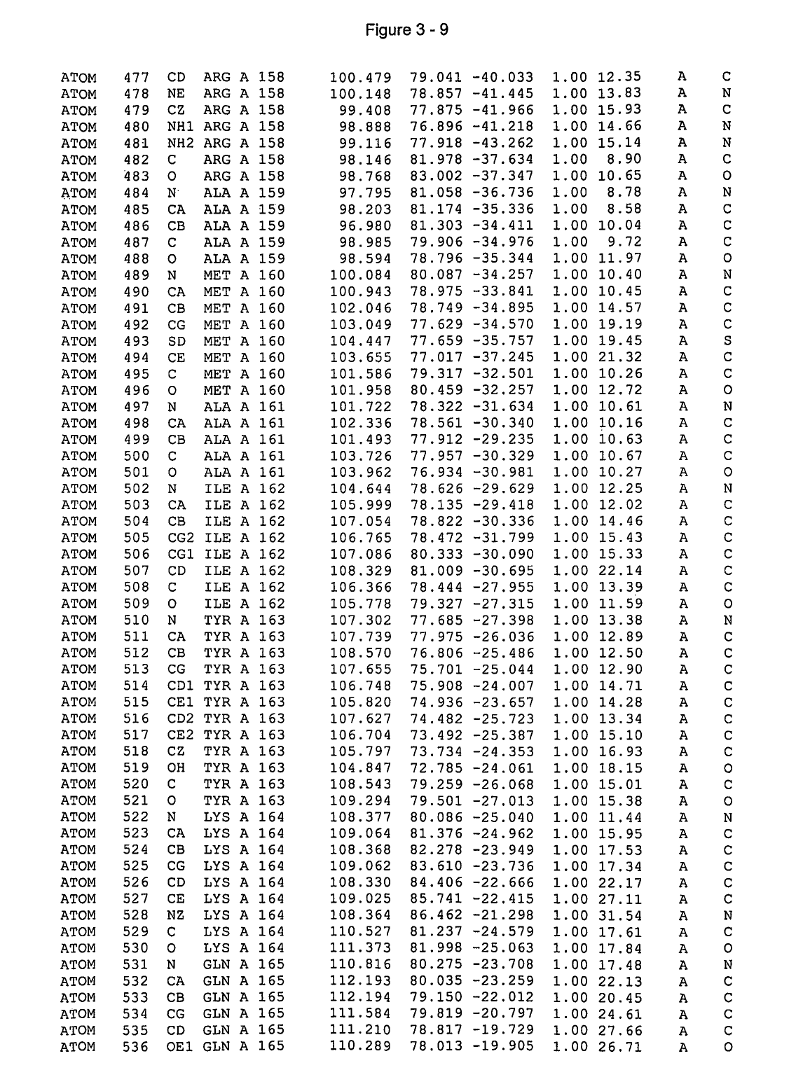

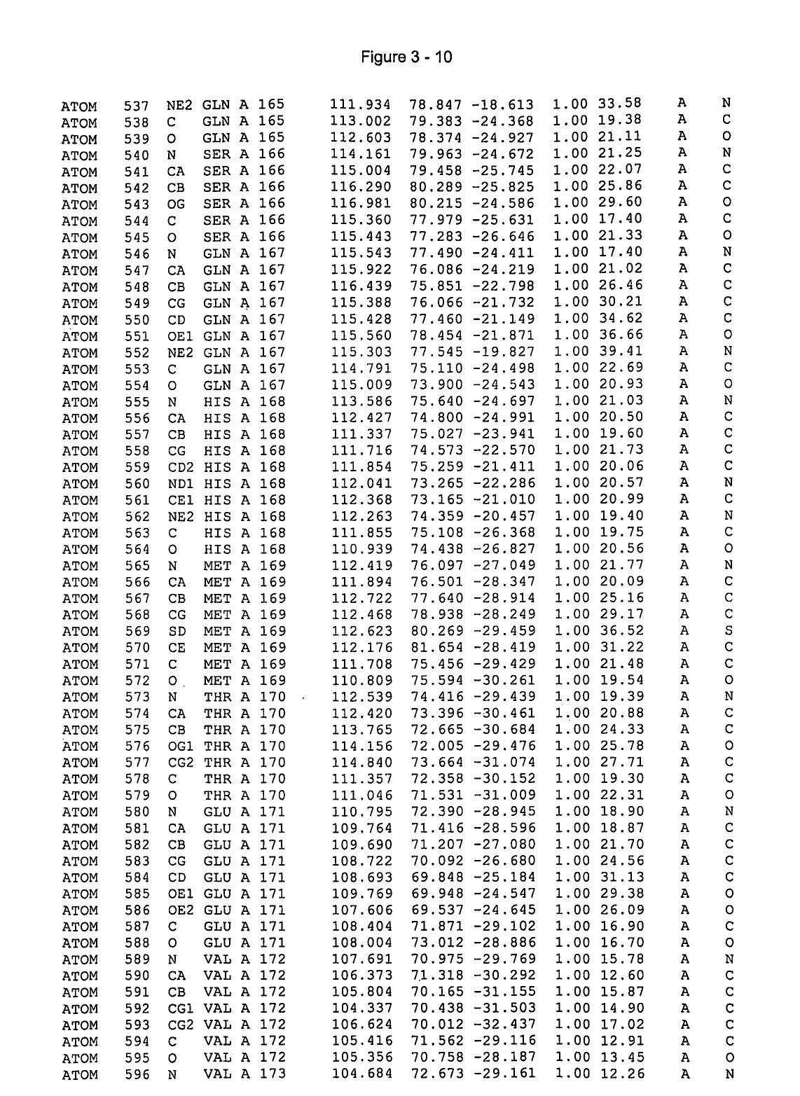

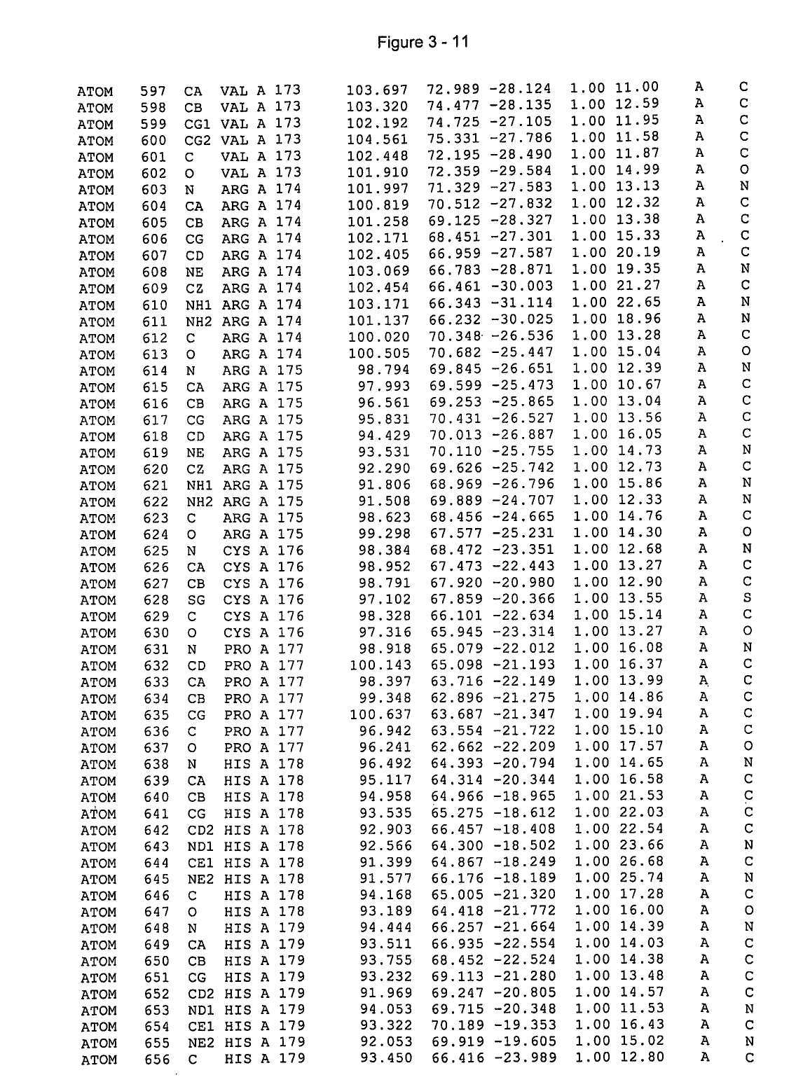

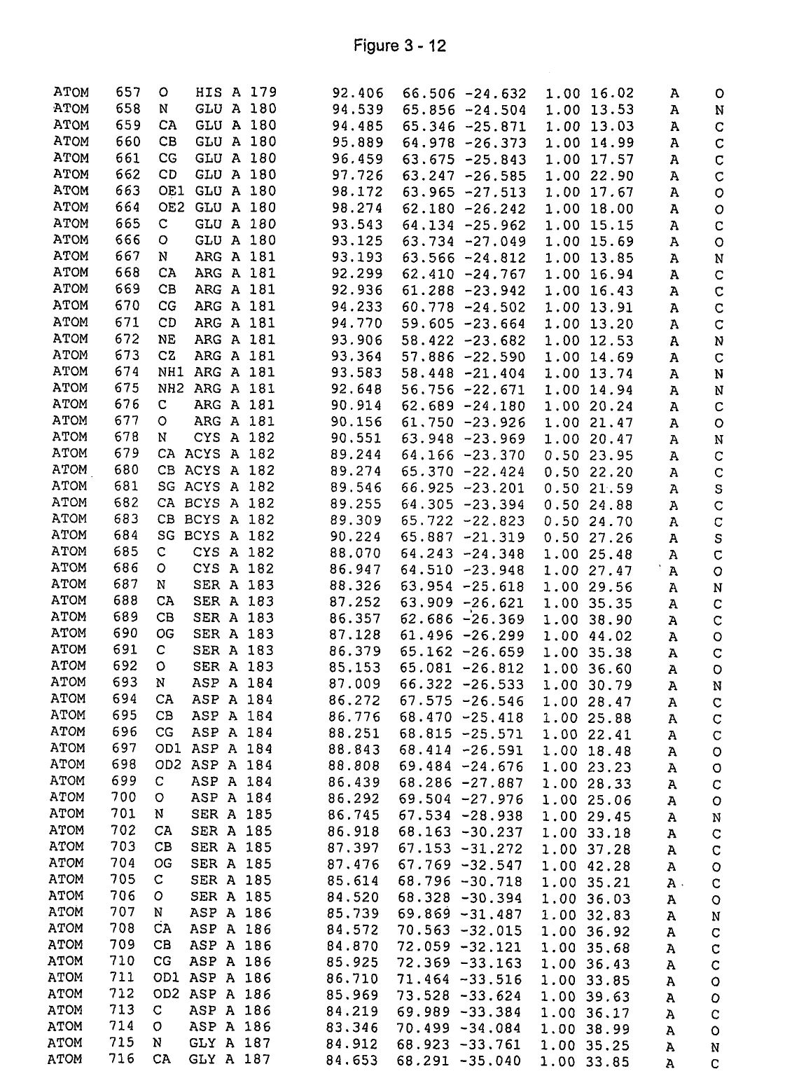

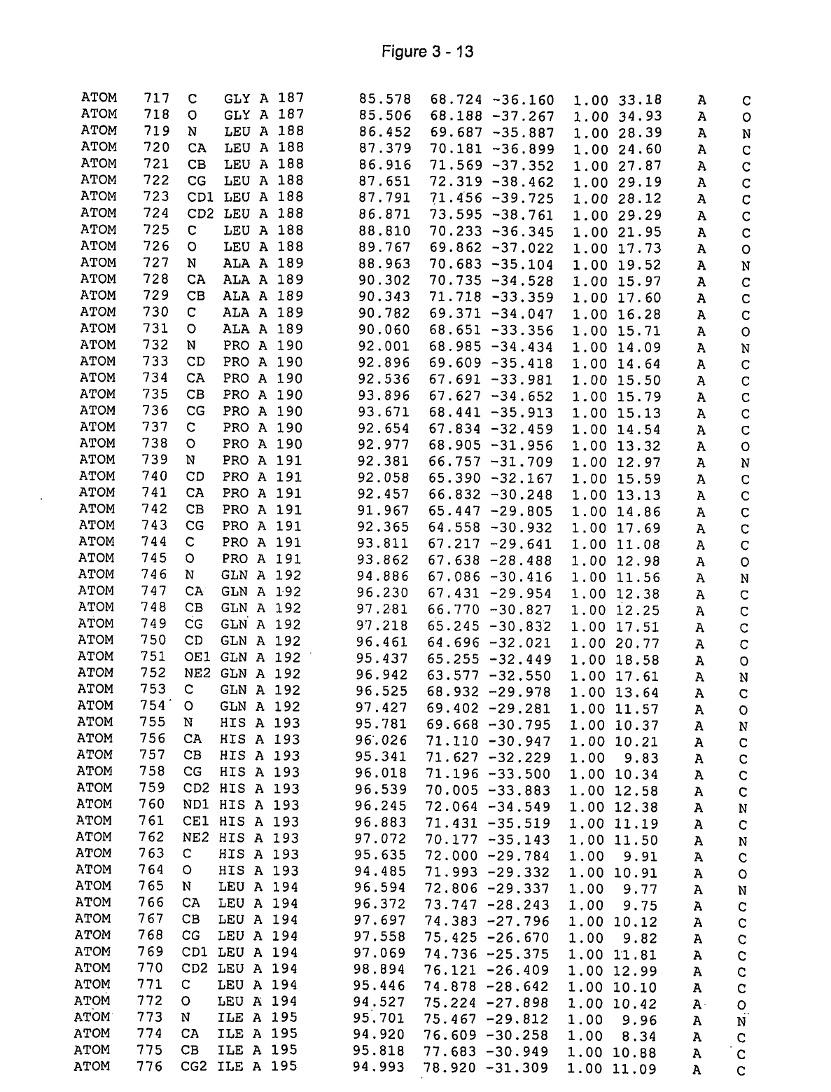

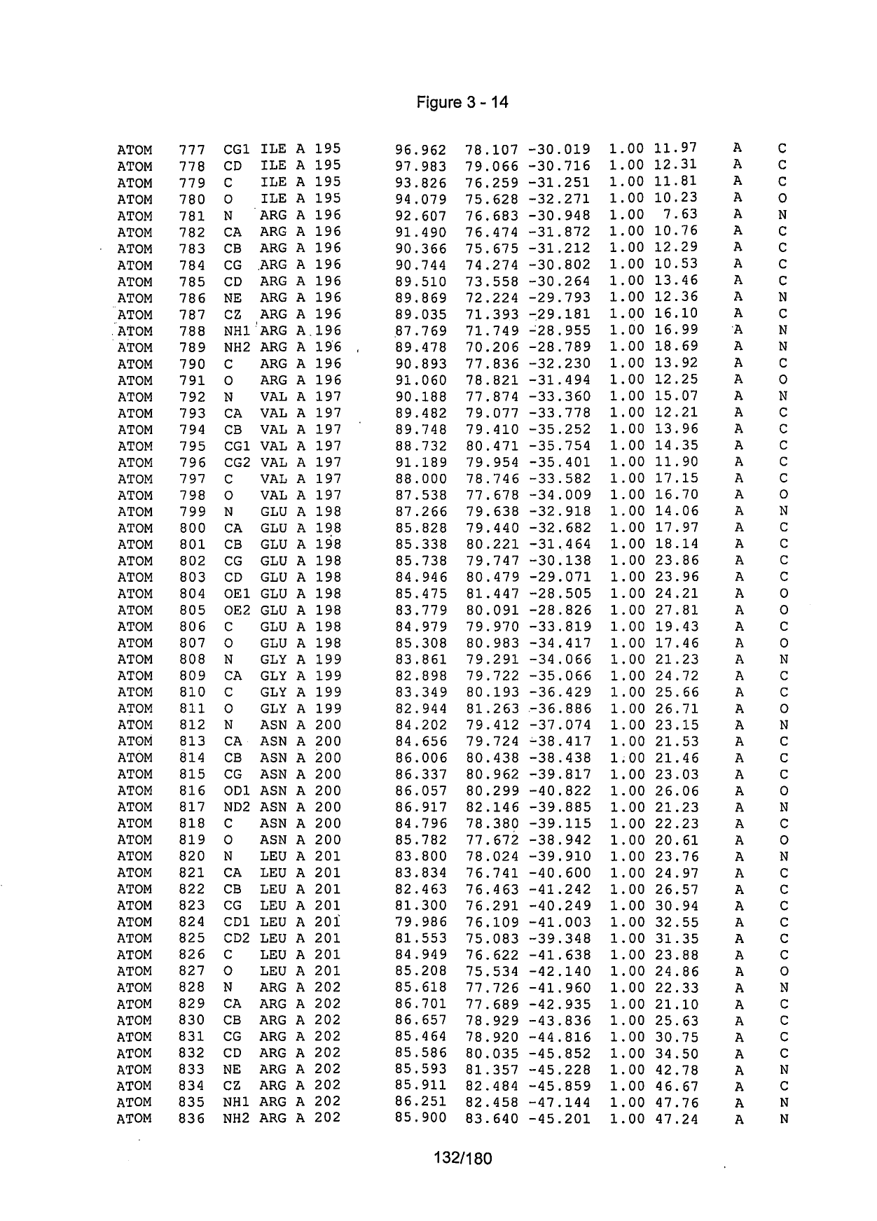

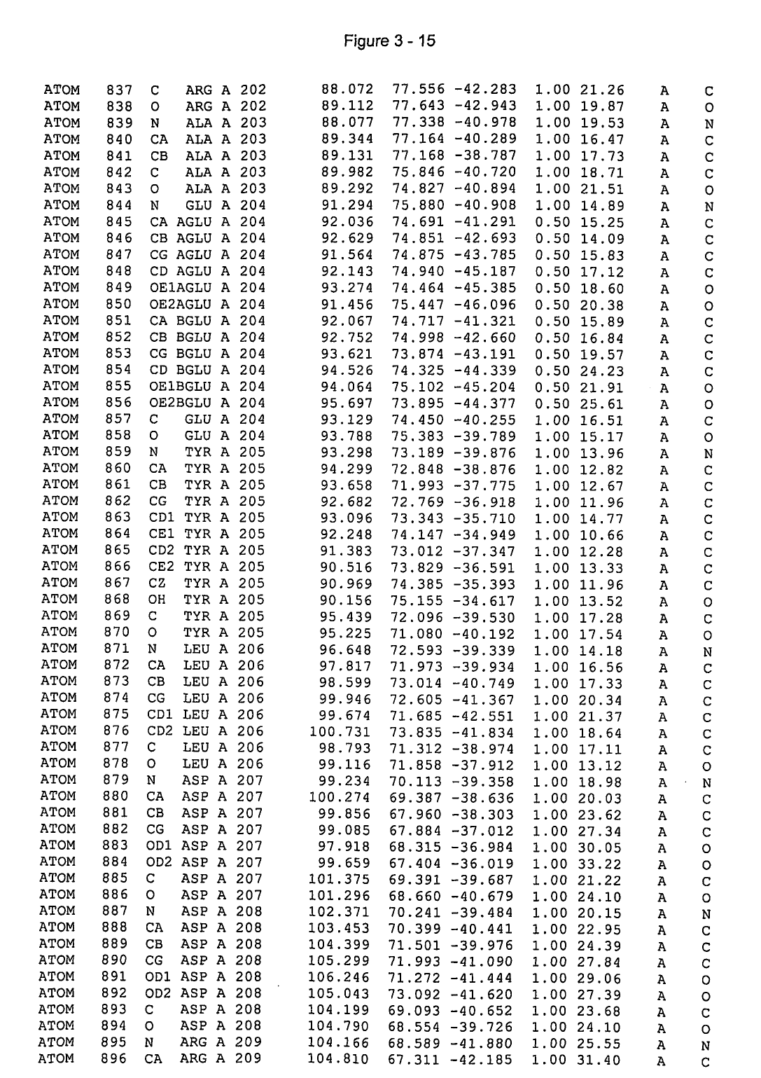

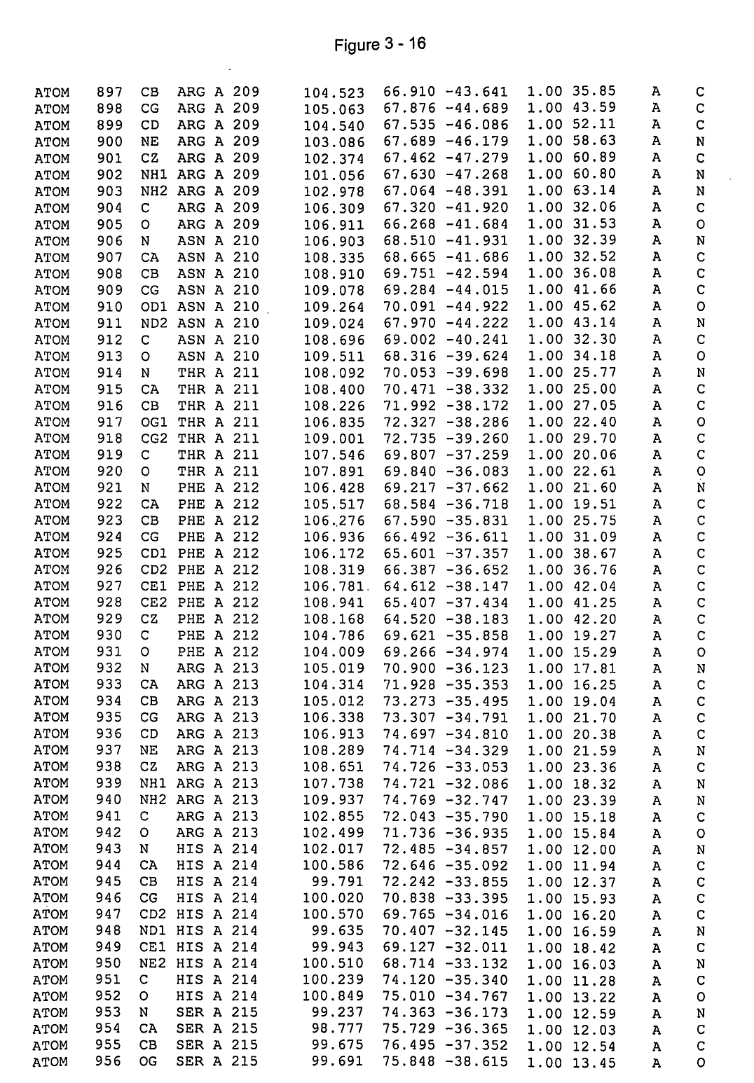

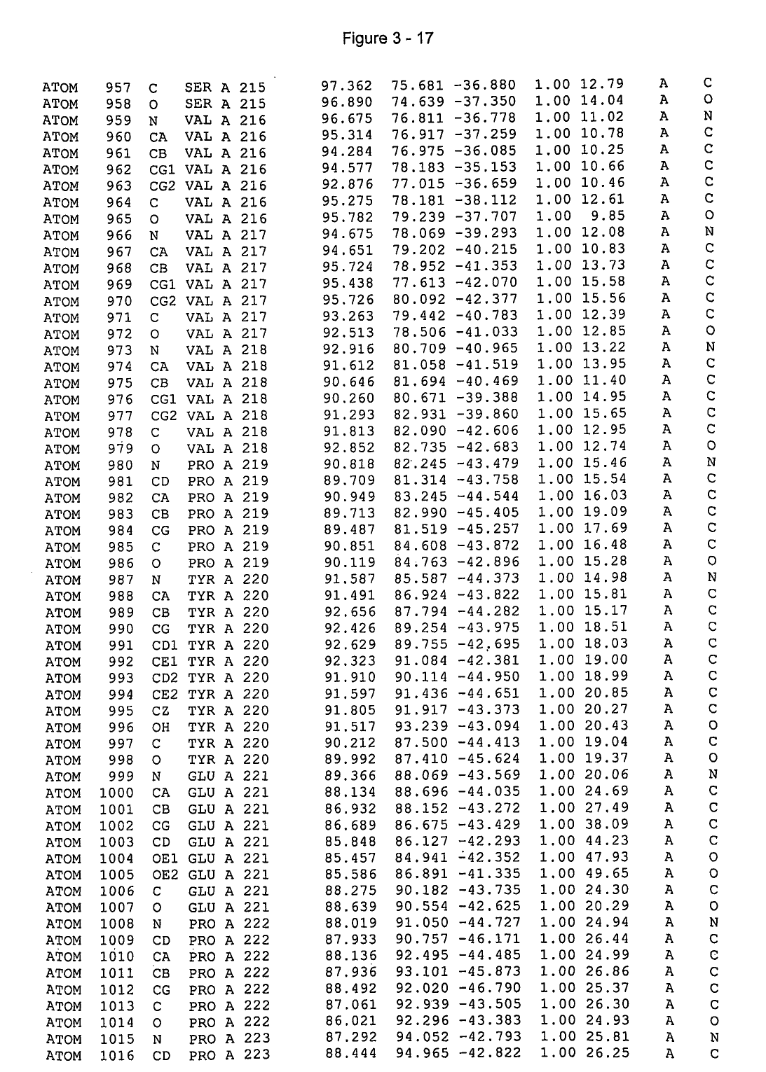

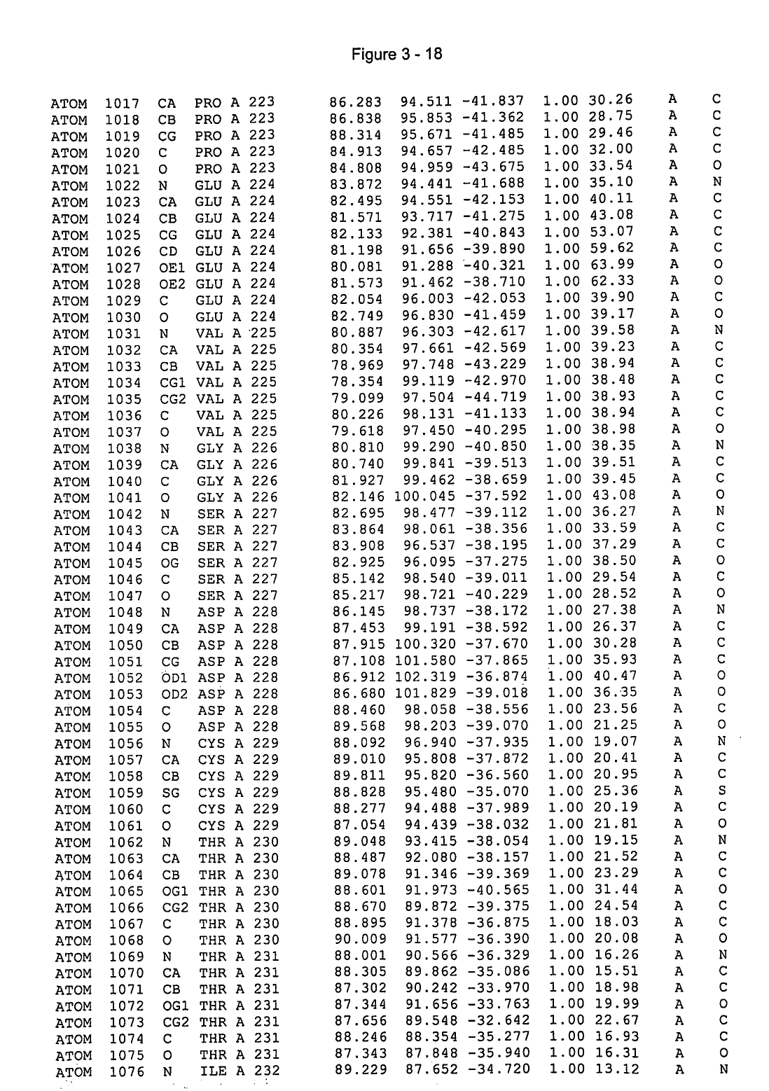

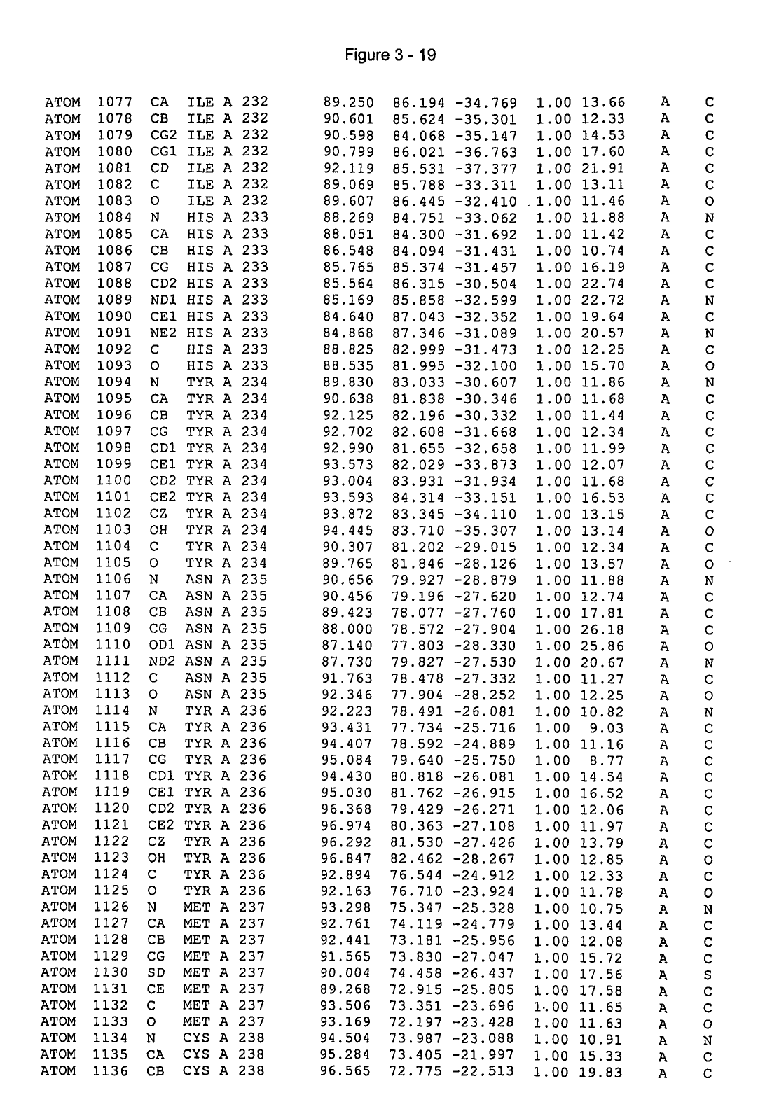

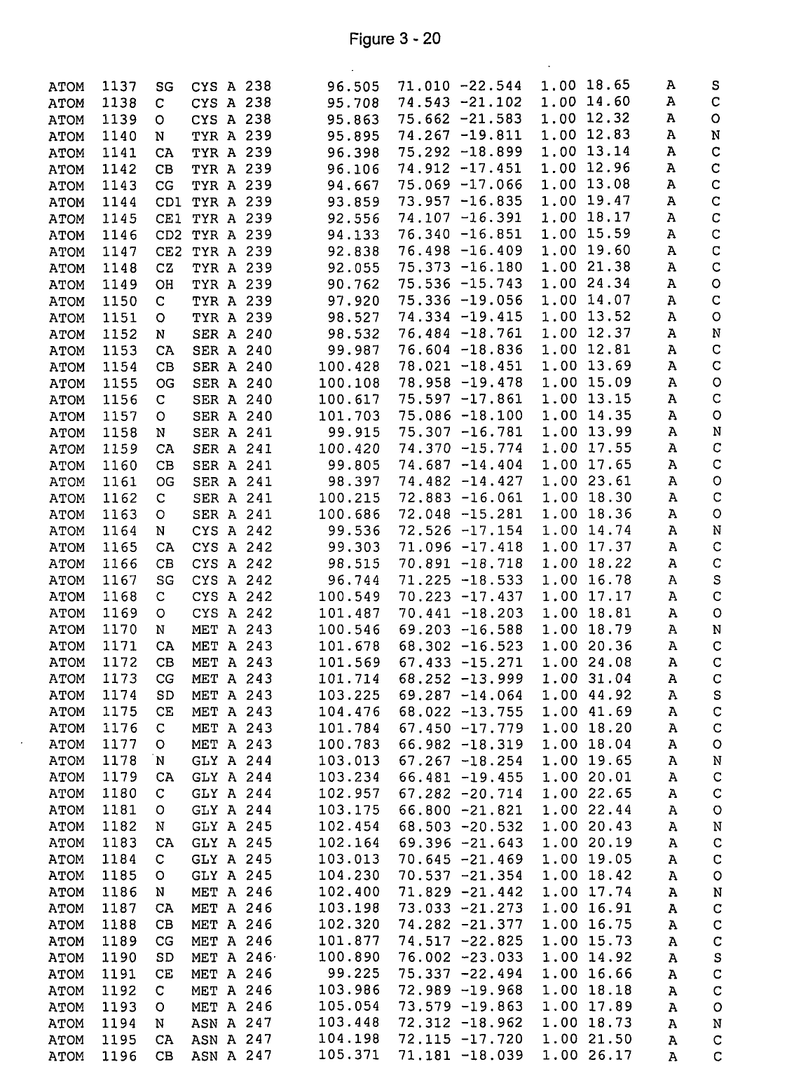

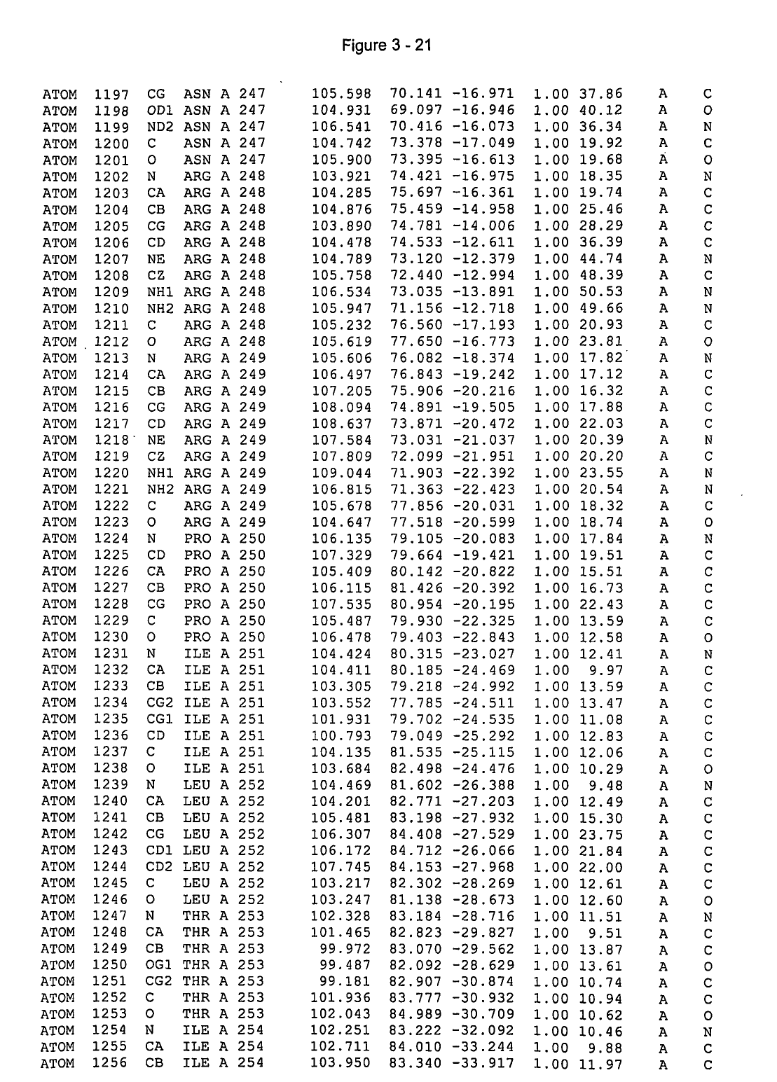

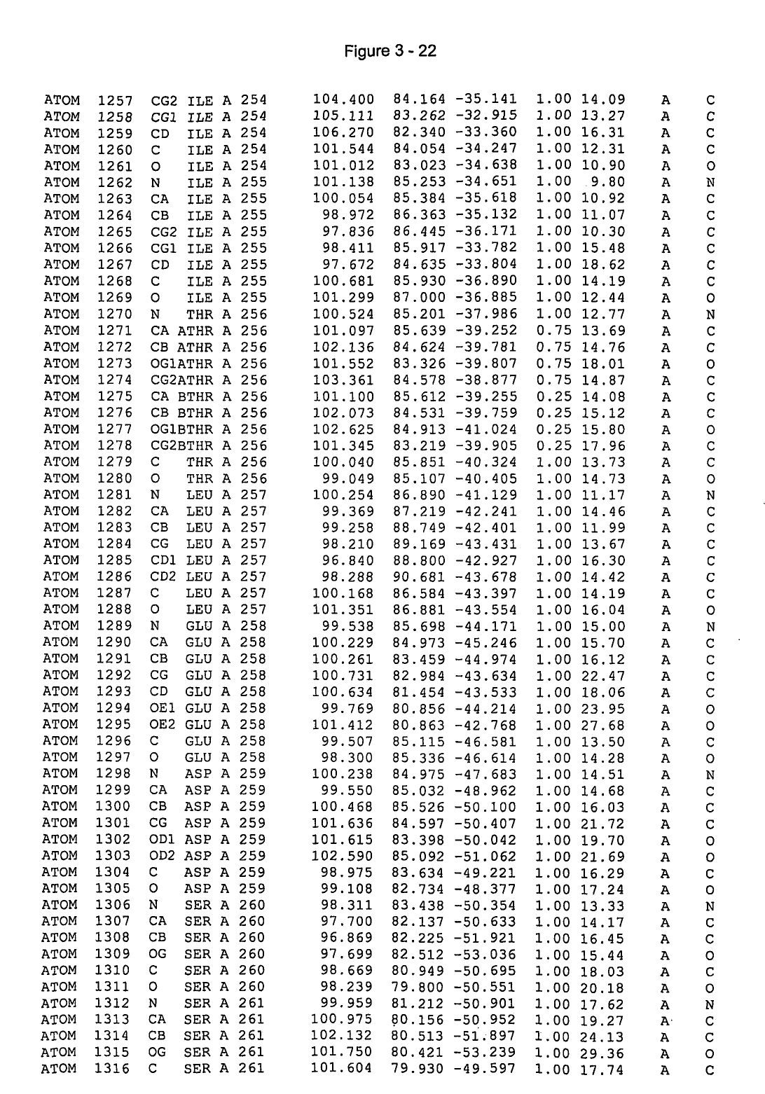

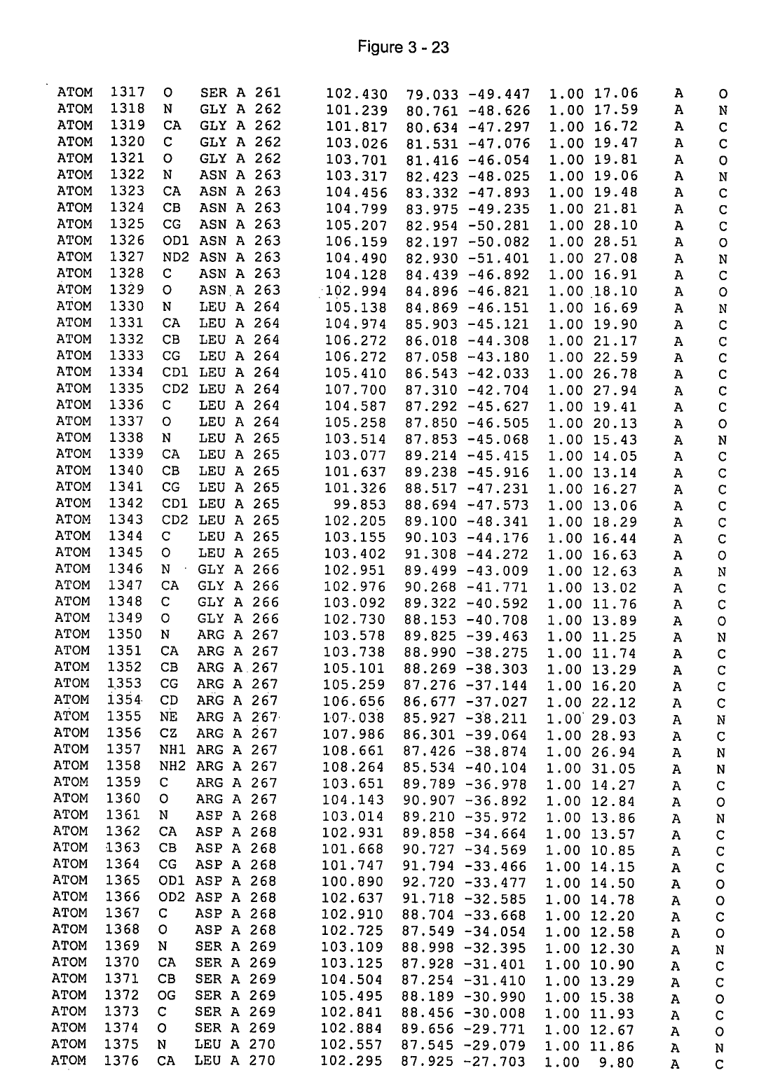

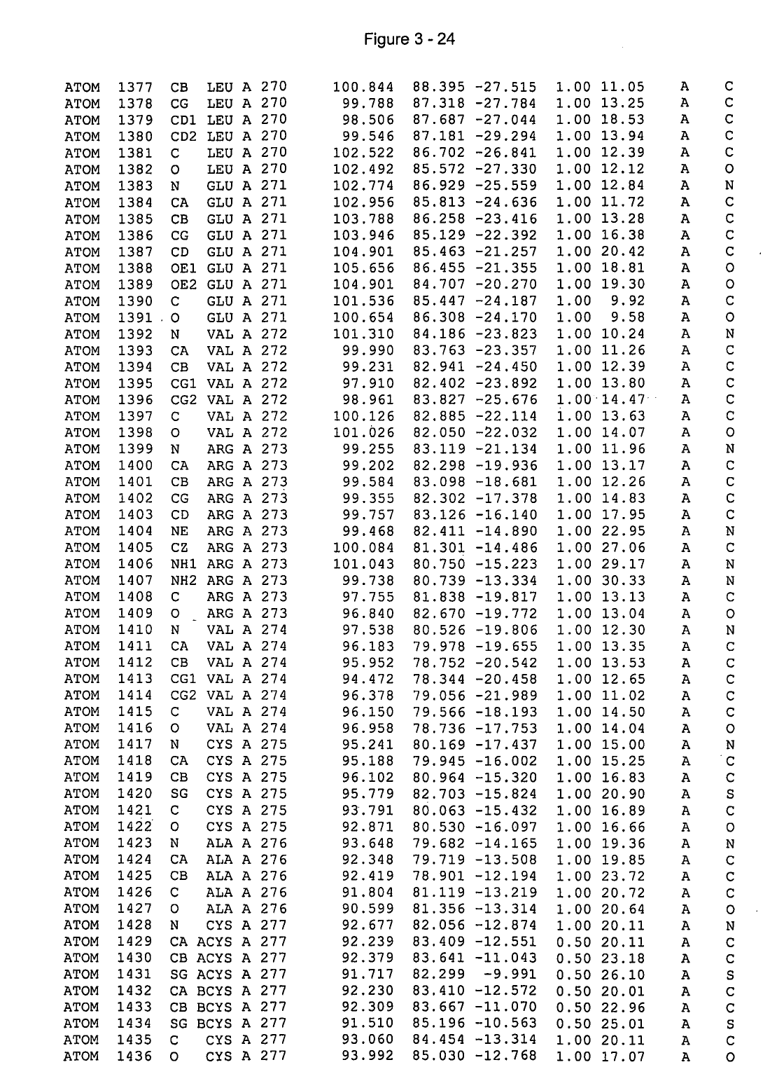

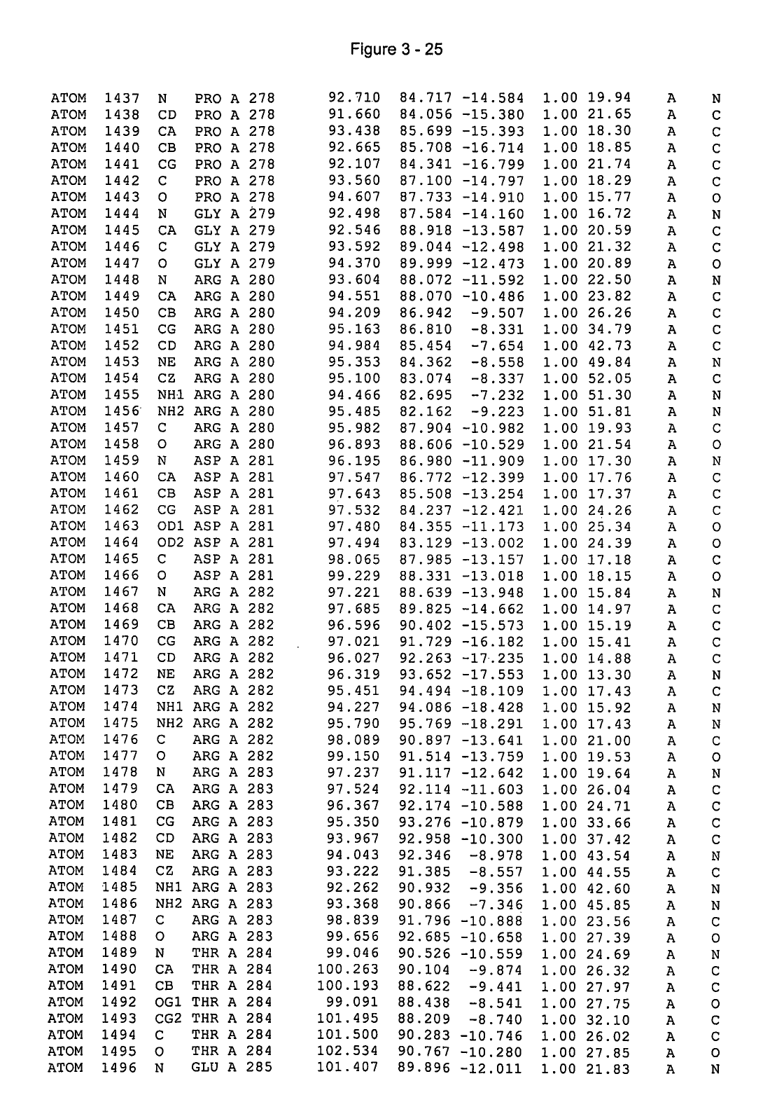

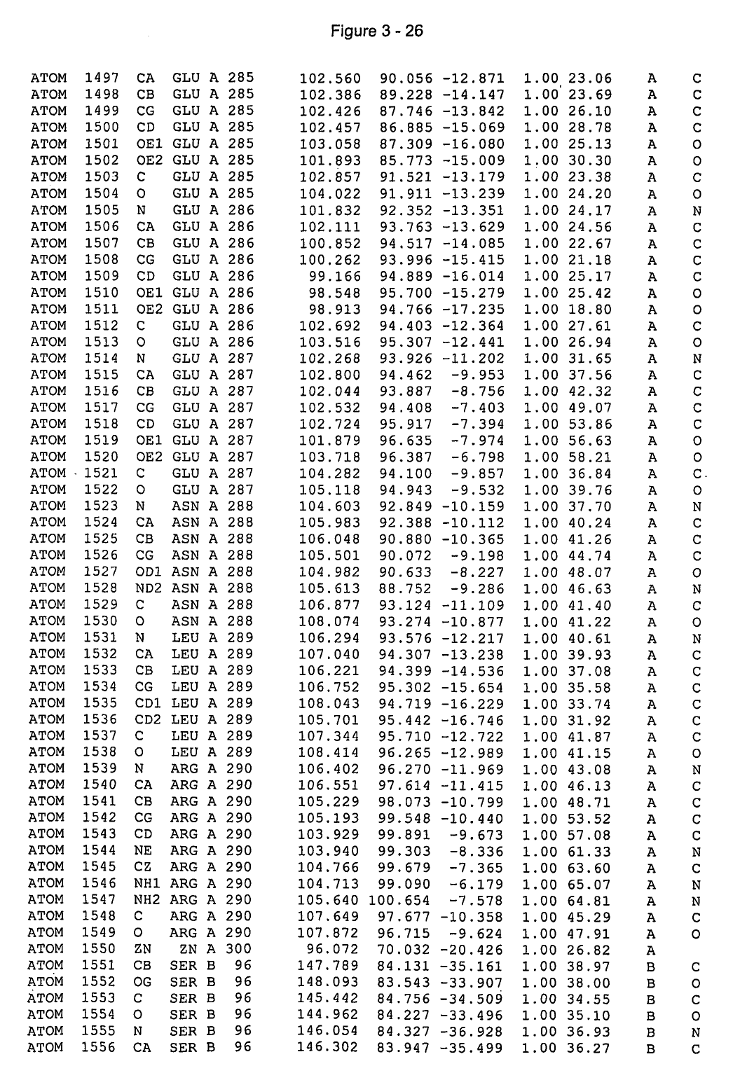

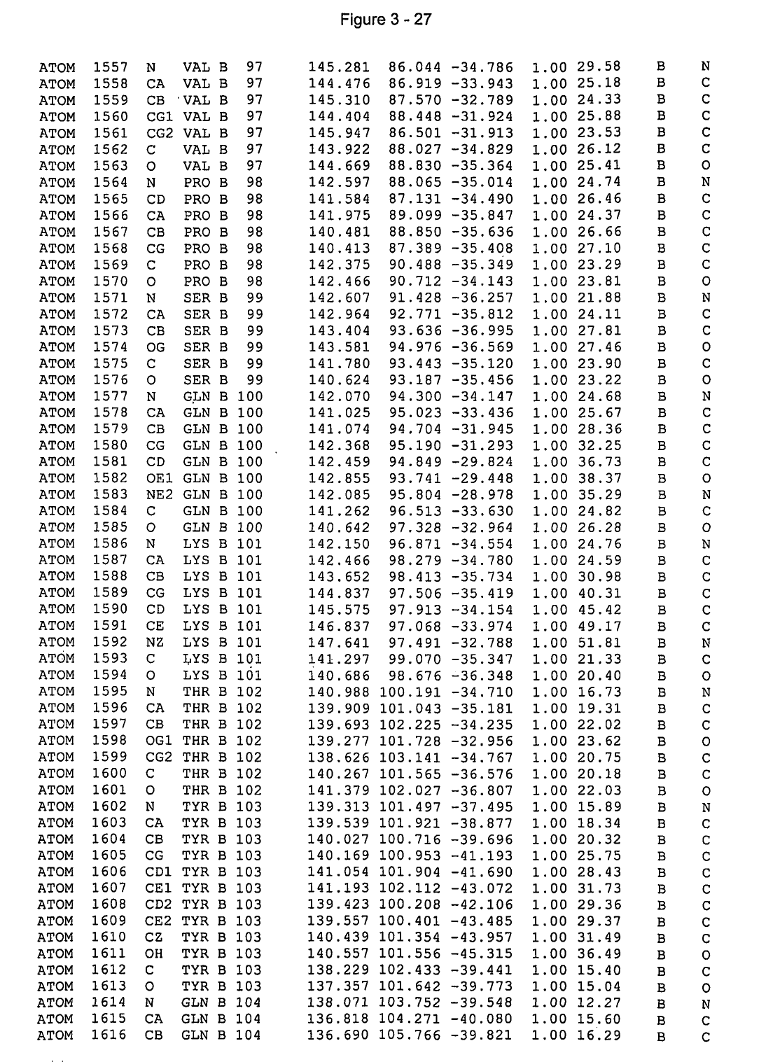

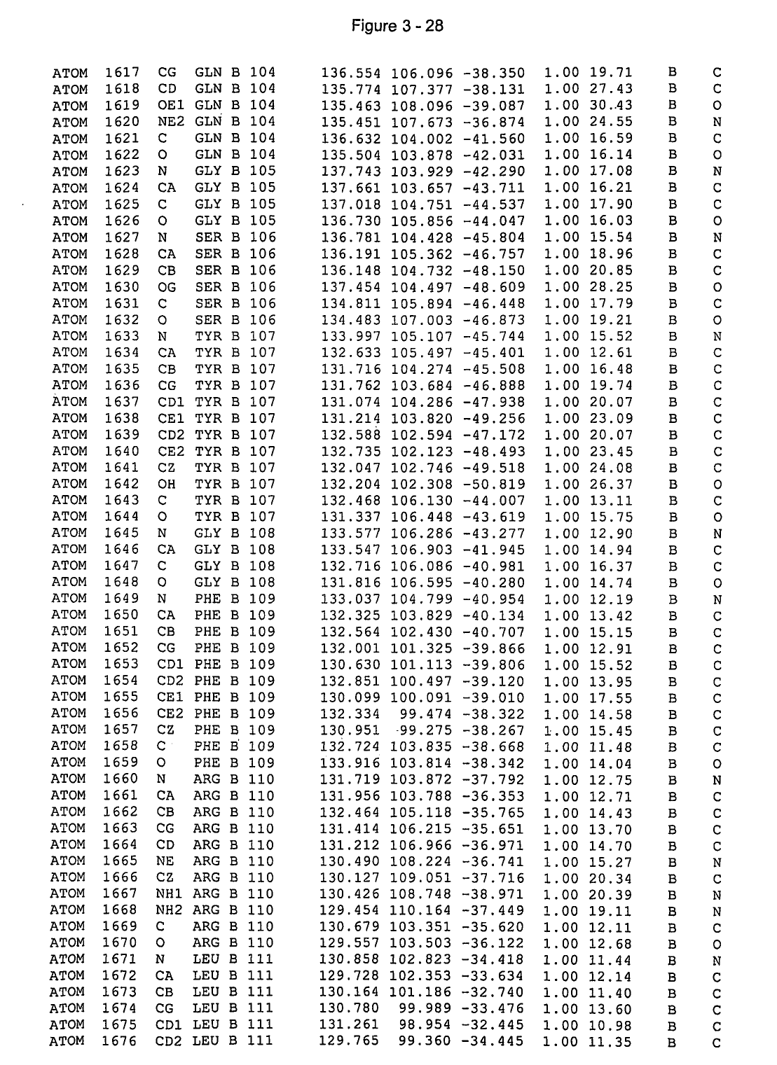

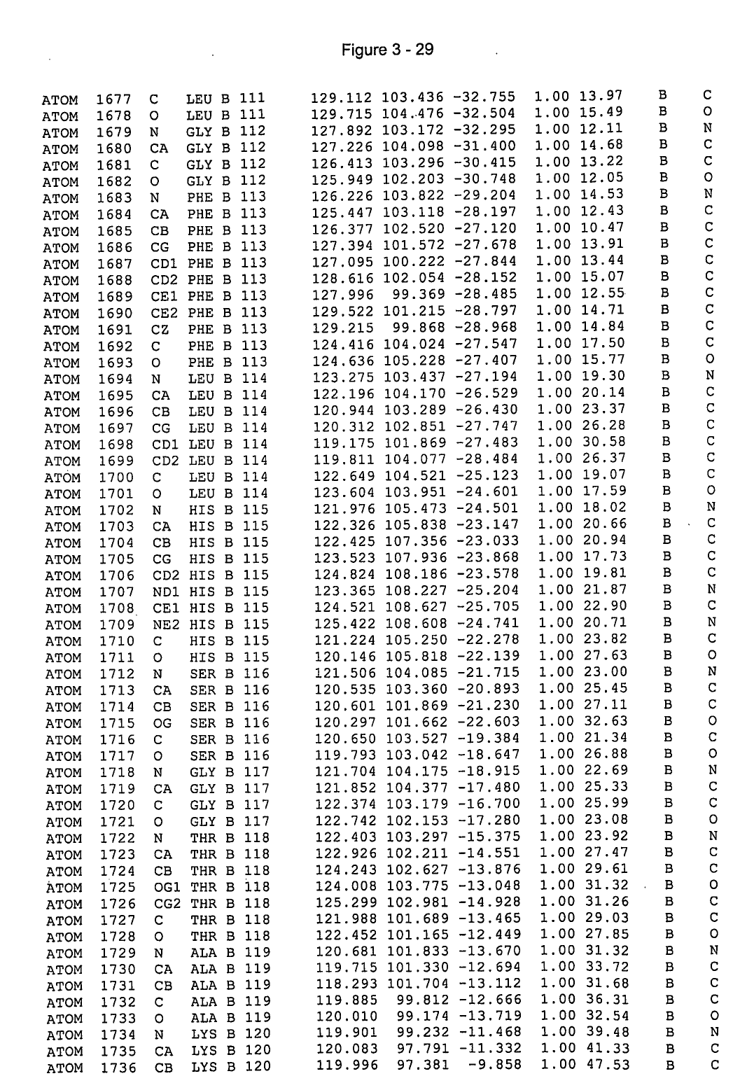

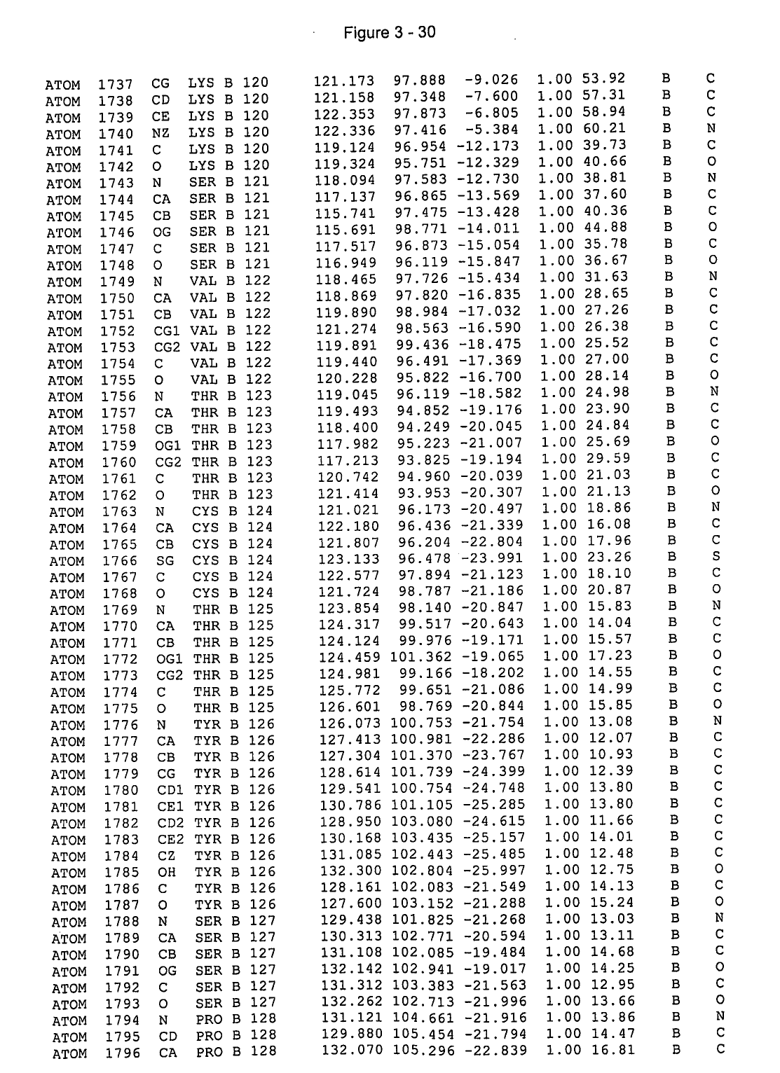

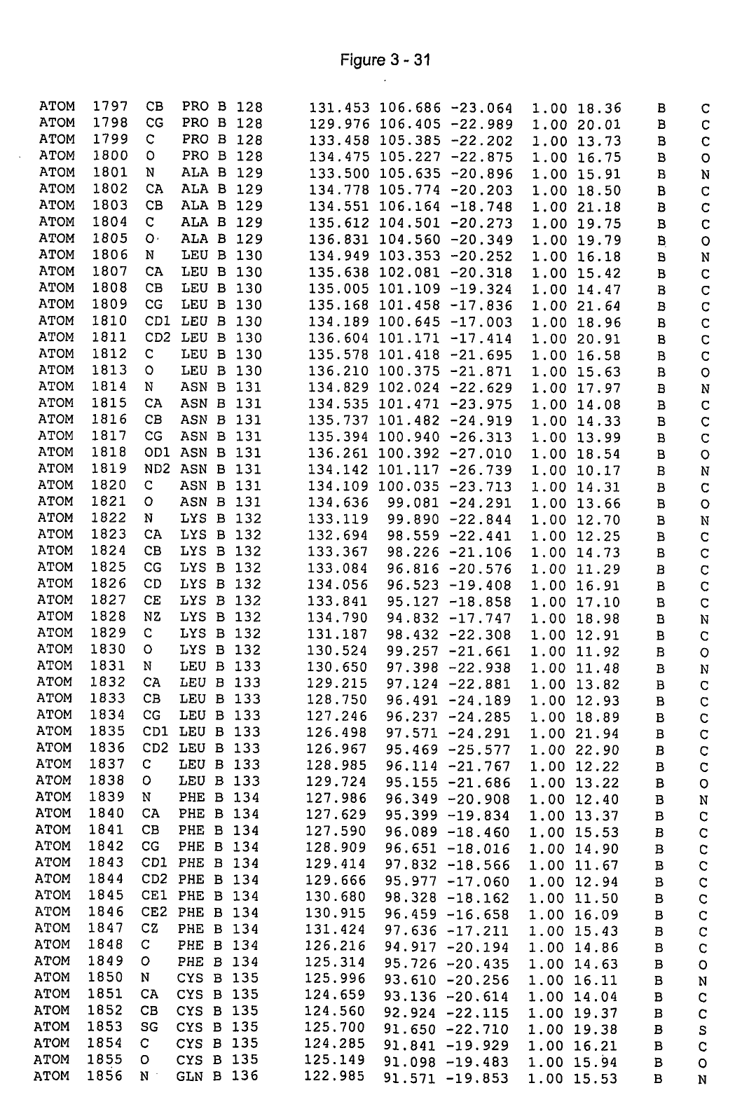

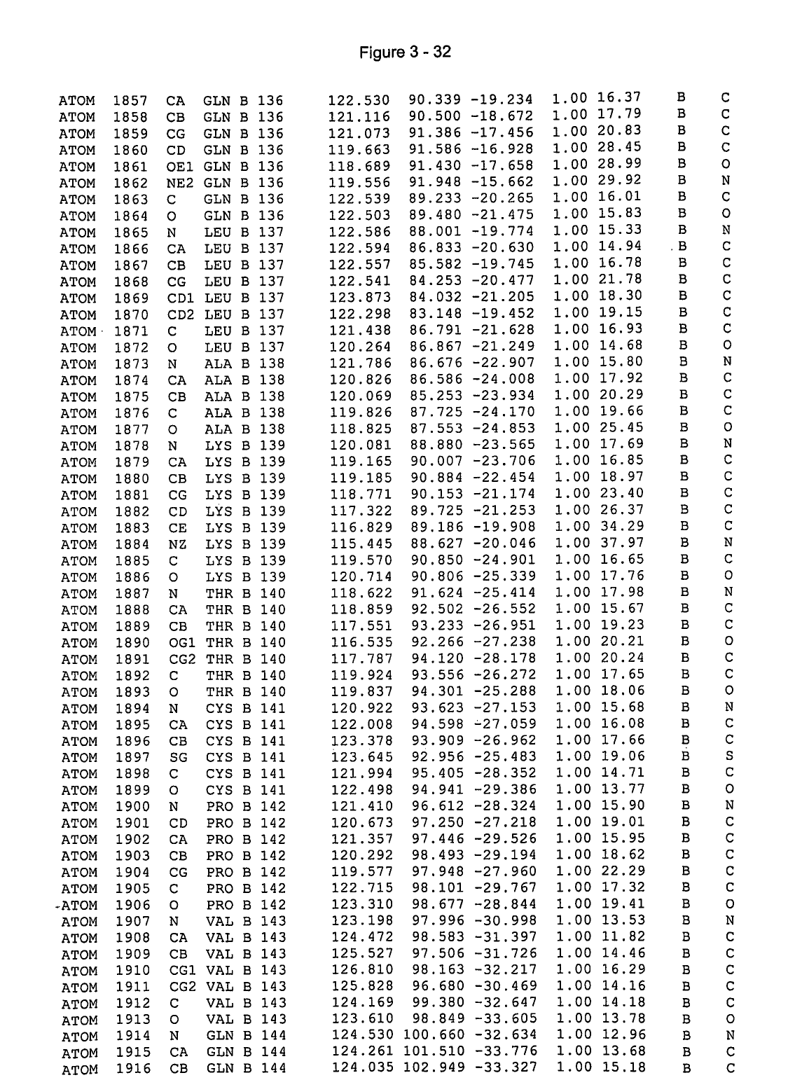

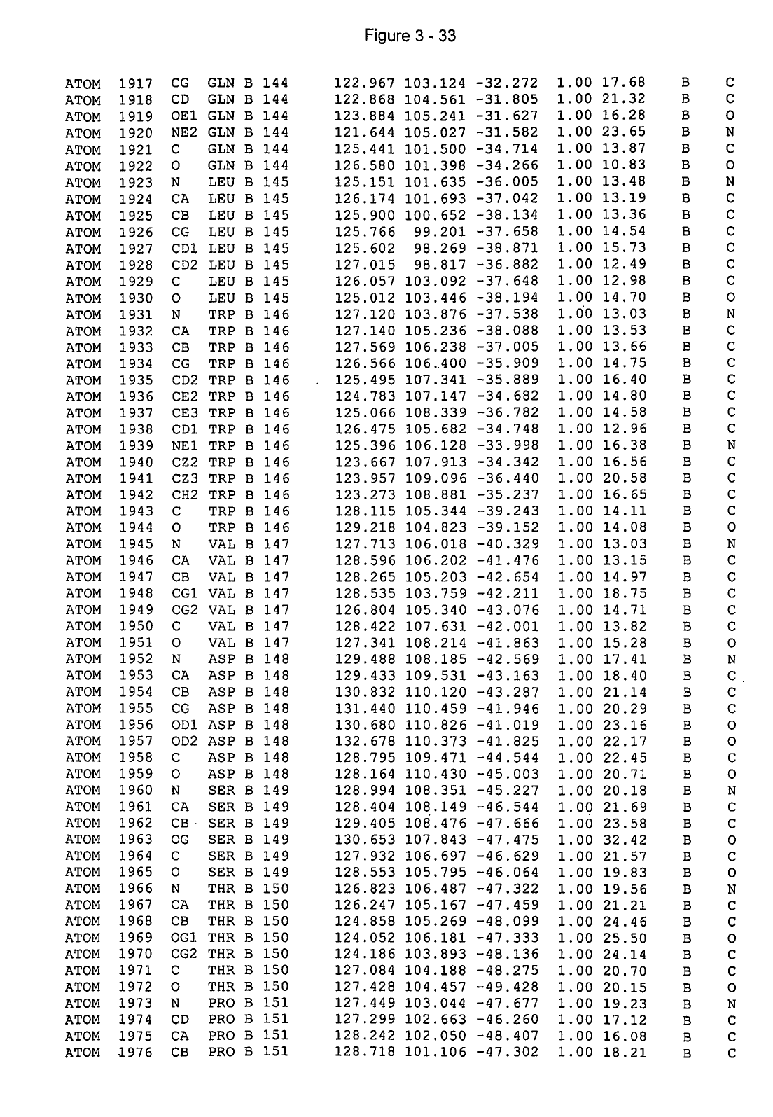

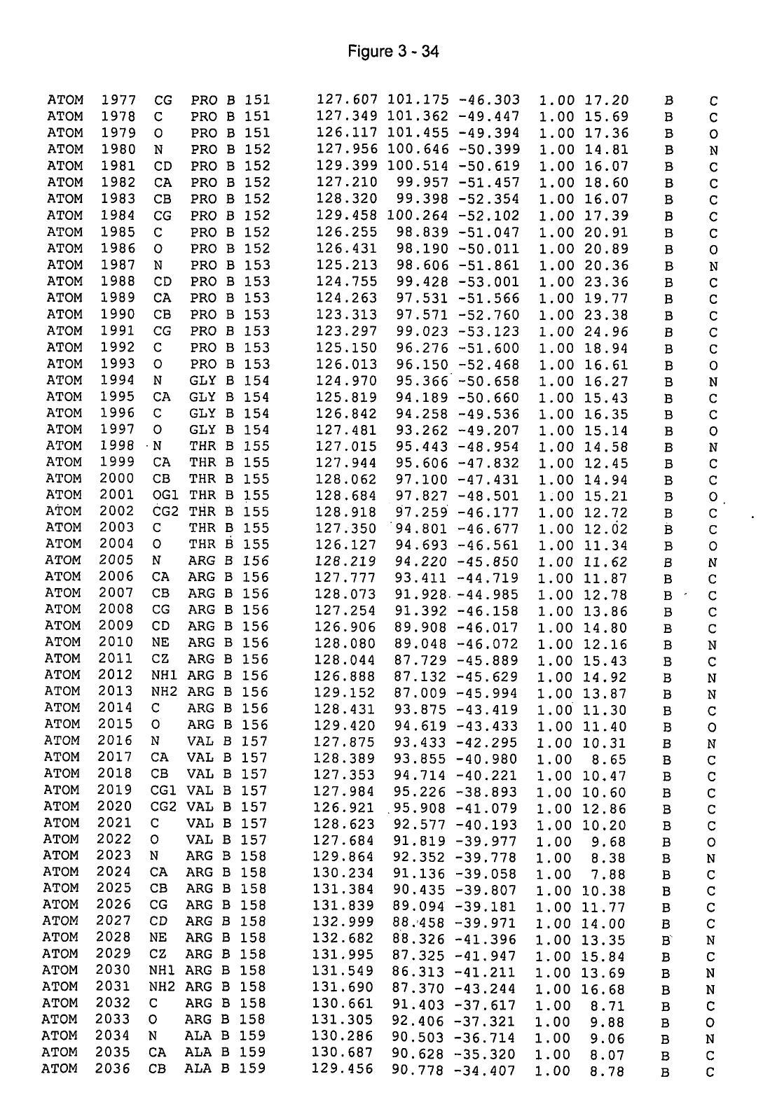

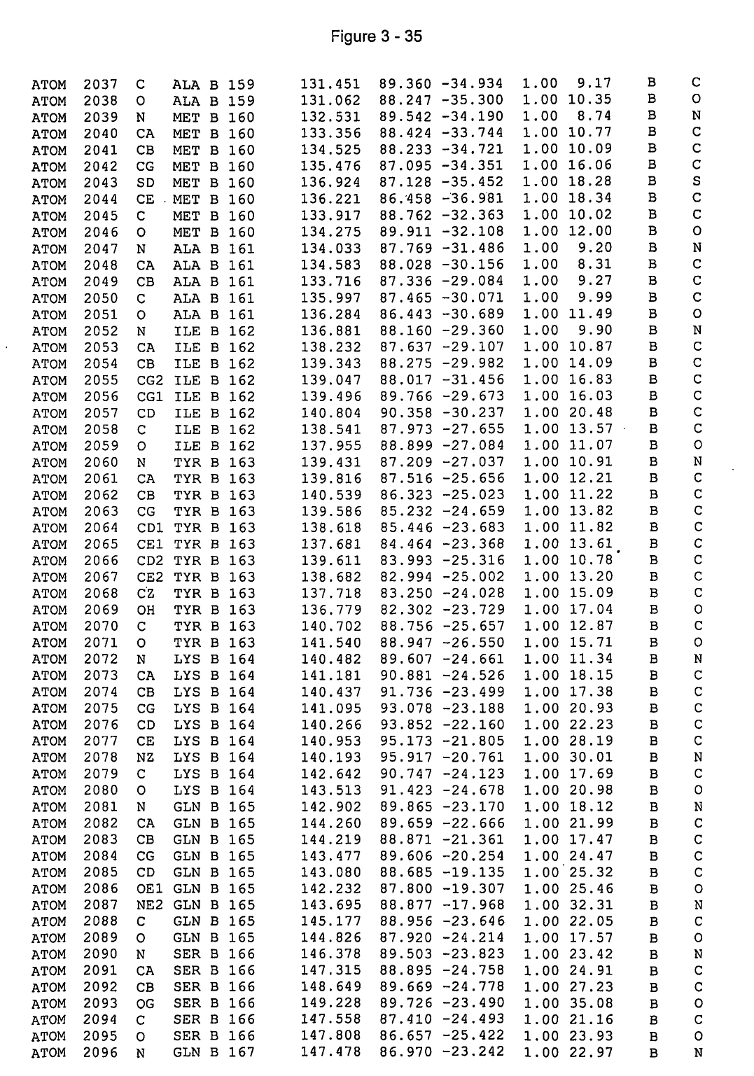

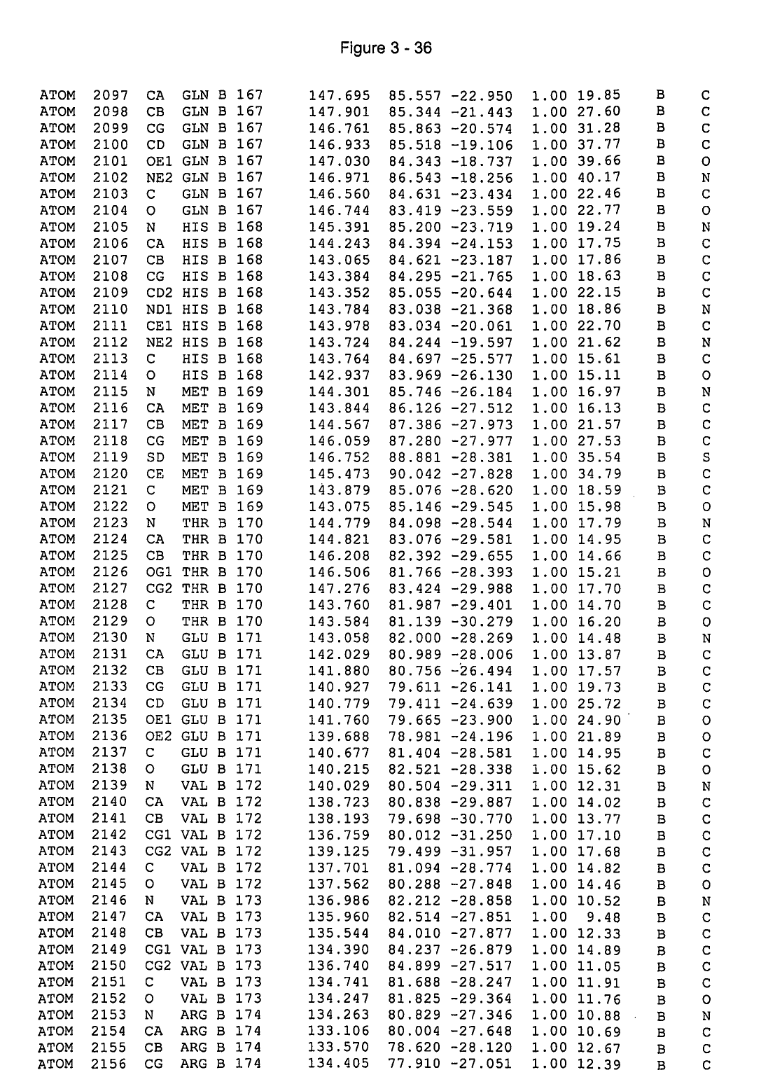

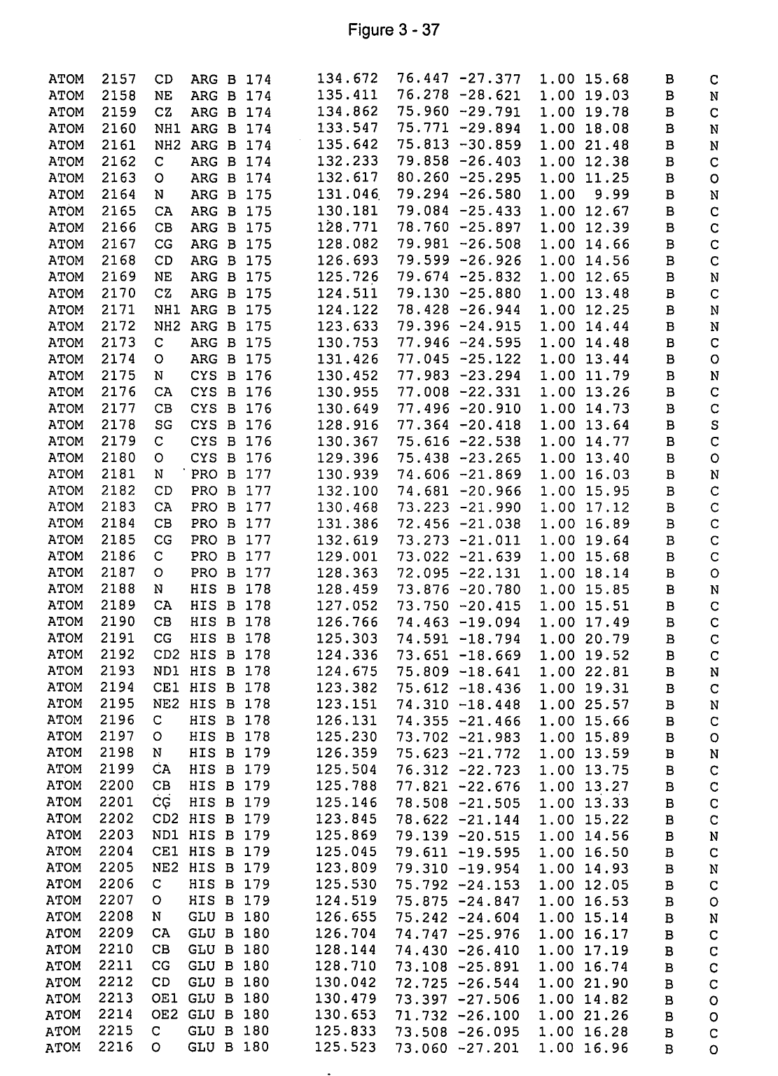

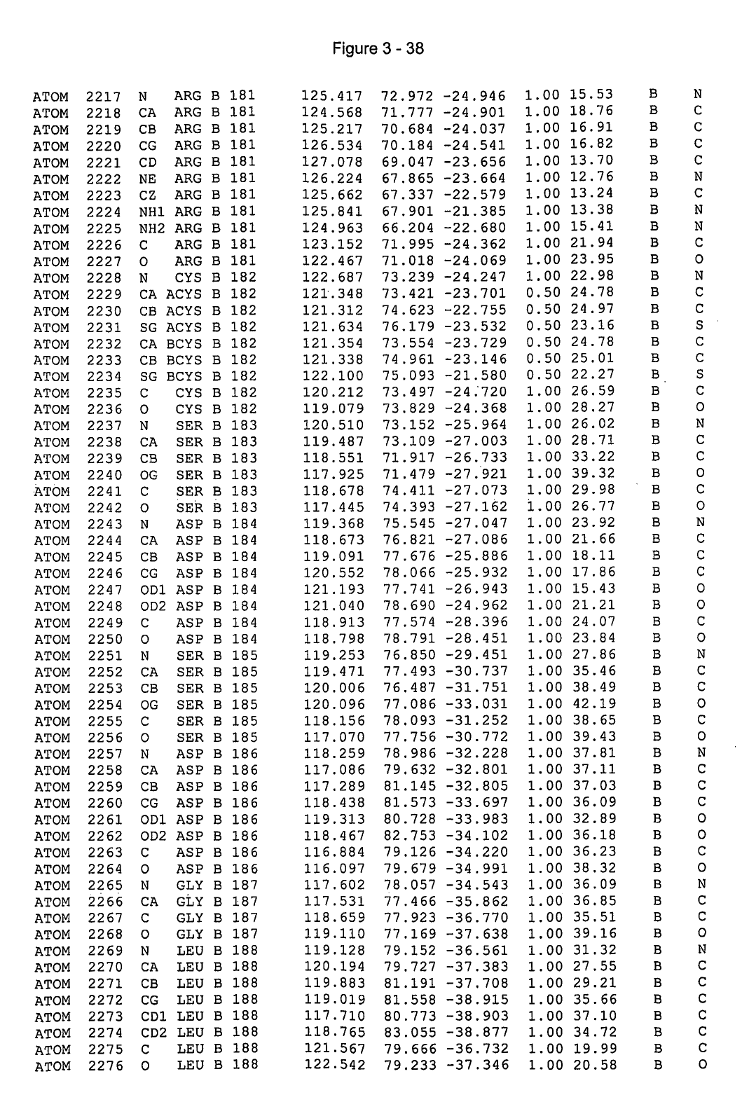

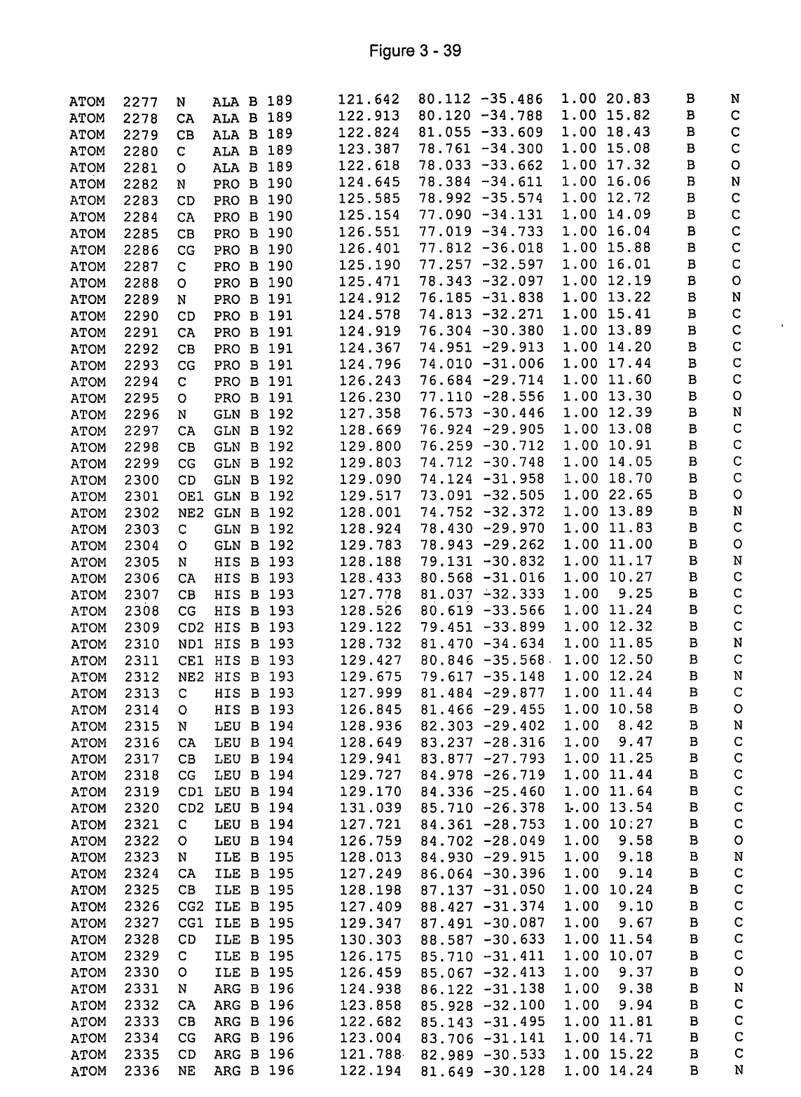

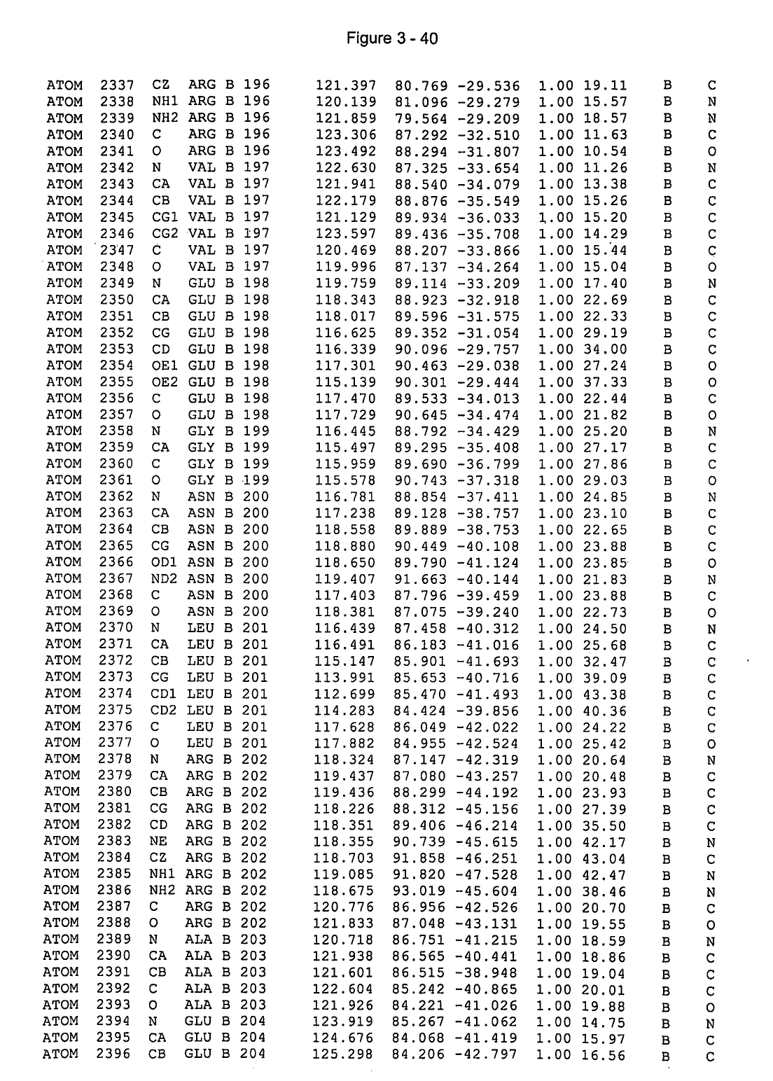

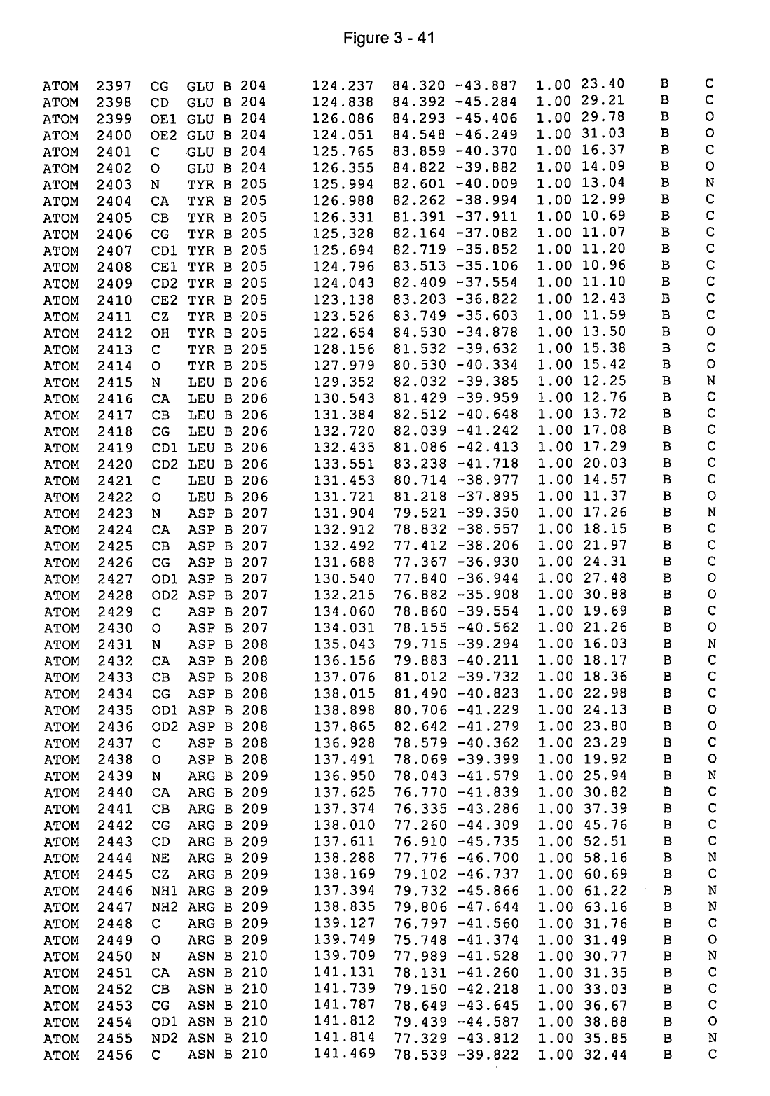

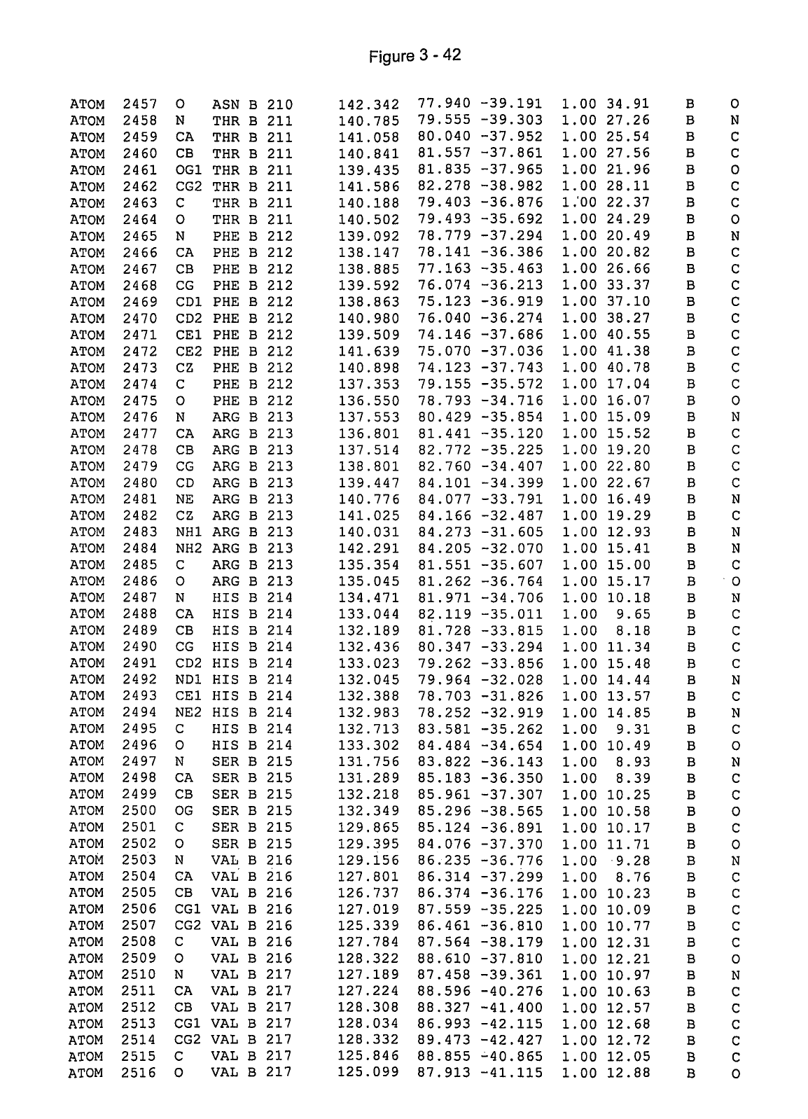

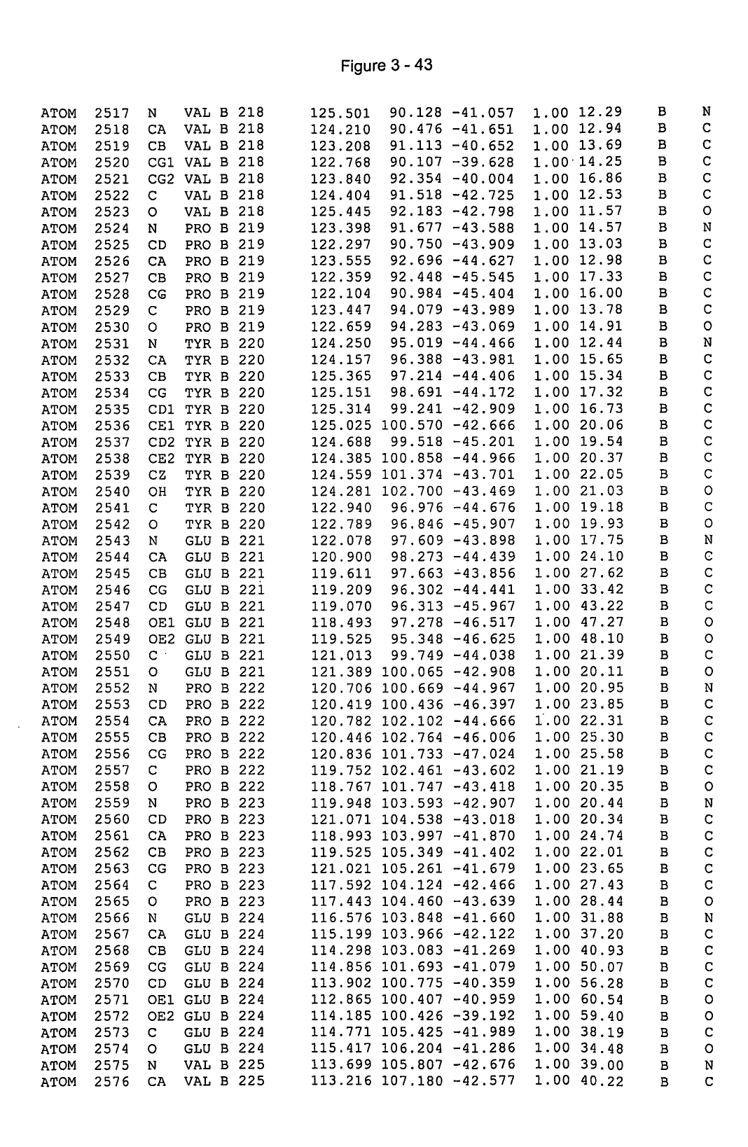

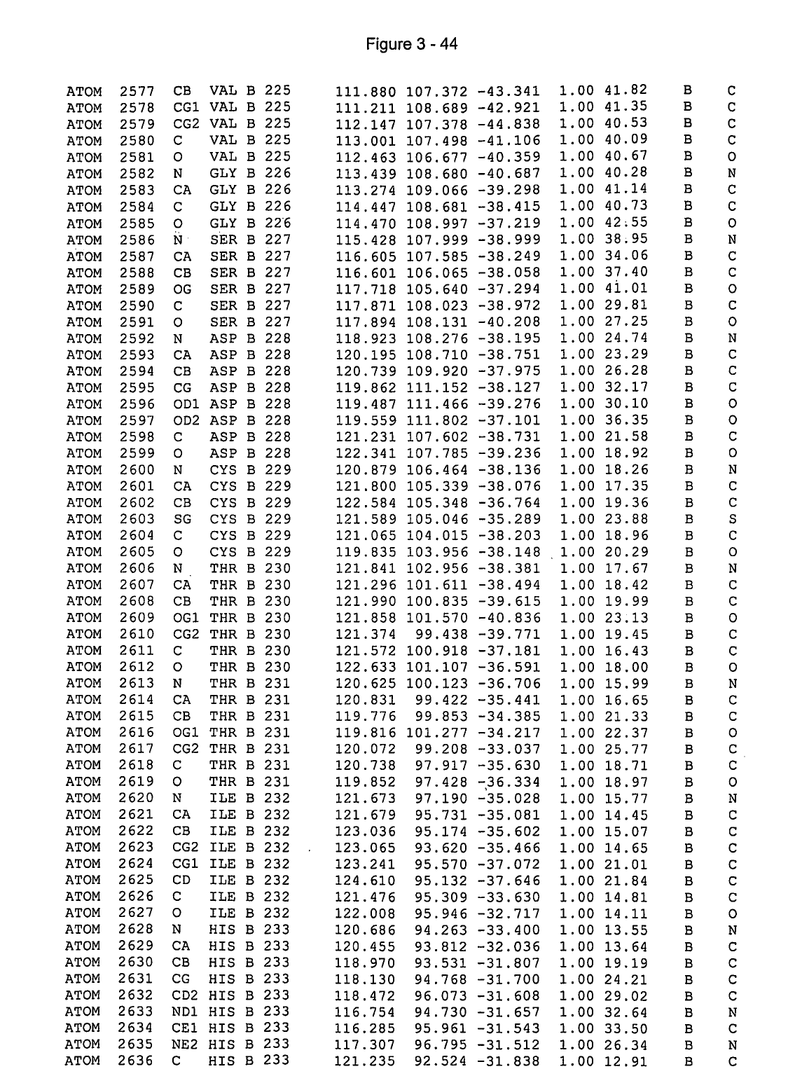

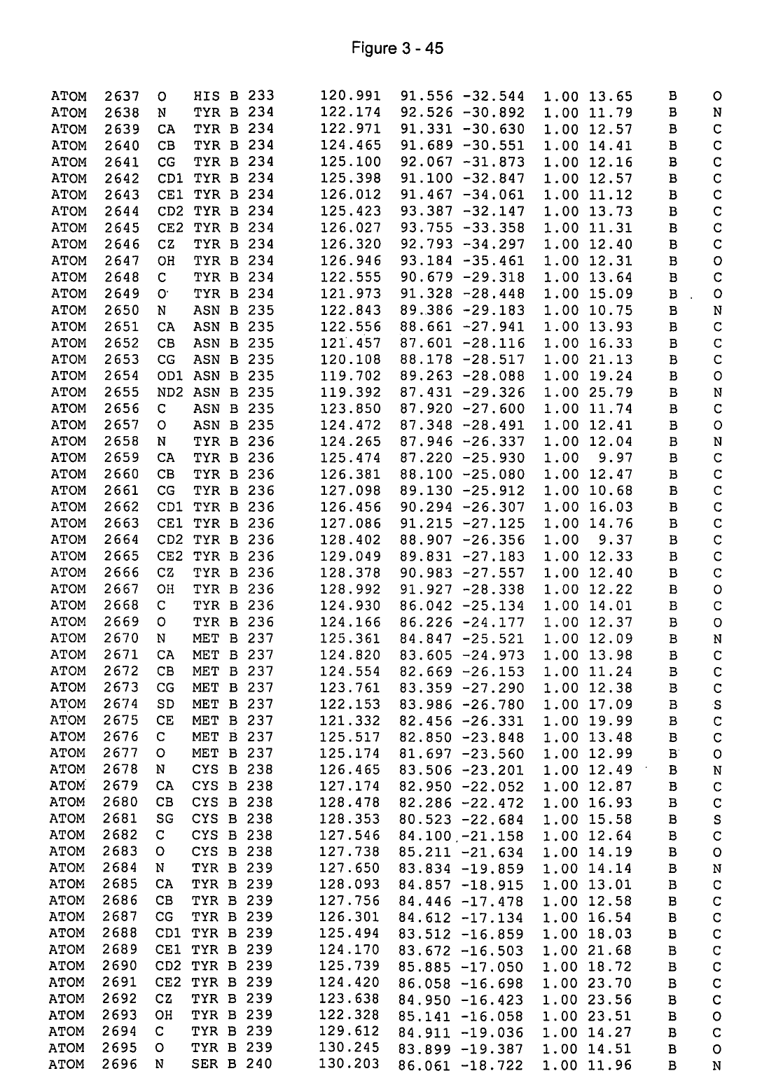

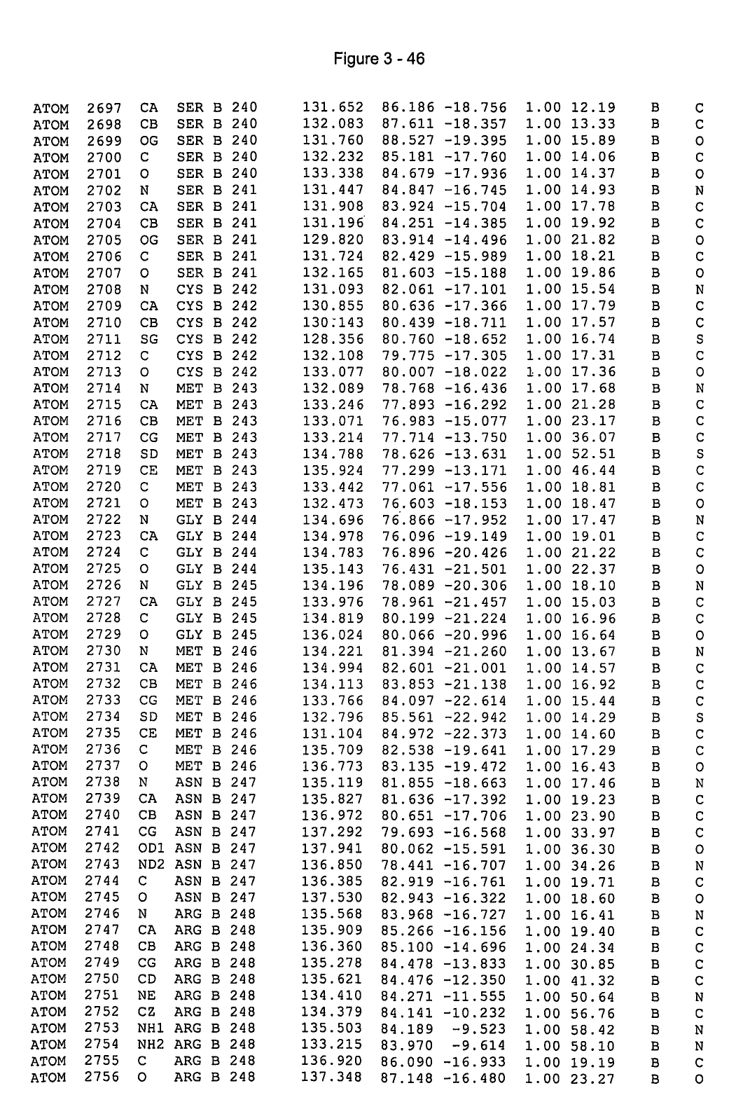

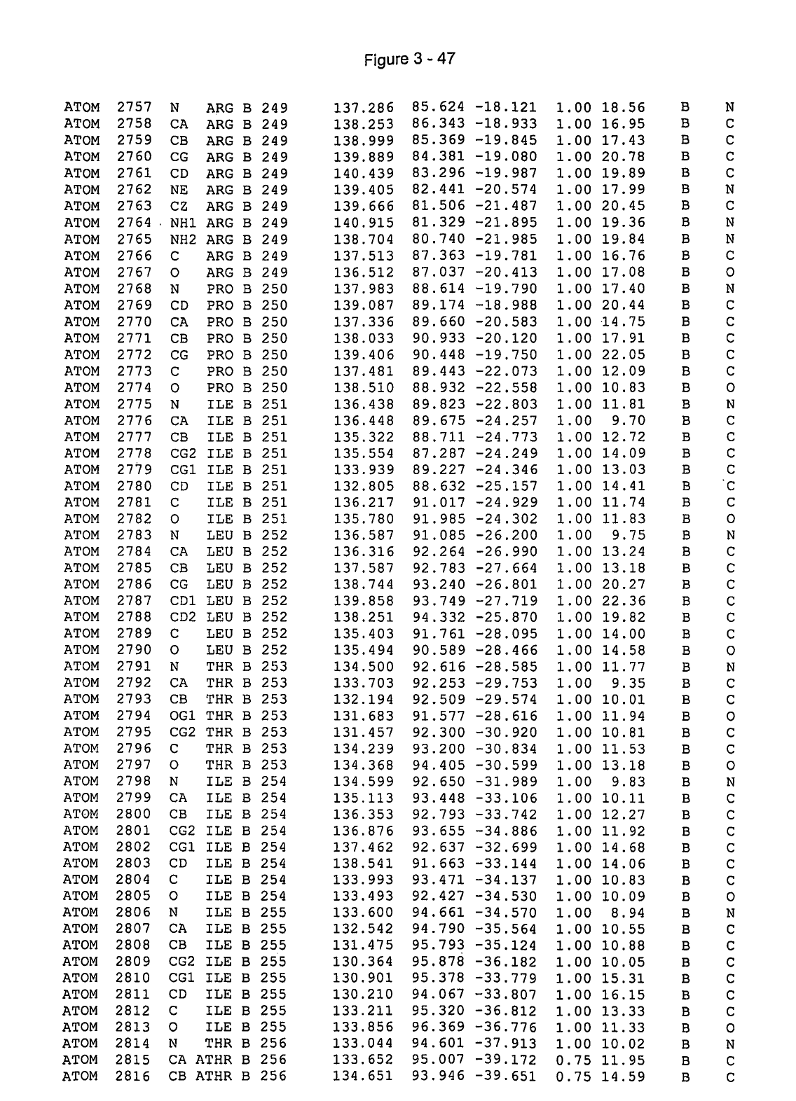

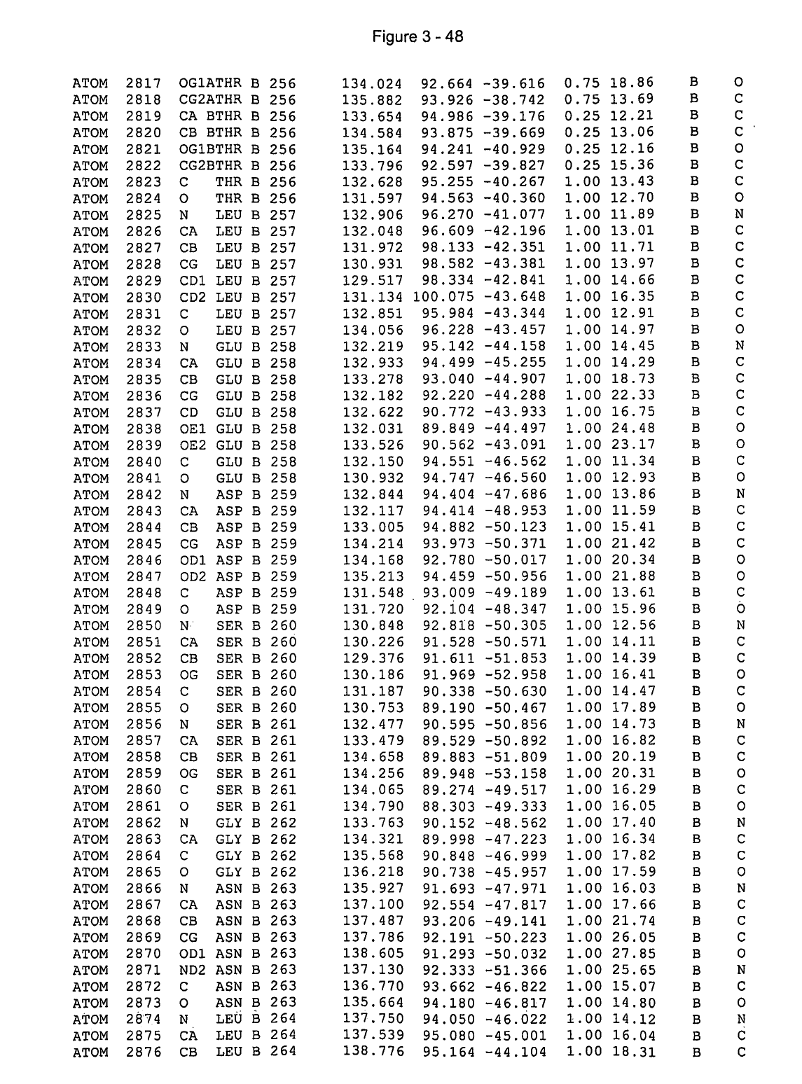

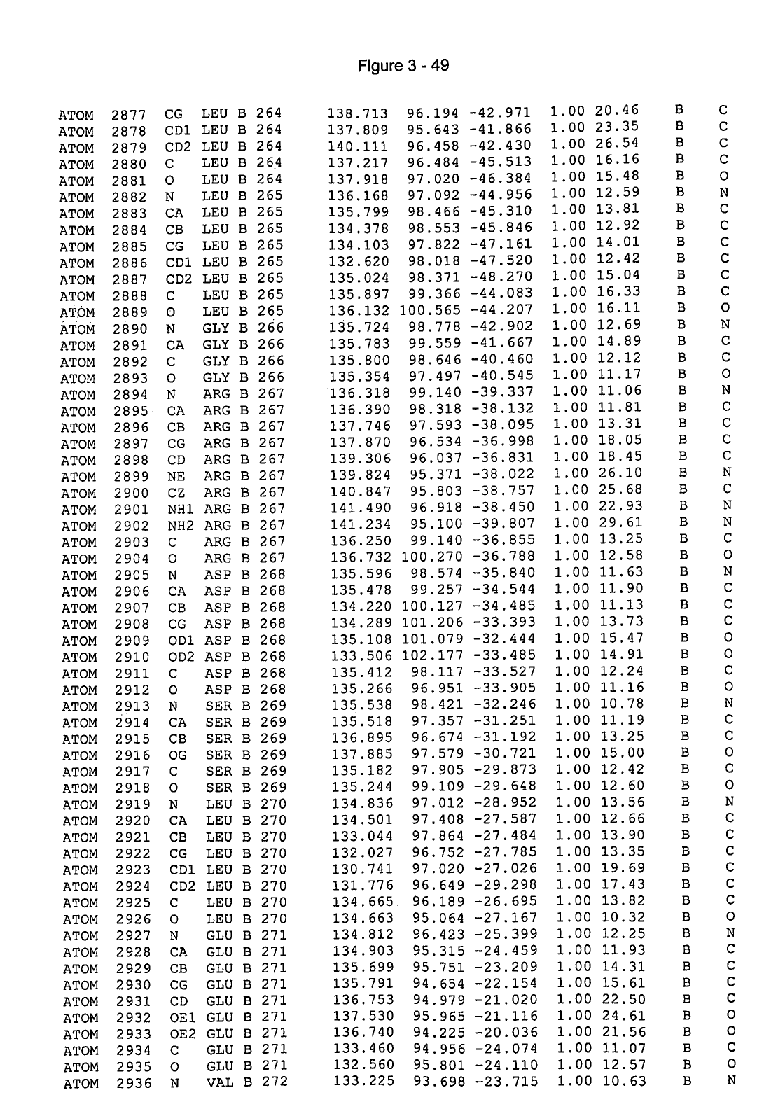

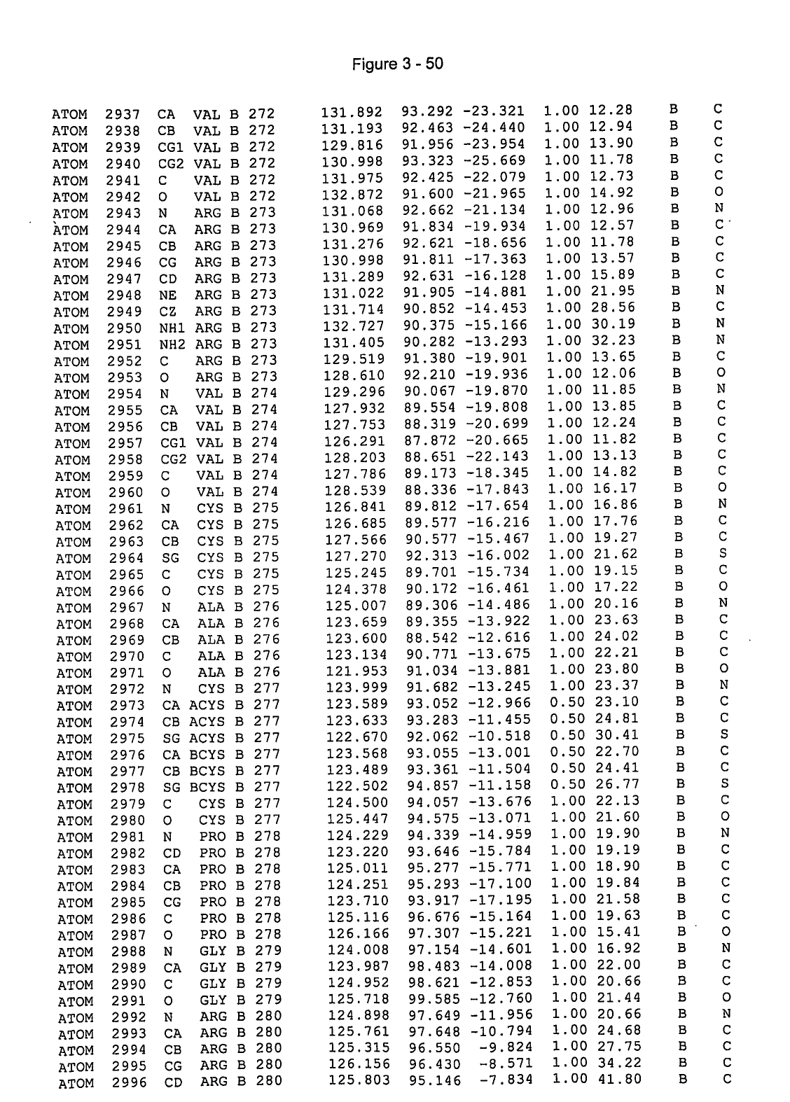

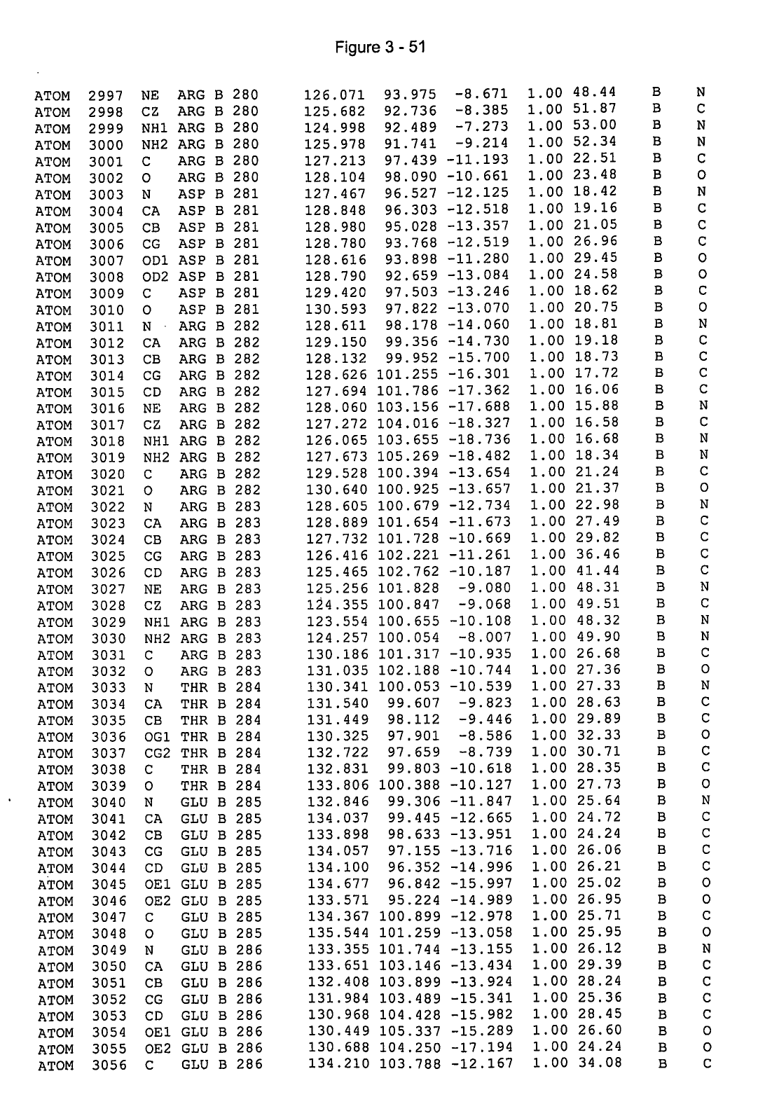

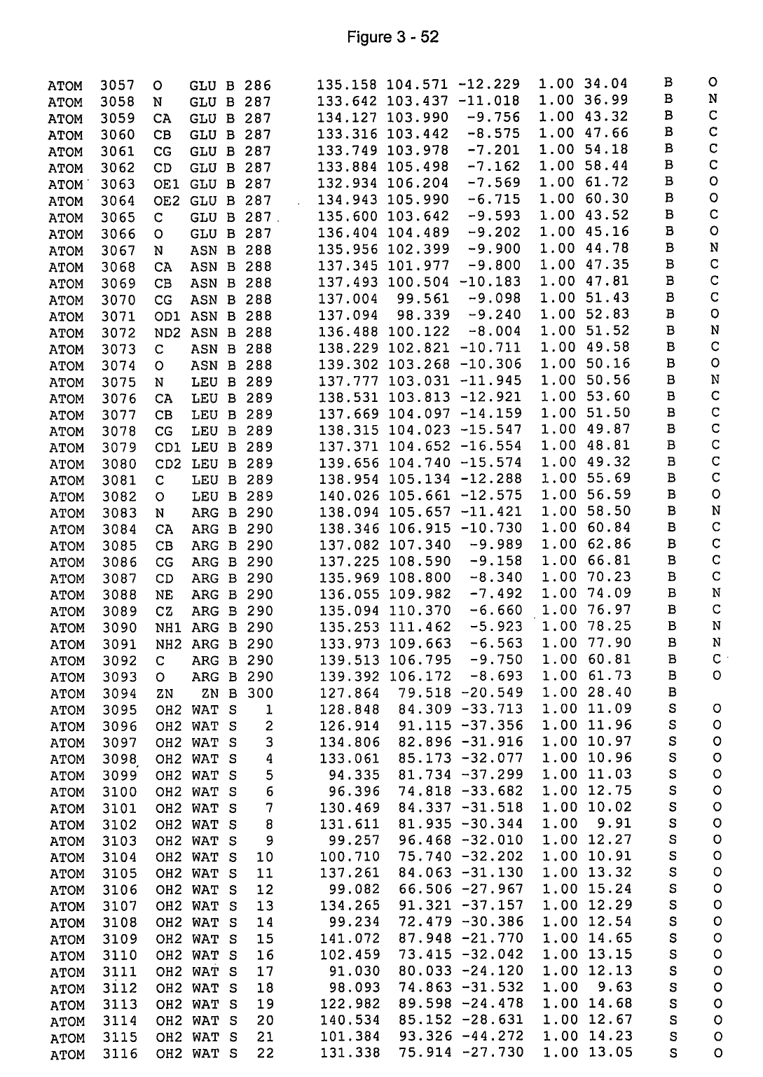

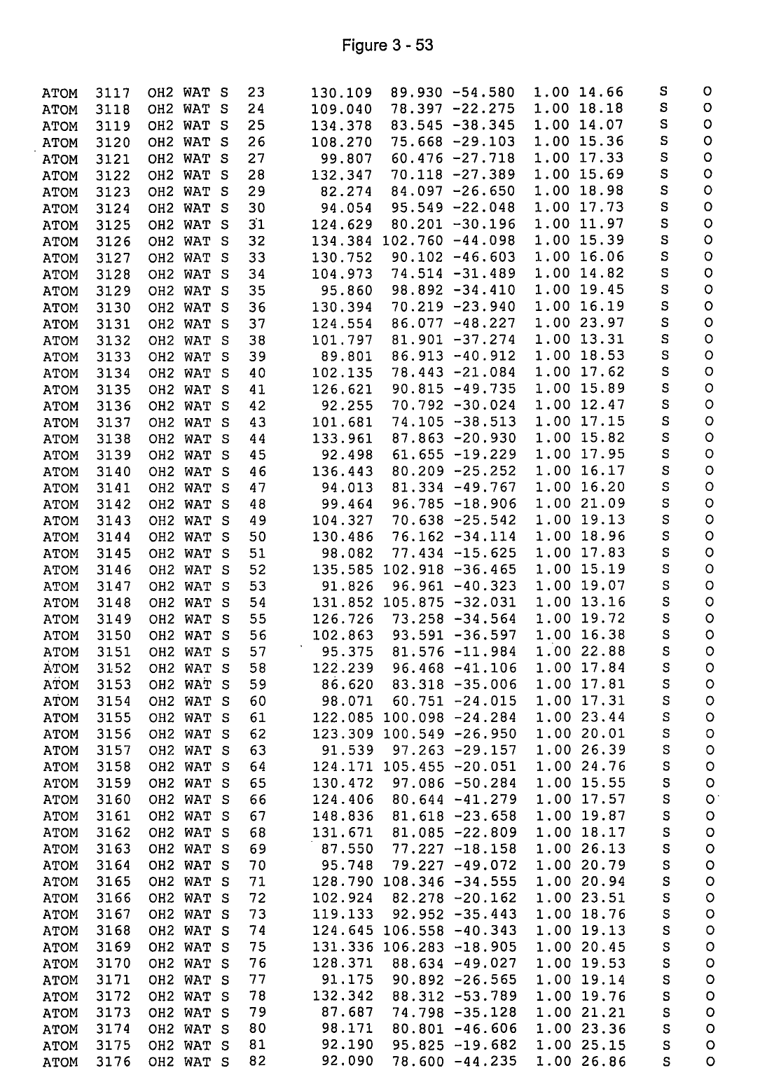

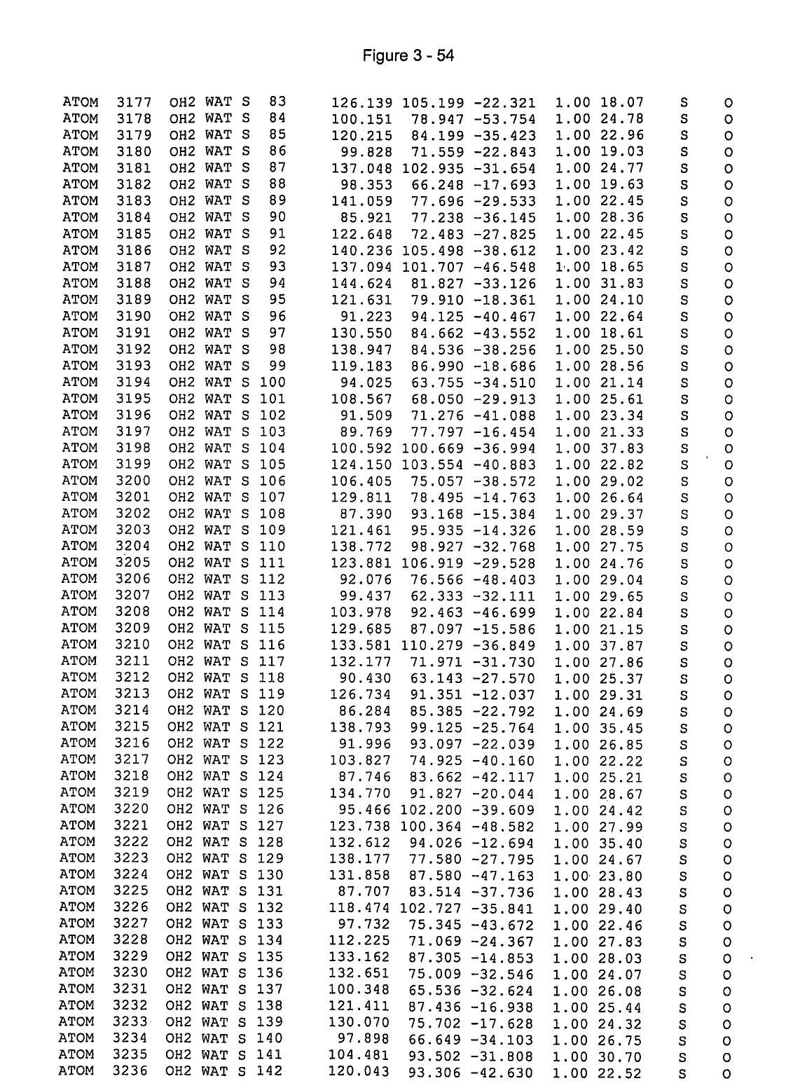

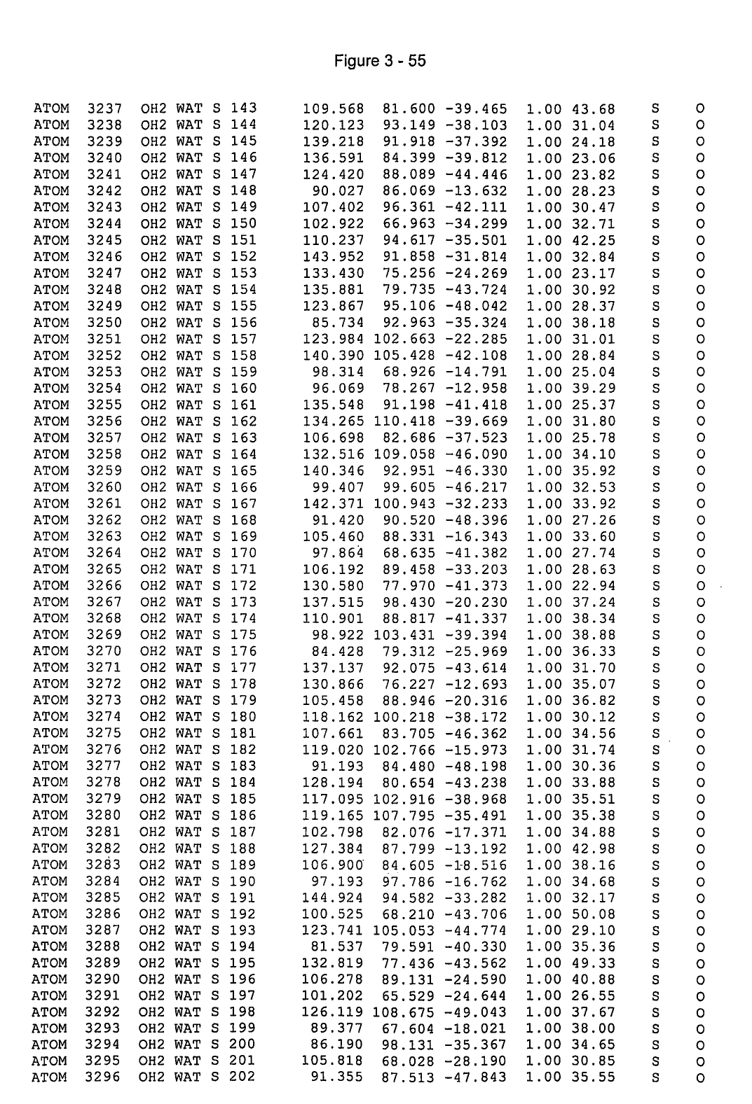

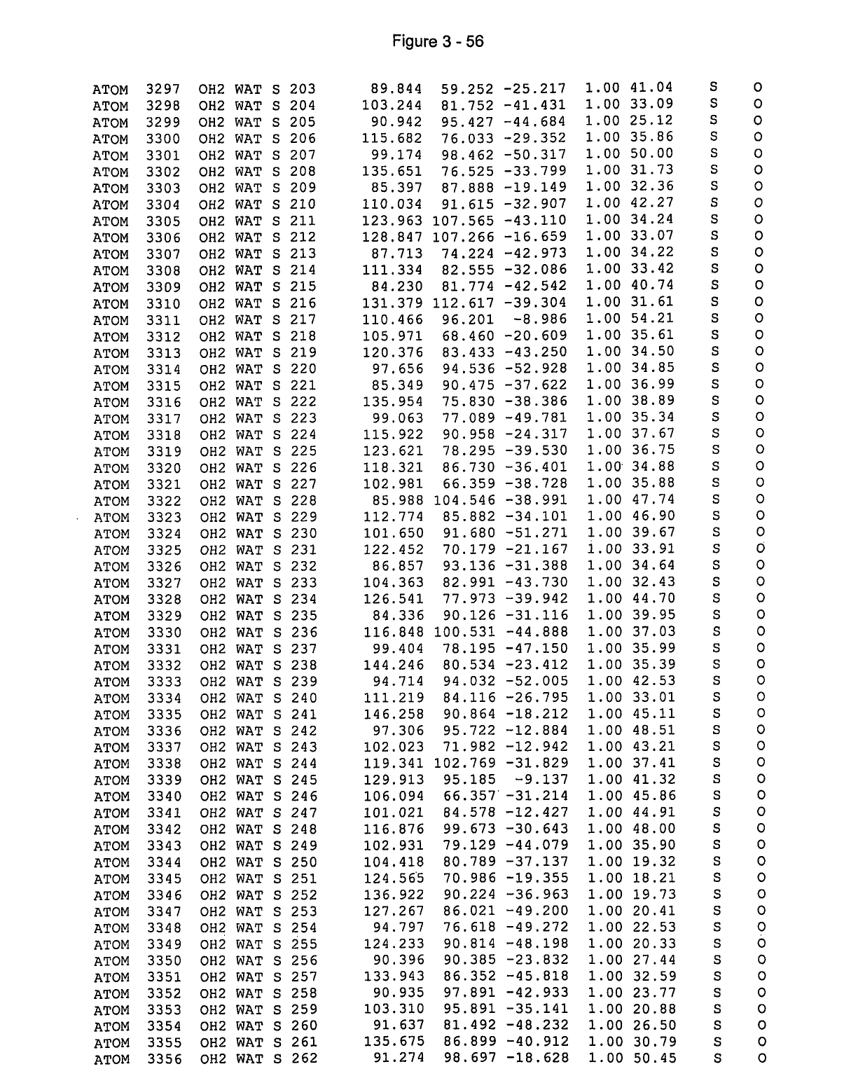

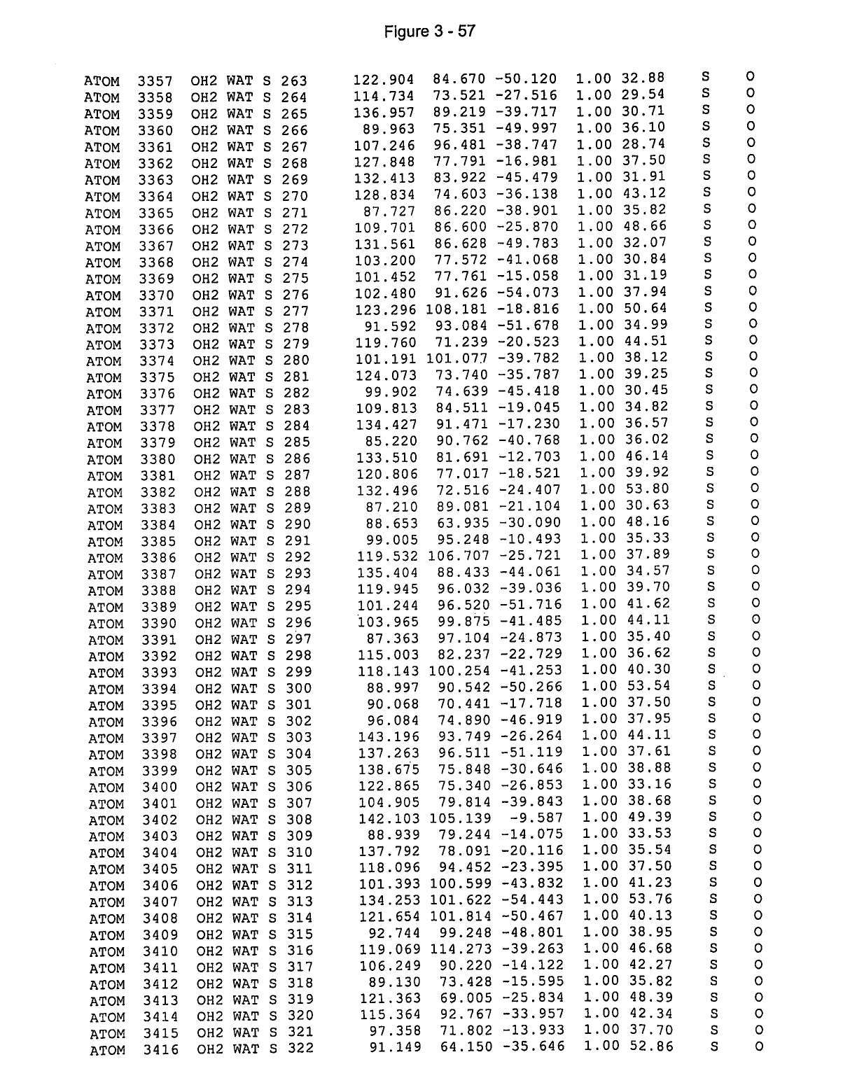

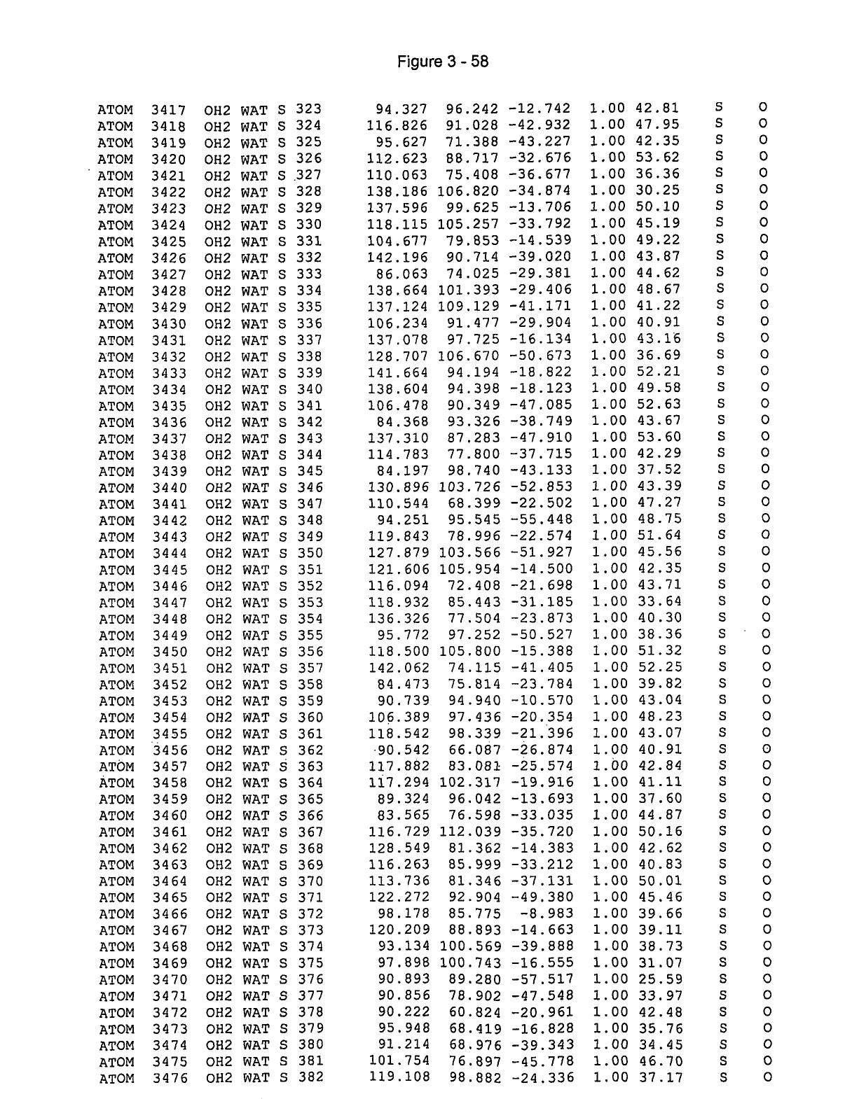

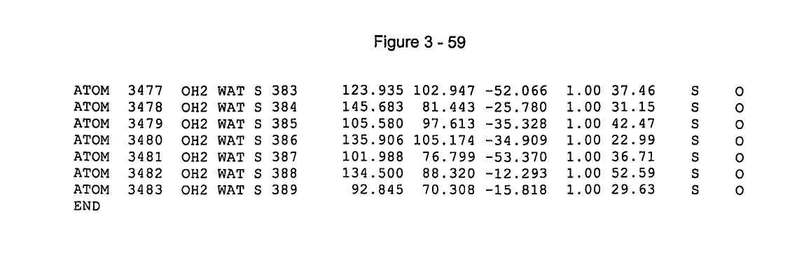

### BRIEF DESCRIPTION OF THE SEQUENCES

SEQ ID NO:1 is the sequence of the protein T-p53C-Y220C.

SEQ ID NO:2 is the sequence of the protein T-p53C-V143A.

SEQ ID NO:3 is the sequence of the protein T-p53C-F270L.

### DETAILED DESCRIPTION OF THE INVENTION

### A. Protein Crystals

The present invention provides a crystal of a T-p53C-Y220C, T-p53C-V143A or a T-p53C-F270L protein. These proteins may be produced as described in the accompanying examples.

Crystals of the invention may be apo crystals or co-crystals of a T-p53C-Y220C, T-p53C-V143A or a T-p53C-F270L protein with a ligand. Thus in a further aspect, the invention provides a co-crystal of a T-p53C-Y220C, T-p53C-V143A or a T-p53C-F270L protein and a ligand.

The ligand may be a compound being screened for its ability to stabilize the protein.

Such co-crystals may be obtained by co-crystallization or soaking.

In a more particular embodiment, the invention provides a crystal of T-p53C-Y220C, T-p53C-V143A or a T-p53C-F270L protein, each crystal having a space group P2₁2₁2₁. Optionally these crystals may be co-crystals of said proteins with a ligand.

The crystal of T-p53C-Y220C may have unit cell dimensions a=64.50 Å, b=71.11 Å, c=104.90 Å, beta=90°, with a unit cell variability of 5% in all dimensions.

The crystal of T-p53C-V143A may have unit cell dimensions a=64.66, Å, b=71.07 Å, c=105.00 Å, beta=90°, with a unit cell variability of 5% in all dimensions.

The crystal of T-p53C-F270L protein may have unit cell dimensions a=64.71 Å, b=71.04 Å, c=104.92 Å, beta=90°, with a unit cell variability of 5% in all dimensions.

More generally, said crystals may have unit cell dimensions of a=64.50-64.71 Å, b=71.04-71.11 Å, c=104.90-105 Å, beta=90°, with a unit cell variability of 5%, preferably 2.5%, preferably 1% in all dimensions (wherein the variability is calculated from the mid-point of each of said ranges).

The proteins which are crystallized may have the sequences shown in Table 4.

In the case of T-p53C-Y220C this comprises residues corresponding to residues 94-312 of p53. However, since the first resolvable residue is 96 and the last 291, truncations of the Table 4 sequence may be used. In particular, the sequence may be truncated by up to 10, preferably up to 5, e.g. up to 2 amino acids at the N-terminus. The sequence may be truncated by up to 25, preferably up to 21, preferably by up to 15, e.g. by up to 10, e.g. by up to 5 amino acids at the C-terminus. Any combination of the above-mentioned N- and C-terminal truncations may be used to produce crystals of the T-p53C-Y220C of the invention. Examples of such combinations are proteins T-p53C-Y220C₁₀₄₋₂₈₇; T-p53C-Y220C₁₀₄₋₂₉₁; T-p53C-Y220C₁₀₄₋₃₀₂; T-p53C-Y220C₁₀₄₋₃₀₇; T-p53C-Y220C₁₀₄₋₃₁₂; T-p53C-Y220C₉₉₋₂₈₇; T-p53C-Y220C₉₉₋₂₉₁; T-p53C-Y220C₉₉₋₃₀₂; T-p53C-Y220C₉₉₋₃₀₇; T-p53C-Y220C₉₉₋₃₁₂; T-p53C-Y220C₉₆₋₂₈₇; T-p53C-Y220C₉₆₋₂₉₁; T-p53C-Y220C₉₆₋₃₀₂; T-p53C-Y220C₉₆₋₃₀₇; and T-p53C-Y220C₉₆₋₃₁₂ (where T-p53C-Y220C_{x-y} represents a fragment of the Table 4 T-p53C-Y220C protein from p53 residue x to p53 residue y).

It is also possible that the T-p53C-Y220C protein may comprise short N- or C-terminal extensions, e.g. of naturally occurring p53 sequences and/or of heterologous sequences, e.g. those associated with the expression or purification of the protein such as short tags. Such sequences may add, independently, up to 5, such as up to 10 amino acid residues to either or both of the N- and C-termini of the Table 4 sequence.

Thus reference herein to a T-p53C-Y220C protein includes proteins which comprise at least residues 104-287 (e.g. up to at least 94-312 and optionally extended as above) and which are capable of forming a crystal. The crystal may have a space group P2₁2₁2₁, and in this form will have unit cell dimensions within 5% in each direction of the T-p53C-Y220C crystal illustrated in the accompanying examples.

In the case of T-p53C-V143A this comprises residues corresponding to residues 94-312 of p53. However, since the first resolvable residue is 96 and the last 290, truncations of the Table 4 sequence may be used. In particular, the sequence may be truncated by up to 10, preferably up to 5, e.g. up to 2 amino acids at the N-terminus. The sequence may be truncated by up to 25, preferably up to 21, preferably by up to 15, e.g. by up to 10, e.g. by up to 5 amino acids at the C-terminus. Any combination of the above-mentioned N- and C-terminal truncations may be used to produce crystals of the T-p53C-V143A of the invention. Examples of such combinations are proteins T-p63C-V143A₁₀₄₋₂₈₇; T-p53C-V143A₁₀₄₋₂₉₀; T-p53C-V143A₁₀₄₋₃₀₂; T-p53C-V143A₁₀₄₋₃₀₇; T-p53C-V143A₁₀₄₋₃₁₂; T-p53C-V143A₉₉₋₂₈₇; T-p53C-V143A₉₉₋₂₉₀; T-p53C-V143A₉₉₋₃₀₂; T-p53C-V143A₉₉₋₃₀₇; T-p53C-V143A₉₉₋₃₁₂; T-p53C-V143A₉₆₋₂₈₇; T-p53C-V143A₉₆₋₂₉₀; T-p53C-V143A₉₆₋₃₀₂; T-p53C-V143A₉₆₋₃₀₇; and T-p53C-V143A₉₅₋₃₁₂ (where T-p53C-V143A_{x-y} represents a fragment of the Table 4 T-p53C-V143A protein from p53 residue x to p53 residue y).

It is also possible that the T-p53C-V143A protein may comprise short N- or C-terminal extensions, e.g. of naturally occurring p53 sequences and/or of heterologous sequences, e.g. those associated with the expression or purification of the protein such as short tags. Such sequences may add, independently, up to 5, such as up to 10 amino acid residues to either or both of the N- and C-termini of the Table 4 sequence.

Thus reference herein to a T-p53C-V143A protein includes proteins which comprise at least residues 104-287 (e.g. up to at least 94-312 and optionally extended as above) and which are capable of forming a crystal. The crystal may have a space group P2₁2₁2₁, and in this form will have unit cell dimensions within 5% in each direction of the T-p53C-V143A crystal illustrated in the accompanying examples.

In the case of T-p53C-F270L this comprises residues corresponding to residues 94-312 of p53. However, since the first resolvable residue is 96 and the last 290, truncations of the Table 4 sequence may be used. In particular, the sequence may be truncated by up to 10, preferably up to 5, e.g. up to 2 amino acids at the N-terminus. The sequence may be truncated by up to 25, preferably up to 21, preferably by up to 15, e.g. by up to 10, e.g. by up to 5 amino acids at the C-terminus. Any combination of the above-mentioned N- and C-terminal truncations may be used to produce crystals of the T-p53C-F270L of the invention. Examples of such combinations are proteins T-p53C-F270L₁₀₄₋₂₈₇; T-p53C-F270L₁₀₄₋₂₉₀; T-p53C-F270L₁₀₄₋₃₀₂; T-p53C-F270L₁₀₄₋₃₀₇; T-p53C-F270L₁₀₄₋₃₁₂; T-p53C-F270L₉₉₋₂₈₇; T-p53C-F270L₉₉₋₂₉₀; T-p53C-F270L₉₉₋₃₀₂; T-p53C-F270L₉₉₋₃₀₇; T-p53C-F270L₉₉₋₃₁₂; T-p53C-F270L₉₆₋₂₈₇; T-p53C-F270L₉₆₋₂₉₀; T-p53C-F270L₉₆₋₃₀₂; T-p53C-F270L₉₆₋₃₀₇; and T-p53C-F270L₉₆₋₃₁₂ (where T-p53C-F270L_{x-y} represents a fragment of the Table 4 T-p53C-F270L protein from p53 residue x to p53 residue y).

It is also possible that the T-p53C-F270L protein may comprise short N- or C-terminal extensions, e.g. of naturally occurring p53 sequences and/or of heterologous sequences, e.g. those associated with the expression or purification of the protein such as short tags. Such sequences may add, independently, up to 5, such as up to 10 amino acid residues to either or both of the N- and C-termini of the Table 4 sequence.

Thus reference herein to a T-p53C-F270L protein includes proteins which comprise at least residues 104-287 (e.g. up to at least 94-312 and optionally extended as above) and which are capable of forming a crystal. The crystal may have a space group P2₁2₁2₁, and in this form will have unit cell dimensions within 5% in each direction of the T-p53C-F270L crystal illustrated in the accompanying examples.

### B. Crystal Coordinates

In further aspects, the invention also provides a crystal of a T-p53C-Y220C protein having the three dimensional atomic coordinates from Table 1; a crystal of a T-p53C-V143A protein having the three dimensional atomic coordinates from Table 2; a crystal of a T-p53C-F270L protein having the three dimensional atomic coordinates from Table 3.

An advantageous feature of the structure defined by the atomic coordinates of Tables 1-3 is that they have a resolution better than about 2.0 Å.

Tables 1-3 give atomic coordinate data for the T-p53C-Y220C, T-p53C-V143A and T-p53C-F270L proteins respectively. In the Tables the third column denotes the atom, the fourth the residue type, the fifth the chain identification, the sixth the residue number, the seventh, eighth and ninth columns are the X, Y, Z coordinates respectively of the atom in question, the tenth column the occupancy of the atom, the eleventh the temperature factor of the atom, the twelfth the chain identifier.

Tables 1-3 are set out in an internally consistent format. For example (apart from the first residue of Table 1), the coordinates of the atoms of each amino acid residue are listed such that the backbone nitrogen atom is first, followed by the C-alpha backbone carbon atom, designated CA, followed by side chain residues (designated according to one standard convention) and finally the carbon and oxygen of the protein backbone. Alternative file formats (e.g. such as a format consistent with that of the EBI Macromolecular Structure Database (Hinxton, UK)) which may include a different ordering of these atoms, or a different designation of the side-chain residues, may be used or preferred by others of skill in the art. However it will be apparent that the use of a different file format to present or manipulate the coordinates of the Table is within the scope of the present invention.

Table 1-3 comprises two protein units of the T-p53C variant proteins. The table further includes a number of water molecules, designated “WAT”, and a zinc ion. A number of residues, e.g. the Cys residues at 182 and 277 were observed in two conformers, so each conformer for each chain is provided.

In the embodiments of the invention described herein which use the crystal structures of the invention, it will be understood that reference to a T-p53C structures of the invention and their use should be interpreted as the structure or use of either individual protein chain, in either conformer. The use of both units is not excluded, but is not required to practice the present invention. Likewise, reference to a T-p53C structure of the invention does not include solvent or ion coordinates, though the use of these is not excluded where these may be beneficial or necessary to a particular application of the invention.

Protein structure similarity is routinely expressed and measured by the root mean square deviation (r.m.s.d.), which measures the difference in positioning in space between two sets of atoms. The r.m.s.d. measures distance between equivalent atoms after their optimal superposition. The r.m.s.d. can be calculated over all atoms, over residue backbone atoms (i.e. the nitrogen-carbon-carbon backbone atoms of the protein amino acid residues), main chain atoms only (i.e. the nitrogen-carbon-oxygen-carbon backbone atoms of the protein amino acid residues), side chain atoms only or more usually over C-alpha atoms only. For the purposes of this invention, the r.m.s.d. can be calculated over any of these, using any of the methods outlined below.

Preferably, rmsd is calculated by reference to the C-alpha atoms, provided that where selected coordinates are used, these comprise at least about 5%, preferably at least about 10%, of such atoms. Where selected coordinates do not include said at least about 5%, rmsd may be calculated by reference to all four backbone atoms, provided these comprise at least about 10%, preferably at least about 20% and more preferably at least about 30% of the selected coordinates. Where selected coordinates comprise 90% or more side chain atoms, rmsd may be calculated by reference to all the selected coordinates.

Thus the coordinates of Tables 1-3 provide a measure of atomic location in Angstroms, given to 3 decimal places. The coordinates are a relative set of positions that define a shape in three dimensions, but the skilled person would understand that an entirely different set of coordinates having a different origin and/or axes could define a similar or identical shape. Furthermore, the skilled person would understand that varying the relative atomic positions of the atoms of the structure so that the root mean square deviation of the residue backbone atoms (i.e. the nitrogen-carbon-carbon backbone atoms of the protein amino acid residues) is less than 2.0 Å, preferably less than 1.5 Å, preferably less than 1.0, such as less than 0.75 Å, more preferably less than 0.5 Å, more preferably less than 0.3 Å, such as less than 0.25 Å, or less than 0.2 Å, and most preferably less than 0.1 Å, when superimposed on the coordinates provided in Table 1 for the residue backbone atoms, will generally result in a structure which is substantially the same as the structure of Table 1 in terms of both its structural characteristics and usefulness for structure-based analysis of a T-p53C protein structure of the invention and its interactivity with molecular structures.

Likewise the skilled person would understand that changing the number and/or positions of the water molecules of the Tables will not generally affect the usefulness of the structures for structure-based analysis of a T-p53C protein-interacting structure. Thus for the purposes described herein as being aspects of the present invention, it is within the scope of the invention if: the coordinates of any one of Tables 1-3 is transposed to a different origin and/or axes; the relative atomic positions of the atoms of the structure are varied so that the root mean square deviation of residue backbone atoms is less than 1.5 Å, preferably less than 1.0, such as less than 0.75 Å, more preferably less than 0.5 Å, more preferably less than 0.3 Å, such as less than 0.25 Å, or less than 0.2 Å, and most preferably less than 0.1 Å when superimposed on the coordinates provided in Tables 1-3 for the residue backbone atoms; and/or the number and/or positions of water molecules is varied.

Reference herein to the coordinate data of or from any one of Tables 1-3, its use, and the like thus includes the coordinate data in which one or more individual values of the Table are varied in this way and will be understood to mean as such unless explicitly stated to the contrary.

Programs for determining rmsd include MNYFIT (part of a collection of programs called COMPOSER, Sutcliffe, M. J., Haneef, I., Carney, D. and Blundell, T. L. (1987) Protein Engineering, 1, 377-384), MAPS (Lu, G. An Approach for Multiple Alignment of Protein Structures (1998, in manuscript and on http://bioinfo1.mbfys.lu.se/TOP/maps.html)).

It is usual to consider C-alpha atoms and the rmsd can then be calculated using programs such as LSQKAB (Collaborative Computational Project 4. The CCP4 Suite: Programs for Protein Crystallography, Acta Ctystallographica, D50, (1994), 760-763), QUANTA (Jones et al., Acta Crystallography A47 (1991), 110-119 and commercially available from Accelerys, San Diego, Calif.), Insight (commercially available from Accelerys, San Diego, Calif.), Sybyl® (commercially available from Tripos, Inc., St Louis), O (Jones et al., Acta Crystallographica, A47, (1991), 110-119), and other coordinate fitting programs.

In, for example the programs LSQKAB and O, the user can define the residues in the two proteins that are to be paired for the purpose of the calculation. Alternatively, the pairing of residues can be determined by generating a sequence alignment of the two proteins, programs for sequence alignment are discussed in more detail herein below. The atomic coordinates can then be superimposed according to this alignment and an r.m.s.d. value calculated. The program Sequoia (C. M. Bruns, I. Hubatsch, M. Ridderstrom, B. Mannervik, and J. A. Tainer (1999) Human Glutathione Transferase A4-4 Crystal Structures and Mutagenesis Reveal the Basis of High Catalytic Efficiency with Toxic Lipid Peroxidation Products, Journal of Molecular Biology 288(3): 427-439) performs the alignment of homologous protein sequences, and the superposition of homologous protein atomic coordinates. Once aligned, the r.m.s.d. can be calculated using programs detailed above. For sequence identical, or highly identical, the structural alignment of proteins can be done manually or automatically as outlined above. Another approach would be to generate a superposition of protein atomic coordinates without considering the sequence.

It is more normal when comparing significantly different sets of coordinates to calculate the rmsd value over C-alpha atoms only. It is particularly useful when analysing side chain movement to calculate the rmsd over all atoms and this can be done using LSQKAB and other programs.

Those of skill in the art will appreciate that in many applications of the invention, it is not necessary to utilise all the coordinates of Tables 1-3, but merely a portion of them. For example, as described below, in methods of modelling molecular structures with a T-p53C-protein of the invention, selected coordinates as referred to herein may be used.

By “selected coordinates” it is meant for example at least 5, preferably at least 10, more preferably at least 50 and even more preferably at least 100, for example at least 500 or at least 1000 atoms of a T-p53C protein structure. Likewise, the other applications of the invention described herein, including homology modelling and structure solution, and data storage and computer assisted manipulation of the coordinates, may also utilise all or a portion of the coordinates (i.e. selected coordinates) of any one of Tables 1-3.

In one aspect, the selected coordinates of Table 1 may include at least one atom from at least one of residues 109, 145-157, 202-204, 219-223, 228-230 and 257. In some aspects, it may be desirable to include at least one atom of Cys 220. In such aspects, the selected coordinates of Table 1 may include:
(i) at least one coordinate of an atom from at least one of residues 109, 145-157, 202-204, 219-223, 228-230 and 257, optionally at least two atoms from said residues wherein at least one is an atom of Cys 220;
(ii) at least one atom from at least one or more of the residues Arg156, Arg158, Arg202, Glu204, Pro219 and Glu258, optionally in combination with at least one atom of Cys220; or
(iii) at least one atom from at least one or more the residues Trp146, Val147, Thr150, and Pro223, optionally in combination with Cys220.

Preferably, the selected coordinates include atoms from at least two, e.g. at least 3, 4, 5, 6, 7, 8 or 9 of the above groups (i)-(iii) of residues.

In another aspect, the selected coordinates of Table 2 may include at least one atom from at least one of residues of the group 111, 113, 124, 133, 141-143, 145, 157, 232, 234, 236, 255 and 270, preferably at least one of residues of the group 113, 124, 133, 141-143, 234, 236, and 270. Said groups may include one or more atoms of 143, or may be combinations of other atoms of other residues.

In a further aspect, the selected coordinates of Table 3 may include at least one atom from at least one of residues of the group 111, 113, 133, 143, 159, 234, 236, 253, 255, 270, and 272. Said group may include one or more atoms of 270, or may be combinations of other atoms of other residues.

Preferably, the selected coordinates include atoms from at least two, e.g. at least 3, 4, 5, 6, 7, 8 or 9 of the above groups of residues. In one embodiment, where the number of selected coordinates is n (where n is a number from 2 to the total number of amino acids in any of the groups above, these may be from at least n different amino acids of the selected group used. The selected residues may be side-chain or main-chain atoms, or any combination thereof.

Further, the identification of the groups of atoms mentioned above, which are associated with the cavities generated by the mutations described herein, allows the identification, design or modification of ligands which bind in these cavities and/or to direct structural neighbours of these residues.

### C. Computer Systems

In another aspect, the present invention provides systems, particularly a computer system, the systems containing one of co-ordinate data of any one of Tables 1-3, said data defining the three-dimensional structure of a T-p53C variant protein of the invention or at least selected coordinates thereof.

For example the computer system may comprise: (i) a computer-readable data storage medium comprising data storage material encoded with the computer-readable data; (ii) a working memory for storing instructions for processing said computer-readable data; and (iii) a central-processing unit coupled to said working memory and to said computer-readable data storage medium for processing said computer-readable data and thereby generating structures and/or performing rational drug design including the computer-based screening of compounds whose ability to interact with the p53 structures of the present invention is unknown. The computer system may further comprise a display coupled to said central-processing unit for displaying said structures.

The invention also provides such systems containing atomic coordinate data of target proteins as referred to above wherein such data has been generated according to the methods of the invention described herein based on the starting data provided the data of Table 1 or selected coordinates thereof.

Such data is useful for a number of purposes, including the generation of structures to analyse the mechanisms of action of p53 proteins and/or to perform rational drug design of compounds, which interact with a p53 protein, particularly a p53 Y220C, a p53 V143A or a p53 F270L protein, such as compounds which are potential stabilizers of such proteins.

In a further aspect, the present invention provides computer readable media with coordinate data of any one of Tables 1-3, said data defining the three-dimensional structure of a T-p53C-variant protein of the invention or at least selected coordinates thereof.

As used herein, “computer readable media” refers to any medium or media, which can be read and accessed directly by a computer. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By providing such computer readable media, the atomic coordinate data of the invention can be routinely accessed to model a T-p53C-variant protein of the invention or selected coordinates thereof. For example, RASMOL (Sayle et al., TIBS, Vol. 20, (1995), 374) is a publicly available computer software package, which allows access and analysis of atomic coordinate data for structure determination and/or rational drug design.

As used herein, “a computer system” refers to the hardware means, software means and data storage means used to analyse the atomic coordinate data of the invention. The minimum hardware means of the computer-based systems of the present invention comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

A further aspect of the invention provides a method of providing data for generating structures and/or performing optimisation of compounds which interact with a T-p53C-Y220C, -V143A or -F270 protein, the method comprising:
(i) establishing communication with a remote device containing computer-readable data comprising a T-p53C-Y220C, -V143A or -F270 structure or selected coordinates thereof from Table 1, optionally varied within a root mean square deviation from the Cα atoms of not more than 1.5 Å; and
(ii) receiving said computer-readable data from said remote device.

Thus the remote device may comprise e.g. a computer system or computer readable media of one of the previous aspects of the invention. The device may be in a different country or jurisdiction from where the computer-readable data is received.

The communication may be via the internet, intranet, e-mail etc, transmitted through wires or by wireless means such as by terrestrial radio or by satellite. Typically the communication will be electronic in nature, but some or all of the communication pathway may be optical, for example, over optical fibres.

Once the data is received from the device, the invention may comprise the further step of using the data in the modelling systems of the invention described herein.

### D. Uses of the Structures of the Invention

Our structural observations have profound implications for novel therapeutic strategies that aim at rescuing the function of p53 with small molecule drugs that stabilize p53. On the basis of our structural studies, β-sandwich mutants, such as V143A and F270L, represent promising targets for rescue by generic small molecule drugs, because in this case stabilizing the protein may be sufficient to restore wild-type-like activity under physiological conditions. Y220C not only has the potential of being rescued by a generic wild-type-binding compound, but also is a target for a specific drug that can bind in the crevice formed by the deletion. The crevice region is particularly attractive because it appears distant from the functional sites and interfaces of the protein.

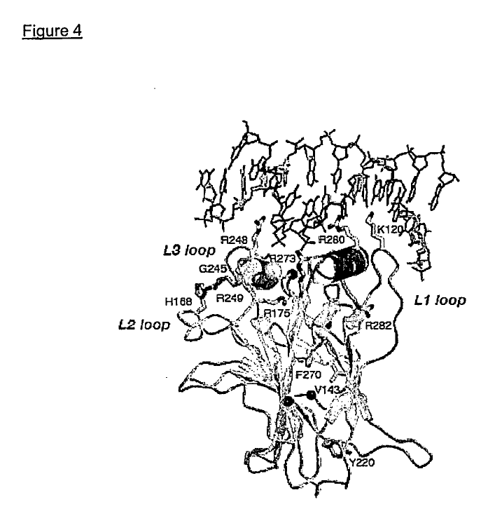

Cancer mutations in the β-sandwich region of the core domain are generally less frequent than those in the DNA-binding region. Nevertheless, taken together, they represent a substantial portion of cancer-related mis-sense mutations. In fact, about one third of the reported cancer mutations in p53 core domain are located outside the structural elements that form the DNA-binding surface (loops L2, L3 and the LSH-motif). The structures of T-p53C-V143A and T-p53C-F270L elucidate the structural effects of two cancer-related (3-sandwich mutations. Val143 and Phe270 are located on opposing strands of the β-sandwich. Their side chains are facing each other and form an integral part of the hydrophobic core of the β-sandwich (FIG. 4). The V143A mutant is of particular interest, because of its well-documented temperature-sensitive behaviour for the binding of many response elements in both yeast and mammalian systems (15,24). A recent study has isolated temperature sensitive p53 mutants from a comprehensive mis-sense mutation library by using a yeast-based functional assay (16). Most mutations were clustered in the β-sheet region of the protein, and the substitutions were mainly from large hydrophobic residues to smaller hydrophobic residues (V143A was not detected in this study, whereas mutations at residue 270 were (F2701 and F270C) were). The structures of T-p53C-V143A and T-p53C-F270L provide the molecular basis for understanding the temperature-sensitive behaviour of many p53 mutants. The V143A and F270L mutations both created cavities in the hydrophobic core of the β-sandwich, without collapse of the surrounding structure. While the overall structure of the core domain was perfectly conserved, the creation of void volumes came at a high energetic cost of 3.7 and 4.1 kcal/mol. These structural and energetic changes are consistent with work on T4-lysozyme and barnase, which showed that the energetic response to a particular type of “large-to-small” substitutions in the hydrophobic core of the protein correlates with the volume of the created cavity and the structural shifts of close neighbours (25-27). Interestingly, the Y220C mutation has also been reported to cause temperature-sensitive behaviour (24). Again, this behaviour is in perfect agreement with the structural data of the present invention. The mutation created a solvent accessible cleft at the far end of the β-sandwich. Removal of the aromatic side chain of Tyr220 leaves several residues at the periphery of the hydrophobic core of the β-sandwich with energetically less favourable packing interactions or partly solvent exposed, resulting in a loss of thermodynamic stability. The structural changes were, however, very localized, far away from the DNA-binding surface.

A common structural feature of the β-sandwich mutants appears to be that there are only minor structural disruptions upon mutation, although the effect on the thermodynamic stability of the protein was generally more severe than for the hotspot mutations in the DNA-binding surface. The much more compact and robust structural framework of the β-sandwich compared with the zinc-binding region and loop-sheet-helix motif renders it generally much less susceptible to mutation-induced structural changes, in particular for “large-to-small” substitutions. The absence of structural changes in surface regions, especially in the DNA-binding surface, however, is key for functionality. Temperature-sensitivity behaviour can be expected for all cancer mutations that destabilize the core domain without compromising the surface complementarity that is crucial for the function of p53, not only for binding to specific promoter sequences, but also for interactions with a whole subset of other proteins and for the correct domain organization in tetrameric full-length p53 (11, 28-31).

Thus, the crystal structures obtained according to the present invention may be used in several ways for drug design which are discussed in further detail below. In a particular embodiment, the structures may be used to identify compounds which interact within the Y220C pocket of a mutant p53 in a manner which stabilizes the pocket. Such a stabilization may allow rescue of the function of p53 in a subject having the Y220C mutation, such that the function of p53 in a tumour cell can be restored. Similarly, the structures of Tables 2 and 3 may be used to identify other compounds which stabilize the cavity created by V143 and F270L mutations. Compounds which stabilize this cavity may be of wider use in stabilizing the p53 p-sandwich region mutants.

Information on the binding of such compounds or potential compounds may be obtained by co-crystallization, soaking or computationally docking the drug in the binding pocket. This will guide specific modifications to the chemical structure designed to mediate or control the interaction of the drug with the protein. Such modifications can be designed to improve its therapeutic and/or prophylactic action.

### (i) Obtaining and Analysing Crystal Complexes.

In one approach, the structure of a compound bound to a T-p53C-Y220C, -V143A or -F270 protein may be determined by experiment. This will provide a starting point in the analysis of the compound bound to a T-p53C-Y220C, -V143A or -F270 protein, thus providing those of skill in the art with a detailed insight as to how that particular compound interacts with a wild-type p53-Y220C, -V143A or -F270 protein and the mechanism by which it works.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map (as explained e.g. by Blundell et al., in Protein Crystallography, Academic Press, New York, London and San Francisco, (1976)), it is necessary to know beforehand the protein 3D structure (or at least the protein structure factors). Therefore, determination of the T-p53C-Y220C, -V143A or -F270 protein structure also allows difference Fourier electron density maps of protein-compound complexes to be produced, determination of the binding position of the drug and hence may greatly assist the process of rational drug design.

Accordingly, the invention provides a method for determining the structure of a compound bound to a T-p53C-Y220C, -V143A or -F270 protein, said method comprising:
providing a crystal of a T-p53C-Y220C, -V143A or -F270 protein according to the invention;
soaking the crystal with said compounds; and
determining the structure of said T-p53C-Y220C, -V143A or -F270 protein compound complex by employing the coordinate data of Tables 1-3 respectively or selected coordinates thereof.

Alternatively, the T-p53C-Y220C, -V143A or -F270 protein and compound may be co-crystallized. Thus the invention provides a method for determining the structure of a compound bound to a T-p53C-Y220C, -V143A or -F270 protein said method comprising;
mixing the protein with the compound(s), crystallizing the protein-compound(s) complex; and determining the structure of said protein-compound(s) complex by reference to the coordinate data of Tables 1-3 respectively or selected coordinates thereof.

The analysis of such structures may employ (i) X-ray crystallographic diffraction data from the complex and (ii) a three-dimensional structure of a T-p53C-Y220C, -V143A or -F270 protein, or at least selected coordinates thereof, to generate a difference Fourier electron density map of the complex, the three-dimensional structure being defined by atomic coordinate data of Tables 1-3 respectively or selected coordinates thereof. The difference Fourier electron density map may then be analysed.

Therefore, such complexes can be crystallized and analysed using X-ray diffraction methods, e.g. according to the approach described by Greer et al., J. of Medicinal Chemistry, Vol. 37, (1994), 1035-1054, and difference Fourier electron density maps can be calculated based on X-ray diffraction patterns of soaked or co-crystallized protein and the solved structure of uncomplexed protein. These maps can then be analysed e.g. to determine whether and where a particular compound binds to a T-p53C-Y220C, -V143A or -F270 protein and/or changes the conformation of said protein.

Electron density maps can be calculated using programs such as those from the CCP4 computing package (Collaborative Computational Project 4. The CCP4 Suite: Programs for Protein Crystallography, Acta Crystallographica, D50, (1994), 760-763.). For map visualization and model building programs such as “O” (Jones et al., Acta Crystallographica, A47, (1991), 110-119) can be used.

All of the complexes referred to above may be studied using well-known X-ray diffraction techniques and may be refined against 1.0 to 3.5 Å resolution X-ray data to an R value of about 0.30 or less using computer software, such as CNX (Brunger et al., Current Opinion in Structural Biology, Vol. 8, Issue 5, October 1998, 606-611, and commercially available from Accelrys, San Diego, Calif.), and as described by Blundell et al, (1976) and Methods in Enzymology, vol. 114 & 115, H. W. Wyckoff et al., eds., Academic Press (1985).

### (ii) In Silico Analysis and Design

Although the invention will facilitate the determination of actual crystal structures comprising a T-p53C-Y220C, -V143A or -F270 protein and a compound, which interacts with the protein, current computational techniques provide a powerful alternative to the need to generate such crystals and generate and analyse diffraction date. Accordingly, a particularly preferred aspect of the invention relates to “in silico” methods directed to the analysis and development of compounds which interact with T-p53C-Y220C, -V143A or -F270 protein structure of the present invention.

Determination of the three-dimensional structure of a T-p53C-Y220C, -V143A or -F270 protein provides important information about the binding sites of this protein, particularly when comparisons are made with similar proteins.

As set out in the accompanying examples, we have significant differences in the β-sandwich region caused by the Y220C alteration, resulting in a significant displacement of some of the residues in this region compared to the wild-type protein.

This information may then be used for rational design and modification of p53 ligands, e.g. by computational techniques which identify possible binding ligands for the binding sites, by enabling linked-fragment approaches to drug design, and by enabling the identification and location of bound ligands (e.g. including those ligands mentioned herein above) using X-ray crystallographic analysis. These techniques are discussed in more detail below.

Thus as a result of the determination of the three-dimensional structure of T-p53C-Y220C, more purely computational techniques for rational drug design may also be used to design structures whose interaction with a p53 carrying the Y220C change is better understood (for an overview of these techniques see e.g. Walters et al (Drug Discovery Today, Vol. 3, No. 4, (1998), 160-178; Abagyan, R.; Totrov, M. Curr. Opin. Chem. Biol. 2001, 5, 375-382). Likewise, the T-p53C-V143A and T-p53C-F270L structures may be used to design ligands which target the residues of the cavities, or residues which are direct structural neighbours, generated by these mutations.

For example, automated ligand-receptor docking programs (discussed e.g. by Jones et al. in Current Opinion in Biotechnology, Vol. 6, (1995), 652-656 and Halperin, I.; Ma, B.; Wolfson, H.; Nussinov, R. Proteins 2002, 47, 409-443), which require accurate information on the atomic coordinates of target receptors may be used.

Accordingly, the invention provides a computer-based method for the analysis of the interaction of a molecular structure with a p53 structure, which comprises:
providing the p53 structure or selected coordinates thereof of Table 1 optionally varied within a root mean square deviation from the Cα atoms of not more than 1.5 Å;
providing a molecular structure to be fitted to said p53 structure or selected coordinates thereof; and
fitting the molecular structure to said p53 structure;
wherein said selected coordinates include at least one coordinate of an atom from residues 109, 145-157, 202-204, 219-223, 228-230 and 257.

In practice, it will be desirable to model a sufficient number of atoms of a T-p53C-Y220C structure as defined by the coordinates from Table 1 or selected coordinates thereof), which represent a binding pocket, e.g. the numbers of atoms or the atoms from preferred residues as defined in section B above. Thus in this aspect of the invention, the selected coordinates may comprise coordinates of some or all of these above-mentioned residues.

Accordingly, the invention provides a computer-based method for the analysis of the interaction of a molecular structure with a p53 structure, which comprises:
providing the p53 structure or selected coordinates thereof of Table 2 optionally varied within a root mean square deviation from the Cα atoms of not more than 1.5 Å;
providing a molecular structure to be fitted to said p53 structure or selected coordinates thereof; and
fitting the molecular structure to said p53 structure;
wherein said selected coordinates include at least one coordinate of an atom from residues 113, 124, 133, 141-143, 234, 236, and 270.

In practice, it will be desirable to model a sufficient number of atoms of a T-p53C-V143A structure as defined by the coordinates from Table 2 or selected coordinates thereof), which represent a binding pocket, e.g. the numbers of atoms or the atoms from preferred residues as defined in section B above. Thus in this aspect of the invention, the selected coordinates may comprise coordinates of some or all of these above-mentioned residues.

Accordingly, the invention provides a computer-based method for the analysis of the interaction of a molecular structure with a p53 structure, which comprises:
providing the p53 structure or selected coordinates thereof of Table 3 optionally varied within a root mean square deviation from the Cα atoms of not more than 1.5 Å;
providing a molecular structure to be fitted to said p53 structure or selected coordinates thereof; and
fitting the molecular structure to said p53 structure;
wherein said selected coordinates include at least one coordinate of an atom from residues 111, 113, 133, 143, 159, 234, 236, 253, 255, 270, and 272.

In practice, it will be desirable to model a sufficient number of atoms of a T-p53C-F270L structure as defined by the coordinates from Table 3 or selected coordinates thereof), which represent a binding pocket, e.g. the numbers of atoms or the atoms from preferred residues as defined in section B above. Thus in this aspect of the invention, the selected coordinates may comprise coordinates of some or all of these above-mentioned residues.

Following the fitting of the molecular structures, a person of skill in the art may seek to use molecular modelling to determine to what extent the structures interact with each other (e.g. by hydrogen bonding, other non-covalent interactions, or by reaction to provide a covalent bond between parts of the structures).

The person of skill in the art may use in silico modelling methods to alter one or more of the structures in order to design new structures which interact in different ways with a T-p53C-Y220C, -V143A or -F270 structure.

Newly designed structures may be synthesised and their interaction with a T-p53C-Y220C, -V143A or -F270 structure may be determined or predicted as to how the newly designed structure is bound by said T-p53C-Y220C, -V143A or -F270 structure. This process may be iterated so as to further alter the interaction between it and the a T-p53C-Y220C, -V143A or -F270 structure.

Further, once a structure which has been fitted is determined to fit in a manner which will stabilize a T-p53C-Y220C, -V143A or -F270 structure of the invention, the structure may be fitted to other p53 proteins, including mutants of the wild-type sequence, either by computer-assisted means or by synthesis and testing of ligand.

By “fitting”, it is meant determining by automatic, or semi-automatic means, at least one interaction between at least one atom of a molecular structure and at least one atom of a T-p53C-Y220C, -V143A or -F270 structure of the invention, and calculating the extent to which such an interaction is stable. Interactions include attraction and repulsion, brought about by hydrophobic, polar, charged, steric, π-π interactions and the like. Various computer-based methods for fitting are described further herein.

More specifically, the interaction of a compound or compounds with a T-p53C-Y220C, -V143A or -F270 structure can be examined through the use of computer modelling using a docking program such as GOLD (Jones et al., J. Mol. Biol., 245, 43-53 (1995), Jones et al., J. Mol. Biol., 267, 727-748 (1997)), GRAMM (Vakser, I. A., Proteins, Suppl., 1:226-230 (1997)), DOCK (Kuntz et al, J.Mol.Biol. 1982 , 161, 269-288, Makino et al, J.Comput.Chem. 1997, 18, 1812-1825), AUTODOCK (Goodsell et al, Proteins 1990, 8, 195-202, Morris et al, J.Comput.Chem. 1998, 19, 1639-1662.), FlexX, (Rarey et al, J.Mol.Biol. 1996, 261, 470-489) or ICM (Abagyan et al, J.Comput.Chem. 1994, 15, 488-506). This procedure can include computer fitting of compounds to a T-p53C-Y220C structure to ascertain how well the shape and the chemical structure of the compound will bind to the structure.

The various computer-based methods of analysis described herein may be performed using computer systems such as those described in the preceding section. Generally, the computer systems used will be configured to display or transmit a model of the structure of Table 1, 2 or 3, or selected coordinates thereof and a molecular structure so as to indicate one or more interactions between the two. A variety of formats of display are known in the art and may be selected by a person of ordinary skill in the art dependent upon a variety of factors including, for example, the nature of the interactions being determined.

Also computer-assisted, manual examination of the active site structure of a T-p53C-Y220C, -V143A or -F270 may be performed. The use of programs such as GRID (Goodford, J. Med. Chem., 28, (1985), 849-857)—a program that determines probable interaction sites between molecules with various functional groups and an protein surface—may also be used to analyse the active site to predict, for example, the types of modifications which will alter the stability of a compound or the protein.

Detailed structural information can then be obtained about the binding of the compound to a T-p53C-Y220C, -V143A or -F270 structure, and in the light of this information adjustments can be made to the structure or functionality of the compound, e.g. to alter its interaction with a T-p53C-Y220C, -V143A or -F270 structure. The above steps may be repeated and re-repeated as necessary.

Molecular structures, which may be used in the present invention, will usually be compounds under development for pharmaceutical use. Generally such compounds will be organic molecules, which are typically from about 100 to 2000 Da, more preferably from about 100 to 1000 Da in molecular weight. Such compounds include peptides and derivatives thereof. In principle, any compound under development in the field of pharmacy can be used in the present invention in order to facilitate its development or to allow further rational drug design to improve its properties.

In another embodiment, the present invention provides a method for modifying the structure of a compound in order to alter its interaction with a T-p53C-Y220C, which method comprises:
fitting a starting compound to one or more coordinates of at least one amino acid residue of the ligand-binding region of a T-p53C-Y220C structure of the present invention;
modifying the starting compound structure so as to increase or decrease its interaction with the ligand-binding region;
wherein said ligand-binding region is defined as including at least one, and preferably more than one, of the residues 109, 145-157, 202-204, 219-223, 228-230 and 257. Preferred numbers and combinations of residues are as defined herein above.

It would be understood by those of skill in the art that modification of the structure will usually occur in silico, allowing predictions to be made as to how the modified structure interacts with a p53 or mutant thereof. Once such a compound has been developed it may be synthesised and tested also as described above.

(iii) Fragment Linking and Growing.

The provision of the crystal structures of the invention will also allow the development of compounds which interact with the binding pocket regions of a T-p53C-Y220C, -V143A or -F270 (for example to act to stabilize the protein) based on a fragment linking or fragment growing approach.

For example, the binding of one or more molecular fragments can be determined in the protein binding pocket by X-ray crystallography. Molecular fragments are typically compounds with a molecular weight between 100 and 200 Da. This can then provide a starting point for medicinal chemistry to optimise the interactions using a structure-based approach. The fragments can be combined onto a template or used as the starting point for ‘growing out’ an inhibitor into other pockets of the protein. The fragments can be positioned in the binding pocket of a T-p53C-Y220C, -V143A or -F270 structure and then ‘grown’ to fill the space available, exploring the electrostatic, van der Waals or hydrogen-bonding interactions that are involved in molecular recognition. The potency of the original weakly binding fragment thus can be rapidly improved using iterative structure-based chemical synthesis.

At one or more stages in the fragment growing approach, the compound may be synthesized and tested in a biological system for its activity. This can be used to guide the further growing out of the fragment.

Where two fragment-binding regions are identified, a linked fragment approach may be based upon attempting to link the two fragments directly, or growing one or both fragments in the manner described above in order to obtain a larger, linked structure, which may have the desired properties.

Where the binding site of two or more ligands are determined they may be connected to form a potential lead compound that can be further refined using e.g. the iterative technique of Greer et al. For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design p53-binding ligand is made possible by the determination of the structures provided by the present invention.

(iv) Analysis of p53-Ligands

In a further aspect, where a molecular structure has been obtained in accordance with the invention, the invention may comprise the further step of fitting said structure to a p53 structure other than the one against which it was designed. For example, such a structure may be that T-p53C (PDB ID code 1UOL), T-p53C-R273H (PDB ID code 2BIM), or wild-type p53 (PDB ID code 1TSR)

A comparison of this type may be performed to determine whether a structure can bind in the β-sandwich region to non-mutated residues such that the stability of the molecule is potentially enhanced.

If necessary or desired, the structure may be modified in the light of its fitting to the further p53 structure and then re-fitted to a p53 mutant structure of the invention. This process may be iterated as necessary to determine further p53-biding structures.

Where the invention is used to provide computer-designed structures which bind to mutant T-p53C structures of the invention as described above, in a further aspect of the invention such structures may be synthesized or obtained and tested in a number of ways.

Thus in one aspect, the invention provides, following the analysis or design of a molecular structure as described herein, one or more of the following steps:
(a) obtaining or synthesizing a compound which has said molecular structure; and
   contacting said compound with a p53 protein to determine the ability of said compound to interact with said p53 protein; or
(b) obtaining or synthesizing a compound which has said molecular structure;
   forming a complex of a p53 protein and said compound; and
   analysing said complex by X-ray crystallography to determine the ability of said compound to interact with p53 protein; or
(c) obtaining or synthesizing a compound which has said molecular structure; and
   determining or predicting how said compound interacts with a p53 structure; and
   modifying the compound structure so as to alter the interaction between it and the p53.

The p53 protein which may be used can be a wild-type, a stabilised variant or a mutant including any of a p53Y220C, T-p53C-Y220C, p53V143A, T-p53C-V143A, p53F270L or a T-p53C-F270L protein.

In determining how the ability of the p53 protein to interact with such a compound, a number of different methods of analysis may be used. For example, the p53 may be expressed in a cell and the rate of apoptosis of the cell in the presence or absence of the compound can be compared. Where the compound stabilizes the p53, this may be reflected in a pro-apoptopic effect. In another embodiment, the compound may be brought into contact with p53 in order to determine its stability, e.g. as measured by the change in free energy of urea-induced unfolding.

Further, since a compound identified by the process of the present invention will stabilize the cavities identified herein, such compounds may be used to stabilize mutants of p53 which occur in the β-sandwich region, such that the mutants may be co-crystallized with the compound.

Thus, in one aspect, the invention provides a method comprising:
mixing a p53 β-sandwich mutant protein with the compound;
crystallizing a protein-compound complex; and
determining the structure of the complex by employing the data from any one of Tables 1 to 3, optionally varied within a root mean square deviation from the Cα atoms of not more than 1.5 Å, or selected coordinates thereof.

This method may be performed following the fitting of a ligand structure to a structure of a p53 mutant of any one of Tables 1-3 in accordance with the invention.

In a preferred aspect, the 13-sandwich mutant is a p53 protein mutated at one of positions 220, 143 or 270. The mutant may be p53 Y200C, p53 V143A or p53 F270L. Where the mutant is at positions 220, 143 or 270, then the data of Tables 1, 2 and 3 respectively is desirably employed in the method of the preceding paragraph.

### (v) Compounds of the Invention.

Where a potential modified compound has been developed by fitting a starting compound to a T-p53CLY220C, -V143A or -F270 structure of the invention and predicting from this a modified compound with an altered rate of action (including a greater or lesser binding affinity to p53), the invention further includes the step of synthesizing the modified compound and testing it in an in vivo or in vitro biological system in order to determine its activity and/or the rate at which it acts, e.g. to modify the stability of p53 or the ability of a p53 mutant to be rescued. This may be determined for example by expressing the mutant p53 in a cell and determining the rate of apoptosis of the cell in the presence or absence of the compound.

In another aspect, the invention includes a compound, which is identified by the methods of the invention described above.

Following identification of such a compound, it may be manufactured and/or used in the preparation, i.e. manufacture or formulation, of a composition such as a medicament, pharmaceutical composition or drug. These may be administered to individuals.

Thus, the present invention extends in various aspects not only to a compound as provided by the invention, but also a pharmaceutical composition, medicament, drug or other composition comprising such a compound. The compositions may be used. for treatment (which may include preventative treatment) of disease, particularly cancer. Such a treatment may comprise administration of such a composition to a patient, e.g. for treatment of disease; the use of such an inhibitor in the manufacture of a composition for administration, e.g. for treatment of disease; and a method of making a pharmaceutical composition comprising admixing such an inhibitor with a pharmaceutically acceptable excipient, vehicle or carrier, and optionally other ingredients.

Thus a further aspect of the present invention provides a method for preparing a medicament, pharmaceutical composition or drug, the method comprising (a) identifying or modifying a compound by a method of any one of the other aspects of the invention disclosed herein; (b) optimising the structure of the molecule; and (c) preparing a medicament, pharmaceutical composition or drug containing the optimised compound.

The above-described processes of the invention may be iterated in that the modified compound may itself be the basis for further compound design.

By “optimising the structure” we mean e.g. adding molecular scaffolding, adding or varying functional groups, or connecting the molecule with other molecules (e.g. using a fragment linking approach) such that the chemical structure of the modulator molecule is changed while its original modulating functionality is maintained or enhanced. Such optimisation is regularly undertaken during drug development programmes to e.g. enhance potency, promote pharmacological acceptability, increase chemical stability etc. of lead compounds.

Modification will be those conventional in the art known to the skilled medicinal chemist, and will include, for example, substitutions or removal of groups containing residues which interact with the amino acid side chain groups of a T-p53C-Y220C, -V143A or -F270 structure of the invention. For example, the replacements may include the addition or removal of groups in order to decrease or increase the charge of a group in a test compound, the replacement of a charge group with a group of the opposite charge, or the replacement of a hydrophobic group with a hydrophilic group or vice versa. It will be understood that these are only examples of the type of substitutions considered by medicinal chemists in the development of new pharmaceutical compounds and other modifications may be made, depending upon the nature of the starting compound and its activity.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The invention is illustrated by the following examples:

### EXAMPLES

### Mutagenesis and Protein Purification

The T-p53C mutants -Y220C, -V143A and -F270L (SEQ ID NOs:1-3 respectively) were made by mutagenesis, expressed and purified as previously described (18). After the final purification step (gel filtration), the mutant proteins were concentrated to 6-7 mg/ml, flash frozen and stored in liquid nitrogen.

### Urea Denaturation

Samples for urea denaturation experiments were prepared using a Hamilton Microlab dispenser from stock solutions of urea, buffer and protein to contain 1 μM protein in 25 mM sodium phosphate buffer, pH 7.2, 150 mM KCl and 5 mM DTT and increasing concentrations of urea. Prior to measurement the samples were incubated for 14 hours at 10° C. The intrinsic fluorescence spectra of p53 core domain, excited at 280 nm, were recorded in the range of 300-400 nm on a Perkin-Elmer LS50B spectrofluorimeter equipped with a Waters 2700 sample manager and controlled by laboratory software. Data analysis was performed as described previously (39).

### Crystallization and Structure Determination

All crystals were grown at 17° C. using the sitting drop vapour diffusion technique. The crystals were grown under the conditions described for T-p53C (19). In all cases, it was necessary to apply seeding techniques to improve crystal quality. Crystals were flash frozen in liquid nitrogen using mother liquor with either 20% PEG200 or 20% glycerol as cryoprotectant. The X-ray data sets for T-p53C-V143A was collected at 100 K on beamline 14.1 at the Synchrotron Radiation Source Daresbury using a wavelength of 1.488 Å. The data sets for T-p53C-Y220C and T-Tp53C-F270L were collected on beamline 10.1 using a wavelength of 1.284 Å. Data processing was performed using Mosflm (40) and Scala (41). All crystals belonged to space group P212121 and were isomorphous to those obtained for Tp53C and T-p53C-R273H (18,19). The cell parameters agreed within 0.6%. Structure solution and refinement was performed with CNS (42). After an initial round of rigid body refinement using either the structure of T-p53C (PDB ID code 1UOL) or T-p53C-R273H (PDB ID code 2BIM) as the starting model, the structures were refined by iterative cycles of refinement with CNS and manual model building with MAIN (43). Water molecules were added to the structure using the waterpick option implemented within CNS. The structure was solved by molecular replacement using the program CNS with diffraction data from 15-3.5 Å and T-p53C chain A (PDB ID code 1UOL) as a search model. The rotation and translation searches gave unambiguous solutions for four molecules in the asymmetric unit. Subsequent refinement was performed as described above. The refinement statistics are shown in Table 5.

### Structure Analysis

Unless otherwise stated, detailed descriptions of mutant structures are based on the comparison of molecule A of a particular mutant with molecule A of T-p53C. Numbering of secondary structure elements is as reported for the wild-type structure in complex with DNA (6). Solvent accessible surfaces were calculated with CNS using a probe radius of 1.4 Å. Solvent accessibility in percent for a particular residue was defined as solvent accessible surface in the parent protein divided by the solvent accessible area in an extended Ala-X-Ala tripeptide (44). Volumes of internal cavities were calculated with the program VOIDOO (45) using the following parameters: initial grid spacing 0.295 Å, VDW growth factor 1.1, atomic fattening factor 1.1, and grid shrink factor 0.9. Cavity volumes were refined by using successively finer grids until convergence was reached (convergence criteria 0.1). Since the results of grid-based methods may depend on the orientation of the molecule relative to the grid, each calculation was repeated nine times with randomly oriented copies of the molecule. Different probe sizes were tried. A probe radius of 1.4 Å mimics the size of a water molecule. Smaller probe sizes will better delineate the shape of a cavity. Hence, the calculated volume will increase with decreasing probe size. At smaller probe sizes however a particular cavity may leak into neighbouring cavities or the solvent and the method becomes much more sensitive to the orientation of the molecule. We therefore used probe sizes of 1.2 Å and 1.4 Å. Cavities were visually inspected with the crystallographic modeling program O (46). Structural figures were prepared using MOLSCRIPT (47) and RASTER3D (48).

TABLE 5
Data collection and refinement statistics

| | T-p53C | T-p53C | T-p53C |
|---|---|---|---|
| | V143A | Y220C | F270L |
| A. Data Collection | | | |
| Space Group | P2₁2₁2₁ | P2₁2₁2₁ | P2₁2₁2₁ |
| Cell (Å) | | | |
| a | 64.44 | 64.50 | 64.71 |
| b | 71.06 | 71.11 | 71.04 |
| c | 105.00 | 104.90 | 104.92 |
| β | 90.00 | 90.00 | 90.00 |
| Molecules/AU | 2 | 2 | 2 |
| Resolution (Å)^{b} | 29.4-1.80 | 26.8-1.65 | 41.1-1.80 |
| | (1.90-1.80) | (1.74-1.65) | (1.90-1.80) |
| Unique reflections | 43,176 | 55,177 | 45,350 |
| Completeness (%)^{a} | 95.2 (90.8) | 94.2 (89.2) | 99.6 (99.2) |
| Multiplicity | 6.9 (6.7) | 8.1 (7.9) | 5.4 (5.4) |
| R_{merge}(%)^{a, b} | 7.3 (28.5) | 5.9 (19.0) | 7.2 (33.0) |
| <I/σ_{I}>^{a} | 21.2 (5.7) | 24.4 (8.2) | 16.1 (4.7) |
| Wilson B factor (Å²) | 20.5 | 16.1 | 17.8 |
| B. Refinement | | | |
| Number of atoms | | | |
| Protein^{c} | 3094 | 3098 | 3092 |
| Water | 391 | 393 | 389 |
| Ions | 2 | 2 | 2 |
| R_{cryst}, (%)^{d} | 18.5 | 18.5 | 18.4 |
| R_{free}, (%)^{d} | 20.6 | 20.6 | 21.3 |
| R.m.s.d. bonds (Å) | 0.008 | 0.008 | 0.009 |
| R.m.s.d. angles (°) | 1.5 | 1.5 | 1.5 |
| Mean B value (Å²) | 23.6 | 18.7 | 21.9 |

^{a}Values in parentheses are for the highest resolution shell.
^{b}R_{merge} = Σ(I_{h,i} − <Iₕ>)/ΣI_{h,i}
^{c}Numbers include alternative conformations.
^{d}R_{cryst} and R_{free} = Σ||F_{obs}| − |F_{calc}||/Σ|F_{obs}| where R_{free} was calculated over 5% of the amplitudes chosen at random and not used in the refinement.

TABLE 6
Changes in free energy of urea-induced unfolding of
p53 core domain mutants

ΔΔG_{D-N}^{H}^{₂}^{O} (kcal/mol)^{a}

| Mutation | T-p53C | Wild type^{b} |
|---|---|---|
| V143A | 3.7 ± 0.12c | 3.5 ± 0.06 |
| Y220C | 4.2 ± 0.06 | 4.0 ± 0.06 |
| F270L | 4.1 ± 0.26 | n.d.^{c} |

^{a}ΔΔG_{D-N}^{H}^{₂}^{O} (kcal/mol) represents the change in the free energy of urea-induced unfolding caused by mutations in either T-p53C or wild type and is defined as: ΔΔG_{D-N}^{H}^{₂}^{O} = ΔG_{D-N}^{T-p53C} − ΔG_{D-N}^{mut} and ΔΔG_{D-N}^{H}^{₂}^{O} = ΔG_{D-N}^{wt} − ΔG_{D-N}^{mut} respectively. Data were collected at 10° C. in 25 mM sodium phosphate, pH 7.2, 150 mM KCl, 5 mM DTT.
^{b}Data for mutations in the wild-type context are taken from (14).
^{c}F270C destabilizes wild-type core domain by 4.5 kcal/mol (14).

TABLE 7
Volumes of mutation-induced internal cavities

| | 1.4-Å probe | No. lining | 1.2-Å probe | No. lining |
|---|---|---|---|---|
| | radius | atoms | radius | atoms |
| Mutant | Volume (Å³)^{a} | (polar atoms) | Volume (Å³)^{a,b} | (polar atoms) |
| T-p53C- | 46.6 (1.6) | 35 (8) | 62.2 (2.2) | 33 (8) |
| V143A | | | 19.3 (1.6) | 19 (2) |
| T-p53C- | 50.8 (0.9) | 29 (2) | 89.4 (3.1) | 43 (4) |
| F270L | | | | |

^{a}Cavity volumes were calculated with different probe sizes (1.2-Å and 1.4-Å radius) using the program VOIDOO. The numbers given are the averages of the size of a mutation-induced cavity (volume occupied by the probe) calculated for ten different orientations of the molecule. Standard deviations are given in parentheses.
^{b}In both mutants, the cavity calculated with a probe radius of 1.2 Å is substantially enlarged because of leaking into smaller cavities pre-existing in T-p53C. In T-p53C-V143A, the large cavity at the mutation site has merged with two smaller pre-existing cavities. Large parts of the smaller cavity pre-exist in T-p53C next to the Cγ1 atom of Val143. In T-p53C-F270L, the cavity comprises 3 smaller pre-existing cavities.

### Y220C Induces Sub-Optimal Packing at the Periphery of the -Sandwich

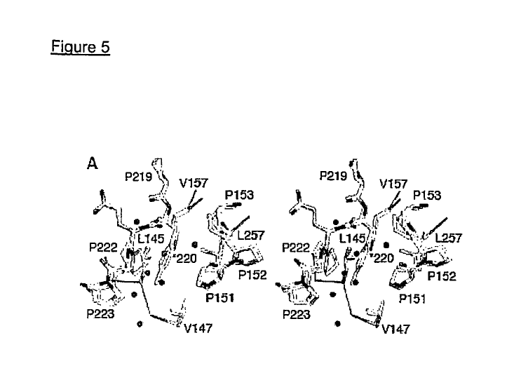

Y220C is the most common cancer mutation outside the DNA-binding surface (cf. release R10 of the p53 mutation database at www-p53.iarc.fr) and has a highly destabilizing effect on the stability of the core domain. It is located at the far end of the n-sandwich at the start of the turn connecting 13-strands S7 and S8 (FIG. 4). The benzene moiety of Tyr220 forms part of the hydrophobic core of the -sandwich, whereas the hydroxyl group is pointing toward the solvent. The crystal structure of T-p53C-Y220C showed that the Y220C mutation creates a solvent accessible cleft that is filled with water molecules at defined positions, while leaving the overall structure of the core domain intact (FIG. 5). Cys220 occupies approximately the position of the equivalent atoms of Tyr220 in the wild type. The structural response of neighbouring residues correlates with their location in the structure. The position of neighbouring hydrophobic side chains that are located in the core of the β-sandwich has not significantly shifted (Leu145, Val157 and Leu257). The mutation, however, results in a loss of hydrophobic interactions and a sub-optimal packing of these hydrophobic core residues. The side chain of Leu145 that was completely buried in wild type becomes partly solvent accessible in T-p53CY220C. The conformation of the rigid proline-rich S3/S4 turn around Pro151, which is packed against the Tyr220 side chain in wild type, is also largely unaffected and exhibits a temperature factor profile that is very similar to that in T-p53C. The largest structural changes are found in the S7/S8 turn itself for Pro222. Throughout the structure there is however no Cα-displacement larger than 0.9 Å.

### V143A and F270L are Cavity Creating-Mutations

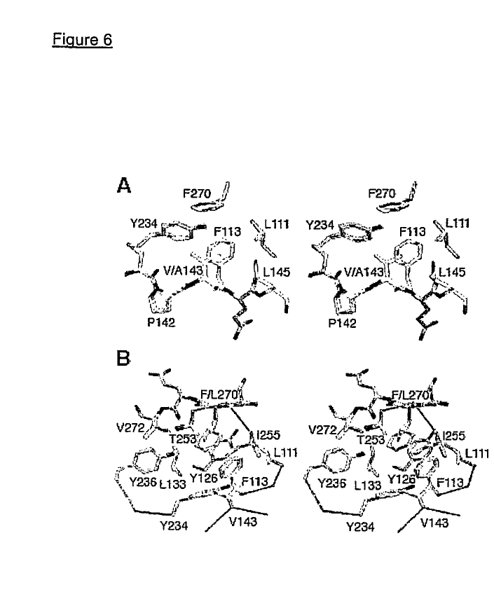

V143A is one of the classic examples of a temperature-sensitive p53 mutant (15). The mutation site is located in the hydrophobic core of the β-sandwich (FIG. 4). Overall, the structures of Tp53C and T-p53C-V143A are virtually identical, and there are only minor structural movements upon mutation (FIG. 6A). Both structures can be superimposed with an r.m.s. deviation of 0.12 Å for the Cq-atoms of equivalent chains. In T-p53C-V143A, the truncation of the two methyl groups of Val143 creates a hydrophobic cavity with a solvent accessible volume of 48 Å³ that is not filled with water (Table 7). There is almost no structural response and hence no collapse of the surrounding structure upon creation of this energetically unfavourable cavity. The mutated residue has only marginally moved toward the newly created cavity, and the largest displacement of individual atoms in the immediate environment of the mutation site is 0.3 Å. The cavity is lined by the hydrophobic side chains of Leu111, Phe113, Leu133, Tyr234, Ile255, and Phe270. The creation of this energetically unfavourable cavity in T-p53C-V143A accounts for the reduction of the thermodynamic stability of the protein by 3.7 kcal/mol.

The average B-factor for protein atoms in T-p53C-V143A is 22.3 Å², which is noticeably higher than the 16.3 Å² that was observed for the structure of T-p53C. Given that both structures were solved at a similar resolution, using isomorphous crystals grown under virtually the same conditions, this may reflect a higher overall mobility of the protein chain in T-p53C-V143A. An analysis of normalized average crystallographic B-factors for the backbone atoms showed an appreciable increase in the relative mobility of residues 143-145 on β-strand S3 that comprises the mutation site. Changes in the relative mobility of residues on the other structural elements lining the cavity was observed to be less pronounced.

The F270L cancer mutation affects the same hydrophobic core as the V143A mutation, and we hypothesized that this mutation should have a similar effect on the structure and stability of p53 core domain. This is confirmed by the structure of T-p53C-F270L, which reveals that the structural response to mutation is basically the same as for V143A. The mutation creates an internal cavity, but does not affect the overall structure of the protein. Again, the mutant structure can be perfectly superimposed onto the structure of T-p53C (r.m.s. deviation=0.09 Å for the Cα atoms of equivalent chains). The conformation of the side chains lining the cavity that is created by the F270L mutation is essentially the same as in T-p53C (FIG. 6B). Maximum atomic shifts within a 6-Å radius of the mutation site are 0.5 Å. Because of the different hybridization of Leu270-Cγ compared to Phe270-γ (sp3 versus sp2) and the resulting differences in bond angles, the leucine side chain has to be accommodated in a different way than the corresponding atoms of the phenylalanine in T-p53C. The Cγ and Cδ2 atoms are slightly off the original ring plane of the phenylalanine as a result of a 10° rotation in X1, whereas the Cδ1 atom points away from this plane and packs against the side chains of Phe113, Tyr126, Leu133 and Val272. The internal cavity created by the F270L mutation is slightly larger than the cavity created by V143A (Table 7). It is highly hydrophobic as 27 out of 29 lining atoms that could theoretically make contact with a buried water molecule are carbons (1.4 Å probe radius). This is consistent with the observation that no buried water molecule was detected in this cavity.

All publications and patents mentioned in the above specification are herein incorporated by reference. Various modifications and variations of the described invention will be apparent to those of skill in the art without departing from the scope and spirit of the invention. Although the Invention has been described in connection with specific preferred embodiments, it should be understood that the invention as claimed should not be unduly limited to such specific embodiments.

TABLE 4
p53 Y220C (SEQ ID NO: 1):

| | |
|---|---|
| 94 | SER SER SER VAL PRO SER GLN LYS THR TYR GLN GLY |
| | SER |
| 107 | TYR GLY PHE ARG LEU GLY PHE LEU HIS SER GLY THR |
| | ALA |
| 120 | LYS SER VAL THR CYS THR TYR SER PRO ALA LEU ASN |
| | LYS |
| 133 | LEU PHE CYS GLN LEU ALA LYS TER CYS PRO VAL GLN |
| | LEU |
| 146 | TRP VAL ASP SER THR PRO PRO PRO GLY THR ARG VAL |
| | ARG |
| 159 | ALA MET ALA ILE TYR LYS GLN SER GLN HIS MET THR |
| | GLU |
| 172 | VAL VAL ARG ARG CYS PRO HIS HIS GLU ARG CYS SER |
| | ASP |
| 185 | SER ASP GLY LEU ALA PRO PRO GLN HIS LEU ILE ARG |
| | VAL |
| 198 | GLU GLY ASN LEU ARG ALA GLU TYP LEU ASP ASP ARG |
| | ASN |
| 211 | THR PHE ARG HIS SER VAL VAL VAL PRO CYS GLU PRO |
| | PRO |
| 224 | GLU VAL GLY SER ASP CYS THR THR ILE HIS TYR ASN |
| | TYR |
| 237 | MET CYS TYR SER SER CYS MET GLY GLY MET ASN ARG |
| | ARG |
| 250 | PRO ILE LEU THR ILE ILE THR LEU GLU ASP SER SER |
| | GLY |
| 263 | ASN LEU LEU GLY ARG ASP SER PHE GLU VAL ARG VAL |
| | CYS |
| 276 | ALA CYS PRO GLY ARG ASP ARG ARG THR GLU GLU GLU |
| | ASN |
| 289 | LEU ARG LYS LYS GLY GLU PRO HIS HIS GLU LEU PRO |
| | PRO |
| 302 | GLY SER THR LYS ARG ALA LEU PRO ASN ASN THR |

T-p53C-V143A (SEQ ID NO: 2):

| | |
|---|---|
| 94 | SER SER SER VAL PRO SER GLN LYS THR TYR GLN GLY |
| | SER |
| 107 | TYP GLY PHE ARG LEU GLY PHE LEU HIS SER GLY THR |
| | ALA |
| 120 | LYS SER VAL THR CYS THR TYP SER PRO ALA LEU ASN |
| | LYS |
| 133 | LEU PHE CYS GLN LEU ALA LYS THR CYS PRO ALA GLN |
| | LEU |
| 146 | TRP VAL ASP SER THR PP0 PRO PRO GLY THR ARG VAL |
| | ARG |
| 159 | ALA MET ALA ILE TYP LYS GLN SER GLN HIS MET THR |
| | GLU |
| 172 | VAL VAL ARG ARG CYS PRO HIS HIS GLU ARG CYS SER |
| | ASP |
| 185 | SER ASP GLY LEU ALA PRO PRO GLN HIS LEU ILE ARG |
| | VAL |
| 198 | GLY GLY ASN LEU ARG ALA GLU TYR LEU ASP ASP ARG |
| | ASN |
| 211 | THR PHE ARG HIS SER VAL VAL VAL PRO Tyr GLU PRO |
| | PRO |
| 224 | GLU VAL GLY SER ASP CYS THR THR ILE HIS TYR ASN |
| | TYP |
| 237 | MET CYS TYR SER SER CYS MET GLY GLY MET ASN ARG |
| | ARG |
| 250 | PRO ILE LEU THR ILE ILE THR LEU GLU ASP SER SER |
| | GLY |
| 263 | ASN LEU LEU GLY ARG ASP SER PHE GLU VAL ARG VAL |
| | CYS |
| 276 | ALA CYS PRO GLY ARG ASP ARG ARG THR GLU GLU GLU |
| | ASN |
| 289 | LEU ARG LYS LYS GLY GLU PRO HIS HIS GLU LEU PRO |
| | PRO |
| 302 | GLY SER THR LYS ARG ALA LEU PRO ASN ASN THR |

T-p53C-F270L (SEQ ID NO: 3):

| | |
|---|---|
| 94 | SER SER SER VAL PRO SER GLN LYS THR TYR GLN GLY |
| | SER |
| 107 | TYR GLY PHE ARG LEU GLY PHE LEU HIS SER GLY THR |
| | ALA |
| 120 | LYS SER VAL THR CYS THR TYR SER PRO ALA LEU ASN |
| | LYS |
| 133 | LEU PHE CYS GLN LEU ALA LYS THR CYS PRO VAL GLN |
| | LEU |
| 146 | TRP VAL ASP SER THR PRO PRO PRO GLY THR ARG VAL |
| | ARG |
| 159 | ALA MET ALA ILE TYR LYS GLN SER GLN HIS MET THR |
| | GLU |
| 172 | VAL VAL ARG ARG CYS PRO HIS HIS GLU ARG CYS SER |
| | ASP |
| 185 | SER ASP GLY LEU ALA PRO PRO GLN HIS LEU ILE ARG |
| | VAL |
| 198 | GLU GLY ASN LEU ARG ALA GLU TYR LEU ASP ASP ARG |
| | ASN |
| 211 | THR PHE ARG HIS SER VAL VAL VAL PRO Tyr GLU PRO |
| | PRO |
| 224 | GLU VAL GLY SER ASP CYS THR THR ILE HIS TYR ASN |
| | TYR |
| 237 | MET CYS TYR SER SER CYS MET GLY GLY MET ASN ARG |
| | ARG |
| 250 | PRO ILE LEU THR ILE ILE THR LEU GLU ASP SER SER |
| | GLY |
| 263 | ASN LEU LEU GLY ARG ASP SER LEU GLU VAL ARG VAL |
| | CYS |
| 276 | ALA CYS PRO GLY ARG ASP ARG ARG THR GLU GLU GLU |
| | ASN |
| 289 | LEU ARG LYS LYS GLY GLU PRO HIS HIS GLU LEU PRO |
| | PRO |
| 302 | GLY SER THR LYS ARG ALA LEU PRO ASN ASN THR |

### REFERENCES

1. Vogelstein, B., Lane, D., and Levine, A. J. (2000) Nature 408, 307-310
2. Ryan, K. M., Phillips, A. C., and Vousden, K. H. (2001) Curr. Opin. Cell Biol. 13, 332-337
3. Vousden, K. H., and Lu, X. (2002) Nat. Rev. Cancer 2, 594-604
4. Olivier, M., Eeles, R., Hollstein, M., Khan, M. A., Harris, C. C., and Hainaut, P. (2002) Hum Mutat 19, 607-614
5. Beroud, C., and Soussi, T. (2003) Hum Mutat 21, 176-181
6. Cho, Y., Gorina, S., Jeffrey, P. D., and Pavletich, N. P. (1994) Science 265, 346-355
7. Clore, G. M., Ernst, J., Clubb, R., Omichinski, J. G., Kennedy, W. M., Sakaguchi, K., Appella, E., and Gronenbom, A. M. (1995) Nat Struct Biol 2, 321-333
8. Jeffrey, P. D., Gorina, S., and Pavletich, N. P. (1995) Science 267, 1498-1502
9. Bell, S., Klein, C., Muller, L., Hansen, S., and Buchner, J. (2002) J Mol Biol 322, 917-927
10. Dawson, R., Muller, L., Dehner, A., Klein, C., Kessler, H., and Buchner, J. (2003) J Mol Biol 332, 1131-1141
11. Veprintsev, D. B., Freund, S. M., Andreeva, A., Rutledge, S. E., Tidow, H., Canadillas, J. M., Blair, C. M., and Fersht, A. R. (2006) Proc Natl Acad Sci USA 103, 2115-2119
12. Canadillas, J. M., Tidow, H., Freund, S. M., Rutherford, T. J., Ang, H. C., and Fersht, A. R. (2006) Proc Natl Acad Sci U S A 103, 2109-2114
13. Bullock, A. N., and Fersht, A. R. (2001) Nat. Rev. Cancer 1, 68-76
14. Bullock, A. N., Henckel, J., and Fersht, A. R. (2000) Oncogene 19, 1245-1256
15. Zhang, W., Guo, X. Y., Hu, G. Y., Liu, W. B., Shay, J. W., and Deisseroth, A. B. (1994) EMBO J. 13, 2535-2544
16. Shiraishi, K., Kato, S., Han, S. Y., Liu, W., Otsuka, K., Sakayori, M., Ishida, T., Takeda, M., Kanamaru, R., Ohuchi, N., and Ishioka, C. (2004) J Biol Chem 279, 348-355
17. Wong, K. B., DeDecker, B. S., Freund, S. M., Proctor, M. R., Bycroft, M., and Fersht, A. R. (1999) Proc. Natl. Acad. Sci. USA 96, 8438-8442
18. Joerger, A. C., Ang, H. C., Veprintsev, D. B., Blair, C. M., and Fersht, A. R. (2005) J Biol Chem 280, 16030-16037
19. Joerger, A. C., Allen, M. D., and Fersht, A. R. (2004) J. Biol. Chem. 279, 1291-1296
22. Pan, Y., Ma, B., Levine, A. J., and Nussinov, R. (2006) Biochemistry 45, 3925-3933
24. Di Como, C. J., and Prives, C. (1998) Oncogene 16, 2527-2539
25. Eriksson, A. E., Baase, W. A., Zhang, X. J., Heinz, D. W., Blaber, M., Baldwin, E. P., and Matthews, B. W. (1992) Science 255, 178-183
26. Buckle, A. M., Cramer, P., and Fersht, A. R. (1996) Biochemistry 35, 4298-4305
27. Xu, J., Baase, W. A., Baldwin, E., and Matthews, B. W. (1998) Protein Sci 7, 158-177
28. Derbyshire, D. J., Basu, B. P., Serpell, L. C., Joo, W. S., Date, T., Iwabuchi, K., and Doherty, A. J. (2002) EMBO J. 21, 3863-3872
29. Joo, W. S., Jeffrey, P. D., Cantor, S. B., Finnin, M. S., Livingston, D. M., and Pavletich, N. P. (2002) Genes Dev. 16, 583-593
30. Gorina, S., and Pavletich, N. P. (1996) Science 274, 1001-1005
31. Friedler, A., Veprintsev, D. B., Rutherford, T., von Glos, K. I., and Fersht, A. R. (2004) J. Biol. Chem.
35. Huyen, Y., Jeffrey, P. D., Derry, W. B., Rothman, J. H., Pavletich, N. P., Stpyridi, E. S., and Halazonetis, T. D. (2004) Structure 12, 1237-1243
36. Tang, K. S., Guralnick, B. J., Wang, W. K., Fersht, A. R., and Itzhaki, L. S. (1999) J Mol Biol 285, 1869-1886
39. Bullock, A. N., Henckel, J., DeDecker, B. S., Johnson, C. M., Nikolova, P. V., Proctor, M. R., Lane, D. P., and Fersht, A. R. (1997) Proc. Natl. Acad. Sci. USA 94, 14338-14342
40. Leslie, A. G. W. (1992) Joint CCP4 and ESF-EACMB Newsletter on Protein Crystallography Vol. 26, Daresbury Laboratory, Warrington, UK
41. Collaborative Computational Project, N. (1994) Acta Crystallogr. D 50, 760-763
42. Brünger, A. T., Adams, P. D., Clore, G. M., DeLano, W. L., Gros, P., Grosse-Kunstleve, R. W., Jiang, J.-S., Kuszewski, J., Nilges, M., Pannu, N. S., Read, R. J., Rice, L. M., Simonson, T., and Warren, G. L. (1998) Acta Crystallogr. D 54, 905-921
43. Turk, D. (1992) Weiterentwicklung eines Programms für Molekülgrafik und Elektrondichte-Manipulation und seine Anwendung auf verschiedene Protein-Strukturaufklärungen, Ph.D. thesis, Technische Universität Munchen, Germany
44. Lee, B., and Richards, F. M. (1971) J Mol Biol 55, 379-400
45. Kleywegt, G. J., and Jones, T. A. (1994) Acta Crystallogr D Biol Crystallogr 50, 178-185
46. Jones, T. A., Zou, J.-Y., Cowan, S. W., and Kjeldgaard, M. (1991) Acta Crystallogr. A 47, 110-119
47. Kraulis, P. J. (1991) J. Appl. Crystallogr. 24, 946-950
48. Merritt, E. A., and Bacon, D. J. (1997) Methods Enzymol. 277, 505-524

1. A computer-based method for the analysis of the interaction of a molecular structure with a p53 structure, which comprises:
providing the p53 structure or selected coordinates thereof of Table 1 optionally varied within a root mean square deviation from the Cα atoms of not more than 2.0 Å;
providing a molecular structure to be fitted to said p53 structure or selected coordinates thereof; and
fitting the molecular structure to said p53 structure;
wherein said selected coordinates include at least one coordinate of an atom from residues 109, 145-157, 202-204, 219-223, 228-230 and 257.

2. The method of claim 1 wherein said selected coordinates include at least one atom from at least one of the residues of Arg156, Arg158, Arg202, Glu204, Pro219 and Glu258, optionally in combination with at least one atom of Cys220.

3. The method of claim 1 wherein said selected coordinates include at least one atom from at least one or more the residues Trp146, Val147, Thr150, and Pro223, optionally in combination with Cys220.

4. A computer-based method for the analysis of the interaction of a molecular structure with a p53 structure, which comprises:
providing the p53 structure or selected coordinates thereof of Table 2 optionally varied within a root mean square deviation from the Cα atoms of not more than 1.5 Å;
providing a molecular structure to be fitted to said p53 structure or selected coordinates thereof; and
fitting the molecular structure to said p53 structure;
wherein said selected coordinates include at least one coordinate of an atom from residues 113, 124, 133, 141-143, 234, 236, and 270.

5. A computer-based method for the analysis of the interaction of a molecular structure with a p53 structure, which comprises:
providing the p53 structure or selected coordinates thereof of Table 3 optionally varied within a root mean square deviation from the Cα atoms of not more than 1.5 Å;
providing a molecular structure to be fitted to said p53 structure or selected coordinates thereof; and
fitting the molecular structure to said p53 structure;
wherein said selected coordinates include at least one coordinate of an atom from residues 111, 113, 133, 143, 159, 234, 236, 253, 255, 270, and 272.

6. The method of claim 1 which further included fitting said structure to a wild-type or thermostable p53 structure.

7. The method of claim 1 which further comprises the steps of:
obtaining or synthesizing a compound which has said molecular structure; and
contacting said compound with a p53 protein to determine the ability of said compound to interact with said p53 protein.

8. The method of claim 1 which further comprises the steps of:
obtaining or synthesizing a compound which has said molecular structure;
forming a complex of a p53 protein and said compound; and
analysing said complex by X-ray crystallography to determine the ability of said compound to interact with p53 protein.

9. The method of claim 1 which further comprises the steps of:
obtaining or synthesizing a compound which has said molecular structure; and
determining or predicting how said compound interacts with a p53 protein; and
modifying the compound structure so as to alter the interaction between it and the p53.

10. The method of claim 7 wherein said p53 protein is a wild-type p53 protein or a p53Y220C, p53V143A or p53F270L protein.

11. A compound having the modified structure identified using the method of claim 1.

12. The method of claim 1 wherein the selected coordinates are of a number of atoms selected from at least 5, 10, 50, 100, 500 or 1000 atoms.

13. A method for determining the structure of a compound bound to a p53 β-sandwich mutant protein, said method comprising:
mixing said mutant protein with the compound;
crystallizing a protein-compound complex; and
determining the structure of the complex by employing the data from any one of Tables 1-3, optionally varied within a root mean square deviation from the Cα atoms of not more than 1.5 Å, or selected coordinates thereof.

14. The method of claim 13 wherein said p53 β-sandwich mutant protein is p53 Y220C, p53 V143A or p53 F270L.

15. A method of providing data for generating structures and/or performing optimisation of compounds which interact with a p53 Y220C mutant protein, the method comprising:
(i) establishing communication with a remote device containing computer-readable data comprising a p53 Y220C mutant structure or selected coordinates thereof of Table 1 optionally varied within a root mean square deviation from the Cα atoms of not more than 2.0 Å; and
(ii) receiving said computer-readable data from said remote device.
wherein said selected coordinates include at least one coordinate of an atom from residues 109, 145-157, 202-204, 219-223, 228-230 and 257.

16. A method of providing data for generating structures and/or performing optimisation of compounds which interact with a p53 V143A mutant protein, the method comprising:
(i) establishing communication with a remote device containing computer-readable data comprising a p53 V143A mutant structure or selected coordinates thereof of Table 2 optionally varied within a root mean square deviation from the Cα atoms of not more than 1.5 Å; and
(ii) receiving said computer-readable data from said remote device. wherein said selected coordinates include at least one coordinate of an atom from residues 111, 113, 124, 133, 141-143, 145, 157, 232, 234, 236, 255 and 270.

17. A method of providing data for generating structures and/or performing optimisation of compounds which interact with a p53 F270L mutant protein, the method comprising:
(i) establishing communication with a remote device containing computer-readable data comprising a p53 F270L mutant structure or selected coordinates thereof of Table 3 optionally varied within a root mean square deviation from the Cα atoms of not more than 1.5 Å; and
(ii) receiving said computer-readable data from said remote device.
wherein said selected coordinates include at least one coordinate of an atom from residues 111, 113, 133, 143, 159, 234, 236, 253, 255, 270, and 272.

18. The method of claim 15 which further comprises performing the method for the analysis of the interaction of a molecular structure with a p53 structure, which comprises:
providing the p53 structure or selected coordinates thereof of Table 1 optionally varied within a root mean square deviation from the Cα atoms of not more than 2.0 Å;
providing a molecular structure to be fitted to said p53 structure or selected coordinates thereof; and
fitting the molecular structure to said p53 structure;
wherein said selected coordinates include at least one coordinate of an atom from residues 109, 145-157, 202-204, 219-223, 228-230 and 257 with said data.

19. A crystal of a T-p53C-Y220C, T-p53C-V143A or T-p53C-F270L protein.

20. A co-crystal of a T-p53C-Y220C, T-p53C-V143A or T-p53C-F270L protein and a ligand.

21. The crystal or co-crystal of claim 19 wherein said p53-Y220C protein comprises residues 104-287 of SEQ ID NO:1, said T-p53C-V143A protein comprises residues 104-287 of SEQ ID NO:2, or said T-p53C-F270L protein comprises residues 104-287 of SEQ ID NO:3.

22. The crystal or co-crystal of claim 19 having space group P2₁2₁2₁.

23. The Crystal or co-crystal of claim 22 having unit cell dimensions a=64.50-64.71 Å, b=71.04-71.11 Å, c=104.90-105 Å, beta=90°, with a unit cell variability of 5% in all dimensions.


SEQUENCE LISTING (3 sequences)

SEQ ID NO: 1 - protein, 219 aa, Artificial Sequence
  Synthetic sequence T-p53C-Y220C
SSSVPSQKTYQGSYGFRLGFLHSGTAKSVTCTYSPALNKLFCQLAKTCPVQLWVDSTPPPGTRVRAMAIYKQSQHMTEVVRRCPHHERCSDSDGLAPPQHLIRVEGNLRAEYLDDRNTFRHSVVVPCEPPEVGSDCTTIHYNYMCYSSCMGGMNRRPILTIITLEDSSGNLLGRDSFEVRVCACPGRDRRTEEENLRKKGEPHHELPPGSTKRALPNNT

SEQ ID NO: 2 - protein, 219 aa, Artificial Sequence
  Synthetic sequence T-p53C-V143A
SSSVPSQKTYQGSYGFRLGFLHSGTAKSVTCTYSPALNKLFCQLAKTCPAQLWVDSTPPPGTRVRAMAIYKQSQHMTEVVRRCPHHERCSDSDGLAPPQHLIRVEGNLRAEYLDDRNTFRHSVVVPYEPPEVGSDCTTIHYNYMCYSSCMGGMNRRPILTIITLEDSSGNLLGRDSFEVRVCACPGRDRRTEEENLRKKGEPHHELPPGSTKRALPNNT

SEQ ID NO: 3 - protein, 219 aa, Artificial Sequence
  Synthetic sequence T-p53C-F270L
SSSVPSQKTYQGSYGFRLGFLHSGTAKSVTCTYSPALNKLFCQLAKTCPVQLWVDSTPPPGTRVRAMAIYKQSQHMTEVVRRCPHHERCSDSDGLAPPQHLIRVEGNLRAEYLDDRNTFRHSVVVPYEPPEVGSDCTTIHYNYMCYSSCMGGMNRRPILTIITLEDSSGNLLGRDSLEVRVCACPGRDRRTEEENLRKKGEPHHELPPGSTKRALPNNT